(19) United States
(12) Patent Application Publication
(10) Pub. No.: US 2009/0048203 A1
(43) Pub. Date: Feb. 19, 2009

(54) SUBSTITUTED ADENINES AND THE USES THEREOF

(76) Inventors: Marta Cavero-Tomas, West Roxbury, MA (US); Madhu Gowravaram, Action, MA (US); Hoan Huynh, Waltham, MA (US); Haihong Ni, Lexington, MA (US); Suzanne Stokes, Holliston, MA (US)

Correspondence Address: ASTRAZENECA R&D BOSTON, 35 GATEHOUSE DRIVE, WALTHAM, MA, 02451-1215, US

(21) Appl. No.: 11/577,278

(22) PCT Filed: Oct. 13, 2005
(86) PCT No.: PCT/GB05/03934

§ 371 Date: Apr. 13, 2007

Related U.S. Application Data
(60) Provisional application No. 60/619,218, filed on Oct. 15, 2004.
(60) Provisional application No. 60/663,459, filed on Mar. 18, 2005.
(60) Provisional application No. 60/699,615, filed on Jul. 15, 2005.

(51) Int. Cl. A61K 31/7076; C07H 19/167; A61K 31/52; C07D 473/34; A61P 31/04

(52) U.S. Cl. 514/46; 536/27.2; 514/263.23; 544/264

(57) ABSTRACT

This invention relates to compounds of Formula (I): and their use in the treatment of bacterial infections.

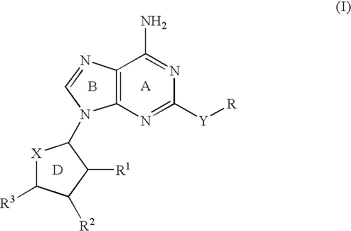

### FIELD OF INVENTION

The present invention relates to novel substituted heterocycles, their pharmaceutical compositions and methods of use. In addition, the present invention relates to therapeutic methods for the treatment of Gram-positive and Gram-negative bacterial infections.

### BACKGROUND OF THE INVENTION

The international microbiological community continues to express serious concern that the evolution of antibacterial resistance could result in bacterial strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity.

Gram-positive pathogens, for example staphylococci, enterococci, streptococci and mycobacteria, are particularly important because of the development of resistant strains that are both difficult to treat and difficult to eradicate from the hospital environment once established. Examples of such strains are methicillin resistant Staphylococcus aureus (MRSA), methicillin resistant coagulase-negative staphylococci (MRCNS), penicillin resistant Streptococcus pneumoniae and multiple resistant Enterococcus faecium. The preferred clinically effective antibiotic of last resort for treatment of such resistant Gram-positive pathogens is vancomycin. Vancomycin is a glycopeptide and is associated with various toxicities, including nephrotoxicity. Furthermore, and most importantly, antibacterial resistance to vancomycin and other glycopeptides is also appearing. This resistance is increasing at a steady rate rendering these agents less effective in the treatment of Gram-positive pathogens. There is also increasing resistance to agents such as β-lactams, quinolones and macrolides used for the treatment of upper respiratory tract infections caused by Gram-negative strains including H. influenzae and M. catarrhalis. Consequently, in order to overcome the threat of widespread multi-drug resistant organisms, there is an on-going need to develop new antibacterials, particularly those with either a novel mechanism of action and/or containing new pharmacophoric groups.

Deoxyribonucleic acid (DNA) ligases catalyze the formation of a phosphodiester linkage at single-strand breaks between adjacent 3′-OH and 5′-phosphate termini in double-stranded DNA (Lehman 1974. Science 186: 790-797). This activity plays an indispensable role in DNA replication where it joins Okazaki fragments. DNA ligase also plays a role in repair of damaged DNA and in recombination (Wilkinson 2001. Molecular Microbiology 40: 1241-1248). An early report describing conditional lethal mutations in the DNA ligase gene (ligA) of Escherichia coli supported the essentiality of this enzyme (Dermody et al. 1979. Journal of Bacteriology 139: 701-704). This was followed by the isolation and characterization of DNA ligase temperature-sensitive or knockout mutants of Salmonella typhimurium, Bacillus subtilis, and Staphylococcus aureus (Park et al. 1989. Journal of Bacteriology 171: 2173-2180, Kaczmarek et al. 2001. Journal of Bacteriology 183: 3016-3024, Petit and Ehrlich. 2000. Nucleic Acids Research 28: 4642-4648). In all species, DNA ligase was shown to be essential.

The DNA ligase family can be divided into two classes: those requiring ATP for adenylation (eukaryotic cells, viruses and bacteriophages), and those requiring NAD⁺ (nicotinamide adenine dinucleotide) for adenylation, which include all known bacterial DNA ligases (Wilkinson 2001, supra). Eukaryotic, bacteriophage, and viral DNA ligases show little sequence homology to DNA ligases from prokaryotes, apart from a conserved KXDG-motif located within the central cofactor-binding core of the enzyme. Amino acid sequence comparisons clearly show that NAD⁺-dependent ligases are phylogenically unrelated to the ATP-dependent DNA ligases. The apparent lack of similarity between the DNA ligases of bacteria and those of higher organisms suggests that bacterial DNA ligase is a good target for developing new antibacterials.

In 2003, Brötz-Oesterhelt et al. (Journal of Biological Chemistry 278:39435-39442) reported pyridochromanones as the first example of a selectively potent class of bacterial DNA ligase inhibitors whose mode of action was confirmed. This publication demonstrated proof-of-principle validation of LigA as an antibacterial target.

### SUMMARY OF THE INVENTION

In accordance with the present invention, the applicants have hereby discovered compounds that are inhibitors of bacterial DNA ligase (LigA) and therefore possess the ability to act as antimicrobials. Accordingly, the present invention relates to compounds that demonstrate antibacterial activity, processes for their preparation, pharmaceutical compositions containing them as the active ingredient, to their use as medicaments and to their use in the manufacture of medicaments for use in the treatment of bacterial infections in warm-blooded animals such as humans. These compounds are effective against a broad spectrum of bacterial pathogens.

The present inventors have found certain adenine derivatives inhibit bacterial DNA ligase and are therefore useful as antibacterials. Some of these adenine derivatives are known compounds for other uses, while others are believed to be novel compounds.

### DETAILED DESCRIPTION OF THE INVENTION

According to the foregoing, in one embodiment the present invention provides adenine derivatives of formula I which inhibit bacterial DNA ligase and are therefore useful as antibacterials.

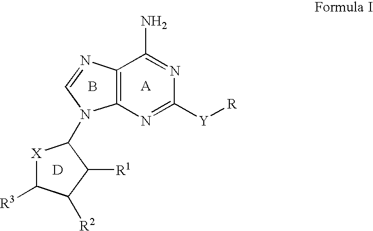

wherein:

A, B and D are used to designate the particular ring;

X is selected from O and —CH₂—;

Y is selected from O, S, —CO—, —CH₂—, —CH═CH—, —C≡C—, —SO—, and —SO₂— or Y and R taken together form a heterocyclic radical provided that the atom of the heterocyclic radical which is directly attached to ring A is not a nitrogen atom and wherein said heterocyclic radical may be optionally substituted on one or more carbon atoms by one or more R³³ and wherein if heterocyclyl contains an —NH— moiety, the nitrogen of said moiety may be optionally substituted by a group selected from R³⁴;

R is selected from C₁₋₁₀alkyl, C₂₋₁₀alkenyl, C₂₋₁₀alkynyl, C₃₋₁₂carbocyclyl, —S(O)ₚR⁴, —C(O)R⁵, and heterocyclyl wherein R may be optionally substituted on one or more carbon atoms by one or more R′ and wherein if heterocyclyl contains an —NH— moiety, the nitrogen of said moiety may be optionally substituted by a group selected from R″;

p is independently at each occurrence 0, 1 or 2;

R¹, R² and R³ are independently selected from hydrogen, hydroxy, cyano, azido, C₁₋₁₀alkyl, C₃₋₁₂carbocyclyl, halo, —C(O)R^{5′}, —OC(O)R¹², S(O)ₚR^{4′}, ═N—O—R⁹, C₂₋₁₀alkenyl, C₂₋₁₀alkynyl, heterocyclyl, —OR²⁴, NR¹⁰R¹¹, alternatively, R¹ and R² or R² and R³ taken together form a cyclic ring containing 3-6 atoms wherein R¹, R² and R³ may be optionally substituted on one or more carbon atoms by one or more R^{1′} and wherein if heterocyclyl contains an —NH— moiety, the nitrogen of said moiety may be optionally substituted by a group selected from R^{3′};

R⁴, R^{4′}, and R^{4″} are each independently selected from hydrogen, hydroxy, —NR⁷R⁸, C₁₋₆alkyl, C₂₋₆alkenyl, C₁₋₆alkoxy, C₃₋₁₀cycloalkyl, heterocyclyl, and aryl wherein R⁴, R^{4′}, and R^{4″} may be optionally substituted on one or more carbon atoms by one or more R¹³ and wherein if heterocyclyl contains an —NH— moiety, the nitrogen of said moiety may be optionally substituted by a group selected from R¹⁴;

R⁵, R^{5′}, R^{5″}, R¹², and R^{12′} are each independently selected from hydrogen, —NR^{7′}R^{8′}, —OR^{24′}, C₁₋₆alkyl, C₂₋₆alkenyl, C₃₋₁₀cycloalkyl, C₃₋₁₀cycloalkenyl, heterocyclyl, and aryl wherein R⁵, R^{5′}, R^{5″}, R¹², and R^{12′} may be optionally substituted on one or more carbon atoms by one or more R¹⁵ and wherein if heterocyclyl contains an —NH— moiety, the nitrogen of said moiety may be optionally substituted by a group selected from R¹⁶;

R⁷, R^{7′}, R^{7″}, R⁸, R^{8′} and R^{8″} are each independently selected from hydrogen, C₁₋₆alkyl, C₂₋₆alkenyl, C₂₋₆alkynyl, —OR^{24′}, C₃₋₁₀cycloalkyl, C₃₋₁₀cycloalkenyl, heterocyclyl, and aryl wherein R⁷, R^{7′}, R^{7″}, R⁸, R^{8′} and R^{8″} may be optionally substituted on one or more carbon atoms by one or more R¹⁷ and wherein if heterocyclyl contains an —NH— moiety, the nitrogen of said moiety may be optionally substituted by a group selected from R¹⁸;

R⁹ and R^{9′} are each independently selected from hydrogen, C₁₋₆alkyl, C₂₋₆alkenyl, C₂₋₆alkynyl, C₃₋₁₀cycloalkyl, C₃₋₁₀cycloalkenyl, heterocyclyl and aryl wherein R⁹ and R^{9′} may be optionally substituted on one or more carbon atoms by one or more R¹⁹;

R¹⁰ and R¹¹ are each independently selected from hydrogen, C₁₋₆alkyl, C₂₋₆alkenyl, C₂₋₆alkynyl, —OR^{24′}, C₃₋₁₀cycloalkyl, C₃₋₁₀cycloalkenyl, heterocyclyl and aryl, wherein R¹⁰ and R¹¹ independently of each other may be optionally substituted on one or more carbon by one or more R²⁰ and wherein if said heterocyclyl contains a —NH— moiety, the nitrogen of said moiety may be optionally substituted by a group selected from R²¹;

R′, R^{1′}, R¹³, R¹⁵, R¹⁷, R¹⁹, R²⁰, R²⁵ and R³³ are each independently selected from halo, nitro, —NR^{7″}R^{8″}, azido, cyano, isocyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, keto(═O), —OR^{24′}, —C(O)R^{5″}, —OC(O)R^{12′}, S(O)ₓR^{4″}; ═N—O—R^{9′}, —NHC(O)NR^{7′}R^{8′}, —N(C₁₋₆alkyl)C(O)NR^{7′}R^{8′}, NHC(O)R^{24″}, —NHCO₂R^{24″}, —NHSO₂(R^{24″}), -amidino i.e. —NHC(NH)NH₂, wherein x is independently 0, 1 or 2 and further wherein R′, R^{1′}, R¹³, R¹⁵, R¹⁷, R¹⁹, R²⁰, R²⁵ and R³³ independent of each other may be optionally substituted on one or more carbon by one or more R²² and wherein if heterocyclyl contains a —NH— moiety, the nitrogen of said moiety may be optionally substituted by a group selected from R²³;

R″, R^{3′}, R¹⁴, R¹⁶, R¹⁸, R²¹, R²³, R²⁶, R²⁸ and R³⁴ are each independently selected from cyano, C₁₋₆alkyl, C₂₋₆alkenyl, C₂₋₆alkynyl, aryl, cycloalkyl, cycloalkenyl, heterocyclyl, hydroxy, —OR^{24′}, —C(O)R^{5″}, —OC(O)R^{12′}, S(O)ₓR^{4″}, -amidino i.e. —NHC(NH)NH₂, wherein x is independently 0, 1 or 2 wherein R″, R^{3′}, R¹⁴, R¹⁶, R¹⁸, R²¹, R²³, R²⁶, R²⁸ and R³⁴ independently of each other may be optionally substituted on one or more carbon by one or more R²⁷ and wherein if said heterocyclyl contains a —NH— moiety, the nitrogen of said moiety may be optionally substituted by a group selected from R⁶;

R²⁴, R^{24′} and R^{24″} are each independently selected from hydrogen, C₁₋₆alkyl, C₂₋₆alkenyl, C₃₋₁₂cycloalkyl, C₃₋₁₂cycloalkenyl, aryl, S(O)ₓR^{4″}, and heterocyclyl wherein x is independently 0, 1 or 2 and further wherein R²⁴, R^{24′} and R^{24″} may be optionally substituted on one or more carbon by one or more R²⁵ and wherein if said heterocyclyl contains a —NH— moiety, the nitrogen of said moiety may be optionally substituted by a group selected from R²⁶;

R²² and R²⁷ are each independently selected from halo, nitro, —NR^{7′}R^{8′}, azido, cyano, isocyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, keto(═O), —OR^{24′}, —C(O)R^{5″}, —OC(O)R^{12′}, S(O)ₓR^{4″}; ═N—O—R^{9′}, —NHC(O)NR^{7′}R^{8′}, —N(C₁₋₆alkyl)C(O)NR^{7′}R^{8′}, —NHC(O)R^{24″}, —NHCO₂R^{24″}, —NHSO₂(R^{24″}), -amidino i.e. —NHC(NH)NH₂, wherein x is independently 0, 1 or 2 and further wherein R²² and R²⁷ independently of each other may be optionally substituted on one or more carbon by one or more R²⁹ and wherein if heterocyclyl contains a —NH— moiety, the nitrogen of said moiety may be optionally substituted by a group selected from R³⁰;

R⁶ and R²³ are each independently selected from cyano, C₁₋₆alkyl, C₂₋₆alkenyl, C₂₋₆alkynyl, aryl, cycloalkyl, cycloalkenyl, heterocyclyl, hydroxy, —OR^{24′}, —C(O)R^{5″}, —OC(O)R^{12′}, S(O)ₓR^{4″} and -amidino i.e. —NHC(NH)NH₂, wherein x is independently 0, 1 or 2 and further wherein R⁶ and R²³ independently of each other may be optionally substituted on one or more carbon by one or more R³¹ and wherein if said heterocyclyl contains a —NH— moiety, the nitrogen of said moiety may be optionally substituted by a group selected from R³²;

R²⁹ and R³¹ are each independently selected from halo, nitro, —NR^{7′}R^{8′}, azido, cyano, isocyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, keto(═O), —OR^{24′}, —C(O)R^{5″}, —OC(O)R^{12′}, S(O)ₓR^{4″}, ═N—O—R^{9′}, —NHC(O)NR^{7′}R^{8′}, —N(C₁₋₆alkyl)C(O)NR^{7′}R^{8′}, —NHC(O)R^{24″}, —NHCO₂R^{24″}, —NHSO₂(R^{24″}), -amidino i.e. —NHC(NH)NH₂, wherein x is independently 0, 1 or 2;

R³⁰ and R³² are each independently selected from cyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, —OR^{24′}, —C(O)R^{5″}, —OC(O)R^{12′}, S(O)ₓR^{4″}, and -amidino i.e. —NHC(NH)NH₂ wherein x is independently 0, 1 or 2;

provided that when ring D is an unsubstituted tetrahydrofuranyl ring, i.e. when X is O and R¹, R² and R³ are hydrogen, and Y is O, then R cannot be a 3-pyrrolidinyl radical or a 7-methylindan-4-yl radical; provided further when ring D is an unsubstituted tetrahydrofuranyl ring and Y is S, then R cannot be an unsubstituted 2-naphthyl radical; and further provided when ring D is an unsubstituted tetrahydrofuranyl ring and Y is a bond, then R cannot be an unsubstituted 3-pyridyl radical; and provided further the compound of formula I is not 9-{5-[4-(carboxymethyl)-1H-imidazol-1-yl]-5-deoxy-β-D-ribofuranosyl}-2-(cyclopentyloxy)-9H-purin-6-amine or 9-[5-(4-acetyl-1H-1,2,3-triazol-1-yl)-5-deoxy-β-D-ribofuranosyl]-2-(cyclopentyloxy)-9H-purin-6-amine.

The compounds of formula I possess one or more asymmetric carbon atoms and therefore can exist as racemates and the (R) and (S) enantiomers thereof and where two or more asymmetric carbons are present there can also exist diastereoisomers and mixtures thereof. The present invention is intended to include all such forms and mixtures thereof.

A particular embodiment of the compounds of formula I of the present invention are compounds of formula Ia and pharmaceutically acceptable salts thereof which inhibit bacterial DNA ligase and are therefore useful as antibacterials

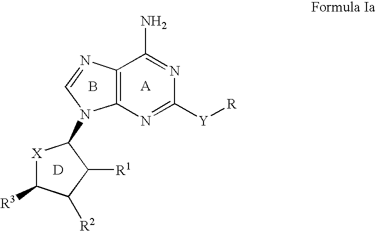

wherein R, R¹, R², R³, X and Y are as defined for the compounds of formula I.

A further particular embodiment of the compounds of formula I of the present invention is compounds of formula Ib and pharmaceutically acceptable salts thereof which inhibit bacterial DNA ligase and are therefore useful as antibacterials

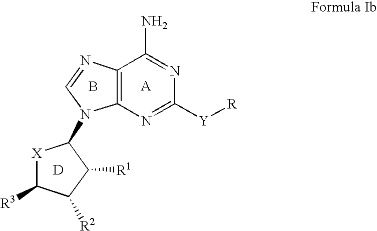

wherein R, R¹, R², R³, X and Y are as defined for the compounds of formula I and provided that R¹ and R² are not both H.

Another embodiment of the present invention is directed to novel compounds embraced within the scope of formula I. These novel compounds are compounds of formula II

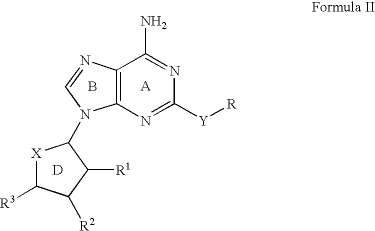

and pharmaceutically acceptable salts thereof wherein:

A, B and D are used to designate the particular ring;

X is selected from O and —CH₂—;

Y is selected from O, S, —CO—, —CH₂—, —CH═CH—, —SO—, and —SO₂— or Y and R taken together form a heterocyclic radical provided that the atom of the heterocyclic radical which is directly attached to ring A is not a nitrogen atom and wherein said heterocyclic radical may be optionally substituted on one or more carbon atoms by one or more R³³ and wherein if heterocyclyl contains an —NH— moiety, the nitrogen of said moiety may be optionally substituted by a group selected from R³⁴;

R is selected from C₁₋₁₀alkyl, C₂₋₁₀alkenyl, C₂₋₁₀alkynyl, C₃₋₁₂carbocyclyl, —S(O)ₚR⁴, —C(O)R⁵, and heterocyclyl wherein R may be optionally substituted on one or more carbon atoms by one or more R′ and wherein if heterocyclyl contains an —NH— moiety, the nitrogen of said moiety may be optionally substituted by a group selected from R″;

p is independently at each occurrence 0, 1 or 2;

R¹, R² and R³ are each independently selected from hydrogen, hydroxy, cyano, azido, C₁₋₁₀alkyl, C₃₋₁₂carbocyclyl, halo, —C(O)R^{5′}, —OC(O)R¹², S(O)ₚR^{4″}, ═N—O—R⁹, C₂₋₁₀alkenyl, C₂₋₁₀alkynyl, heterocyclyl, —OR²⁴, and NR¹⁰R¹¹ alternatively, R¹ and R² or R² and R³ taken together form a cyclic ring containing 3-6 atoms and further wherein R¹, R² and R³ may be optionally substituted on one or more carbon atoms by one or more R^{1′} and wherein if heterocyclyl and/or said cyclic ring contains an —NH— moiety, the nitrogen of said moiety may be optionally substituted by a group selected from R³;

R⁴, R^{4′}, and R^{4″} are each independently selected from hydrogen, hydroxy, —NR⁷R⁸, C₁₋₆alkyl, C₂₋₆alkenyl, C₁₋₆alkoxy, C₃₋₁₀cycloalkyl, heterocyclyl, and aryl wherein R⁴, R^{4′}, and R^{4″} may be optionally substituted on one or more carbon atoms by one or more R¹³ and wherein if heterocyclyl contains an —NH— moiety, the nitrogen of said moiety may be optionally substituted by a group selected from R¹⁴;

R⁵, R^{5′}, R^{5″}, R¹², and R^{12′} are each independently selected from hydrogen, —NR^{7′}R^{8′}, —OR^{24′}, C₁₋₆alkyl, C₂₋₆alkenyl, C₃₋₁₀cycloalkyl, C₃₋₁₀cycloalkenyl, heterocyclyl, and aryl wherein R⁵, R^{5′}, R^{5″}, R¹², and R^{12′} may be optionally substituted on one or more carbon atoms by one or more R¹⁵ and wherein if heterocyclyl contains an —NH— moiety, the nitrogen of said moiety may be optionally substituted by a group selected from R¹⁶;

R⁷, R^{7′}, R^{7″}, R⁸, R^{8′} and R^{8″} are each independently selected from hydrogen, C₁₋₆alkyl, C₂₋₆alkenyl, C₂₋₆alkynyl, —OR^{24′}, C₃₋₁₀cycloalkyl, C₃₋₁₀cycloalkenyl, heterocyclyl, and aryl wherein R⁷, R^{7′}, R^{7″}, R⁸, R^{8′} and R^{8″} may be optionally substituted on one or more carbon atoms by one or more R¹⁷ and wherein if heterocyclyl contains an —NH— moiety, the nitrogen of said moiety may be optionally substituted by a group selected from R¹⁸;

R⁹ and R^{9′} are each independently selected from hydrogen, C₁₋₆alkyl, C₂₋₆alkenyl, C₂₋₆alkynyl, C₃₋₁₀cycloalkyl, C₃₋₁₀cycloalkenyl, heterocyclyl and aryl wherein R⁹ and R^{9′} may be optionally substituted on one or more carbon atoms by one or more R¹⁹;

R¹⁰ and R¹¹ are each independently selected from hydrogen, C₁₋₆alkyl, C₂₋₆alkenyl, C₂₋₆alkynyl, —OR^{24′}, C₃₋₁₀cycloalkyl, C₃₋₁₀cycloalkenyl, heterocyclyl and aryl, wherein R¹⁰ and R¹¹ independently of each other may be optionally substituted on one or more carbon by one or more R²⁰, and wherein if said heterocyclyl contains a —NH— moiety, the nitrogen of said moiety may be optionally substituted by a group selected from R²¹;

R′, R^{1′}, R¹³, R¹⁵, R¹⁷, R¹⁹, R²⁰, R²⁵ and R³³ are each independently selected from halo, nitro, —NR^{7″}R^{8″}, azido, cyano, isocyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, keto(═O), —OR^{24′}, —C(O)R^{5″}, —OC(O)R^{12′}, S(O)₂R^{4″}; N—O—R^{9′}, —NHC(O)NR^{7′}R^{8′}, —N(C₁₋₆alkyl)C(O)NR^{7′}R^{8′}, NHC(O)R^{24″}, —NHCO₂R^{24″}, —NHSO₂(R^{24″}), -amidino i.e. —NHC(NH)NH₂, wherein x is independently 0, 1 or 2 and further wherein R′, R^{1′}, R¹³, R¹⁵, R¹⁷, R¹⁹, R²⁰, R²⁵ and R³³ independent of each other may be optionally substituted on one or more carbon by one or more R²² and wherein if heterocyclyl contains a —NH— moiety, the nitrogen of said moiety may be optionally substituted by a group selected from R²³;

R″, R^{3′}, R¹⁴, R¹⁶, R¹⁸, R²¹, R²³, R²⁶, R²⁸ and R³⁴ are each independently selected from cyano, C₁₋₆alkyl, C₂₋₆alkenyl, C₂₋₆alkynyl, aryl, cycloalkyl, cycloalkenyl, heterocyclyl, hydroxy, —OR^{24′}, —C(O)R^{5″}, —OC(O)R^{12′}, S(O)ₓR^{4″}, -amidino i.e. —NHC(H)NH₂, wherein x is independently 0, 1 or 2 wherein R″, R^{3′}, R¹⁴, R¹⁶, R¹⁸, R²¹, R²³, R²⁶, R²⁸ and R³⁴ independently of each other may be optionally substituted on one or more carbon by one or more R²⁷ and wherein if said heterocyclyl contains a —NH— moiety, the nitrogen of said moiety may be optionally substituted by a group selected from R⁶;

R²⁴, R^{24′} and R^{24″} are each independently selected from hydrogen, C₁₋₆alkyl, C₂₋₆alkenyl, C₃₋₁₂cycloalkyl, C₃₋₁₂cycloalkenyl, aryl, S(O)ₓR^{4″}, and heterocyclyl wherein x is independently 0, 1 or 2 and further wherein R²⁴, R^{24′} and R^{24″} may be optionally substituted on one or more carbon by one or more R²⁵ and wherein if said heterocyclyl contains a —NH— moiety, the nitrogen of said moiety may be optionally substituted by a group selected from R²⁶;

R²² and R²⁷ are each independently selected from halo, nitro, —NR^{7′}R^{8′}, azido, cyano, isocyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, keto(═O), —OR^{24′}, —C(O)R^{5″}, —OC(O)R^{12′}, S(O)ₓR^{4″}; ═N—O—R^{9′}, —NHC(O)NR^{7′}R^{8′}, —N(C₁₋₆alkyl)C(O)NR^{7′}R^{8′}, —NHC(O)R^{24″}, —NHCO₂R^{24″}, —NHSO₂(R^{24″}), -amidino i.e. —NHC(NH)NH₂, wherein x is independently 0, 1 or 2 and further wherein R²² and R²⁷ independently of each other may be optionally substituted on one or more carbon by one or more R²⁹ and wherein if heterocyclyl contains a —NH— moiety, the nitrogen of said moiety may be optionally substituted by a group selected from R³⁰;

R⁶ and R²³ are each independently selected from cyano, C₁₋₆alkyl, C₂₋₆alkenyl, C₂₋₆alkynyl, aryl, cycloalkyl, cycloalkenyl, heterocyclyl, hydroxy, —OR^{24′}, —C(O)R^{5″}, —OC(O)R^{12′}, S(O)ₓR^{4″} and -amidino i.e. —NHC(NH)NH₂, wherein x is independently 0, 1 or 2 and further wherein R⁶ and R²³ independently of each other may be optionally substituted on one or more carbon by one or more R³¹ and wherein if said heterocyclyl contains a —NH— moiety, the nitrogen of said moiety may be optionally substituted by a group selected from R³²;

R²⁹ and R³¹ are each independently selected from halo, nitro, —NR^{7′}R^{8′}, azido, cyano, isocyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, keto(═O), —OR^{24′}, —C(O)R^{5″}, —OC(O)R^{12′}, S(O)ₓR^{4″}, ═N—O—R^{9′}, —NHC(O)NR^{7′}R^{8′}, —N(C₁₋₆alkyl)C(O)NR^{7′}R^{8′}, —NHC(O)R^{24″}, —NHCO₂R^{24″}, —NHSO₂(R^{24″}), -amidino i.e. —NHC(NH)NH₂, wherein x is independently 0, 1 or 2;

R³⁰ and R³² are each independently selected from cyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, —OR^{24′}, —C(O)R^{5″}, —OC(O)R^{12′}, S(O)ₓR^{4″}, and -amidino i.e. —NHC(NH)NH₂ wherein x is independently 0, 1 or 2;

provided that when ring D is an unsubstituted tetrahydrofuranyl ring, i.e. when X is O and R¹, R² and R³ are hydrogen, and Y is O, then R cannot be a 3-pyrrolidinyl radical or a 7-methylindan-4-yl radical; provided further when ring D is an unsubstituted tetrahydrofuranyl ring and Y is S, then R cannot be an unsubstituted 2-naphthyl radical; and further provided when ring D is an unsubstituted tetrahydrofuranyl ring, Y and R taken together cannot be an unsubstituted 3-pyridyl radical; and provided further the compound is not
9-{5-[4-(carboxymethyl)-1H-imidazol-1-yl]-5-deoxy-β-D-ribofuranosyl}-2-(cyclopentyloxy)-9H-purin-6-amine,
9-[5-(4-acetyl-1H-1,2,3-triazol-1-yl)-5-deoxy-β-D-ribofuranosyl]-2-(cyclopentyloxy)-9H-purin-6-amine,
3-(6-amino-2-propylthio-9H-purin-9-yl)-5-(hydroxymethyl)-1,2-cyclopentanediol, or
9-cyclopentyl-2-(cyclopentylthio)-9H-purin-6-amine;

and further provided that when X is O, R¹ and R² are both hydroxy, and ring D has the stereochemistry indicated in formulas IIIa or IIIb,

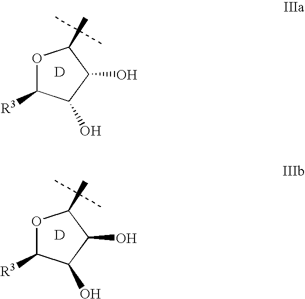

then R³ is not HO—CH₂— (hydroxymethyl group) or CH₃CH₂NHC(O)— (N-ethylcarboxamido group).

In another embodiment, the present invention is directed to compounds of formula II and pharmaceutically acceptable salts thereof wherein when X is O; Y is O or S; and R¹ and R² are both hydroxy, then R³ is not HO—CH₂—.

In another embodiment, the present invention is directed to compounds of formula II and pharmaceutically acceptable salts thereof wherein when X is O and R¹ and R² are both hydroxy, then R³ is not HOCH₂—.

In a further embodiment, the present invention is directed to compounds of formula II and pharmaceutically acceptable salts thereof wherein when X is O and R¹ and R² are both hydroxy, then R³ is not CH₃CH₂NHC(O)—.

In a still further embodiment, the present invention is directed to compounds of formula II and pharmaceutically acceptable salts thereof wherein when Y is CH₂, and R is unsubstituted alkyl, then alkyl represents a monovalent straight or branched chain hydrocarbon radical comprising from 1 to 6 carbon atoms optionally substituted on one or more carbons by one or more R′.

A particular embodiment of the novel compounds of formula II of the present invention is compounds of formula IIa and pharmaceutically acceptable salts thereof

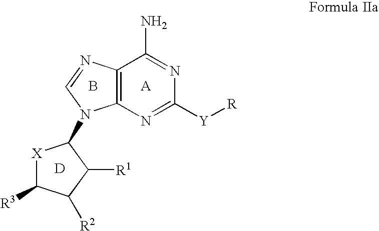

wherein R, R¹, R², R³, X and Y are as defined for the compounds of formula II.

A further particular embodiment of the compounds of formula II of the present invention is compounds of formula IIb and pharmaceutically acceptable salts thereof

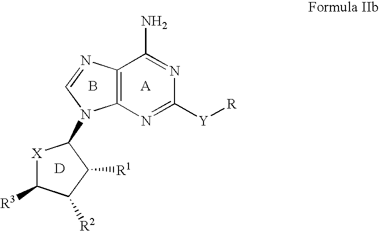

wherein R, R¹, R², R³, X and Y are as defined for the compounds of formula II provided R¹ and R² are not both H.

A still further particular embodiment of the compounds of formula II of the present invention is compounds of formula IIc and pharmaceutically acceptable salts thereof

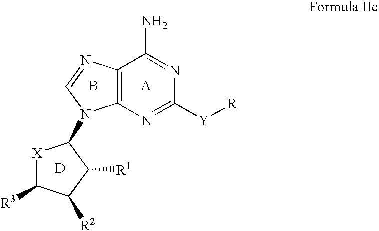

wherein R, R¹, R²R³, X and Y are as defined for the compounds of formula II provided R² is not H.

In a further embodiment, the present invention is directed to novel compounds of formula IIb and pharmaceutically acceptable salts thereof wherein Y is O and R³ is methyl.

Another embodiment of the instant invention is directed to novel compounds of formula IIb and pharmaceutically acceptable salts thereof wherein when Y is O and R³ is methyl, then R¹ and R² are not both hydroxy.

Another embodiment of the instant invention is directed to novel compounds of formula IIb and pharmaceutically acceptable salts thereof wherein when Y is O and R³ is methyl, then R¹ and R² are both hydroxy.

In a further embodiment, the present invention is directed to compounds of formula II, IIa and IIb and pharmaceutically acceptable salts thereof wherein R³ is halo substituted C₁₋₆alkyl, particularly fluoro or chloro substituted alkyl and more particularly R³ is a fluoromethyl or chloromethyl group.

Another embodiment of the instant invention is directed to novel compounds of formula IIb and pharmaceutically acceptable salts thereof wherein R¹ and R² are both hydroxy; and R³ is methyl or fluoromethyl.

A further embodiment of the instant invention is directed to novel compounds of formula IIb and pharmaceutically acceptable salts thereof wherein Y is O; R¹ and R² are both hydroxy; and R³ is methyl or fluoromethyl.

It should be clear to one of skill in the art that for the compounds of formulas Ia, Ib, IIa and IIb, when R³ is H the stereochemistry as indicated in those formulas no longer applies with respect to the carbon atom to which the H is attached since the carbon atom is no longer an asymmetric carbon atom. Similarly, in formulas Ib and IIb, if one of R¹ and R² is H, then the carbon atom to which that H is attached is no longer an asymmetric carbon atom and therefore, the stereochemistry indicated at that position in formulas Ib and IIb would not apply. For further clarity, in formulas Ia, Ib, IIa and IIb, if R³ is H, then the bond between ring B and ring D can have both the R and S configurations. If R³ is not H, then the configuration of the bond between rings B and D and the bond to R³ in the formulas is analogous to the β-D-ribose stereochemistry of the natural product adenosine, shown below.

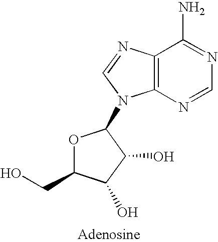

In another embodiment the present invention is directed to the compounds of formula II and pharmaceutically acceptable salts thereof wherein R¹, R² and R³ are all H.

In another embodiment the present invention is directed to the compounds of formula II and pharmaceutically acceptable salts thereof wherein X is O and R¹, R² and R³ are all H.

Additional embodiments of the invention are as follows. These additional embodiments relate to compounds of formulas I, Ia, Ib, II, IIa, IIb, and IIc and it is to be understood where compounds of anyone of these formulas are referred to, this also applies in the alternative to compounds of any of the other formulas. Such specific substituents may be used, where appropriate, with any of the definitions, claims or embodiments defined hereinbefore or hereinafter.

X is O.

X is —CH₂—.

Y is O.

Y is S.

Y is —SO₂⁻.

Y is —CH₂⁻.

Y is —CO—.

Y is —CH═CH—.

Y and R taken together form a heterocyclic radical provided that the atom of the heterocyclic radical which is directly attached to ring A is not a nitrogen atom and wherein said heterocyclic radical may be optionally substituted on one or more carbon atoms by one or more R³³ and wherein if heterocyclyl contains an —NH— moiety, the nitrogen of said moiety may be optionally substituted by a group selected from R³⁴.

R is C₁₋₆alkyl optionally substituted on one or more carbon by R′.

R is C₃₋₁₀cycloalkyl optionally substituted on one or more carbon by R′.

R is selected from cyclopropyl, cyclobutyl, cyclopentyl, cyclohexyl, spiro[2.2]cyclopentyl, and bicyclo[3.1.0]hexanyl wherein R may be optionally substituted on carbon by one or more groups selected from C₁₋₆alkyl, halo, cyano, —S(O)ₚR^{4″}, and ═N—O—R^{9′} and wherein said C₁₋₆alkyl may be optionally substituted on carbon by one or more halo groups.

R is selected from C₁₋₆alkyl and C₃₋₁₀cycloalkyl wherein R may be optionally substituted on carbon by one or more groups selected from halo, C₃₋₁₂cycloalkyl, C₃₋₁₂cycloalkenyl, heterocyclyl and aryl wherein said C₃₋₁₂cycloalkyl, C₃₋₁₂cycloalkenyl, heterocyclyl and aryl may be optionally substituted on carbon by one or more groups selected from halo, C₁₋₄alkyl, C₁₋₆alkoxy, cyano, S(O)ₓR^{4″} and ═N—O—R^{9′}; and further wherein said C₁₋₄alkyl and C₁₋₆alkoxy may be optionally substituted on carbon by one or more groups selected from halo and C₁₋₄alkoxy.

R is C₁₋₆alkyl wherein R may be optionally substituted on carbon by one or more fluoro.

R is selected from C₁₋₆alkyl, C₃₋₁₂cycloalkyl, C₃₋₁₂cycloalkenyl, C₃₋₁₂cycloalkylC₁₋₆alkyl and C₃₋₁₂cycloalkenylC₁₋₆alkyl wherein R may be optionally substituted on one or more carbon by one or more groups selected from fluoro and trifluoromethyl.

R is heterocyclyl optionally substituted on one or more carbon by one or more R′ and wherein if said heterocyclyl contains an —NH— moiety, the nitrogen of said moiety may be optionally substituted by a group selected from R″.

R¹ is selected from halo and hydroxy.

R¹ is hydroxy.

R² is selected from hydroxy, halo, cyano, azido, C₁₋₆alkyl and C₁₋₆alkoxy wherein said C₁₋₆alkyl and C₁₋₆alkoxy are optionally substituted on one or more carbons by one or more R^{1′}.

R² is selected from hydroxy, halo and cyano.

R² is NR¹⁰R¹¹.

R² is selected from fluoro and chloro.

R² is hydroxy.

R² is cyano.

R³ is selected from methyl, hydroxymethyl, halomethyl.

R³ is fluoromethyl.

R³ is hydroxymethyl.

R¹ and R² or R² and R³ taken together form a cyclic ring containing 3-6 atoms wherein R¹, R² and R³ may be optionally substituted on one or more carbon atoms by one or more R^{1′} and wherein if said cyclic ring contains an —NH— moiety, the nitrogen of said moiety may be optionally substituted by a group selected from R^{3′}.

R¹ and R² or R² and R³ taken together form a saturated cyclic ring containing 36 atoms wherein R¹, R² and R³ may be optionally substituted on one or more carbon atoms by one or more R^{1′} and wherein if said saturated cyclic ring contains an —NH— moiety, the nitrogen of said moiety may be optionally substituted by a group selected from R^{3′}.

R¹ and R² or R² and R³ taken together form a cyclic ether optionally substituted on carbon by one or more R^{1′}.

In a further embodiment the present invention is directed to compounds of formula II and pharmaceutically acceptable salts thereof wherein Y is O and R is selected from C₃₋₁₂cycloalkyl and C₃₋₁₂cycloalkylC₁₋₆alkyl wherein said C₃₋₁₂cycloalkyl and C₃₋₁₂cycloalkylC₁₋₆alkyl are optionally substituted on one or more carbons by R′.

In another embodiment the present invention is directed to compounds of formula II or IIb wherein

X and Y are O;

R is selected from C₃₋₁₀cycloalkyl and C₃₋₁₀cycloalkenyl wherein said R is optionally substituted on one or more carbon atoms by one or more C₁₋₄alkyl, halo, haloC₁₋₄alkyl, C₁₋₄alkoxy and haloC₁₋₄alkoxy, cyano, S(O)ₓR^{4″}, and ═N—OR^{9′};

R¹ is hydroxy;

R² is selected from hydroxy, halo, NR¹⁰R¹¹, cyano and C₁₋₃alkoxy wherein said C₁₋₃alkoxy is optionally substituted on one or more carbon by one or more halo, hydroxy, heteroaryl, and aryl and wherein said heteroaryl and aryl are optionally substituted on one or more carbon by one or more halo, C₁₋₄alkyl, halo substituted C₁₋₄alkyl and C₁₋₃alkoxy;

R³ is C₁₋₃alkyl wherein said C₁₋₃alkyl is optionally substituted on one or more carbon by one or more halo or hydroxy; and pharmaceutically acceptable salts thereof.

In a still further embodiment the present invention is directed to compounds of formula IIc wherein

X and Y are O;

R is selected from C₁₋₄alkyl, C₃₋₇cycloalkyl, and C₃₋₇cycloalkenyl wherein said R is optionally substituted on one or more carbon atoms by one or more C₁₋₄alkyl, halo, haloC₁₋₄alkyl, C₁₋₄alkoxy and haloC₁₋₄alkoxy, cyano, S(O)ₓR^{4″}, and ═N—OR^{9′};

R¹ is hydroxy;

R² is selected from hydroxy, NR¹⁰R¹¹, halo, cyano and C₁₋₃alkoxy wherein said C₁₋₃alkoxy is optionally substituted on one or more carbon by one or more halo, hydroxy, heteroaryl and aryl and wherein said heteroaryl and aryl are optionally substituted on one or more carbon by one or more halo, C₁₋₄alkyl, halo substituted C₁₋₄alkyl and C₁₋₃alkoxy;

R³ is selected from hydroxy and C₁₋₃alkyl wherein said C₁₋₃alkyl is optionally substituted on one or more carbon by one or more halo or hydroxy; and pharmaceutically acceptable salts thereof.

In another embodiment, the present invention provides compounds of formula II encompassed by the Examples, each of which provides a further independent aspect of the invention.

It is to be understood that combinations of substituents and/or variables are permissible only if such combinations result in stable compounds.

When any variable (e.g. R⁵, R^{5′}, R⁶, R⁷, etc.) occurs more than one time in any formula for a compound, its definition at each occurrence is independent of its definition at every other occurrence.

The definitions set forth herein are intended to clarify terms used throughout this application unless specifically indicated otherwise. The term “herein” means the entire application.

The term “carbocyclyl” refers to saturated, partially saturated and unsaturated, mono, bi or polycyclic carbon rings. These may include fused or bridged bi- or polycyclic systems. Carbocyclyls may have from 3 to 12 carbon atoms in their ring structure, i.e. C₃₋₁₂carbocyclyl, and in a particular embodiment are monocyclic rings have 3 to 7 carbon atoms or bicyclic rings having 7 to 10 carbon atoms in the ring structure. Examples of suitable carbocyclyls include cyclopropyl, cyclobutyl, cyclopentyl, cyclohexyl, cycloheptyl, cyclohexenyl, cyclopentadienyl, indanyl, phenyl and naphthyl.

The term “hydrocarbon” used alone or as a suffix or prefix, refers to any structure comprising only carbon and hydrogen atoms and containing up to 12 carbon atoms.

In this specification the term alkyl, used alone or as a suffix or prefix, includes both monovalent straight and branched chain hydrocarbon radicals but references to individual alkyl radicals such as propyl are specific for the straight chain version only. An analogous convention applies to other generic terms. Unless otherwise specifically stated, the term alkyl refers to hydrocarbon radicals comprising 1 to 12 carbon atoms, in another embodiment 1 to 10 carbon atoms, and in a still further embodiment, 1 to 6 carbon atoms.

The term “alkenyl” used alone or as suffix or prefix, refers to a monovalent straight or branched chain hydrocarbon radical having at least one carbon-carbon double bond which, unless otherwise specifically stated, comprises at least 2 up to 12 carbon atoms, in another embodiment 2-10 carbon atoms and in a still further embodiment 2-6 carbon atoms.

The term “alkynyl” used alone or as suffix or prefix, refers to a monovalent straight or branched chain hydrocarbon radical having at least one carbon-carbon triple bond which, unless otherwise specifically stated, comprises at least 2 up to 12 carbon atoms, in another embodiment 2-10 carbon atoms and in a still further embodiment 2-6 carbon atoms.

In this specification, the terms alkenyl and cycloalkenyl include all positional and geometrical isomers.

The term “cycloalkyl,” used alone or as suffix or prefix, refers to a monovalent ring-containing hydrocarbon radical which, unless otherwise specifically stated, comprises at least 3 up to 12 carbon atoms, in another embodiment 3 up to 10 carbon atoms and includes monocyclic as well as bicyclic and polycyclic ring systems. When a cycloalkyl ring contains more than one ring, the rings may be fused or unfused. Fused rings generally refer to at least two rings sharing two atoms there between. Suitable examples include C₃-C₁₀ cycloalkyl rings, e.g. cyclopropyl, cyclobutyl, cyclopentyl, cyclohexyl and cyclooctyl radicals, adamantanyl, norbornyl, decahydronapthyl, octahydro-1H-indenyl, spiro[2.2]pentanyl, and bicyclo[3.1.0]hexanyl.

The term “cycloalkenyl” used alone or as suffix or prefix, refers to a monovalent ring-containing hydrocarbon radical having at least one carbon-carbon double bond and unless otherwise specifically stated comprising at least 3 up to 12 carbon atoms, in another embodiment 3 up to 10 carbon atoms. Suitable examples include cyclopentenyl and cyclohexenyl.

The term “aryl” used alone or as suffix or prefix, refers to a hydrocarbon radical having one or more polyunsaturated carbon rings having aromatic character, (e.g., 4n+2 delocalized electrons) and comprising 5 up to 14 carbon atoms, wherein the radical is located on a carbon of the aromatic ring. Examples of suitable aryl radicals include phenyl, napthyl, and indanyl.

The term “alkoxy” used alone or as a suffix or prefix, refers to radicals of the general formula —O—R, wherein —R is selected from an optionally substituted hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The terms “heterocyclic radical” or “heterocyclyl” (both referred to herein as “heterocyclyl”) used alone or as a suffix or prefix, refer to a ring-containing structure or molecule having one or more multivalent heteroatoms, independently selected from N, O, and S, as a part of the ring structure and, unless otherwise specifically stated, including at least 3 and up to 14 atoms in the ring(s), or from 3-10 atoms in the ring, or from 3-6 atoms in the ring. Heterocyclyl groups may be saturated or unsaturated, containing one or more double bonds, and heterocyclyl groups may contain more than one ring. When a heterocyclyl contains more than one ring, the rings may be fused or unfused. Fused rings generally refer to at least two rings sharing two atoms therebetween. Heterocycle groups also include those having aromatic character. Examples of suitable heterocycles include, but are not limited to, indazole, pyrrolidonyl, dithiazinyl, pyrrolyl, indolyl, piperidonyl, carbazolyl, quinolizinyl, thiadiazinyl, acridinyl, azepane, azetidine, aziridine, azocinyl, benzimidazolyl, benzofuran, benzofuranyl, benzothiofuranyl, benzothiophenyl, benzoxazole, benzoxazolyl, benzothiophene, benzthiazolyl, benzotriazolyl, benzotetrazolyl, benzisoxazolyl, benzthiazole, benzisothiazolyl, benzimidazoles, benzimidazalonyl, carbazolyl, β-carbolinyl, chromanyl, chromenyl, cinnolinyl, decahydroquinolinyl, dioxanyl, dioxolanyl, furyl, dihydrofuranyl, tetrahydrothiopyranyl, furanyl, furazanyl, homopiperidinyl, imidazole, imidazolidine, imidazolidinyl, imidazolinyl, imidazolyl, indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, keto, morpholinyl, naphthyridinyl, octahydroisoquinolinyl, oxadiazolyl, oxazolidinyl, oxazolyl, oxirane, oxazolidinylperimidinyl, oxetanyl, phenanthridinyl, phenanthrolinyl, phenarsazinyl, phenazinyl, phenothiazinyl, phenoxathiinyl, phenoxazinyl, phthalazinyl, piperazinyl, piperidine, piperidinyl, pteridinyl, piperidonyl, 4-piperidonyl, purinyl, pyranyl, pyrrolidine, pyrroline, pyrrolidine, pyrrolidine-2-onyl, pyrazinyl, pyrazolidinyl, pyrazolinyl, pyrazolyl, pyridazinyl, pyridooxazole, pyridoimidazole, pyridothiazole, pyridinyl, N-oxide-pyridinyl, pyridyl, pyrimidinyl, pyrrolidinyl, pyrrolinyl, pyrrolyl, pyridine, quinazolinyl, quinolinyl, 4H-quinolizinyl, quinoxalinyl, quinuclidinyl, carbolinyl, tetrahydrofuranyl, tetrahydroisoquinolinyl, tetrahydropyranyl, thiophane, thiotetrahydroquinolinyl, thiadiazinyl, thiadiazolyl, thianthrenyl, thiazolyl, thienyl, thienothiazolyl, thienooxazolyl, thienoimidazolyl, thiophenyl, thiirane, triazinyl, triazolyl, and xanthenyl.

“Halo” includes fluorine, chlorine, bromine and iodine.

As used herein, the term “optionally substituted,” means that substitution is optional and therefore it is possible for the designated substituent to be unsubstituted. In the event a substitution is desired then such substitution means that any number of hydrogens on the designated substituent is replaced with a selection from the indicated group, provided that the normal valency of the atoms on a particular substituent is not exceeded, and that the substitution results in a stable compound. For example when a substituent is keto (i.e., ═O), then 2 hydrogens on the atom are replaced. In the case of cyclic substituents, e.g. cycloalkyl and aryl, two hydrogens may be replaced to form a second ring resulting in an overall fused or spiro ring system which may be partially or fully saturated, unsaturated or aromatic. Suitable substituents include alkylamido, e.g. acetamido, propionamido; alkyl; alkylhydroxy; alkenyl; alkenyloxy; alkynyl; alkoxy; halo; haloalkyl; hydroxy; alkylhydroxy; carboxyl; cycloalkyl; alkylcycloalkyl; acyl; aryl; acyloxy; amino; amido; carboxy; carboxy derivatives e.g. —CONH₂, —CO₂H, —COalkyl, —COaryl, —COcycloalkyl, —COcycloalkenyl, —COheterocyclyl; substituted —NH₂; aryloxy; alkoxy; nitro; cyano; azido, heterocyclyl; thiol; imine; sulfonic acid; sulfate; sulfonyl; sulfinyl; sulfanyl; sulfamoyl; carboxylic acid; amide; thioester; thioether; acid halide; anhydride; oxime i.e. ═N—OH; hydrazine; carbamate; or any other viable functional group provided that the resulting compound is stable and exhibits bacterial DNA ligase inhibitory activity. These optional substituents may themselves be optionally substituted again as long as the resulting compound is stable and exhibits a bacterial DNA ligase inhibitory effect.

When a particular substituent is indicated to be “substituted”, then that substituent can be substituted with any of the optional substituents listed above provided the resulting compound is stable and exhibits a bacterial DNA ligase inhibitory effect.

Where R¹ and R² or R² and R³ together form an optionally substituted cyclic ring containing 3-6 atoms, the cyclic ring can be a carbocyclic or heterocyclic ring. Suitable optionally substituted carbocyclic and heterocyclic rings include, cyclic ethers e.g. epoxide, oxetanyl, dioxanyl, e.g. 2,2-dimethyl-1,3-dioxanyl; cycloalkyl rings e.g. cyclopropyl, cyclobutyl, cyclopentyl and cyclohexyl, cyclohexanonyl rings; heterocyclyl rings e.g. azetidinyl, oxazolidonyl ring, oxathiolanyl ring, oxazinonyl ring, pyranonyl ring, piperidinonyl, tetrahydrothiophenyl ring, pyrrolidinyl ring, dioxolanyl ring, dioxanonyl ring, triazolyl ring, tetrazolyl ring, morpholinyl ring, 1,3,2-dioxathiolane-2,2-dioxidyl ring and piperidinyl ring. These rings may be optionally substituted by R^{1′}.

“Pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Compounds of the foregoing formulas I, Ia, Ib, II, IIa, IIb and IIc may form stable acid or basic salts, and in such cases administration of a compound as a salt may be appropriate, and pharmaceutically acceptable salts may be made by conventional methods well-known in the art.

Suitable pharmaceutically-acceptable salts include acid addition salts such as methanesulfonate, trifluoroacetate, tosylate, α-glycerophosphate fumarate, hydrochloride, citrate, maleate, tartrate and hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl D-glucamine and amino acids such as lysine. There may be more than one cation or anion depending on the number of charged functions and the valency of the cations or anions.

However, to facilitate isolation of the salt during preparation, salts which are less soluble in the chosen solvent may be preferred whether pharmaceutically-acceptable or not.

Within the present invention it is to be understood that a compound of any one of formula I, Ia, Ib, II, IIa, IIb and IIc or a salt thereof may exhibit the phenomenon of tautomerism and that the formula drawings within this specification can represent only one of the possible tautomeric forms. It is to be understood that the invention encompasses all tautomeric forms that inhibit bacterial DNA ligase and is not to be limited merely to any one tautomeric form utilized within the formula drawings.

It will be appreciated by those skilled in the art that in addition to the asymmetric carbon atoms specifically indicated in formulas Ia, Ib, IIa IIb and IIc, the compounds of these formulas may contain additional asymmetrically substituted carbon and/or sulphur atoms, and accordingly may exist in, and be isolated in, optically-active and racemic forms. Some compounds may exhibit polymorphism. It is to be understood that the present invention encompasses any racemic, optically-active, polymorphic or stereoisomeric form, or mixtures thereof, which possesses properties useful in the inhibition of bacterial DNA ligase, it being well known in the art how to prepare optically-active forms (for example, by resolution of the racemic form by recrystallization techniques, by synthesis from optically-active starting materials, by chiral synthesis, by enzymatic resolution, by biotransformation, or by chromatographic separation using a chiral stationary phase) and how to determine efficacy for the inhibition of bacterial DNA ligase by the standard tests described hereinafter.

When an optically active form of a compound of the invention is required, it may be obtained as specifically exemplified above or by carrying out one of the above procedures for racemic compounds but using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

According to a further feature of the invention, there is provided a compound of formulas I, Ia, Ib, II, IIa, IIb and IIc or a pharmaceutically-acceptable salt thereof for use in a method of treatment of the human or animal body by therapy.

It is also to be understood that certain compounds of the formulas I, Ia, Ib, II, IIa, IIb and IIc and salts thereof can exist in solvated as well as unsolvated forms such as, for example, hydrated forms. It is to be understood that the invention encompasses all such solvated forms that inhibit bacterial DNA ligase.

The removal of any protecting groups and the formation of pharmaceutically acceptable salts are within the skill of an ordinary organic chemist using standard techniques.

We have found that compounds of the present invention inhibit bacterial DNA ligase and are therefore of interest for their antibacterial effects.

According to a further feature of the present invention there is provided a method for producing an antibacterial effect in a warm-blooded-animal, such as man, in need of such treatment, which comprises administering to said animal an effective amount of a compound of the present invention represented by anyone of formulas I, Ia, Ib, II, IIa, IIb and IIc, or a pharmaceutically-acceptable salt thereof.

According toga further feature of the invention there is provided a method for inhibition of bacterial DNA ligase in a warm-blooded animal, such as a human being, in need of such treatment which comprises administering to said animal an effective amount of a compound of any one of formulas I, Ia, Ib, II, IIa, IIb and IIc or a pharmaceutically acceptable salt thereof as defined hereinbefore.

According to a further feature of the invention there is provided a method of treating a bacterial infection in a warm-blooded animal, such as a human being, in need of such treatment which comprises administering to said animal an effective amount of a compound of any one of formulas I, Ia, Ib, II, IIa, IIb and IIc or a pharmaceutically acceptable salt thereof as defined hereinbefore.

A further feature of the present invention is a compound of formulas II, IIa, IIb and IIc and pharmaceutically acceptable salts thereof for use as a medicament. Suitably, the medicament is an antibacterial agent.

A still further feature of the present invention is a compound of formulas I, Ia, Ib, II, IIa, IIb and IIc, or a pharmaceutically acceptable salt thereof, for use as a medicament for producing an antibacterial effect in a warm-blooded animal such as a human being. Particularly, this is a compound of formulas I, Ia, Ib, II, IIa, IIb and IIc or a pharmaceutically acceptable salt thereof, for use as a medicament for treating a bacterial infection in a warm-blooded animal such as a human being.

According to a further aspect of the invention there is provided the use of a compound of formulas I, Ia, Ib, II, IIa, IIb and IIc, or a pharmaceutically acceptable salt thereof in the manufacture of a medicament for use in inhibition of bacterial DNA ligase in a warm-blooded animal such as a human being.

Thus, according to a further aspect of the invention there is provided the use of a compound of formulas I, Ia, Ib, II, IIa, IIb and IIc or a pharmaceutically acceptable salt thereof in the manufacture of a medicament for use in the treatment of a bacterial infection in a warm-blooded animal such as a human being.

In order to use a compound of the formulas I, Ia, Ib, II, IIa, IIb and IIc or a pharmaceutically-acceptable salt thereof, (hereinafter in this section relating to pharmaceutical composition “a compound of this invention”) for the therapeutic, including prophylactic, treatment of mammals including humans, in particular in treating infection, it is normally formulated in accordance with standard pharmaceutical practice as a pharmaceutical composition.

Therefore in another aspect the present invention provides a pharmaceutical composition that comprises a compound of the formula II, IIa, IIb and IIc or a pharmaceutically acceptable salt thereof, and a pharmaceutically acceptable diluent or carrier.

According to a further aspect of the invention there is provided a pharmaceutical composition which comprises a compound of formulas I, Ia, Ib, II, IIa, IIb and IIc as defined hereinbefore or a pharmaceutically acceptable salt thereof, in association with a pharmaceutically acceptable excipient or carrier for use in inhibition of bacterial DNA ligase in an warm-blooded animal, such as a human being.

According to a further aspect of the invention there is provided a pharmaceutical composition which comprises a compound of formulas I, Ia, Ib, II, IIa, IIb and IIc as defined hereinbefore or a pharmaceutically acceptable salt thereof, in association with a pharmaceutically acceptable excipient or carrier for use in the treatment of a bacterial infection in a warm-blooded animal, such as a human being.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients well known in the art. Thus, compositions intended for oral use may contain, for example, one or more coloring, sweetening, flavoring and/or preservative agents.

Suitable pharmaceutically acceptable excipients for a tablet formulation include, for example, inert diluents such as lactose, sodium carbonate, calcium phosphate or calcium carbonate; granulating and disintegrating agents such as corn starch or algenic acid; binding agents such as starch; lubricating agents such as magnesium stearate, stearic acid or talc; preservative agents such as ethyl or propyl p-hydroxybenzoate; and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form or in the form of nano or micronized particles together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives such as ethyl or propyl p-hydroxybenzoate; anti-oxidants such as ascorbic acid); colouring agents; flavouring agents; and/or sweetening agents such as sucrose, saccharine or aspartame.

Oily suspensions may be formulated by suspending the active ingredient in a vegetable oil such as arachis oil, olive oil, sesame oil or coconut oil or in a mineral oil such as liquid paraffin. The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

In addition to the compounds of the present invention, the pharmaceutical composition of this invention may also contain or be co-administered (simultaneously, sequentially or separately) with one or more known drugs selected from other clinically useful antibacterial agents (for example, macrolides, quinolones, β-lactams or aminoglycosides) and/or other anti-infective agents (for example, an antifungal triazole or amphotericin). These may include carbapenems, for example meropenem or imipenem, to broaden the therapeutic effectiveness. Compounds of this invention may also contain or be co-administered with bactericidal/permeability-increasing protein (BPI) products or efflux pump inhibitors to improve activity against gram negative bacteria and bacteria resistant to antimicrobial agents.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. Accordingly, the optimum dosage may be determined by the practitioner who is treating any particular patient.

In addition to its use in therapeutic medicine, compounds of formulas I, Ia, Ib, II, IIa and IIb and their pharmaceutically acceptable salts are also useful as pharmacological tools in the development and standardization of in vitro and in vivo test systems for the evaluation of the effects of inhibitors of DNA ligase in laboratory animals such as cats, dogs, rabbits, monkeys, rats and mice, as part of the search for new therapeutic agents.

In any of the above-mentioned pharmaceutical composition, process, method, use, medicament, and manufacturing features of the instant invention, any of the alternate embodiments of the compounds of the invention described herein also apply.

### Enzyme Potency Testing Methods

Compounds were tested for inhibition of DNA ligase using a Fluorescence Resonance Energy Transfer (FRET) detection assay as previously described (Chen et al. 2002. Analytical Biochemistry 309: 232-240; Benson et al. 2004. Analytical Biochemistry 324:298-300). Assays were performed in 384-well polystyrene flat-bottom black plates in 30 μl reactions containing 3 μl compound dissolved in dimethylsulfoxide, 20 μl 1.5× Enzyme Working Solution (25% glycerol, 45 mM potassium chloride, 45 mM ammonium sulfate, 15 mM dithiothreitol, 1.5 mM ethylenediaminetetraacetic acid (EDTA), 0.003% Brij 35, 75 mM MOPS pH 7.5, 150 nM bovine serum albumin, 1.5 μM NAD⁺, 60 nM DNA substrate, 0.375 nM enzyme in water) and 7 μl 70 mM magnesium chlorine solution (96 mM magnesium chloride, 20% glycerol in water) to initiate the reaction. The DNA substrate is similar to that described in Benson et al. (2004. Analytical Biochemistry 324:298-300). The assay reactions were incubated at room temperature for approximately 20 minutes before being terminated by the addition of 30 μl Quench reagent (8 M Urea, 1 M Trizma base, 20 mM EDTA in water). Plates were read in a Tecan Ultra plate reader at two separate wavelengths—Read 1: excitation 485, emission 535, Read 2: excitation 485, emission 595. Data is initially expressed as a ratio of the 595/535 emission values and percent inhibition values were calculated using 0.2% dimethylsulfoxide (no compound) as the 0% inhibition and EDTA-containing (50 mM) reactions as 100% inhibition controls. Compound potency was based on IC₅₀ measurements determined from reactions performed in the presence of ten different compound concentrations.

The compounds described have a measured IC₅₀ in this assay against at least one isozyme (S. pneumoniae, S. aureus, H. influenzae, E. coli, or M. pneumoniae) of <400 μM or the compounds inhibited the ligation reaction by >20% at the limit of their solubility in the assay medium. Solubility is determined under assay conditions using a nephelometer to detect a change in turbidity as the concentration of compound increases. The limit of solubility is defined as the maximum concentration before a detectable increase in turbidity is measured.

Representative bacterial DNA ligase inhibition by the compounds of the instant invention is indicated below.

DNA Ligase Inhibitory Activity:

| Example Number | IC₅₀ (μM) against S. pneumoniae LigA |
|---|---|
| 6 | 1.8 |
| 25 | 4.0 |
| 128 | 0.7 |
| 172 | 0.145 |
| 259 | 0.5 |

### Bacterial Susceptibility Testing Methods

Compounds were tested for antimicrobial activity by susceptibility testing using microbroth dilution methods recommended by NCCLS. Compounds were dissolved in dimethylsulfoxide and tested in 10 doubling dilutions in the susceptibility assays such that the final dimethylsulfoxide concentration in the assay was 2% (v/v). The organisms used in the assay were grown overnight on appropriate agar media and then suspended in the NCCLS-recommended liquid susceptibility-testing media. The turbidity of each suspension was adjusted to be equal to a 0.5 McFarland standard, a further 1-in-10 dilution was made into the same liquid medium to prepare the final organism suspension, and 100 μL of this suspension was added to each well of a microtiter plate containing compound dissolved in 2 μL of dimethylsulfoxide. Plates were incubated under appropriate conditions of atmosphere and temperature and for times according to NCCLS standard methods prior to being read. The Minimum Inhibitory Concentration (MIC) was determined as the lowest drug concentration able to reduce growth by 80% or more.

Representative antibacterial activity for the compounds of the instant invention is indicated below.

### Antibacterial Activity:

| Example Number | MIC (μg/ml) against S. pneumoniae |
|---|---|
| 27 | 8 |
| 62 | 4 |
| 129 | 8 |
| 172 | 2 |
| 356 | 4 |

### Process

If not commercially available, the necessary starting materials for the procedures such as those described herein may be made by procedures which are selected from standard organic chemical techniques, techniques which are analogous to the synthesis of known, structurally similar compounds, or techniques which are analogous to the described procedure or the procedures described in the Examples.

It is noted that many of the starting materials for synthetic methods as described herein are commercially available and/or widely reported in the scientific literature, or could be made from commercially available compounds using adaptations of processes reported in the scientific literature. The reader is further referred to Advanced Organic Chemistry, 4^{th} Edition, by Jerry March, published by John Wiley & Sons 1992, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, published by John Wiley and Sons, 1991).

Examples of suitable protecting groups for a hydroxy group are, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric, phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid, for example boron tris(trifluoroacetate). A suitable alternative protecting group for a primary amino group is, for example, a phthaloyl group, which may be removed by treatment with an alkylamine, for example dimethylaminopropylamine or 2-hydroxyethylamine, or with hydrazine. Another suitable protecting group for an amine is, for example, a cyclic ether such as tetrahydrofuran, which may be removed by treatment with a suitable acid such as trifluoroacetic acid.

The protecting groups may be removed at any convenient stage in the synthesis using conventional techniques well known in the chemical art, or they may be removed during a later reaction step or work-up.

The skilled organic chemist will be able to use and adapt the information contained and referenced within the above references, and accompanying Examples therein and also the Examples herein, to obtain necessary starting materials and products.

Another aspect of the present invention provides a process for preparing a compound of formula I or a pharmaceutically acceptable salt thereof which process (wherein R, R¹, R² and R³ are, unless otherwise specified, as defined in formula I) comprises:

a) reacting a purine base of formula (1):

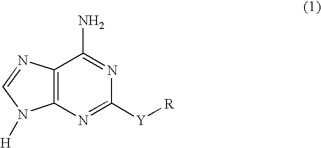

or a suitably protected derivative thereof; with an electrophile of formula (2) wherein L is a suitable leaving group such as acetate, methoxy, benzoyl, or chloro; or

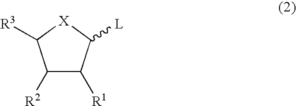

reacting a purine base of formula (3):

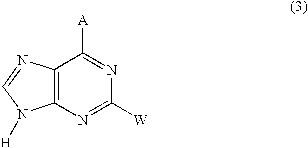

wherein A is Cl, NH₂, or a suitably protected amino group and W is halo, with an electrophile of formula (2) followed by reaction with a compound of formula (4), and if A is Cl, a subsequent reaction with the appropriate amine, such as ammonia;
and thereafter if necessary: converting a compound of formula I into another compound of formula I; removing any protecting groups; and optionally forming a pharmaceutically acceptable salt.

Specific reaction conditions for the above reactions are as follows:

Purine bases of formula (1) and electrophiles of formula (2) may be coupled together using standard coupling conditions known in the art. These include, but are not limited to glycosylation conditions such as those described in Vorbrueggen, H. and Bennua, B. Chem. Ber., 1981, 114, 1279-1286, and Dudycz, L. V. and Wright, G. E. Nucleosides and Nucleotides, 1984, 3, 33-44. Other coupling methods include but are not limited to nucleophilic substitution reactions catalyzed by, for example bases, Lewis acids or palladium, and substitution using reagents such as triphenylphosphine and diethylazodicarboxylate. Alternative methods of synthesizing compounds of formula I, for example starting from the appropriately substituted pyrimidine or imidazole, can be utilized as described in Joule, J. A. and Mills, K., Heterocyclic Chemistry, Fourth Edition, published by Blackwell Publishing, 2000, for analogous compounds.

A compound of formula (1) can be prepared by functionalization of a substituted purine compound which is commercially available or is a known compound or is prepared by processes known in the art, for example by processes such as those shown in Scheme 1 for Y is O.

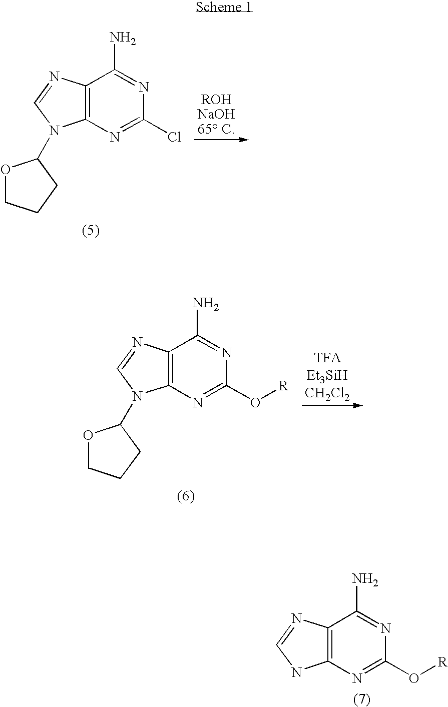

Displacement of chloro or other displaceable group such as bromo, fluoro or iodo by the appropriate nucleophile, for example an alcohol, or thiol, can be done either neat or in a suitable solvent such as tetrahydrofuran, DCM, DMF, or N-methylpyrrolidinone in temperatures ranging from 65-200° C. Bases such as sodium hydroxide, potassium carbonate, n-butyl lithium, potassium tert-butoxide, or sodium hydride can be used as necessary according to one skilled in the art. If necessary, a suitable protecting group, for example benzoyl, can be installed prior to deprotection of the tetrahydrofuran.

A compound of formula I with Y is —CH₂— or —CH═CH— can be made using methods known to one skilled in the art. The compounds can be made by, for example, a metal-catalyzed coupling between a purine base and a carbon-containing substituent with each of the molecules containing a leaving group useful in metal-catalyzed couplings, for example, boronate, trialkyltin, iodo or bromo, as in J. Med. Chem., 1998, 39, 4211-4217, Bioorg. Med. Chem. Lett., 1995, 3, 1377-1382, J. Org. Chem., 1997, 62, 6833-6841, and Tetrahedron Lett., 1995, 36, 6507-6510, and the examples and references contained therein and the non-limiting Examples herein. Other methods include but are not limited to nucleophilic displacement and reaction using reagents such as triphenylphosphine and diethylazodicarboxylate.

Compounds of formula (2) are prepared by processes known in the art using procedures found in the literature such as those modifying an appropriately protected ribose derivative. The reader is referred to Preparative Carbohydrate Chemistry, edited by S. Hanessian, published by Marcel Dekker, 1997 for general guidance on transformations and reaction conditions. For example, one method to synthesize compound (11) is shown in Scheme 2. The reaction of compound (8) or other suitably protected ribose derivative with a displaceable group can be carried out by a number of fluorinating reagents such as tetrabutylammonium fluoride, (diethylamino)sulfur trifluoride (DAST), potassium fluoride, or Amberlyst A-26 (F⁻ 40 nm) to give compound (9). Following deprotection and reprotection, compound (11) is obtained and can be coupled with a compound of formula (1).

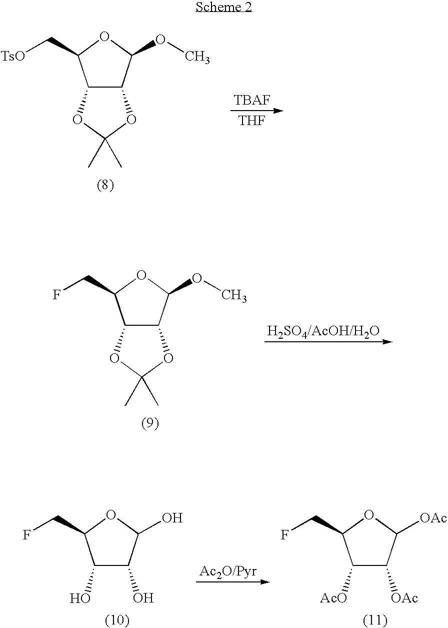

Alternatively, compounds of formula I can be prepared by converting a particular compound of formula I to a different compound of formula I using the appropriate protecting groups, reactions, and deprotections using methods known to one skilled in the art. One non-limiting example of how the 5′-position of the ribose can be modified is shown in Scheme-3, and one non-limiting example of how the 2′- and 3′-positions of the ribose can be modified is shown in Scheme 4. Appropriate chemistry can be applied to modify the 5′ and 2′ and 3′-positions of the ribose, in each case using the appropriate combination of protecting groups. Further manipulations can be made using techniques known to one skilled in the art.

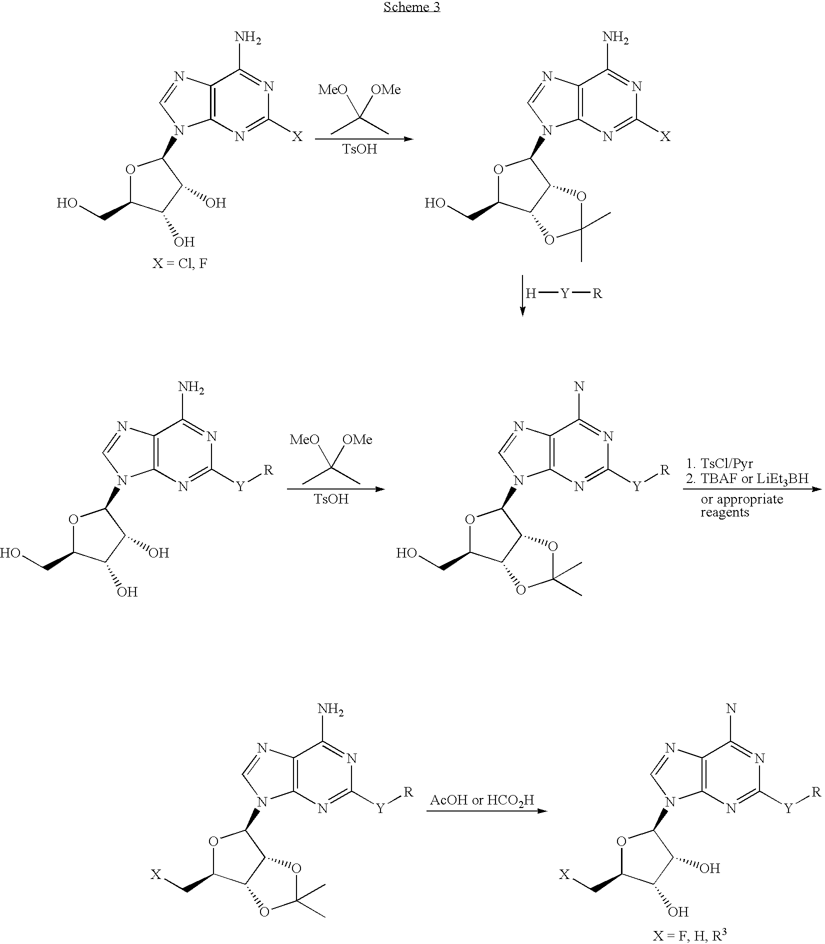

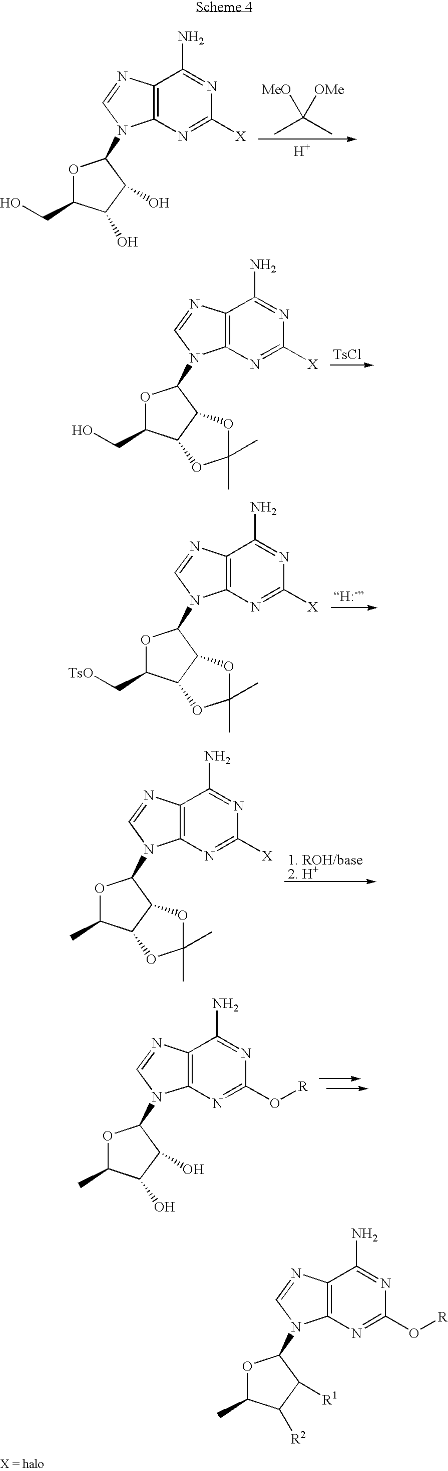

The alcohols used in the displacement reaction on the 2-haloadenosine may be commercially available. Those that aren't can be synthesized by methods well known to those of skill in the art. One non-limiting example is shown in Scheme 5.

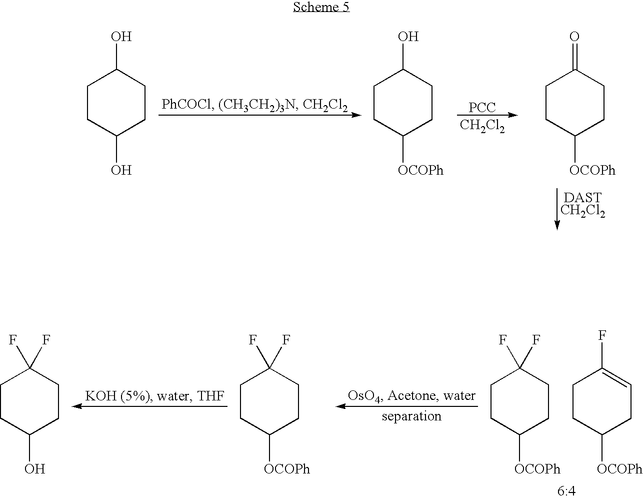

The invention is now illustrated but not limited by the following Examples in which unless otherwise stated:

(i) evaporations were carried out by rotary evaporation in vacuo and work-up procedures were carried out after removal of residual solids by filtration;
(ii) temperatures are quoted as ° C.; operations were carried out at room temperature, that is typically in the range 18-26° C. and without exclusion of air unless otherwise stated, or unless the skilled person would otherwise work under an inert atmosphere;
(iii) column chromatography (by the flash procedure) was used to purify compounds and was performed on Merck Kieselgel silica (Art. 9385) unless otherwise stated; Jones Flashmaster and Biotage refer to automated normal phase chromatography instruments using silica cartridges for the stationary phase; the instruments were used according to the manufacturers instructions;
(iv) in general, the course of reactions was followed by TLC, HPLC, or LC/MS and reaction times are given for illustration only; yields are given for illustration only and are not necessarily the maximum attainable;
(v) the structure of the end-products of the invention were generally confirmed by NMR and mass spectral techniques. Proton magnetic resonance spectra were generally determined in DMSO-d₆ unless otherwise stated, using a Bruker DRX-300 spectrometer operating at a field strength of 300 MHz. In cases where the NMR spectrum is complex, only diagnostic signals are reported. Chemical shifts are reported in parts per million downfield from tetramethylsilane as an internal standard (δ scale) and peak multiplicities are shown thus: s, singlet; d, doublet; dd, doublet of doublets; dt, doublet of triplets; dm, doublet of multiplets; t, triplet, m, multiplet; br, broad. Fast-atom bombardment (FAB) mass spectral data were generally obtained using a Platform spectrometer (supplied by Micromass) run in electrospray and, where appropriate, either positive ion data or negative ion data were collected or using Agilent 1100series LC/MSD equipped with Sedex 75ELSD, and where appropriate, either positive ion data or negative ion data were collected. The lowest mass major ion is reported for molecules where isotope splitting results in multiple mass spectral peaks (for example when chlorine is present). Reverse Phase HPLC was carried out using YMC Pack ODS-AQ (100×20 mmID, S-5μ particle size, 12 nm pore size) on Agilent instruments;
(vi) each intermediate was purified to the standard required for the subsequent stage and was characterized in sufficient detail to confirm that the assigned structure was correct; purity was assessed by HPLC, TLC, or NMR and identity was determined by infra-red spectroscopy (IR), mass spectroscopy or NMR spectroscopy as appropriate;
(vii) the following abbreviations may be used:
TLC is thin layer chromatography; HPLC is high pressure liquid chromatography; MPLC is medium pressure liquid chromatography; NMR is nuclear magnetic resonance spectroscopy; DMSO is dimethylsulfoxide; CDCl₃ is deuterated chloroform; MeOD is deuterated methanol, i.e. D₃COD; MS is mass spectroscopy; ESP (or ES) is electrospray; EI is electron impact; APCI is atmospheric pressure chemical ionization; THF is tetrahydrofuran; DCM is dichloromethane; MeOH is methanol; DMF is dimethylformamide; EtOAc is ethyl acetate; LC/MS is liquid chromatography/mass spectrometry; h is hour(s); min is minute(s); d is day(s); TFA is trifluoroacetic acid; v/v is ratio of volume/volume; Boc denotes t-butoxycarbonyl; Cbz denotes benzyloxycarbonyl; Bz denotes benzoyl; atm denotes atmospheric pressure; rt denotes room temperature; mg denotes milligram; g denotes gram; μL denotes microliter; mL denotes milliliter; L denotes liter; μM denotes micromolar; mM denotes millimolar; M denotes molar; N denotes normal; nm denotes nanometer;
(viii) microwave reactor refers to a Smith Microwave Synthesizer, equipment that uses microwave energy to heat organic reactions in a short period of time; it was used according to the manufacturers instructions and was obtained from Personal Chemistry Uppsala AB; and
(ix) Kugelrohr distillation refers to a piece of equipment that distills liquids and heats sensitive compounds using air-bath oven temperature; it was used according to the manufacturers instruction and was obtained from Büchi, Switzerland or Aldrich, Milwaukee, USA.

### EXAMPLE 1

### 2-(butylthio)-9-(tetrahydrofuran-2-yl)-9H-purin-6-amine

2-Chloro-9-(tetrahydrofuran-2-yl)-9H-purin-6-amine (50 mg, 0.21 mmol), n-butanethiol (0.2 ml, 2 mmol), and potassium carbonate (61 mg, 0.44 mmol) were suspended in DMF (1.5 ml). The reaction was heated to 150° C. overnight. Volatiles were removed in vacuo, and the residue was purified by flash chromatography using 2% MeOH in DCM as eluent. Relevant fractions were combined to give 42 mg of the desired product.

MS (ESP): 294 (MH⁺) for C₁₃H₁₉N₅OS

¹H NMR δ: 0.89 (t, 3H) 1.33-1.47 (m, 2H) 1.63 (m, 2H) 2.02 (m, 1H) 2.14-2.28 (m, 1H) 2.38 (m, 2H) 3.05 (t, 2H) 3.81-3.95 (m, 1H) 4.11 (m, 1H) 6.16 (dd, 1H) 7.29 (s, 2H) 8.09 (s, 1H).

Intermediates for this compound were prepared as follows:—

### 2-Chloro-9-(tetrahydrofuran-2-yl)-9H-purin-6-amine

2,6-Dichloro-9-(tetrahydrofuran-2-yl) purine (0.6 g, 2.3 mmol) was suspended in 7N ammonia in MeOH (5 ml) in a microwave reaction vessel. The vessel was sealed and the reaction mixture heated in the microwave reactor at 120° C. for 0.5 h. Volatiles were removed in vacuo and the resulting product was purified by flash chromatography using 2% MeOH in chloroform as the eluent to give a white solid (63% yield).

MS (APCI-pos): 170.11 (MH⁺-THF) for C₉H₁₀ClN₅O

¹H NMR δ: 2.02 (m, 1H); 2.15 (m, 1H); 2.38 (m, 2H); 3.88 (dt, 1H); 4.10 (dt, 1H); 6.17 (t, 1H); 7.79 (br s, 2H); 8.26 (s, 1H)

### 2,6-dichloro-9-(tetrahydrofuran-2-yl) purine

To a solution of 2,6-dichloropurine (1.0 g, 5.3 mmol) in EtOAc (30 ml) was added p-toluenesulfonic acid monohydrate (100 mg, 0.5 mmol) followed by 2,3-dihydrofuran (1.0 ml, 13.2 mmol). The reaction mixture was heated to 45° C. for 3 h. After cooling to rt, the reaction was diluted with EtOAc (50 ml) and washed successively with 50 mL each of saturated sodium carbonate and water, then the organics were dried over sodium sulfate. Purification by flash chromatography using 100% EtOAc as the eluent resulted in an off-white solid (87% yield).

MS (APCI-pos): 189.11 (MH⁺-THF) for C₉H₈Cl₂N₄O

¹H NMR δ: 2.06, 2.14 (2); 3.93 m, 2H); 2.44 (m, 2H (dt, 1H); 4.18 (dt, 1H); 6.33 (t, 1H); 8.82 (s, 1H)

Using an analogous procedure to that described in Example 1, the appropriate commercially available thiol was reacted with 2-chloro-9-(tetrahydrofuran-2-yl)-9H-purin-6-amine to give the compounds described in Table I.

TABLE I

| EX | IUPAC Name | MH+ | ¹H NMR |
|---|---|---|---|
| 2 | 4-{[6-amino-9-(tetrahydrofuran-2-yl)- | 308 | 1.45-1.58 (m, 2H) 1.61-1.72 (m, 2H) |
| | 9H-purin-2-yl]thio}butan-1-ol | | 1.93-2.07 (m, 1H) 2.14-2.29 (m, 1H) |
| | | | 2.31-2.45 (m, 2H) 3.06 (t, 2H) 3.39 (s, 2H) |
| | | | 3.85-3.94 (m, 1H) 4.12 (m, 1H) 4.40 (s, 1H) 6.16 (dd, 1H) |
| | | | 7.27 (s, 2H) 8.09 (s, 1H) |
| 3 | 2-(benzylthio)-9-(tetrahydrofuran-2-yl)- | 258 (−THF) | 1.91-2.06 (m, 1H) 2.09-2.24 (m, 1H) |
| | 9H-purin-6-amine | | 2.32-2.47 (m, 2H) 3.80-3.94 (m, 1H) 4.10 (m, 1H) |
| | | | 4.35 (s, 2H) 6.20 (dd, 1H) 7.18-7.31 (m, |
| | | | 3H) 7.40 (d, 2H) 7.44 (s, 2H) 8.11 (s, 1H) |
| 4 | 1-{[6-amino-9-(tetrahydrofuran-2-yl)- | 296 | 1.14 (d, 3H) 2.01 (m, 1H) 2.24 (m, 1H) |
| | 9H-purin-2-yl]thio}propan-2-ol | | 2.48 (mm, 2H) 3.12 (d, 3H) 3.88 (m, 2H) 4.13 (m, |
| | | | 1H) 4.84 (d, 1H) 6.18 (m, 1H) 7.31 (s, 2H) |
| | | | 8.11 (s, 1H) |
| 5 | 2-(cyclohexylthio)-9-(tetrahydrofuran- | 320 | 1.40 (mm, 5H) 1.58 (m, 1H) 1.73 (m, 2H) |
| | 2-yl)-9H-purin-6-amine | | 2.04 (mm, 3H) 2.30 (mm, 2H) 2.55 (m, 1H) |
| | | | 3.69 (m, 1H) 3.92 (m, 1H) 4.13 (m, 1H) |
| | | | 6.17 (m, 1H) 7.28 (bs, 2H) 8.09 (s, 1H) |
| 6 | 2-[(2-furylmethyl)thio]-9- | 318 | 2.01 (m, 1H) 2.19 (m, 1H) 2.42 (m, 2H) |
| | (tetrahydrofuran-2-yl)-9H-purin-6- | | 3.89 (m, 1H) 4.11 (m, 1H) 4.40 (s, 2H) 6.20 (m, 1H) |
| | amine | | 6.34 (m, 2H) 7.39 (s, 2H) 7.55 (m, 1H) |
| | | | 8.12 (s, 1H) |
| 7 | 2-[(4-methoxybenzyl)thio]-9- | 358 | 2.03 (m, 1H) 2.18 (m, 1H) 2.41 (m, 2H) |
| | (tetrahydrofuran-2-yl)-9H-purin-6- | | 3.71 (s, 3H) 3.82-3.95 (m, 1H) 4.04-4.17 (m, 1H) |
| | amine | | 4.30 (s, 2H) 6.21 (t, 3H) 6.84 (d, 2H) |
| | | | 7.35 (m, 4H) 8.11 (s, 1H) |
| 8 | 2-[(4-chlorobenzyl)thio]-9- | 362 | 1.99 (m, 2H) 2.39 (m, 2H) 3.87 (m, 1H) |
| | (tetrahydrofuran-2-yl)-9H-purin-6- | | 4.08 (m, 1H) 4.34 (s, 2H) 6.19 (t, 1H) |
| | amine | | 7.34 (overlapping m, 4H) 7.47 (m, 2H) 8.11 (s, 1H) |
| 9 | 2-[(4-methylbenzyl)thio]-9- | 342 | 1.92-2.07 (m, 1H) 2.11-2.21 (m, 1H) |
| | (tetrahydrofuran-2-yl)-9H-purin-6- | | 2.24 (s, 3H) 2.40 (m, 2H) 3.88 (m, 1H) 4.10 (m, 1H) |
| | amine | | 4.31 (s, 2H) 6.20 (m, 1H) 7.08 (d, 2H) |
| | | | 7.29 (bs, 2H) 7.33 (d, 2H) 8.11 (s, 1H) |
| 10 | 2-(cyclopentylthio)-9-(tetrahydrofuran- | 306 | 1.62 (overlapping m, 6H) 2.00 (m, 1H) |
| | 2-yl)-9H-purin-6-amine | | 2.15 (m, 3H) 2.35 (m, 1H) 3.91 (overlapping m, 2H) |
| | | | 4.09 (m, 2H) 6.16 (m, 1H) 7.25 (bs, 2H) |
| | | | 8.10 (s, 1H) |
| 11 | 2-[(4-fluorobenzyl)thio]-9- | 346 | 1.99 (m, 1H) 2.14 (m, 1H) 2.40 (m, 2H) |
| | (tetrahydrofuran-2-yl)-9H-purin-6- | | 3.88 (m, 1H) 4.08 (m, 1H) 4.34 (s, 2H) 6.19 (m, 1H) |
| | amine | | 7.10 (t, 2H) 7.38 (bs, 2H) 7.47 (m, 2H) |
| | | | 8.11 (s, 1H) |
| 12 | 2-(allylthio)-9-(tetrahydrofuran-2-yl)- | 278 | 2.02 (m, 1H) 2.20 (m, 2H) 2.39 (mm, 2H) |
| | 9H-purin-6-amine | | 3.75 (d, 2H) 3.90 (m, 1H) 4.11 (m, 1H) |
| | | | 5.06 (d, 1H) 5.29 (d, 1H) 5.95 (mm, 1H) 6.17 (m, |
| | | | 1H) 7.34 (bs, 2H) 8.11 (s, 1H) |
| 13 | 2-[(2,4-dichlorobenzyl)thio]-9- | 397 | 1.99 (m, 1H) 2.12 (m, 1H) 2.38 (m, 2H) |
| | (tetrahydrofuran-2-yl)-9H-purin-6- | | 3.87 (m, 1H) 4.07 (m, 1H) 4.41 (s, 2H) 6.20 (t, 1H) |
| | amine | | 7.34 (dd, 1H) 7.44 (bs, 2H) 7.62 (d, 1H) |
| | | | 7.71 (d, 1H) 8.12 (s, 1H) |
| 14 | 2-[(2-chlorobenzyl)thio]-9- | 362 | 2.00 (m, 1H) 2.15 (m, 1H) 2.40 (m, 2H) |
| | (tetrahydrofuran-2-yl)-9H-purin-6- | | 3.87 (m, 1H) 4.09 (m, 1H) 4.44 (s, 2H) 6.20 (m, 1H) |
| | amine | | 7.26 (m, 2H) 7.39-7.48 (mm, 3H) |
| | | | 7.67 (bs, 1H) 8.12 (s, 1H) |
| 15 | 2-[(2-methylbenzyl)thio]-9- | 342 | 2.01 (m, 1H) 2.16 (m, 1H) 2.35 (s, 3H) |
| | (tetrahydrofuran-2-yl)-9H-purin-6- | | 2.42 (m, 2H) 3.88 (m, 1H) 4.10 (m, 1H) 4.36 (s, 2H) |
| | amine | | 6.21 (m, 1H) 7.14 (m, 3H) 7.39 (mm, 3H) |
| | | | 8.12 (s, 1H) |
| 16 | 2-(phenylthio)-9-(tetrahydrofuran-2-yl)- | 314 | 1.70 (m, 2H) 2.22 (mm, 2H) 3.65 (t, 2H) |
| | 9H-purin-6-amine | | 5.98 (m, 1H) 7.44 (m, 5H) 7.65 (s, 2H) 8.06 (s, 1H) |
| 17 | 2-[(2-phenylethyl)thio]-9- | 342 | 2.01 (m, 1H) 2.17 (m, 1H) 2.40 (m, 2H) |
| | (tetrahydrofuran-2-yl)-9H-purin-6- | | 2.97 (t, 2H) 3.3 (m, 2H) 3.89 (m, 1H) 4.12 (m, 1H) |
| | amine-2-(phenylthio)-9- | | 6.21 (m, 1H) 7.23 (m, 1H) 7.32 (m, 6H) |
| | (tetrahydrofuran-2-yl)-9H-purin-6- | | 8.12 (s 1H) |
| | amine (1:1) | | |
| 18 | 2-{[6-amino-9-(tetrahydrofuran-2-yl)- | 371 | 1.88 (m, 1H) 2.13 (m, 2H) 2.34 (m, 1H) |
| | 9H-purin-2-yl]thio}-N-phenylacetamide | | 3.81 (m, 1H) 3.97 (s, 2H) 4.06 (m, 1H) 6.14 (m, |
| | | | 1H) 7.04 (t, 1H) 7.30 (t, 2H) 7.42 (bs, 2H) |
| | | | 7.59 (d, 2H) 8.23 (s, 1H) 10.14 (s, 1H) |
| 19 | 2-[(pyridin-2-ylmethyl)thio]-9- | 329 | 0.96 (m, 1h) 2.06 (m, 1H) 2.22 (m, 1H) |
| | (tetrahydrofuran-2-yl)-9H-purin-6- | | 2.45 (m, 3H) 3.93 (m, 1H) 4.14 (m, 1H) 4.53 (s 2H) |
| | amine | | 6.24 (m, 1H) 7.32 (m, 1H) 7.48 (bs, 2H) |
| | | | 7.63 (d, 1H) 8.18 (s, 1H) 8.56 (d 1H) |
| 20 | 2-[(3,4-dichlorobenzyl)thio]-9- | 397 | 2.04 (m, 1H) 2.15 (m, 1H) 2.42 (m 2H) |
| | (tetrahydrofuran-2-yl)-9H-purin-6- | | 3.91 (m, 1H) 4.11 (m, 1H) 4.36 (s, 2H) 6.23 (t, 1H) |
| | amine | | 7.47 (bs, 2H) 7.51 (d, 1H) 7.57 (d, 1H) 7.78 (d, |
| | | | 1H) 8.16 s, 1H) |
| 21 | 9-(tetrahydrofuran-2-yl)-2-{[3- | 396 | 1.99 (m, 1H) 2.11 (m, 1H) 2.38 (m, 2H) |
| | (trifluoromethyl)benzyl]thio}-9H-purin- | | 3.86 (m, 1H) 4.07 (m, 1H) 4.43 (s, 2H) 6.18 (t, 1H) |
| | 6-amine | | 7.43 (bs, NH2) 7.55 (m, 2H) 7.79 (m, 2H) |
| | | | 8.12 (s, 1H) |

### EXAMPLE 22

### 2-(butylsulfonyl)-9-(tetrahydrofuran-2-yl)-9H-purin-6-amine

2-(Butylthio)-9-(tetrahydrofuran-2-yl)-9H-purin-6-amine, prepared as in Example 1 (0.1 g, 0.34 mmol) was dissolved in DCM (10 ml) and cooled to 0° C. A solution of m-chloroperbenzoic acid (0.25 g, 1.02 mmol) in DCM (5 ml) was added dropwise. The solution was allowed to warm to rt and stir overnight. A solution of saturated sodium bisulfite (10 ml) was added and the mixture stirred for 10 min. The layers were separated and the aqueous layer was extracted with DCM (2×), washed with 1N sodium bicarbonate, saturated sodium bicarbonate, and brine, and dried over sodium sulfate. Volatiles were removed in vacuo and the residue was purified by flash chromatography using 2% MeOH in DCM as eluent. Relevant fractions were combined to give 70 mg of the desired product.

MS (ESP): 326 (MH⁺) for C₁₃H₁₉N₅O₃S

¹H NMR δ: 0.89 (t, 3H) 1.34-1.48 (m, 2H) 1.67 (m, 2H) 1.97-2.12 (m, 1H) 2.17-2.32 (m, 1H) 2.42-2.55 (m, 2H) 3.41-3.55 (m, 2H) 3.86-4.01 (m, 1H) 4.15 (m, 1H) 6.29 (t, 1H) 8.01 (s, 2H) 8.47 (s, 1H).

### EXAMPLE 23

### 2-(butylthio)-9-(2-deoxy-β-D-erythro-pentofuranosyl)-9H-purin-6-amine

Using essentially the same procedure described for Example 1, starting with 2-chloro-2′-deoxyadenosine (50 mg, 0.17 mmol) and n-butanethiol, the desired compound was obtained following purification by flash chromatography using 10% MeOH in chloroform as eluent. Relevant fractions were combined to give 50 mg of the desired product.

MS (ESP): 340 (MH⁺) for C₁₄H₂₁N₅O₃S

¹H NMR δ: 0.90 (t, 3H) 1.40 (m, 2H) 1.63 (m, 2H) 2.23 (m, 1H) 2.74 (m, 1H) 2.99-3.12 (m, 2H) 3.53 (m, 2H) 3.83 (m, 1H) 4.37 (br s, 1H) 4.94 (br s, 1H) 5.30 (br s, 1H) 6.25 (t, 1H) 7.31 (s, 2H) 8.18 (s, 1H)

### EXAMPLE 24

### 2-(butylthio)-9-(5-deoxy-β-D-ribofuranosyl)-9H-purin-6-amine

Using essentially the same procedure as described in Example 1, starting with 25 mg of 2-chloro-9-(5-deoxy-β-D-ribofuranosyl)-9H-purin-6-amine (made from 2-chloroadenosine using a procedure analogous to that described in Can. J. Chem. 1991, 69, 1468-74) and n-butanethiol (0.05 ml, 0.4 mmol), the desired compound was obtained following purification by flash chromatography using 10% MeOH in chloroform as eluent. Relevant fractions were combined to give 17 mg of the desired product.

MS (ESP): 340 (MH⁺) for C₁₄H₂₁N₅O₃S

¹H NMR δ: 0.89 (t, 3H) 1.28 (m, 3H) 1.34-1.49 (m, 2H) 1.63 (s, 2H) 3.05 (s, 2H) 3.94 (s, 2H) 4.70 (s, 1H) 5.13 (s, 1H) 5.44 (s, 1H) 5.74 (d, 1H) 7.34 (s, 2H) 8.18 (s, 1H)

### EXAMPLE 25

### 2-(benzyloxy)-9-(tetrahydrofuran-2-yl)-9H-purin-6-amine

2-Chloro-9-(tetrahydrofuran-2-yl)-9H-purin-6-amine (0.12 g, 0.5 mmol), sodium hydroxide (0.1 g, 2.5 mmol), and benzyl alcohol (0.51 ml, 4.5 mmol) (neat) were stirred at 85° C. for 3 h. After LC/MS indicated that the reaction was complete, it was cooled to rt and diluted with chloroform (40 ml). The organic layer was washed with water (5×10 ml), dried over sodium sulfate, and concentrated in vacuo. The yellow oil was dissolved in DCM and purified by column chromatography using 0-5% MeOH in DCM as eluent. The relevant fractions were combined and concentrated in vacuo to give 0.08 g (69%) of a white solid.

MS (ESP): 312 (MH⁺) for C₁₆H₁₇N₅O₂

¹H NMR δ: 1.97 (m, 1H) 2.18 (m, 1H) 2.38 (m, 2H) 3.85 (m, 1H) 4.07 (m, 1H) 5.30 (s, 2H) 6.12 (m, 1H) 7.36 (mm, 7H) 8.04 (s, 1H)

Using an analogous procedure to that described in Example 25, the appropriate commercially available alcohol was reacted with 2-chloro-9-(tetrahydrofuran-2-yl)-9H-purin-6-amine to give the compounds described in Table II. Reactions were either done neat or in an appropriate solvent. The reactions can be heated from 80-200° C. or reacted in a microwave reactor at 150° C. for 10 min to 8 h.

TABLE II

| EX | IUPAC Name | MH⁺ | ¹H NMR |
|---|---|---|---|
| 26 | 2-butoxy-9-(tetrahydrofuran-2- | 278 | 0.80 (t, 3H) 1.26 (hex, 2H) 1.53 (m, 2H) 1.88 (m, 1H) |
| | yl)-9H-purin-6-amine | | 2.08 (mm, 1H) 2.28 (mm, 2H) 3.75 (m, 1H) |
| | | | 3.98 (m, 1H) 4.07 (t, 2H) 6.00 (m, 1H) 7.08 (bs, 2H) |
| | | | 7.91 (s, 1H) |
| 27 | 2-(cyclohexyloxy)-9- | 304 | 1.25 (mm, 6H) 1.57 (m, 2H) 1.81 (m, 3H) 2.10 (m, 1H) |
| | (tetrahydrofuran-2-yl)-9H-purin- | | 2.24 (m, 2H) 3.76 (dd, 1H) 3.97 (m, 2H) 4.72 (m, |
| | 6-amine | | 1H) 5.97 (m, 1H) 7.01 (bs, 2H) 7.87 (s, 1H) |
| 28 | 2-(cyclohexylmethoxy)-9- | 318 | 1.01 (m, 2H) 1.19 (m, 3H) 1.71 (m, 6H) 1.99 (m, 1H) |
| | (tetrahydrofuran-2-yl)-9H-purin- | | 2.21 (m, 1H) 2.40 (m, 2H) 3.88 (dd, 1H) |
| | 6-amine | | 3.99 (dd, 1H) 4.01 (d, 1H)4.10 (dd, 1H) 6.11 (m, 1H) |
| | | | 7.20 (bs, 2H) 8.02 (s, 1H) |
| 29 | 2-(cyclopentyloxy)-9- | 290 | 1.55 (mm, 6H) 1.82 (m, 2H) 1.93 (m, 1H) 2.17 (m, 1H) |
| | (tetrahydrofuran-2-yl)-9H-purin- | | 2.30 (m, 2H) 3.82 (dd, 1H) 4.05 (dd, 1H) |
| | 6-amine | | 5.20 (m, 1H) 6.05 (m, 1H) 7.08 (bs, 2H) 7.94 (s, 1H) |
| 30 | 2-(cyclobutyloxy)-9- | 276 | 1.60 (m, 1H) 1.72 (m, 1H) 2.01 (m, 3H) 2.22 (m, 1H) |
| | (tetrahydrofuran-2-yl)-9H-purin- | | 2.36 (m, 4H) 3.88 (dd, 1H) 4.10 (dd, 1H) |
| | 6-amine | | 5.02 (m, 1H) 6.10 (dd, 1H) 7.18 (bs, 2H) 8.01 (s, 1H) |
| 31 | 2-(cyclopropylmethoxy)-9- | 276 | 0.29 (m, 2H) 0.51 (m, 2H) 1.21 (m, 1H) 1.99 (m, 1H) |
| | (tetrahydrofuran-2-yl)-9H-purin- | | 2.22 (m, 2H) 3.87 (dd, 1H) 4.03 (d, 2H) 4.09 (dd, |
| | 6-amine | | 1H) 6.11 (m, 1H) 7.20 (bs, 2H) 8.02 (s, 1H) |
| 32 | 2-(cyclopentylmethoxy)-9- | 304 | 1.27 (m, 2H) 1.54 (m, 4H) 1.72 (m, 2H) 2.01 (m, 1H) |
| | (tetrahydrofuran-2-yl)-9H-purin- | | 2.24 (m, 2H) 2.40 (m, 2H) 3.86 (dd, 1H) 4.06 (m, |
| | 6-amine | | 3H) 6.12 (m, 2H) 7.21 (bs, 2H) 8.02 (s, 1H) |
| 33 | 2-(cycloheptyloxy)-9- | 318 | 1.54 (mm, 10H) 1.94 (s, 3H) 2.22 (m, 1H) 2.37 (m, 2H) |
| | (tetrahydrofuran-2-yl)-9H-purin- | | 3.87 (dd, 1H) 4.09 (dd, 1H) 5.05 (m, 1H) |
| | 6-amine | | 6.11 (ms, 1H) 7.14 (bs, 1H) 8.00 (s, 1H) |
| 34 | 2-(cyclobutylmethoxy)-9- | 290 | 1.83 (mm, 4H) 2.02 (m, 3H) 2.22 (m, 1H) 2.40 (m, 2H) |
| | (tetrahydrofuran-2-yl)-9H-purin- | | 2.68 (dd, 1H) 3.88 (dd, 1H) 4.09 (dd, 1H) |
| | 6-amine | | 4.17 (d, 2H) 6.12 (m, 1H) 7.21 (bs, 2H) 8.03 (s, 1H) |
| 35 | 2-(cyclooctylmethoxy)-9- | 346 | 1.49 (mm, 14H) 1.97 (m, 2H) 2.22 (m, 1H) 2.40 (m, |
| | (tetrahydrofuran-2-yl)-9H-purin- | | 2H) 3.87 (dd, 1H) 3.97 (d, 2H) 4.11 (dd, 1H) |
| | 6-amine | | 6.12 (m, 1H) 7.21 (bs, 2H) 8.03 (s, 1H) |
| 36 | 2-phenoxy-9-(tetrahydrofuran-2- | 298 | 1.85 (m, 2H) 2.30 (m, 2H) 3.75 (dd, 1H) 3.88 (dd, 1H) |
| | yl)-9H-purin-6-amine | | 6.03 (t, 1H) 7.14 (m, 3H) 7.38 (m, 4H) 8.07 (s, 1H) |
| 37 | 2-[(1-ethylprop-2-en-1-yl)oxy]-9- | 290 | 0.89 (t, 3H) 1.56 (s, 1H) 1.68 (m, 2H) 2.01 (m, 1H) |
| | (tetrahydrofuran-2-yl)-9H-purin- | | 2.22 (m, 1H) 2.39 (m, 2H) 3.89 (m, 1H) 4.09 (m, 1H) |
| | 6-amine | | 5.15 (dd, 2H) 5.38 (m, 1H) 5.88 (m, 2H) 6.10 (m, |
| | | | 1H) 7.20 (bs, 2H) 8.02 (s, 1H) |
| 38 | 2-(2-methoxyethoxy)-9- | 280 | 2.01 (m, 1H) 2.21 (m, 1H) 2.39 (m, 2H) 3.28 (s, 3H) |
| | (tetrahydrofuran-2-yl)-9H-purin- | | 3.61 (m, 2H) 3.88 (dd, 1H) 4.10 (m, 1H) 4.31 (m, 2H) |
| | 6-amine | | 6.11 (m, 1H) 7.25 (bs, 2H) 8.03 (s, 1H) |
| 39 | 9-(tetrahydrofuran-2-yl)-2- | 292 | 1.55 (s, 2H) 1.99 (s, 2H) 2.19 (s, 2H) 2.37 (s, 2H) |
| | (tetrahydrofuran-3-yloxy)-9H- | | 3.74 (s, 3H) 3.89 (s, 2H) 4.10 (s, 1H) 5.37 (s, 1H) |
| | purin-6-amine | | 6.11 (s, 1H) 7.24 (s, 1H) 8.03 (s, 1H) |
| 40 | 2-[(3-methylcyclopentyl)oxy]-9- | 304 | 1.00 (mm, 3H) 1.25 (m, 2H) 1.92 (overlapping m, 9H) |
| | (tetrahydrofuran-2-yl)-9H-purin- | | 3.88 (m, 1H) 4.11 (m, 1H) 5.25 (m, 1H) 6.11 (m, |
| | 6-amine | | 1H) 7.15 (bs, 2H) 8.01 (s, 1H) |
| 41 | 2-(1-phenylethoxy)-9- | 236 | 1.53 (d, 2H) 1.97 (m, 1H) 2.16 (m, 1H) 2.31 (m, 1H) |
| | (tetrahydrofuran-2-yl)-9H-purin- | | 3.92 (m, 1H) 6.06 (m, 2H) 7.23 (m, 3H) 7.32 (t, 2H) |
| | 6-amine | | 7.38 (d, 2H) 7.99 (s, 1H) |
| 42 | 2-[(2,3-difluorobenzyl)oxy]-9- | 348 | 2.00 (m, 1H) 2.18 (m, 1H) 2.38 (m, 2H) 3.86 (m, 1H) |
| | (tetrahydrofuran-2-yl)-9H-purin- | | 4.07 (m, 1H) 5.39 (s, 2H) 6.12 (t, 1H) 7.21 (m, 2H) |
| | 6-amine | | 7.33 (m, 3H) 8.05 (s, 1H) |
| 43 | 2-(pyridin-2-ylmethoxy)-9- | 313 | 1.93 (m, 1H) 2.15 (m, 1H) 2.34 (m, 2H) 3.83 (m, 1H) |
| | (tetrahydrofuran-2-yl)-9H-purin- | | 4.01 (m, 1H) 5.38 (s, 2H) 6.09 (m, 1H) |
| | 6-amine | | 7.36 (mm, 4H) 7.78 (t, 1H) 8.04 (s, 1H) 8.54 (d, 1H) |
| 44 | 2-[2-(methylthio)ethoxy]-9- | 296 | 2.01 (m, 1H) 2.13 (s, 3H) 2.20 (m, 1H) 2.39 (m, 2H) |
| | (tetrahydrofuran-2-yl)-9H-purin- | | 2.80 (t, 2H) 3.88 (m, 1H) 4.10 (m, 1H) 4.36 (t, 2H) |
| | 6-amine | | 6.13 (m, 1H) 7.27 (bs, 2H) 8.04 (s, 1H) |
| 45 | N-(2-{[6-amino-9- | 307 | 1.80 (s, 3H) 2.01 (m, 1H) 2.21 (m, 1H) 2.39 (m, 2H) |
| | (tetrahydrofuran-2-yl)-9H-purin- | | 3.37 (s, 2H) 3.88 (m, 1H) 4.10 (m, 1H) 4.19 (m, 2H) |
| | 2-yl]oxy}ethyl)acetamide | | 6.11 (m, 1H) 7.24 (bs, 2H) 8.04 (s, 1H) 8.09 (bs, 1H) |
| 46 | 1-(2-{[6-amino-9- | 333 | 1.89 (m, 3H) 2.01 (m, 1H) 2.19 (t, 3H) 2.40 (m, 2H) |
| | (tetrahydrofuran-2-yl)-9H-purin- | | 3.33 (t, 2H) 3.51 (t, 2H) 3.88 (m, 1H) 4.10 (m, 1H) |
| | 2-yl]oxy}ethyl)pyrrolidin-2-one | | 4.29 (t, 2H) 6.13 (m, 1H) 7.28 (bs, 2H) 8.04 (s, 1H) |
| 47 | 2-(octahydro-1H-inden-5-yloxy)- | 344 | 1.7 (overlapping mm, 17H) 2.22 (m, 1H) 2.37 (m, 1H) |
| | 9-(tetrahydrofuran-2-yl)-9H- | | 3.87 (m, 1H) 4.10 (m, 1H) 7.15 (bs, 2H) 8.00 (s, |
| | purin-6-amine | | 1H) |
| 48 | 2-[(2-methylprop-2-en-1-yl)oxy]- | 276 | 1.75 (s, 3H) 2.01 (m, 1H) 2.22 (m, 1H) 2.40 (m, 2H) |
| | 9-(tetrahydrofuran-2-yl)-9H- | | 2.22 (m, 1H) 2.40 (m, 2H) 3.88 (dd, 1H) 4.11 (dd, 1H) |
| | purin-6-amine | | 4.66 (s, 2H) 4.95 (d, 1H) 6.12 (m, 1H) 7.27 (bs, 2H) |
| | | | 8.04 (s, 1H) |
| 49 | 2-(bicyclo[2.2.1]hept-2- | 330 | 0.72 (m, 1H) 1.33 (overlapping mm, 8H) 1.84 (m, 3H) |
| | ylmethoxy)-9-(tetrahydrofuran-2- | | 2.40 (mm, 6H) 4.09 (mm, 4H) 6.12 (s, 1H) |
| | yl)-9H-purin-6-amine | | 7.21 (bs, 2H) 8.03 (s, 1H) |
| 50 | 9-(tetrahydrofuran-2-yl)-2-(3,4,5- | 352 | 1.93 (mm, 2H) 2.34 (m, 2H) 3.81 (m, 1H) 3.94 (m, 1H) |
| | trifluorophenoxy)-9H-purin-6- | | 6.07 (m, 1H) 7.52 (bs, 2H) 8.11 (s, 1H) |
| | amine | | |
| 51 | 2-(2-cyclohexylethoxy)-9- | 332 | 0.93 (m, 2H) 1.17 (m, 3H) 1.40 (m, 1H) 1.62 (m, 1H) |
| | (tetrahydrofuran-2-yl)-9H-purin- | | 1.75 (m, 1H) 2.00 (m, 1H) 2.20 (m, 1H) 2.42 (m, |
| | 6-amine | | 2H) 3.87 (m, 1H) 4.10 (m, 1H) 4.22 (m, 2H) |
| | | | 6.11 (m, 1H) 7.21 (bs, 2H) 8.00 (s, 1H) |
| 52 | 2-[(1-methylbut-2-yn-1-yl)oxy]-9- | 288 | 1.47 (d, 3H) 1.57 (s, 1H) 1.77 (s, 3H) 2.01 (m, 1H) |
| | (tetrahydrofuran-2-yl)-9H-purin- | | 2.23 (m, 1H) 2.37 (m, 1H) 3.90 (m, 1H) 4.13 (m, 1H) |
| | 6-amine | | 5.60 (m, 1H) 6.13 (m, 1H) 7.28 (bs, 2H) 8.04 (s, |
| | | | 1H) |
| 53 | 9-(tetrahydrofuran-2-yl)-2- | 306 | 1.63 (m, 1H) 1.90 (mm, 4H) 2.22 (m, 1H) 2.40 (m, 2H) |
| | (tetrahydrofuran-2-ylmethoxy)- | | 3.65 (m, 1H) 3.76 (m, 1H) 3.88 (m, 1H) |
| | 9H-purin-6-amine | | 4.06-4.18 (m, 4H) 6.12 (m, 1H) 7.24 (bs, 2H) 8.03 (s, 1H) |
| 54 | 9-(tetrahydrofuran-2-yl)-2-(4,4,4- | 332 | 1.90 (m, 3H) 2.21 (m, 1H) 2.39 (m, 4H) 3.88 (m, 1H) |
| | trifluorobutoxy)-9H-purin-6- | | 4.10 (m, 1H) 4.25 (t, 2H) 6.13 (m, 1H) 7.26 (bs, 2H) |
| | amine | | 8.04 (s, 1H) |
| 55 | 2-(2-furylmethoxy)-9- | 302 | 2.02 (m, 1H) 2.23 (m, 1H) 2.41 (m, 2H) 3.89 (m, 1H) |
| | (tetrahydrofuran-2-yl)-9H-purin- | | 4.12 (m, 1H) 5.24 (s, 2H) 6.14 (m, 1H) 6.45 (m, 1H) |
| | 6-amine | | 6.55 (d, 1H) 7.30 (bs, 2H) 7.67 (s, 1H) 8.05 (s, 1H) |
| 56 | 2-[(2,2-dimethyl-1,3-dioxolan-4- | 336 | 1.31 (d, 6H) 1.99 (m, 1H) 2.21 (m, 1H) 2.40 (m, 2H) |
| | yl)methoxy]-9-(tetrahydrofuran-2- | | 3.73 (m, 1H) 3.88 (m, 1H) 4.08 (s, 2H) 4.23 (m, 2H) |
| | yl)-9H-purin-6-amine | | 4.37 (m, 1H) 6.12 (m, 1H) 7.27 (bs, 2H) 8.04 (s, |
| | | | 1H) |
| 57 | 2-(4-methoxyphenoxy)-9- | 328 | 1.85 (m, 1H) 1.93 (m, 1H) 2.29 (m, 2H) |
| | (tetrahydrofuran-2-yl)-9H-purin- | | 3.72-3.81 (overlapping m, 4H) 3.90 (m, 1H) 6.02 (m, 1H) |
| | 6-amine | | 6.89-6.96 (mm, 2H) 7.02-7.09 (m, 2H) 7.34 (bs, 2H) |
| | | | 8.05 (s, 1H) |
| 58 | 2-[(4-methylcyclohexyl)oxy]-9- | 318 | 0.88 (d, 3H) 1.05 (m, 2H) 1.34 (m, 2H) 1.72 (m, 2H) |
| | (tetrahydrofuran-2-yl)-9H-purin- | | 2.03 (m, 3H) 2.25 (s, 1H) 2.37 (s, 3H) 3.90 (m, 1H) |
| | 6-amine | | 4.12 (m, 1H) 4.75 (s, 1H) 6.10 (s, 1H) 7.13 (s, 2H) |
| | | | 8.00 (s, 1H) |
| 59 | 9-(tetrahydrofuran-2-yl)-2- | 306 | 1.62 (m, 1H) 2.00 (m, 2H) 2.20 (m, 1H) 2.40 (s, 2H) |
| | (tetrahydrofuran-3-ylmethoxy)- | | 2.62 (m, 1H) 3.49 (m, 1H) 3.64 (m, 1H) 3.75 (m, 2H) |
| | 9H-purin-6-amine | | 3.88 (m, 1H) 4.16 (mm, 3H) 6.12 (m, 1H) |
| | | | 7.25 (bs, 2H) 8.04 (s, 1H) |
| 60 | 2-(3-cyclopentylpropoxy)-9- | 332 | 1.07 (bs, 2H) 1.39 (m, 2H) 1.54 (mm, 4H) 1.74 (mm, |
| | (tetrahydrofuran-2-yl)-9H-purin- | | 5H) 1.99 (m, 1H) 2.22 (m, 1H) 2.39 (mm, 2H) |
| | 6-amine | | 3.87 (m, 1H) 4.15 (mm, 3H) 6.11 (m, 1H) 7.20 (bs, 2H) |
| | | | 8.02 (s, 1H) |
| 61 | 9-(tetrahydrofuran-2-yl)-2- | 306 | 1.61 (m, 2H) 1.99 (m, 3H) 2.22 (m, 1H) 2.40 (mm, 2H) |
| | (tetrahydro-2H-pyran-4-yloxy)- | | 3.46 (m, 2H) 3.86 (mm, 3H) 4.10 (m, 1H) |
| | 9H-purin-6-amine | | 5.04 (m, 1H) 6.11 (m, 1H) 7.21 (bs, 2H) 8.02 (s, 1H) |
| 62 | 2-(decahydronaphthalen-2-yloxy)- | 358 | 1.22-1.75 (overlapping mm, 14H) 1.80 (overlapping |
| | 9-(tetrahydrofuran-2-yl)-9H- | | mm, 2H) 1.99 (m, 1H) 2.22 (m, 1H) 2.40 (m, 2H) |
| | purin-6-amine | | 3.87 (m, 1H) 4.09 (m, 1H) 4.83-5.06 (m, 1H) |
| | | | 6.11 (m, 1H) 7.15 (bs, 2H) 8.00 (s, 1H) |
| 63 | 2-(2,3-dihydro-1H-inden-1- | 338 | 1.94-2.12 (mm, 2H) 2.16-2.27 (m, 1H) |
| | yloxy)-9-(tetrahydrofuran-2-yl)- | | 2.32-2.45 (mm, 3H) 2.86 (m, 1H) 3.04 (m, 1H) 3.88 (m, 1H) |
| | 9H-purin-6-amine | | 4.12 (mm, 1H) 6.15 (m, 1H) 6.37 (mm, 1H) |
| | | | 7.25 (mm, 5H) 7.45 (m, 1H) 8.05 (s, 1H) |
| 64 | 2-[(2E)-oct-2-en-1-yloxy]-9- | 332 | 0.83 (t, 3H) 1.24 (m, 3H) 1.34 (m, 3H) 2.01 (m, 3H) |
| | (tetrahydrofuran-2-yl)-9H-purin- | | 2.22 (m, 1H) 2.40 (m, 2H) 3.88 (m, 1H) 4.11 (m, 1H) |
| | 6-amine | | 4.67 (d, 2H) 5.73 (mm, 2H) 6.11 (m, 1H) |
| | | | 7.23 (bs, 2H) 8.02 (s, 1H) |
| 65 | 9-(tetrahydrofuran-2-yl)-2- | 352 | 1.99 (mm, 3H) 2.20 (mm, 1H) 2.39 (s, 2H) |
| | (1,2,3,4-tetrahydronaphthalen-2- | | 2.85 (mm, 3H) 3.15-3.22 (m, 1H) 3.87 (m, 1H) 4.11 (m, |
| | yloxy)-9H-purin-6-amine | | 1H) 5.32 (bs, 1H) 6.11 (m, 1H) 7.09 (m, 4H) |
| | | | 7.20 (bs, 2H) 8.03 (s, 1H) |
| 66 | 9-(tetrahydrofuran-2-yl)-2-[(3,3,5- | 346 | 0.81-0.97 (mm, 12H) 1.09 (m, 1H) 1.33 (d, 1H) |
| | trimethylcyclohexyl)oxy]-9H- | | 1.68 (bs, 1H) 1.78 (d, 1H) 2.03 (mm, 2H) 2.22 (m, 1H) |
| | purin-6-amine | | 2.37 (m, 1H) 3.89 (m, 1H) 4.10 (m, 1H) 5.01 (m, |
| | | | 1H) 6.09 (m, 1H) 7.14 (bs, 2H) 7.99 (s, 1H) |
| 67 | 2-(2-phenylethoxy)-9- | 326 | 1.99 (m, 1H) 2.20 (mm, 1H) 2.39 (mm, 2H) 2.99 (t, |
| | (tetrahydrofuran-2-yl)-9H-purin- | | 2H) 3.87 (m, 1H) 4.10 (m, 1H) 4.39 (t, 2H) 6.12 (m, |
| | 6-amine | | 1H) 7.18-7.32 (mm, 7H) 8.03 (s, 1H) |
| 68 | 2-(3-phenylpropoxy)-9- | 340 | 1.98 (m, 3H) 2.20 (m, 1H) 2.37 (mm, 2H) 2.70 (t, 2H) |
| | (tetrahydrofuran-2-yl)-9H-purin- | | 3.87 (m, 1H) 4.09 (m, 1H) 4.18 (t, 2H) 6.11 (m, 1H) |
| | 6-amine | | 7.23 (mm, 7H) 8.02 (s, 1H) |
| 69 | 2-(2-cyclopentylethoxy)-9- | 318 | 1.13 (m, 2H) 1.53 (m, 4H) 1.69 (mm, 4H) 1.87 (m, 1H) |
| | (tetrahydrofuran-2-yl)-9H-purin- | | 1.99 (m, 1H) 2.22 (m, 1H) 2.40 (m, 2H) 3.88 (m, |
| | 6-amine | | 1H) 4.10 (m, 1H) 4.20 (t, 2H) 6.11 (m, 1H) 7.20 (bs, |
| | | | 2H) 8.02 (s, 1H) |
| 70 | 2-(1-cyclopentylethoxy)-9- | 318 | 1.21 (d, 3H) 1.29 (m, 2H) 1.53 (m, 4H) 1.68 (m, 2H) |
| | (tetrahydrofuran-2-yl)-9H-purin- | | 2.01 (m, 2H) 2.21 (m, 1H) 2.40 (m, 2H) 3.89 (m, |
| | 6-amine | | 1H) 4.08 (m, 1H) 4.92 (m, 1H) 6.12 (m, 1H) |
| | | | 7.16 (bs, 2H) 8.01 (s, 1H) |
| 71 | 2-(cyclohex-3-en-1-ylmethoxy)-9- | 316 | 1.30 (m, 1H) 1.80 (m, 2H) 2.01 (m, 5H) 2.16 (m, 1H) |
| | (tetrahydrofuran-2-yl)-9H-purin- | | 2.40 (m, 2H) 3.86 (m, 1H) 4.10 (m, 3H) 5.66 (d, |
| | 6-amine | | 2H) 6.13 (m, 1H) 7.22 (bs, 2H) 8.03 (s, 1H) |
| 72 | 2-(1-cyclobutylethoxy)-9- | 304 | 1.13 (m, 3H) 1.76 (m, 2H) 1.84 (mm, 7H) 2.23 (m, 1H) |
| | (tetrahydrofuran-2-yl)-9H-purin- | | 2.42 (m, 3H) 3.88 (m, 1H) 4.10 (m, 1H) 4.99 (m, |
| | 6-amine | | 1H) 6.12 (m, 1H) 7.18 (bs, 2H) 8.01 (s, 1H) |
| 73 | 9-(tetrahydrofuran-2-yl)-2-({4- | 374 | 1.00 (m, 3H) 1.48 (m, 2H) 1.79 (m, 3H) 1.99 (m, 1H) |
| | [(vinyloxy)methyl]cyclohexyl}methoxy)- | | 2.30 (mm, 3H) 3.54 (m, 3H) 4.02 (mm, 7H) |
| | 9H-purin-6-amine | | 6.12 (m, 1H) 6.48 (m, 1H) 7.21 (bs, 2H) 8.02 (s, 1H) |
| 74 | 2-(pent-4-yn-1-yloxy)-9- | 288 | 1.85 (m, 2H) 2.01 (m, 1H) 2.20 (m, 1H) 2.29 (m, 2H) |
| | (tetrahydrofuran-2-yl)-9H-purin- | | 2.40 (m, 2H) 2.81 (m, 1H) 3.88 (m, 1H) 4.11 (m, |
| | 6-amine | | 1H) 4.24 (t, 2H) 6.12 (m, 1H) 7.23 (bs, 2H) 8.03 (s, |
| | | | 1H) |
| 75 | 2-[2-(isopropylthio)ethoxy]-9- | 324 | 1.20 (d, 6H) 1.99 (m, 1H) 2.19 (m, 1H) 2.42 (m, 2H) |
| | (tetrahydrofuran-2-yl)-9H-purin- | | 2.84 (t, 2H) 3.04 (m, 1H) 3.88 (m, 1H) 4.09 (m, 1H) |
| | 6-amine | | 4.32 (t, 2H) 6.12 (m, 1H) 7.25 (bs, 2H) 8.03 (s, 1H) |
| 76 | 2-[2-(4-methyl-1,3-thiazol-5- | 347 | 1.99 (m, 1H) 2.21 (m, 2H) 2.33 (s, 3H) 2.41 (m, 2H) |
| | yl)ethoxy]-9-(tetrahydrofuran-2- | | 3.19 (t, 2H) 3.88 (m, 1H) 4.10 (m, 1H) 4.35 (t, 2H) |
| | yl)-9H-purin-6-amine | | 6.12 (m, 1H) 7.28 (bs, 2H) 8.04 (s, 1H) 8.82 (s, 1H) |
| 77 | 2-{[(2Z)-2-methyl-3-phenylprop- | 352 | 1.90 (s, 3H) 2.02 (m, 1H) 2.24 (m, 1H) 2.41 (m, 2H) |
| | 2-en-1-yl]oxy}-9- | | 3.89 (m, 1H) 4.12 (m, 1H) 4.81 (s, 2H) 6.14 (m, 1H) |
| | (tetrahydrofuran-2-yl)-9H-purin- | | 6.60 (s, 1H) 7.31 (mm, 7H) 8.05 (s, 1H) |
| | 6-amine | | |
| 78 | 2-(diphenylmethoxy)-9- | 312 | 1.97 (m, 1H) 2.19 (m, 1H) 2.33 (m, 2H) 3.85 (m, 1H) |
| | (tetrahydrofuran-2-yl)-9H-purin- | | 4.02 (m, 1H) 6.09 (m, 1H) 7.02 (s, 1H) |
| | 6-amine | | 7.30 (mm, 8H) 7.45 (m, 4H) 8.01 (s, 1H) |
| 79 | 2-{[(2Z)-3,7-dimethylocta-2,6- | 358 | 1.56 (d, 6H) 1.69 (s, 3H) 2.02 (bm, 5H) 2.22 (m, 1H) |
| | dien-1-yl]oxy}-9- | | 2.42 (m, 2H) 3.88 (m, 1H) 4.11 (m, 1H) 4.73 (d, |
| | (tetrahydrofuran-2-yl)-9H-purin- | | 2H) 5.05 (bs, 1H) 5.42 (m, 1H) 6.12 (m, 1H) |
| | 6-amine | | 7.22 (bs, 1H) 8.02 (s, 1H) |
| 80 | 2-(2,3-dihydro-1H-inden-2- | 338 | 1.99 (m, 1H) 2.24 (m, 1H) 2.37 (m, 2H) 2.99 (m, 2H) |
| | yloxy)-9-(tetrahydrofuran-2-yl)- | | 3.36 (m, 2H) 3.88 (m, 1H) 4.13 (m, 1H) 5.62 (m, |
| | 9H-purin-6-amine | | 1H) 6.12 (m, 1H) 7.20 (mm, 6H) 8.04 (s, 1H) |
| 81 | 2-(cyclohex-2-en-1-yloxy)-9- | 302 | 1.61 (mm, 1H) 1.73 (mm, 2H) 1.95 (mm, 4H) |
| | (tetrahydrofuran-2-yl)-9H-purin- | | 2.22 (mm, 1H) 2.41 (m, 2H) 3.89 (m, 1H) 4.12 (m, 1H) |
| | 6-amine | | 5.39 (bs, 1H) 5.88 (mm, 2H) 6.12 (m, 1H) 7.21 (bs, |
| | | | 1H) 8.03 (s, 1H) |
| 82 | 2-[(2-methylcyclopentyl)oxy]-9- | 304 | 1.00 (d, 3H) 1.19 (m, 1H) 1.63 (mm, 3H) |
| | (tetrahydrofuran-2-yl)-9H-purin- | | 1.81-1.93 (m, 1H) 2.02 (mm, 3H) 2.20 (m, 1H) 2.39 (mm, 2H) |
| | 6-amine | | 3.88 (m, 1H) 4.10 (m, 1H) 4.79 (m, 1H) 6.11 (m, 1H) |
| | | | 7.16 (bs, 1H) 8.01 (s, 1H) |
| 83 | 2-(2-cyclopropylethoxy)-9- | 290 | 0.09 (m, 2H) 0.41 (m, 2H) 0.78 (m, 1H) 1.57 (m, 2H) |
| | (tetrahydrofuran-2-yl)-9H-purin- | | 1.99 (m, 1H) 2.22 (m, 1H) 2.41 (m, 2H) 3.88 (m, |
| | 6-amine | | 1H) 4.10 (m, 1H) 4.23 (t, 2H) 6.12 (m, 1H) 7.22 (bs, |
| | | | 2H) 8.03 (s, 1H) |
| 84 | 9-(tetrahydrofuran-2-yl)-2-[2- | 396 | 2.00 (mm, 1H) 2.29 (mm, 1H) 2.39 (m, 2H) 3.88 (m, |
| | (2,3,6-trifluorophenoxy)ethoxy]- | | 1H) 4.10 (m, 1H) 4.49 (s, 4H) 6.11 (m, 1H) 7.19 (m, |
| | 9H-purin-6-amine | | 2H) 7.29 (bs, 2H) 8.04 (s, 1H) |
| 85 | 9-(tetrahydrofuran-2-yl)-2- | 320 | 1.28 (mm, 2H) 1.63 (m, 2H) 1.99 (mm, 2H) |
| | (tetrahydro-2H-pyran-4- | | 2.20 (mm, 1H) 2.40 (m, 2H) 3.31 (m, 2H) 3.86 (mm, 3H) |
| | ylmethoxy)-9H-purin-6-amine | | 4.06 (mm, 3H) 6.12 (m, 1H) 7.24 (bs, 1H) 8.03 (s, 1H) |
| 86 | 2-[(2-methylcyclohexyl)oxy]-9- | 318 | 0.90 (d, 3H) 1.08 (m, 1H) 1.24 (mm, 3H) 1.60 (m, 2H) |
| | (tetrahydrofuran-2-yl)-9H-purin- | | 1.68-1.80 (m, 2H) 2.01 (m, 1H) 2.10 (m, 1H) |
| | 6-amine | | 2.23 (m, 1H) 2.40 (m, 2H) 3.89 (m, 1H) 4.10 (m, 1H) |
| | | | 4.52 (m, 1H) 6.11 (m, 1H) 7.16 (bs, 2H) 8.01 (s, |
| | | | 1H) |
| 87 | 2-[(4-chlorocyclohexyl)oxy]-9- | 338 | 0.85-2.39 (overlapping mm, 13H) 3.89 (m, 1H) |
| | (tetrahydrofuran-2-yl)-9H-purin- | | 4.11 (m, 1H) 4.32 (m, 1H) 4.80-5.0 (mm, 1H) 6.14 (m, 1H) |
| | 6-amine | | 7.24 (bs, 2H) 8.04 (s, 1H) |
| 88 | 2-[(1- | 290 | 0.35 (m, 2H) 0.49 (m, 2H) 1.14 (m, 3H) 2.00 (m, 1H) |
| | methylcyclopropyl)methoxy]-9- | | 2.20 (m, 1H) 3.88 (m, 1H) 4.00 (s, 2H) 4.09 (m, 1H) |
| | (tetrahydrofuran-2-yl)-9H-purin- | | 6.12 (m, 1H) 7.23 (bs, 2H) 8.03 (s, 1H) |
| | 6-amine | | |
| 89 | 2-[(2- | 290 | 0.27 (m, 1H) 0.45 (m, 1H) 0.71 (m, 1H) 0.93 (mm, 1H) |
| | methylcyclopropyl)methoxy]-9- | | 1.01 (d, 3H) 2.00 (m, 1H) 2.20 (m, 1H) 2.39 (m, |
| | (tetrahydrofuran-2-yl)-9H-purin- | | 2H) 3.87 (m, 1H) 3.96-4.05 (overlapping mm, 2H) |
| | 6-amine | | 4.10 (mm, 1H) 6.11 (m, 1H) 7.20 (bs, 2H) 8.02 (s, 1H) |
| 90 | 9-(tetrahydrofuran-2-yl)-2- | 322 | 1.49 (m, 1H) 1.75 (m, 1H) 1.95-2.45 (overlapping |
| | (tetrahydro-2H-thiopyran-3- | | mm, 7H) 2.58 (m, 2H) 2.92 (m, 1H) 3.89 (m, 1H) |
| | yloxy)-9H-purin-6-amine | | 4.11 (m, 1H) 4.96 (m, 1H) 6.11 (m, 1H) 7.24 (bs, 2H) |
| | | | 8.03 (s, 1H) |
| 91 | 2-[(5-methyl-1,3-dioxan-5- | 336 | 0.87 (s, 3H) 2.01 (m, 1H) 2.20 (m, 1H) 2.39 (m, 2H) |
| | yl)methoxy]-9-(tetrahydrofuran-2- | | 3.48-3.82 (dd, 4H) 3.88 (m, 1H) 4.10 (m, 1H) |
| | yl)-9H-purin-6-amine | | 4.23 (s, 2H) 4.65-4.86 (dd, 2H) 6.15 (m, 1H) 7.29 (bs, 2H) |
| | | | 8.05 (s, 1H) |
| 92 | 2-[2-ethoxy-1- | 352 | 1.07 (s, 6H) 1.99 (d, J = 7.16 Hz, 1H) 2.20 (d, J = 6.59 Hz, |
| | (ethoxymethyl)ethoxy]-9- | | 1H) 2.39 (s, 2H) 3.39-3.50 (m, 4H) 3.56 (d, |
| | (tetrahydrofuran-2-yl)-9H-purin- | | J = 4.90 Hz, 4H) 3.88 (d, J = 6.03 Hz, 1H) 4.10 (d, |
| | 6-amine | | J = 7.54 Hz, 1H) 5.21 (s, 1H) 6.11 (d, J = 2.83 Hz, 1H) |
| | | | 7.25 (s, 2H) 8.03 (s, 1H) |
| 93 | 2-[(4-ethylcyclohexyl)oxy]-9- | 332 | 0.86 (t, 3H) 1.00 (m, 2H) 1.13-1.39 (overlappin |
| | (tetrahydrofuran-2-yl)-9H-purin- | | mm, 5H) 1.79 (m, 2H) 2.06 (mm, 3H) 2.23 (m, 2H) |
| | 6-amine | | 2.37 (mm, 2H) 3.89 (m, 1H) 4.10 (m, 1H) 4.76 (mm, |
| | | | 1H) 6.10 (m, 1H) 7.12 (bs, 2H) 8.00 (s, 1H) |
| 94 | 2-[(3-methylcyclohexyl)oxy]-9- | 318 | 0.91 (overlapping mm, 5H) 1.25 (mm, 2H) 1.48 (bs, 1H) |
| | (tetrahydrofuran-2-yl)-9H-purin- | | 1.61 (m, 1H) 1.75 (m, 1H) 2.03 (mm, 3H) |
| | 6-amine | | 2.23 (m, 1H) 2.38 (mm, 2H) 3.89 (m, 1H) 4.11 (m, 1H) |
| | | | 4.79 (m, 1H) 6.10 (m, 1H) 7.14 (bs, 1H) 8.00 (s, 1H) |
| 95 | 2-[(cis-4-methylcyclohexyl)oxy]- | 318 | 0.89 (d, 3H) 1.29 (m, 2H) 1.49 (mm, 5H) 1.99 (mm, |
| | 9-(tetrahydrofuran-2-yl)-9H- | | 3H) 2.22 (mm, 1H) 2.32-2.44 (overlapping m, 2H) |
| | purin-6-amine | | 3.88 (m, 1H) 4.10 (m, 1H) 5.08 (bs, 1H) 6.11 (m, 1H) |
| | | | 7.17 (bs, 2H) 8.01 (s, 1H) |
| 96 | 2-[(trans-4- | 318 | 0.88 (d, 3H) 1.04 (m, 2H) 1.36 (mm, 3H) 1.72 (m, 2H) |
| | methylcyclohexyl)oxy]-9- | | 2.04 (m, 3H) 2.24 (m, 1H) 2.38 (mm, 1H) |
| | (tetrahydrofuran-2-yl)-9H-purin- | | 3.89 (m, 1H) 4.11 (m, 1H) 4.75 (mm, 1H) 6.10 (m, 1H) |
| | 6-amine | | 7.14 (bs, 2H) 8.00 (s, 1H) |
| 97 | 2-[2-(dimethylamino)ethoxy]-9- | 293 | 2.20 (m, 10H) 2.57 (m, 2H) 3.89 (m, 1H) 4.11 (m, 1H) |
| | (tetrahydrofuran-2-yl)-9H-purin- | | 4.28 m 2H) 6.12 (m, 1H) 7.23 (bs, 2H) 8.03 (s, 1H) |
| | 6-amine | | |
| 98 | 2-(2-methoxy-4-methylphenoxy)- | 342 | 1.82 (m, 2H) 2.30 (bs, 5H) 3.21 (s, 3H) 3.81 (mm, 2H) |
| | 9-(tetrahydrofuran-2-yl)-9H- | | 6.01 (m, 1H) 6.74 (m, 1H) 6.94 (m, 2H) 7.29 (bs 2H) |
| | purin-6-amine | | 8.03 (s1H) |
| 99 | 2-[(1-methylpyrrolidin-3-yl)oxy]- | 305 | 1.80 (m, 1H) 2.01 (m, 1H) 2.25 (mm, 5H) 2.40 (mm, |
| | 9-(tetrahydrofuran-2-yl)-9H- | | 3H) 2.60 (mm, 2H) 2.84 (m, 1H) 3.88 (m, 1H) |
| | purin-6-amine | | 4.11 (m, 1H) 5.24 (m, 1H) 6.11 (m, 1H) 7.21 (bs 2H) |
| | | | 8.02 (s, 1H) |
| 100 | 2-(piperidin-3-yloxy)-9- | 305 | 1.33 (m, 2H) 1.64 (m, 1H) 1.95 (mm, 2H) 2.35 (mm, |
| | (tetrahydrofuran-2-yl)-9H-purin- | | 3H) 2.62 (m, 1H) 2.77 (t, 1H) 3.39 (m, 1H) 3.86 (m, |
| | 6-amine | | 1H) 4.09 (m, 1H) 4.45 (dd, 2H) 4.81 (s, 1H) 6.08 (m, |
| | | | 1H) 6.77 (bs, 2H) 7.84 (s, 1H) |
| 101 | 2-{[(2S)-1-methylpyrrolidin-2- | 319 | 1.65 (m, 4H) 1.95 (mm, 2H) 2.15 (m, 2H) 2.34 (s, 3H) |
| | yl]methoxy}-9-(tetrahydrofuran- | | 2.40 (m, 2H) 2.93 (m, 1H) 3.88 (m, 1H) 4.08 (m, 2H) |
| | 2-yl)-9H-purin-6-amine | | 4.19 (m, 1H) 6.13 (m, 1H) 7.24 (bs, 2H) 8.04 (s, 1H) |
| 102 | 2-(piperidin-4-ylmethoxy)-9- | 319 | 1.03 (m, 2H) 1.63 (m, 3H) 1.98 (m, 1H) 2.22 (m, 1H) |
| | (tetrahydrofuran-2-yl)-9H-purin- | | 2.34 (m, 2H) 2.72 (m, 2H) 3.24 (m, 2H) 3.86 (m, 1H) |
| | 6-amine | | 4.08 (m, 2H) 4.43 (m, 1H) 4.65 (m, 2H) 6.07 (m, 1H) |
| | | | 6.74 (bs, 2H) 7.82 (s, 1H) |
| 103 | 2-(pyridin-3-yloxy)-9- | 299 | 1.88 (m, 2H) 2.31 (m, 2H) 3.77 (m, 1H) 3.86 (m, 1H) |
| | (tetrahydrofuran-2-yl)-9H-purin- | | 6.05 (m, 1H) 7.45 (mm, 3H) 7.64 (m, 1H) 8.09 (s, 1H) |
| | 6-amine | | 8.39 (m, 1H) 8.46 (m, 1H) |
| 104 | 2-(quinolin-5-yloxy)-9- | 349 | 1.56 (mm, 2H) 2.13 (m, 2H) 3.55 (m, 2H) 5.97 (m, |
| | (tetrahydrofuran-2-yl)-9H-purin- | | 1H) 7.14 (mm, 4H) 7.77 (m, 1H) 7.90 (m, 1H) |
| | 6-amine | | 7.77 (m, 1H) 8.05 (s, 1H) 8.20 (d, 1H) 8.92 (m, 1H) |
| 105 | 2-(2-morpholin-4-ylethoxy)-9- | 335 | 1.99 (m, 1H) 2.22 (m, 1H) 2.44-2.30 (m, 7H) 2.63 (m, |
| | (tetrahydrofuran-2-yl)-9H-purin- | | 2H) 3.55 (m, 4H) 3.88 (m, 1H) 4.10 (m, 1H) 4.31 (m, |
| | 6-amine | | 2H) 6.11 (m, 1H) 7.23 (bs, 2H) 8.03 (s, 1H) |
| 106 | 2-(2,3-dihydro-1H-inden-5- | 338 | 1.86 (mm, 2H) 2.03 (m, 2H) 2.83 (m, 4H) 3.78 (m, |
| | yloxy)-9-(tetrahydrofuran-2-yl)- | | 1H) 3.92 (m, 1H) 6.05 (m, 1H) 6.84 (d, 1H) 6.96 (s, |
| | 9H-purin-6-amine | | 1H) 7.19 (d, 1H) 7.36 (bs, 1H) 8.05 (s, 1H) |
| 107 | 2-(2-naphthyloxy)-9- | 348 | 1.84 (m, 2H) 2.31 (m, 2H) 3.87 (m, 3H) 6.03 (m, 1H) |
| | (tetrahydrofuran-2-yl)-9H-purin- | | 7.42 (mm, 4H) 7.64 (bs, 1H) 7.92 (m, 3H) 8.09 (s, 1H) |
| | 6-amine | | |
| 108 | 2-(3-tert-butoxypropoxy)-9- | 336 | 1.11 (s, 9H) 1.82 (m, 2H) 2.23 (m, 1H) 2.37 (m, 2H) |
| | (tetrahydrofuran-2-yl)-9H-purin- | | 3.41 (m, 2H) 3.88 (m, 1H) 4.11 (m, 1H) 4.23 (t, 2H) |
| | 6-amine | | 6.11 (m, 1H) 7.22 (bs, 2H) 8.02 (s, 1H) |
| 109 | 2-[(1-methyl-1H-imidazol-5- | 316 | 2.00 (m, 1H) 2.23 (m, 1H) 2.41 (m, 2H) 3.64 (s, 3H) |
| | yl)methoxy]-9-(tetrahydrofuran-2- | | 3.88 (m, 1H) 4.11 (m, 1H) 5.27 (s, 2H) 6.15 (m, 1H) |
| | yl)-9H-purin-6-amine | | 7.03 (s, 1H) 7.32 (bs, 2H) 7.64 (s, 1H) 8.06 (s, 1H) |
| 110 | 2-(hex-3-yn-1-yloxy)-9- | 302 | 1.02 (t, 3H) 1.99 (m, 2H) 2.13 (m, 2H) 2.23 (m, 1H) |
| | (tetrahydrofuran-2-yl)-9H-purin- | | 2.40 (mm, 2H) 2.56 (m, 2H) 3.88 (m, 1H) 4.10 (m, 1H) |
| | 6-amine | | 4.23 (t, 2H) 6.11 (m, 1H) 7.26 (bs, 2H) 8.04 (s, 1H) |

### EXAMPLE 111

### 1-[6-amino-9-(tetrahydrofuran-2-yl)-9H-purin-2-yl]pentan-1-one

1-[6-chloro-9-(tetrahydrofuran-2-yl)-9H-purin-2-yl]pentan-1-one (18 mg) in MeOH (1 ml) was treated with 7N ammonia in MeOH (4 ml). The reaction was heated to 120° C. for 0.5 h in the microwave reactor. The volatiles were removed in vacuo. The residue was purified by flash chromatography using 10% MeOH in EtOAc as the eluent. Relevant fractions were pooled giving 6.5 mg of the desired product.

MS (ESP): 290.15 (MH⁺) for C₁₄H₁₉N₅O₂

¹H NMR (300 MHz, MeOD) δ ppm 0.88-1.00 (m, 3H) 1.35-1.48 (m, 2H) 1.63-1.76 (m, 2H) 2.09-2.28 (m, 2H) 2.46-2.60 (m, 2H) 3.14-3.27 (m, 2H) 3.98-4.10 (m, 1H) 4.31 (m, 1H) 6.38 (dd, 1H) 8.31-8.40 (m, 1H)

The intermediates for this compound were made as follows:—

### 1-[6-chloro-9-(tetrahydrofuran-2-yl)-9H-purin-2-yl]pentan-1-one

6-Chloro-9-(tetrahydrofuran-2-yl)-2-(tributylstannyl)-9H-purine (204 mg), pentanoyl chloride (64 mg), and dichloro-bis(triphenylphosphine)palladium (II) in toluene (5 ml) were heated at 100° C. for 0.5 h. The reaction mixture was then poured into a solution of EtOAc (15 ml) and 5% aqueous potassium fluoride (10 ml). The salts were removed by filtration and the filtrate was extracted with EtOAc. After removing the volatiles in vacuo, the resulting material was purified by Gilson reverse phase HPLC. Relevant fractions were combined to give 19 mg of the desired product.

MS (ESP): 309.10 (MH⁺) for C₁₄H₁₇ClN₄O₂

¹H NMR (300 MHz, MeOD) δ ppm 0.98 (t, 3H) 1.38-1.52 (m, 2H) 1.68-1.80 (m, 2H) 2.15-2.32 (m, 3H) 2.52-2.66 (m, 3H) 4.08 (q, 1H) 4.36 (m, 1H) 6.50 (dd, 1H) 8.79 (s, 1H)

### 6-chloro-9-(tetrahydrofuran-2-yl)-2-(tributylstannyl)-9H-purine

n-Butyllithium (1.6M in hexane) (15.6 ml) was added dropwise to a solution of tetramethylpiperidine (3.52 ml) in THF (40 ml), at rt. After stirring for 10 min, the solution was cooled to −78° C. and a solution of 6-chloro-9-(tetrahydrofuran-2-yl)-purine (made using essentially the procedure described in Example 1 for 2,6-dichloro-9-(tetrahydrofuran-2-yl) purine starting from 6-chloro-9-(tetrahydrofuran-2-yl)-9H-purine) (1.12 g)) in THF (15 ml) was added dropwise. The reaction mixture was stirred at −78° C. for 1 h then tri-n-butyltinchloride (8.125 g) was added dropwise while maintaining the temperature at −78° C. The reaction mixture was stirred at −78° C. for 0.5 h then quenched by addition of aqueous ammonium chloride. The organic layer was washed with 5% aqueous sodium bicarbonate and dried over sodium sulfate. Purification by flash chromatography using 20% EtOAc in hexanes resulted in 2.17 g of the desired product.

MS (ESP): 515.15 (MH⁺) for C₂₁H₃₅ClN₄OSn

¹H NMR δ: 0.76-0.89 (m, 9H) 1.03-1.17 (m, 5H) 1.20-1.35 (m, 7H) 1.50-1.64 (m, 6H) 1.98-2.10 (m, 1H) 2.21-2.33 (m, 1H) 2.36-2.47 (m, 1H) 2.52-2.64 (m, 1H) 3.91-4.00 (m, 1H) 4.18 (m, 1H) 6.36 (dd, 1H) 8.67 (s, 1H).

### EXAMPLE 112

### 1-[6-amino-9-(tetrahydrofuran-2-yl)-9H-purin-2-yl]-2-methylbutan-1-one

Using essentially the same procedure described for Example 111, starting with 6-chloro-9-(tetrahydrofuran-2-yl)-2-(tributylstannyl)-9H-purine (306 mg) and 3-methylbutanoyl chloride (96 mg), 14 mg of the desired compound was obtained.

MS (ESP): 290.15 (MH⁺) for C₁₄H₁₉N₅O₂

¹H NMR (300 MHz, MeOD) δ ppm 0.85-0.98 (m, 3H) 1.08-1.22 (m, 3H) 1.49 (m, 1H) 1.81 (m, 1H) 2.09-2.32 (m, 2H) 2.46-2.62 (m, 2H) 3.93-4.08 (m, 2H) 4.30 (m, 1H) 6.37 (dd, J=6.41, 3.96 Hz, 1H) 8.33 (s, 1H)

### EXAMPLE 113

### [6-amino-9-(tetrahydrofuran-2-yl)-9H-purin-2-yl](cyclopropyl)methanone

Using essentially the same procedure described for Example 111, starting with 6-chloro-9-(tetrahydrofuran-2-yl)-2-(tributylstannyl)-9H-purine (306 mg) and cyclopropanecarbonyl chloride (90 mg), 8 mg of the desired compound was obtained.

MS (ESP): 274.15 (MH⁺) for C₁₃H₁₅N₅O₂

¹H NMR (300 MHz, MeOD) δ ppm 1.16 (m, 4H) 2.15-2.26 (m, 2H) 2.53 (m, 2H) 3.50 (m, 1H) 4.05 (m, 1H) 4.30 (m, 1H) 6.40 (dd, 1H) 8.34 (s, 1H)

### EXAMPLE 114

### 2-pyridin-2-yl-9-(tetrahydrofuran-2-yl)-9H-purin-6-amine

6-Chloro-9-(tetrahydrofuran-2-yl)-2-(tributylstannyl)-9H-purine (prepared as in Example 111) (200 mg), 2-iodopyridine (0.25 ml), palladium tetrakis(triphenylphosphine) (115 mg), and copper (I) iodine (40 mg) were suspended in toluene (5 ml). The reaction mixture was heated to 100° C. for 5 h. The mixture was then diluted with DCM and filtered through diatomaceous earth. The residue was chromatographed using 0-20% MeOH in EtOAc as eluent giving 6-chloro-2-pyridin-2-yl-9-(tetrahydrofuran-2-yl)-9H-purine. 6-Chloro-2-pyridin-2-yl-9-(tetrahydrofuran-2-yl)-9H-purine in MeOH (3 ml) was treated with 7N ammonia in MeOH (15 ml). The reaction was heated to 110° C. for 0.5 h in the microwave reactor. The volatiles were removed in vacuo. The residue was purified by Gilson reverse phase HPLC. Relevant fractions were combined giving 2.5 mg of the desired product.

MS (ESP): 283.16 (MH⁺) for C₁₄H₁₄N₆O

¹H NMR (300 MHz, MeOD) δ ppm 2.13-2.20 (m, 1H) 2.22-2.33 (m, 1H) 2.48-2.61 (m, 2H) 4.00-4.09 (m, 1H) 4.27-4.39 (m, 1H) 6.45-6.54 (m, 1H) 7.48 (m, 1H) 7.95 (m, 1H) 8.26 (s, 1H) 8.49 (d, 1H) 8.68 (d, 1H)

### EXAMPLE 115

### 2-[(E)-2-phenylvinyl]-9-β-D-ribofuranosyl-9H-purin-6-amine

2-Chloroadenosine (200 mg), [(E)-2-phenylvinyl]boronic acid (196 mg), palladium tetrakis(triphenylphosphine) (152 mg), and sodium carbonate (425 mg) were suspended in a mixture of dioxane (5 ml) and water (1 ml). The reaction was heated to 150° C. for 0.5 h in a microwave reactor. The resulting reaction mixture was diluted with DCM and filtered through diatomaceous earth; the filtrate was washed with water and dried over sodium sulfate. The residue was purified by Gilson reverse phase HPLC. Relevant fractions were combined to give the desired product.

MS (ESP): 370.11 (MH⁺) for C₁₈H₁₉N₅O₄

¹H NMR (300 MHz, MeOD) δ ppm 2.10 (d, 1H) 3.70-3.84 (dd, 1H) 3.89-3.98 (dd, 1H) 4.21 (q, 1H) 4.37 (dd, 1H) 4.62 (s, 1H) 5.97 (d, 1H) 7.05 (d, 1H) 7.29-7.42 (m, 3H) 7.51-7.64 (m, 4H) 7.70-7.82 (m, 1H) 7.88 (d, 1H) 8.25 (s, 1H)

Using an analogous procedure to that described in Example 115, the appropriate commercially available boronic acid was reacted with either 2-chloro-9-(tetrahydrofuran-2-yl)-9H-purin-6-amine or 2-chloroadenosine to give the compounds described in Table III. The products were isolated by RP-HPLC or flash chromatography.

TABLE III

| EX | IUPAC Name | MH+ | ¹H-NMR (300 MHz, MeOD) δ ppm |
|---|---|---|---|
| 116 | 2-(2-furyl)-9-(tetrahydrofuran-2- | 272 | 2.01-2.63 (m, 4H) 3.93-4.13 (m, 1H) |
| | yl)-9H-purin-6-amine | | 4.24-4.41 (m, 1H) 6.30-6.45 (m, 1H) 6.59 (dd, 1H) 7.19 (d, |
| | | | 1H) 7.60-7.72 (m, 1H) 8.17 (s, 1H). |
| 117 | 9-(tetrahydrofuran-2-yl)-2-(2- | 288 | 2.03-2.77 (m, 4H) 3.95-4.16 (m, 1H) |
| | thienyl)-9H-purin-6-amine | | 4.28-4.46 (m, 1H) 6.31 (dd, 1H) 7.09 (dd, 1H) 7.47 (dd, 1H) |
| | | | 7.87 (dd, 1H) 8.13 (s, 1H) |
| 118 | 2-[(1E)-hex-1-en-1-yl]-9- | 288 | 0.86 (t, 3H) 1.22-1.59 (m, 4H) 1.95-2.55 (m, 6H) |
| | (tetrahydrofuran-2-yl)-9H-purin-6- | | 3.82-4.02 (m, 1H) 4.10-4.27 (m, 1H) |
| | amine | | 6.06-6.41 (m, 2H) 6.80-7.10 (m, 1H) 8.04 (s, 1H). |
| 119 | 2-[(E)-2-phenylvinyl]-9- | 308 | 2.08-2.36 (m, 2H) 2.46-2.63 (m, 2H) |
| | (tetrahydrofuran-2-yl)-9H-purin-6- | | 3.92-4.13 (m, 1H) 4.21-4.41 (m, 1H) 6.35 (dd, 1H) 7.09 (d, |
| | amine | | 1H) 7.25-7.45 (m, 3H) 7.53-7.68 (m, 2H) |
| | | | 7.86 (d, 1H) 8.17 (s, 1H) |
| 120 | 2-[(1E)-pent-1-en-1-yl]-9- | 274.23 | 0.98 (t, 3H) 1.48-1.60 (m, 2H) 2.09-2.16 (m, 1H) |
| | (tetrahydrofuran-2-yl)-9H-purin-6- | | 2.23 (m, 3H) 2.43-2.54 (m, 2H) 3.96-4.05 (m, |
| | amine | | 1H) 4.27 (m, 1H) 6.27-6.40 (m, 2H) |
| | | | 6.96-7.07 (dt, 1H) 8.13 (s, 1H) |
| 121 | 2-[(1E)-pent-1-en-1-yl]-9-b-D- | 336.20 | 0.87-1.00 (t, 3H) 1.46-1.60 (m, 2H) |
| | ribofuranosyl-9H-purin-6-amine | | 2.16-2.28 (m, 2H) 3.64-3.78 (dd, 1H) 3.83-3.93 (dd, 1H) |
| | | | 4.16 (m, 1H) 4.32 (dd, 1H) 4.79 (dd, 2H) 5.92 (d, |
| | | | 1H) 6.28-6.39 (dm, 1H) 6.96-7.08 (m, 1H) |
| | | | 8.19 (s, 1H) |

### EXAMPLE 122

### 2-(2-phenylethyl)-9-β-D-ribofuranosyl-9H-purin-6-amine

2-[(E)-2-phenylvinyl]-9-β-D-ribofuranosyl-9H-purin-6-amine (residue as prepared in Ex. 115) was dissolved in EtOAc and MeOH. Palladium (10%) on carbon (100 mg) was added and the mixture hydrogenated using 1 atm hydrogen at rt overnight. The catalyst was removed by filtration and the solvent removed in vacuo. The residue was purified by Gilson reverse phase HPLC. Relevant fractions were combined to give the desired product.

MS (ESP): 372.17 (MH⁺) for C₁₈H₂₁N₅O₄

¹H NMR (300 MHz, MeOD) δ ppm 2.95-3.09 (m, 4H) 3.67-3.78 (dd, 1H) 3.80-3.93 (dd 1H) 4.10-4.22 (m, 1H) 4.31 (m, 1H) 4.62 (s, 1H) 4.76 (dd, 2H) 5.90 (d, 1H) 7.11-7.25 (m, 5H) 8.19 (s, 1H)

Using an analogous procedure to that described in Example 122, the appropriate unsaturated substrate (Examples in Table III) was hydrogenated to give the compounds described in Table IV.

TABLE IV

| EX | IUPAC Name | MH+ | ¹H-NMR (300 MHz, MeOD) d ppm |
|---|---|---|---|
| 123 | 2-hexyl-9-(tetrahydrofuran-2- | 390 | (0.80 (t, 3H) 1.08-1.41 (m, 6H) 1.57-1.81 (m, 2H) |
| | yl)-9H-purin-6-amine | | 1.93-2.25 (m, 2H) 2.32-2.49 (m, 2H) |
| | | | 2.53-2.74 (m, 2H) 3.84-4.02 (m, 1H) 4.05-4.33 (m, 1H) |
| | | | 6.20 (t, 1H) 8.04 (s, 1H) |
| 124 | 2-(2-phenylethyl)-9- | 310 | 1.99-2.29 (m, 2H) 2.45-2.60 (m, 2H) |
| | (tetrahydrofuran-2-yl)-9H- | | 2.90-3.21 (m, 4H) 3.89-4.10 (m, 1H) 4.18-4.36 (m, 1H) |
| | purin-6-amine | | 6.27 (t, 1H) 7.02-7.35 (m, 5H) 8.12 (s, 1H) |
| 125 | 2-pentyl-9-(tetrahydrofuran-2- | 276 | 0.90 (t, 3H) 1.23-1.49 (m, 4H) 1.71-1.88 (m, 2H) |
| | yl)-9H-purin-6-amine | | 2.03-2.30 (m, 2H) 2.43-2.59 (m, 2H) |
| | | | 2.67-2.87 (m, 2H) 3.88-4.10 (m, 1H) 4.22-4.36 (m, 1H) |
| | | | 6.29 (t, 1H) 8.13 (s, 1H). |
| 126 | 2-pentyl-9-beta-D- | 338 | 0.90 (t, 3H) 1.22-1.49 (m, 4H) 1.63-1.89 (m, 2H) |
| | ribofuranosyl-9H-purin-6- | | 2.60-2.80 (m, 2H) 3.62-3.98 (m, 2H) 4.18 (d, H) |
| | amine | | 4.31 (dd, 1H) 4.80 (dd, 1H) 5.90 (d, 1H) 8.18 (s, 1H) |

### EXAMPLE 127

### 9-(3-chlorotetrahydrofuran-2-yl)-2-(cyclopentyloxy)-9H-purin-6-amine

A suspension of N-[2-(cyclopentyloxy)-9H-purin-6-yl]benzamide (100 mg, 0.31 mmol), 2,3-dichlorotetrahydrofuran (86.6 mg, 0.62 mmol) and N,O-bis(trimethylsilyl)acetamide (382 μl, 1.55 mmol) in 4 mL dry acetonitrile was warmed to 60° C. After stirring for 30 min, 253 μl (2.2 mmol) tin (IV) chloride was added dropwise and stirring was continued for another 60 min. The reaction mixture was cooled to rt and poured into a mixture of cold saturated sodium bicarbonate and EtOAc (1:1, v/v, 100 ml). The aqueous phase was extracted with EtOAc (50 ml). The organic phases were combined and washed with saturated sodium bicarbonate, dried (sodium sulfate) and evaporated to dryness. This intermediate was taken up in a 1:1 mixture of methylamine (2 ml, 2 M in MeOH) and ammonia (2 ml, 30% in water), then stirred for 4 h. The solution was concentrated and the residue was purified by chromatography eluting with 5% MeOH in DCM to give desired product (20 mg).

MS (ESP): 324 (MH⁺) for C₁₄H₁₈ClN₅O₂

¹H NMR δ: 1.58 (s, 2H) 1.71 (s, 4H) 1.90 (s, 2H) 2.27 (s, 2H) 2.81 (s, 1H) 4.16 (s, 1H) 4.40 (s, 1H) 5.13 (s, 1H) 5.26 (s, 1H) 6.12 (s, 1H) 7.25 (s, 2H) 8.04 (s, 1H)

The intermediate for this compound was prepared as follows:—

### N-[2-(cyclopentyloxy)-9H-purin-6-yl]benzamide

To a solution of 2-(cyclopentyloxy)-9-(tetrahydrofuran-2-yl)-9H-purin-6-amine (4 g, 13.4 mmol) in dry pyridine was added benzoyl chloride at 4° C. The solution was stirred at rt overnight, then quenched with MeOH. The solution was diluted with DCM (500 ml), and washed with brine. The organic phase was dried (sodium sulfate), filtered and concentrated in vacuo. The resulting residue was taken up DCM (20 ml) and trifluoroacetic acid (10 ml). The solution was stirred for 1 h at rt and concentrated in vacuo. The residue was purified by chromatography eluting with 80% EtOAc in hexane to give the desired product (2.5 g).

MS (ESP): 324 (MH⁺) for C₁₇H₁₇N₅O₂

¹H NMR δ: 1.41-1.72 (m, 8H) 5.19 (m, 1H) 7.32-7.45 (m, 3H) 7.88 (m, 2H) 8.16 (s, 1H) 11.23 (s, 1H)

### EXAMPLE 128

### 9-(3-aminotetrahydrofuran-2-yl)-2-(cyclopentyloxy)-9H-purin-6-amine

A suspension of 9-(3-chlorotetrahydrofuran-2-yl)-2-(cyclopentyloxy)-9H-purin-6-amine (79 mg, 0.24 mM), sodium azide (37.6 mg, 0.73 mmol) and sodium iodide (50 mg, 0.33 mmol)) in 1-methyl-2-pyrrolidinone (1 ml) was heated in a microwave reactor for 1 h at 160° C. The reaction mixture was diluted with DCM (40 ml) and washed with water. The organic phase was dried (sodium sulfate) and evaporated to dryness. This intermediate was taken up in ethanol (2 ml) and 10% palladium on charcoal (50 mg) was added. The reaction mixture was stirred under hydrogen (1 atm) for 3 h, diluted with ethanol, filtered through diatomaceous earth and evaporated. The residue was purified using Gilson reverse phase HPLC with 10 mM ammonium acetate and acetonitrile as the mobile phases with a gradient of 10-70% in 15 min. Relevant fractions were combined to give 10 mg of the desired product.

MS (ESP): 305 (MH⁺) for C₁₄H₂₀N₆O₂

¹H NMR δ: 1.50-1.86 (m, 10H) 3.88-4.01 (m, 2H) 4.11 (q, 1H) 5.11-5.24 (m, 1H) 5.52 (d, 1H) 7.05-7.19 (m, 2H) 7.93 (s, 1H)

### EXAMPLE 129

### 2-(cyclopentyloxy)-9-[5-O-(2-hydroxyethyl)-β-D-ribofuranosyl]-9H-purin-6-amine

A solution of 2-(cyclopentyloxy)-9-[5-O-(2-hydroxyethyl)-2,3-O-(1-methylethylidene)-β-D-ribofuranosyl]-9H-purin-6-amine (115 mg, 0.26 mmol) in acetic acid (5 ml, 80% in water) was heated at 80° C. for 7 h. The reaction mixture was concentrated to dryness and the residue was purified by chromatography eluting with 7% MeOH in DCM to give the desired product (40 mg).

MS (ESP): 396 (MH⁺) for C₁₇H₂₅N₅O₆

¹H NMR δ: 1.51-1.69 (m, 6H) 1.80-1.93 (m, 2H) 3.39-3.60 (m, 6H) 3.81-3.86 (m, 1H) 4.04-4.09 (m, 1H) 4.53 (q, 1H) 4.66-4.71 (m, 1H) 5.04 (t, 1H) 5.10 (d, 1H) 5.21 (s, 1H) 5.34 (d, 1H) 5.70 (d, 1H) 7.58 (s, 1H) 8.06 (s, 1H)

The intermediates for this compound were prepared as follows:—

### 2-(cyclopentyloxy)-9-[5-O-(2-hydroxyethyl)-2,3-O-(1-methylethylidene)-β-D-ribofuranosyl]-9H-purin-6-amine

A suspension of N-benzoyl-2-(cyclopentyloxy)-9-[2,3-O-(1-methylethylidene)-β-D-ribofuranosyl]-9H-purin-6-amine (200 mg, 0.40 mmol), ethylene carbonate (53 mg, 0.61 mmol) and potassium carbonate (112 mg, 0.81 mmol) in DMF was heated at 110° C. for 2 h. The reaction mixture was cooled to rt and diluted with DCM (10 ml). The white solid was filtered off and the filtrated was concentrated to dryness. The resulting product was taken up in a 1:1 mixture of methylamine (2 ml, 2 M in MeOH) and ammonia (2 ml, 30% in water), and then stirred for 4 h. The solution was concentrated and the residue was purified by chromatography eluting with 5% MeOH in DCM to give the desired product (117 mg).

MS (ESP): 436 (MH⁺) for C₂₀H₂₉N₅O₆

¹H NMR δ: 1.34 (s, 3H) 1.55 (s, 3H) 1.55-1.73 (m, 8H) 3.44-3.59 (m, 7H) 4.13-4.19 (m, 1H) 4.97 (dd, 1H) 5.10 (t, 1H) 5.24-5.31 (m, 1H) 5.40 (dd, 1H) 6.06 (d, 1H) 7.71 (s, 1H) 8.13 (s, 1H)

### N-benzoyl-2-(cyclopentyloxy)-9-[2,3-O-(1-methylethylidene)-β-D-ribofuranosyl]-9H-purin-6-amine

To a solution of N-benzoyl-2-(cyclopentyloxy)-9-β-D-ribofuranosyl-9H-purin-6-amine (1.5 g, 3.3 mmol) and dimethoxypropane (408 μl, 3.3 mmol) in dry acetone (20 ml) was added perchloric acid (400 μl) at 4° C. The solution was stirred for 6 h at 4° C., then neutralized with ammonium hydroxide and concentrated to dryness. The residue was purified by chromatography eluting with 7% MeOH in DCM to give the desired product (1.0 g).

MS (ESP): 496 (MH⁺) for C₂₂H₂₅N₅O₆

¹H NMR (300 MHz, CDCl₃) δ ppm 1.31 (s, 3H) 1.45-1.60 (m, 2H) 1.57 (s, 3H) 1.70-1.98 (m, 6H) 3.74 (dd, 1H) 3.90 (dd, 1H) 4.42 (d, 1H) 5.05 (dd, 1H) 5.22 (dd, 1H) 5.32 (tt, 1H) 5.86 (d, 1H) 7.40-7.48 (m, 2H) 7.50-7.57 (m, 1H) 7.85-7.95 (m, 2H) 8.01 (s, 1H)

### N-benzoyl-2-(cyclopentyloxy)-9-β-D-ribofuranosyl-9H-purin-6-amine

To a solution of N-benzoyl-2-(cyclopentyloxy)-9-(2,3,5-tri-O-benzoyl-β-D-ribofuranosyl)-9H-purin-6-amine (6.1 g, 7.9 mmol) in 90 mL of a mixture of THF/MeOH/water (5:4:1) was added dropwise 15 mL of aqueous sodium hydroxide (1N) at 4° C. The solution was stirred for 3 h at rt then neutralized with Amberlite (IR-120⁺). The mixture was filtered and the filtrate was concentrated to dryness. The residue was purified by chromatography eluting with 5% MeOH in DCM to give the desired product (1.8 g).

MS (ESP): 456 (MH⁺) for C₂₂H₂₅N₅O₆

¹H NMR (300 MHz, CDCl₃) δ ppm 1.44-1.92 (m, 8H) 3.76 (m, 2H) 4.10 (m, 1H) 4.45 (m, 1H) 4.57-4.72 (m, 1H) 5.25 (m, 1H) 6.14 (d, 1H) 7.23-7.31 (m, 3H) 7.33-7.89 (m, 3H) 9.98 (s, 1H)

### N-benzoyl-2-(cyclopentyloxy)-9-(2,3,5-tri-O-benzoyl-β-D-ribofuranosyl)-9H-purin-6-amine

A suspension of N-[2-(cyclopentyloxy)-9H-purin-6-yl]benzamide (5.0 g, 15.5 mmol), 1-O-acetyl-2,3,5-tri-O-benzoyl-D-ribofuranose (4.68 g, 9.2 mmol) and N,O-bis(trimethylsilyl)acetamide (9.5 ml, 36.7 mmol) in 100 mL dry acetonitrile was warmed to 60° C. After stirring for 30 min, 6.8 mL (58.0 mmol) tin (IV) chloride was added dropwise and stirring was continued for another 60 min. The reaction mixture was cooled to rt and poured into a mixture of cold saturated sodium bicarbonate and EtOAc (1:1, v/v, 1000 ml). The aqueous phase was extracted with EtOAc (500 ml). The organic phase were combined and washed with saturated sodium bicarbonate, dried (sodium sulfate) and evaporated to dryness. The residue was purified by chromatography eluting with 50% EtOAc in hexane to give the desired product (6.1 g).

MS (ESP): 768 (MH⁺) for C₄₃H₃₇N₅O₉

¹H NMR δ: 1.63-2.06 (m, 8H) 4.71-4.86 (m, 2H) 4.89-4.99 (m, 1H) 5.48-5.60 (m, 1H) 6.40 (m, 1H) 6.59-6.71 (m, 1H) 6.68 (d, 1H) 7.49-8.04 (m, 20H) 8.53 (s, 1H) 11.23 (s, 1H)

### EXAMPLE 130

### 2-(cyclopentyloxy)-9-[5-O-(5-hydroxypentyl)-β-D-ribofuranosyl]-9H-purin-6-amine

A suspension of N-benzoyl-2-(cyclopentyloxy)-9-[2,3-O-(1-methylethylidene)-β-D-ribofuranosyl]-9H-purin-6-amine (100 mg, 0.2 mmol), 5-bromo-1-pentanol (169 mg, 1.01 mmol) and potassium carbonate (84 mg, 0.61 mmol) in DMF was heated at 100° C. for 4 h. The reaction mixture was cooled to rt and diluted with DCM (10 ml). The white solid was filtered off and the filtrated was concentrated to dryness. The resulting product was taken up in a 1:1 mixture of methylamine (2 ml, 2M in MeOH) and ammonia (2 ml, 30% in water), and stirred for 5 h. The solution was concentrated to dryness and the residue was dissolved in acetic acid (5 ml, 80% in water). The reaction mixture was heated at 80° C. for 7 h, concentrated to dryness, and the residue was purified by chromatography over silica gel eluting with 10% MeOH in DCM to give the desired product (10 mg).

MS (ESP): 438 (MH⁺) for C₂₀H₃₁N₅O₆

¹H NMR (300 MHz, DMSO-D6) δ ppm 1.22-1.86 (m, 16H) 3.38-3.61 (m, 2H) 3.83 (q, 1H) 4.06 (dt, 1H) 4.29 (t, 1H) 4.46-4.59 (dq, 1H) 5.11 (m, 2H) 5.21 (m, 1H) 5.35 (d, 1H) 5.69 (d, 1H) 7.79 (s, 2H) 8.05 (s, 1H).

### EXAMPLE 131

### 2-(cyclopentyloxy)-9-(5′-deoxy-β-D-ribofuranosyl)-9H-purin-6-amine

2-(Cyclopentyloxy)-9-[5-deoxy-2,3-O-(1-methylethylidene)-β-D-ribofuranosyl]-9H-purin-6-amine (0.6 mmol) was dissolved in 1:1 water/acetic acid (4 mL total). Formic acid (0.2 ml) was added and the reaction mixture heated to 95° C. for 2 h. After cooling to rt, the solution was neutralized with sodium bicarbonate. The residue was purified by flash chromatography using 10% MeOH in chloroform as eluent. The product was further purified using Gilson reverse phase HPLC with 10 mM ammonium acetate and acetonitrile as the mobile phases with a gradient of 5-75% in 10 min. Relevant fractions were combined to give 20 mg of the desired product.

MS (ESP): 336 (MH⁺) for C₁₅H₂₁N₅O₄

¹H NMR δ: 1.27 (d, 3H) 1.59 (m, 2H) 1.69 (m, 4H) 1.89 (m, 2H) 3.87-4.02 (m, 2H) 4.66 (m, 1H) 5.16 (m, 1H) 5.25 (m, 1H) 5.42 (m, 1H) 5.70 (d, 1H) 7.19 (s, 2H) 8.08 (s, 1H)

The intermediates for this compound were prepared as follows:—

### 2-(cyclopentyloxy)-9-[5-deoxy-2,3-O-(1-methylethylidene)-β-D-ribofuranosyl]-9H-purin-6-amine

Cyclopentanol (2 ml) was added to 2-chloro-9-[5-deoxy-2,3-O-(1-methylethylidene)-β-D-ribofuranosyl]-9H-purin-6-amine (0.2 g, 0.61 mmol) and sodium hydroxide (0.25 g, 6.1 mmol). The flask was sealed and the reaction was heated to 65° C. for 2 days. After cooling to rt, excess sodium hydroxide was filtered off and the volatiles removed in vacuo. The resulting residue was purified by using 0-5% MeOH in DCM as eluent. The resulting brown oil was used directly in the subsequent step.

MS (ESP): 376 (MH⁺) for C₁₈H₂₅N₅O₄

### 2-chloro-9-[5-deoxy-2,3-O-(1-methylethylidene)-β-D-ribofuranosyl]-9H-purin-6-amine

2-Chloro-9-{2,3-O-(1-methylethylidene)-5-O-[(4-methylphenyl)sulfonyl]-β-D-ribofuranosyl}-9H-purin-6-amine (J. Med. Chem. 1974, 17(11), 1197-1207) (0.29 g, 0.58 mmol) was treated with lithium triethylborohydride (1M in THF) (5 ml). After 25 min, the reaction was quenched by addition of 0.5 mL water. The volatiles were concentrated in vacuo. The resulting residue was dissolved in DCM (50 ml) and washed with water (2×) and brine and dried over sodium sulfate. Purification by chromatography using 0-4% MeOH in DCM as eluent gave the desired product as a yellow solid.

MS (ESP): 326 (MH⁺) for C₁₃H₁₆ClN₅O₃

¹H NMR δ: ppm 1.25 (d, 3H) 1.31 (s, 3H) 1.51 (s, 3H) 4.24 (m, 1H) 4.76 (dd, 1H) 5.37 (dd, 1H) 6.02 (d, 1H) 7.88 (s, 2H) 8.34 (s, 1H)

### EXAMPLE 132

### 2-(cyclobutylmethoxy)-9-(5-deoxy-β-D-ribofuranosyl)-9H-purin-6-amine

Cyclobutane MeOH (1 ml) was added to 2-chloro-9-[5-deoxy-2,3-O-(1-methylethylidene)-β-D-ribofuranosyl]-9H-purin-6-amine (200 mg, 0.62 mmol) and sodium hydroxide (246 mg, 6.2 mmol). The flask was sealed and the reaction was heated to 75° C. for 3 days. After cooling to rt, excess sodium hydroxide was filtered off and washed with DCM. The solution was washed with water, dried (sodium sulfate), filtered and concentrated in vacuo. The resulting residue was dissolved in 1:1.5 water/acetic acid (10 mL total). Formic acid (1 ml) was added and the reaction mixture heated to 85° C. for 3 h. The reaction mixture was concentrated in vacuo and the residue was purified using Gilson reverse phase HPLC with 10 mM ammonium acetate and acetonitrile as the mobile phases with a gradient of 10-50% in 10 min. Relevant fractions were combined to give 90 mg of the desired product.

MS (ESP): 336 (MH⁺) for C₁₅H₂₁N₅O₄

¹H NMR δ: 1.22 (d, 3H) 1.70-1.86 (m, 4H) 1.90-2.02 (m, 2H) 2.56-2.68 (m, 1H) 3.83-3.93 (m, 2H) 4.11 (d, 2H) 4.54-4.60 (m, 1H) 5.08 (d, 1H) 5.34 (d, 1H) 5.66 (d, 1H) 7.19 (s, 2H) 8.04 (s, 1H)

### EXAMPLE 133

### 2-(decahydronaphthalen-2-yloxy)-9-(5-deoxy-β-D-ribofuranosyl)-9H-purin-6-amine

Using essentially the same procedure as Example 132, starting with decahydronaphthalen-2-ol (1 ml), 2-chloro-9-[5-deoxy-2,3-O-(1-methylethylidene)-β-D-ribofuranosyl]-9H-purin-6-amine (250 mg, 0.77 mmol), and sodium hydroxide (308 mg, 7.7 mmol), the desired product (75 mg) was obtained.

MS (ESP): 404 (MH⁺) for C₂₀H₂₉N₅O₄

¹H NMR δ: 0.82-1.84 (m, 16H) 1.20 (d, 3H) 3.81-3.96 (m, 2H) 4.56-4.77 (m, 2H) 5.03 (d, 1H) 5.36 (m, 1H) 5.56-5.70 (m, 1H) 7.04-7.19 (m, 2H) 8.01 (s, 1H)

### EXAMPLE 134

### 9-(5-deoxy-β-D-ribofuranosyl)-2-[(cis-4-methylcyclohexyl)oxy]-9H-purin-6-amine

Using essentially the same procedure described for Example 132, starting with cis-methylcyclohexanol (1 ml), 2-chloro-9-[5-deoxy-2,3-O-(1-methylethylidene)-β-D-ribofuranosyl]-9H-purin-6-amine (250 mg, 0.77 mmol) and sodium hydroxide (308 mg, 7.7 mmol), 46 mg of the desired product was obtained.

MS (ESP): 364 (MH⁺) for C₁₇H₂₅N₅O₄

¹H NMR δ: 0.84 (d, 3H) 0.99-2.02 (m, 9H) 1.21 (d, 3H) 3.81-3.96 (m, 2H) 4.55-4.62 (m, 1H) 4.98-5.09 (m, 2H) 5.35 (t, 1H) 5.64 (t, 1H) 7.15 (s, 2H) 8.00-8.10 (m, 1H)

### EXAMPLE 135

### 9-(5-deoxy-β-D-ribofuranosyl)-2-[(trans-4-methylcyclohexyl)oxy]-9H-purin-6-amine

Using essentially the same procedure described for Example 132, starting with trans-methylcyclohexanol (1 ml), 2-chloro-9-[5-deoxy-2,3-O-(1-methylethylidene)-β-D-ribofuranosyl]-9H-purin-6-amine (250 mg, 0.77 mmol) and sodium hydroxide (308 mg, 7.7 mmol), 38 mg of the desired product was obtained.

MS (ESP): 364 (MH⁺) for C₁₇H₂₅N₅O₄

¹H NMR δ: 0.83 (d, 3H) 1.01 (m, 2H) 1.22 (d, 3H) 1.23-1.35 (m, 3H) 1.66 (m, 2H) 1.97 (m, 2H) 3.82-3.96 (m, 2H) 4.57-4.73 (m, 2H) 5.04 (d, 1H) 5.36 (d, 1H) 5.63 (d, 1H) 7.12 (s, 2H) 8.01 (s, 1H)

### EXAMPLE 136

### 9-(5-deoxy-β-D-ribofuranosyl)-2-[3,3,3-trifluoro-2-methyl-2-(trifluoromethyl)propoxy]-9H-purin-6-amine

A mixture of 9-[5-deoxy-2,3-O-(1-methylethylidene)-β-D-ribofuranosyl]-2-chloro-9H-purin-6-amine (200 mg) and 2,2-bis(trifluoromethyl)propanol (0.8 g) in THF (2 ml) was stirred at 80° C. in the presence of sodium hydroxide (250 mg; solid) for 48 h. The reaction was diluted with DCM and filtered through diatomaceous earth. The organic layer was washed with water, dried, and concentrated. The residue was purified by chromatography using 80-100% EtOAc in hexanes as eluent to give the intermediate, 9-[5-deoxy-2,3-O-(1-methylethylidene)-β-D-ribofuranosyl]-2-[3,3,3-trifluoro-2-methyl-2-(trifluoromethyl)propoxy]-9H-purin-6-amine (200 mg). The acetonide protecting group was removed by reacting this intermediate with a 1:1 mixture of formic acid and water (6 mL total) at rt for 48 h. At the end of reaction mixture was concentrated and the residue was purified using Gilson reverse phase preparative HPLC to get the desired product (164 mg).

MS (ESP): 446 (MH⁺) for C₁₅H₁₇F₆N₅O₄

¹H NMR (300 MHz, MeOD) δ ppm 1.41 (d, 3H) 1.55 (s, 3H) 4.03 (t, 1H) 4.06-4.16 (m, 1H) 4.62-4.72 (m, 3H) 5.88 (d, 1H) 8.07 (s, 1H)

All alcohols described herein were either obtained from commercial sources or made using procedures described herein.

Using analogous procedure to that described in Example 136, by reacting the appropriate alcohol with 9-[5-deoxy-2,3-O-(1-methylethylidene)-β-D-ribofuranosyl]-2-chloro-9H-purin-6-amine, followed by removal of the protecting group, the following compounds described in Table V were obtained. The reactions can be heated from 80-100° C. for 24 to 48 h.

TABLE V

| | | | ¹HNMR (300 MHz, MeOD, unless otherwise |
|---|---|---|---|
| EX | IUPAC NAME | MH+ | indicated) δ ppm |
| 137 | 9-(5-deoxy-β-D-ribofuranosyl)-2- | 390 | 0.98-1.14 (m, 4H) 1.40 (d, 3H) 3.98-4.15 (m, |
| | {[1-(trifluoromethyl)cyclo- | | 2H) 4.48 (s, 2H) 4.66 (t, 1H) 5.85 (d, 1H) |
| | propyl]methoxy}-9H-purin-6- | | 8.04 (s, 1H) |
| | amine | | |
| 138 | 9-(5-deoxy-β-D-ribofuranosyl)-2- | 418 | 1.41 (d, 3H) 1.45-1.56 (m, 3H) 1.77 (d, 1H) |
| | {[4-(trifluoromethyl)- | | 1.97-2.39 (m, 5H) 4.02-4.15 (m, 2H) 4.70 (q, |
| | cyclohexyl]oxy}-9H-purin-6- | | 1H) 4.91-5.30 (m, 1H) 5.82 (d, 1H) 8.02 (s, 1H) |
| | amine | | |
| 139 | 9-(5-deoxy-β-D-ribofuranosyl)-2- | 386 | 1.39 (d, 3H) 1.85-2.24 (m, 8H) 4.02-4.16 (m, |
| | [(4,4-difluorocyclohexyl)oxy]-9H- | | 2H) 4.69 (t, 1H) 5.11-5.28 (m, 1H) 5.83 (d, 1H) |
| | purin-6-amine | | 8.02 (s, 1H) |
| 140 | 2-{[1-(4- | 432 | 0.90-0.98 (m, 2H) 1.01-1.09 (m, 2H) 1.39 (d, |
| | chlorophenyl)cyclopropyl]methoxy}- | | 3H) 4.00-4.15 (m, 2H) 4.42 (d, 2H) 4.63 (t, 1H) |
| | 9-(5-deoxy-β-D- | | 5.82 (d, 1H) 7.21-7.28 (m, 2H) |
| | ribofuranosyl)-9H-purin-6-amine | | 7.34-7.39 (m, 2H) 7.99 (s, 1H) |
| 141 | 2-{[3,5-bis(trifluoromethyl)- | 486 | 1.30-1.72 (m, 6H) 2.15 (t, 1H) 2.32 (t, 1H) |
| | cyclohexyl]oxy}-9-(5-deoxy-β-D- | | 2.45 (d, 1H) 2.50-2.64 (m, 1H) 2.65-2.84 (m, |
| | ribofuranosyl)-9H-purin-6-amine | | 1H) 4.02-4.13 (m, 2H) 4.66-4.75 (m, 1H) |
| | | | 5.01-5.59 (m, 1H) 5.84 (dd, 1H) 8.04 (d, 1H) |
| 142 | 9-(5-deoxy-β-D-ribofuranosyl)-2- | 400 | 1.31-1.48 (m, 4H) 1.64-2.18 (m, 8H) 4.09 (q, |
| | [(4,4-difluorocyclohexyl)- | | 2H) 4.20 (d, 2H) 4.70 (t, 1H) 5.84 (d, 1H) |
| | methoxy]-9H-purin-6-amine | | 8.02 (s, 1H) |
| 143 | 9-(5-deoxy-β-D-ribofuranosyl)-2- | 338 | 1.04 (s, 9H) 1.41 (d, 3H) 4.00 (s, 2H) |
| | (2,2-dimethylpropoxy)-9H-purin- | | 4.04-4.17 (m, 2H) 4.70 (t, 1H) 5.86 (d, 1H) 8.01 (s, 1H) |
| | 6-amine | | |
| 144 | 9-(5-deoxy-β-D-ribofuranosyl)-2- | 418 | 1.39 (d, 3H) 1.43-1.60 (m, 3H) 1.60-1.72 (m, |
| | {[2-(trifluoromethyl)cyclo- | | 1H) 1.76-2.05 (m, 3H) 2.19 (d, 1H) |
| | hexyl]oxy}-9H-purin-6-amine | | 2.38-2.54 (m, 1H) 4.00-4.14 (m, 2H) 4.70 (q, 1H) |
| | | | 5.65 (s, 1H) 5.84 (dd, 2.54 Hz, 1H) 8.02 (s, 1H) |
| 145 | 9-(5-deoxy-β-D-ribofuranosyl)-2- | 340 | 0.72-1.00 (m, 2H) 1.40 (d, 3H) 1.42-1.53 (m, |
| | {[(1S,2S)-2-fluorocyclopropyl]- | | 1H) 1.89-1.95 (m, 1H) 4.09 (q, 2H) 4.28 (dd, 1H) |
| | methoxy}-9H-purin-6-amine | | 4.56 (dd, 1H) 4.68-4.76 (m, 1H) 5.85 (dd, 1H) |
| | | | 8.02 (s, 1H) |
| 146 | 9-(5-deoxy-β-D-ribofuranosyl)-2- | 358 | 1.25-1.37 (m, 1H) 1.40 (d, 3H) 1.51-1.67 (m, |
| | [(2,2-difluorocyclopropyl)- | | 1H) 2.10-2.29 (m, 1H) 4.03-4.14 (m, 2H) |
| | methoxy]-9H-purin-6-amine | | 4.24-4.34 (m, 1H) 4.42-4.52 (m, 1H) 5.85 (d, |
| | | | 1H) 8.03 (s, 1H) |
| 147 | 2-(1-adamantylmethoxy)-9-(5- | 416 | 1.41 (d, 3H) 1.66-1.70 (m, 5H) 1.70-1.84 (m, |
| | deoxy-β-D-ribofuranosyl)-9H- | | 7H) 1.96-2.02 (m, 3H) 3.87-3.92 (m, 2H) |
| | purin-6-amine | | 4.09 (q, 2H) 4.69 (t, 1H) 5.85 (d, 1H) 8.00 (s, 1H) |
| 148 | 9-(5-deoxy-β-D-ribofuranosyl)-2- | 432 | 1.41 (d, 3H) 1.52-1.87 (m, 8H) 2.06-2.30 (m, |
| | {[4-(trifluoromethyl)cyclohexyl]- | | 2H) 4.02-4.19 (m, 2H) 4.29 (dd, 2H) 4.73 (t, 1H) |
| | methoxy}-9H-purin-6-amine | | 5.85 (d, 1H) 8.02 (s, 1H) |
| 149 | 9-(5-deoxy-β-D-ribofuranosyl)-2- | 418 | 1.41 (d, 3H) 1.45-1.59 (m, 4H) 1.96-2.11 (m, |
| | {[trans-4-(trifluoromethyl)cyclo- | | 2H) 2.12-2.40 (m, 3H) 4.06-4.13 (m, 2H) |
| | hexyl]oxy}-9H-purin-6-amine | | 4.72 (t, 1H) 4.88-5.01 (m, 1H) 5.83 (d, 1H) |
| | | | 8.02 (s, 1H) |
| 150 | 9-(5-deoxy-β-D-ribofuranosyl)-2- | 418 | 1.39 (d, 3H) 1.74 (m, 2H) 2.20 (d, 3H) 4.08 (q, |
| | {[cis-4-(trifluoromethyl)cyclo- | | 2H) 4.69 (t, 1H) 5.21-5.35 (m, 1H) 5.83 (d, 1H) |
| | hexyl]oxy}-9H-purin-6-amine | | 8.02 (s, 1H) |
| 151 | 9-(5-deoxy-β-D-ribofuranosyl)-2- | 340 | 0.69-0.81 (m, 1H) 1.03-1.23 (m, 1H) 1.40 (d, |
| | {[(1S,2R)-2- | | 3H) 1.63-1.86 (m, 1H) 3.99-4.30 (m, 4H) |
| | fluorocyclopropyl]methoxy}-9H- | | 4.47-4.78 (m, 2H) 5.84 (d, 1H) 8.03 (s, 1H) |
| | purin-6-amine | | |
| 152 | 2-[(4-tert-butylcyclohexyl)oxy]-9- | 406 | 0.90 (s, 9H) 0.96-1.35 (m, 5H) 1.41 (d, 3H) |
| | (5-deoxy-β-D-ribofuranosyl)-9H- | | 1.82-1.96 (m, 2H) 2.17-2.31 (m, 2H) |
| | purin-6-amine | | 4.03-4.14 (m, 2H) 4.58-4.83 (m, 2H) 5.81 (d, 1H) |
| | | | 8.01 (s, 1H) |
| 153 | 9-(5-deoxy-β-D-ribofuranosyl)-2- | 348 | 0.68-0.84 (m, 4H) 0.85-0.95 (m, 1H) 1.07 (dd, |
| | (spiro[2.2]pent-1-ylmethoxy)-9H- | | 1H) 1.40 (d, 3H) 1.59-1.70 (m, 1H) |
| | purin-6-amine | | 4.03-4.13 (m, 2H) 4.17 (dd, 1H) 4.35 (dd, 1H) 4.71 (t, 1H) |
| | | | 5.84 (d, 1H) 8.02 (s, 1H) |
| 154 | 2-(bicyclo[3.1.0]hex-3-yloxy)-9- | 348 | 0.03-0.74 (m, 2H) 1.27-1.50 (m, 5H) |
| | (5-deoxy-β-D-ribofuranosyl)-9H- | | 1.85-2.05 (m, 2H) 2.19-2.46 (m, 2H) 3.99-4.20 (m, |
| | purin-6-amine | | 2H) 4.65-4.81 (m, 1H) 4.97-5.48 (m, 1H) |
| | | | 5.79-5.84 (m, 1H) 8.01 (d, 1H) |
| 155 | 2-(cyclopent-3-en-1-yloxy)-9-(5- | 334 | 1.40 (d, 3H) 2.50 (d, 2H) 2.84 (dd, 2H) |
| | deoxy-β-D-ribofuranosyl)-9H- | | 3.99-4.19 (m, 2H) 4.75 (t, 1H) 5.53-5.65 (m, 1H) |
| | purin-6-amine | | 5.74 (s, 2H) 5.83 (d, 1H) 8.02 (s, 1H) |
| 156 | 9-(5-deoxy-β-D-ribofuranosyl)-2- | 418 | 1.41 (d, 3H) 4.02 (t, 1H) 4.06-4.17 (m, 1H) |
| | [2,2,2-trifluoro-1-(trifluoro- | | 4.62 (t, 1H) 5.88 (d, 1H) 6.65-6.76 (m, 1H) |
| | methyl)ethoxy]-9H-purin-6-amine | | 8.12 (s, 1H) |
| 157 | 2-[(4-chlorocyclohexyl)oxy]-9-(5- | 384 | (DMSO-d₆) 0.61 (m, 4H) 1.00-1.40 (m, 8H) |
| | deoxy-β-D-ribofuranosyl)-9H- | | 2.37 (m, 3H) 3.88 (m, 2H) 4.27 (m, 1H) 5.03 (m, 2H) |
| | purin-6-amine | | 7.21 (s, 2H) |
| 158 | 9-(5-deoxy-β-D-ribofuranosyl)-2- | 366 | (DMSO-d₆) 1.22 (m, 4H) 1.49-1.90 (m, 8H) |
| | [(3-hydroxycyclohexyl)oxy]-9H- | | 2.17 (m, 2H) 3.87 (m, 2H) 4.58 (m, 1H) 5.64 (m, 2H) |
| | purin-6-amine | | 7.11 (s, 2H) 8.01 (s, 2H) |
| 159 | 9-(5-deoxy-β-D-ribofuranosyl)-2- | 386 | (DMSO-d₆) 2.12 (m, 4H) 1.40-1.99 (m, 8H) |
| | [(3,3-difluorocyclohexyl)oxy]-9H- | | 3.85 (m, 2H) 4.64 (m, 1H) 4.95 (m, 1H) 5.62 (m, 1H) |
| | purin-6-amine | | 7.19 (s, 2H) 8.04 (s, 2H) |
| 160 | 9-(5-deoxy-β-D-ribofuranosyl)-2- | 486 | (DMSO-d₆) 1.27 (m, 8H) 1.82 (m, 2H) 2.08 (m, |
| | ({3-[(4-methoxybenzyl)oxy]- | | 2H) 3.41 (m, 2H) 3.75 (s, 3H) 4.45 (m, 2H) |
| | cyclohexyl}oxy)-9H-purin-6- | | 4.67 (m, 1H) 4.81 (m, 1H) 5.11 (m, 1H) 5.41 (m, 1H) |
| | amine | | 5.72 (m, 1H) 6.87 (m, 2H) 7.22 (s, 4H) 8.09 (s, |
| | | | 1H) |
| 161 | 9-(5-deoxy-β-D-ribofuranosyl)-2- | 354 | (DMSO-d₆) 0.59 (m, 4H) 1.21 (d, 3H) 2.37 (m, 2H) |
| | {[1-(fluoromethyl)cyclopropyl]- | | 3.88 (m, 2H) 4.09 (m, 2H) 4.22 (m, 1H) |
| | methoxy}-9H-purin-6-amine | | 4.38 (m, 1H) 4.55 (m, 1H) 5.68 (d, 1H) 7.28 (s, 2H) |
| | | | 8.07 (s, 1H) |
| 162 | 9-(5-deoxy-beta-D-ribofuranosyl)- | 426 | 1.42 (d, 3H) 3.98-4.26 (m, 2H) 4.68-4.79 (m, |
| | 2-[(2,6-dichlorobenzyl)oxy]-9H- | | 1H) 5.61 (d, 2H) 5.89 (d, 1H) 7.20-7.53 (m, 3H) |
| | purin-6-amine | | 8.05 (s, 1H) |
| 163 | 9-(5-deoxy-beta-D-ribofuranosyl)- | 352 | 1.41 (d, 3H) 1.67-1.89 (m, 1H) 2.00-2.24 (m, |
| | 2-(tetrahydrofuran-3-ylmethoxy)- | | 1H) 2.62-2.87 (m, 1H) 3.60-3.97 (m, 4H) |
| | 9H-purin-6-amine | | 4.01-4.40 (m, 4H) 4.70 (t, 1H) 5.85 (d, 1H) |
| | | | 8.03 (s, 1H) |
| 164 | 9-(5-deoxy-beta-D-ribofuranosyl)- | 338 | 1.40 (d, 3H) 2.08-2.37 (m, 2H) 3.81-4.17 (m, |
| | 2-(tetrahydrofuran-3-yloxy)-9H- | | 6H) 4.70 (q, 1H) 5.45-5.59 (m, 1H) 5.83 (d, 1H) |
| | purin-6-amine | | 8.03 (s, 1H) |
| 165 | 9-(5-deoxy-beta-D-ribofuranosyl)- | 352 | 1.39 (d, 3H) 1.67-1.87 (m, 2H) 2.03-2.17 (m, |
| | 2-(tetrahydro-2H-pyran-4-yloxy)- | | 2H) 3.51-3.69 (m, 2H) 3.85-4.18 (m, 4H) |
| | 9H-purin-6-amine | | 4.69 (t, 1H) 5.04-5.28 (m, 1H) 5.82 (d, 1H) |
| | | | 8.02 (s, 1H) |
| 166 | 2-(1-cyclopropyl-2,2,2- | 390 | 0.50-0.80 (m, 4H) 1.20-1.35 (m, 1H) 1.40 (d, |
| | trifluoroethoxy)-9-(5-deoxy-beta- | | 3H) 3.88-4.18 (m, 2H) 4.49-4.71 (m, 1H) |
| | D-ribofuranosyl)-9H-purin-6- | | 5.19-5.42 (m, 1H) 5.83 (d, 1H) 8.06 (s, 1H) |
| | amine | | |
| 167 | 9-(5-deoxy-β-D-ribofuranosyl)-2- | 386 | 1.39 (d, 3H) 1.56-1.77 (m, 1H) 1.84.-2.37 (m, |
| | [(3,3- | | 5H) 2.52-2.71 (m, 1H) 4.00-4.14 (m, 2H) |
| | difluorocyclopentyl)methoxy]- | | 4.16-4.35 (m, 2H) 4.69 (t, 1H) 5.84 (d, 1H) |
| | 9H-purin-6-amine | | 8.02 (s, 1H) |
| 168 | 9-(5-deoxy-β-D-ribofuranosyl)-2- | 350 | 0.28 (q, 1H) 0.53-0.60 (m, 1H) 1.12 (d, 6H) |
| | [(2,2- | | 1.20-1.34 (m, 1H) 1.40 (d, 3H) 4.04-4.17 (m, |
| | dimethylcyclopropyl)methoxy]- | | 3H) 4.50 (dd, 1H) 4.71 (t, 1H) 5.84 (d, 1H) |
| | 9H-purin-6-amine | | 8.02 (s, 1H) |
| 169 | 9-(5-deoxy-β-D-ribofuranosyl)-2- | 404 | 0.26-0.44 (m, 1H) 0.85-1.04 (m, 9H) |
| | {[(1S,4R,SR)-1-isopropyl-4- | | 1.05-1.16 (m, 2H) 1.22-1.47 (m, 4H) 1.71-1.98 (m, |
| | methylbicyclol[3.1.0]hex-3- | | 1H) 2.04-2.72 (m, 2H) 3.98-4.17 (m, 2H) |
| | yl]oxy}-9H-purin-6-amine | | 4.65-4.77 (m, 1H) 4.91-5.06 (m, 1H) 5.82 (t, 1H) |
| | | | 8.00 (d, 1H) |
| 170 | 9-(5-deoxy-β-D-ribofuranosyl)-2- | 372 | 1.35-1.45 (m, 3H) 2.02-2.43 (m, 5H) |
| | [(3,3-difluorocyclopentyl)oxy]- | | 2.53-2.76 (m, 1H) 4.01-4.14 (m, 2H) 4.64-4.75 (m, |
| | 9H-purin-6-amine | | 1H) 5.43 (d, 1H) 5.83 (d, 1H) 8.04 (s, 1H) |
| 171 | 9-(5-deoxy-beta-D-ribofuranosyl)- | 384 | 1.40 (d, 3H) 1.79-1.93 (m, 1H) 1.98-2.24 (m, |
| | 2-[(6,6-difluorobicyclo[3.1.0]hex- | | 3H) 2.40-2.58 (m, 1H) 2.59-2.80 (m, 1H) |
| | 3-yl)oxy]-9H-purin-6-amine | | 3.95-4.16 (m, 2H) 4.62-4.79 (m, 1H) |
| | | | 5.19-5.36 (m, 1H) 5.54 (dd, 1H) 5.76-5.91 (m, 1H) |

### EXAMPLE 172

### 2-(Cyclopentyl)methoxy-9-β-D-ribofuranosyl-9H-purine-6-amine

Formic acid (0.5 mL) was added to a suspension of 59 mg (0.145 mmol) of 2-(cyclopentyl)methoxy-9-[2,3-O-(1-methylethylidene)-β-ribofuranosyl-9H-purine-6-amine in 0.5 mL of water. The resulting solution was stirred at rt for 23 h, concentrated in vacuo, and the residue was purified by flash chromatography using 10% MeOH in chloroform as the eluent to provide 38 mg (72%) of the title compound as a colorless oil

MS (ES+) 366 (MH+) for C₁₆H₂₃N₅O₅

¹H NMR δ: 1.50 (m, 2H), 1.78 (m, 4H), 1.94 (m, 2H), 2.48 (m, 1H), 3.38 (d, 2H), 3.79 (m, 2H), 4.11 (m, 1H), 4.30 (m, 2H), 4.78 (m, 1H), 5.36 (m, 1H), 5.63 (d, 1H), 5.98 (d, 1H), 7.49 (s, 2H), 8.35 (s, 1H)

The intermediate was prepared as follows:—

### 2-(Cyclopentyl)methoxy-9-[2,3-O-(1-methylethylidene)-β-ribofuranosyl-9H-purine-6-amine

Potassium tert-butoxide (230 mg, 2.05 mmol) was added to a suspension of 72 mg (0.21 mmol) of 2-chloro-9-[2,3-O-(1-methylethylidene)-β-ribofuranosyl-9H-purine-6-amine and 1.0 mL (9.2 mmol) of cyclopentane MeOH in 4.0 mL of tert-butanol, under a nitrogen atmosphere. The resulting suspension was heated to 70° C. and stirred for 24 h. After cooling to rt, the reaction was diluted with 30 mL of aqueous ammonium chloride and extracted with chloroform (3×20 mL); the combined organic extract was dried over anhydrous sodium sulfate and concentrated in vacuo. Purification by flash chromatography using 5% MeOH in chloroform as the eluent gave 18.7 mg (22%) of the title compound as a white solid.

MS (ES+) 406 (MH+) for C₁₉H₂₇N₅O₅

¹H NMR (300 MHz, CDCl₃) δ 1.21 (m, 2H), 1.24 (s, 3H), 1.46 (m, 4H), 1.50 (s, 3H), 1.69 (m, 2H), 2.24 (m, 1H), 3.67 (m, 1H), 3.83 (d, 1H, J=12.6 Hz), 4.06 (d, 2H), 4.34 (m, 1H), 4.97 (d, 1H), 5.13 (m, 1H), 5.68 (d, 1H), 5.72 (br s, 1H), 7.12 (s, 2H), 7.60 (s, 1H)

Using an analogous procedure to that described for Example 172, starting with the 5′-deoxy-2′3′-aceontide protected adenosine derivatives (described in Table A), the following compounds in Table VI were prepared and purified either by flash chromatography or reverse phase HPLC:

TABLE VI

| | | | | ¹⁹F NMR |
|---|---|---|---|---|
| EX | IUPAC Name | MH+ | ¹H NMR δ | (300 MHz) δ |
| 173 | 2-(1-trifluoromethyl- | 404 | 1.38 (d, 3H), 2.02-2.33 (m, 6H), 4.04 (m, | −75.11 (s) |
| | cyclobutyl)methoxy-9- | | 2H), 4.70 (m, 1H), 5.25 (d, 1H), 5.49 (d, | |
| | β-D-ribofuranosyl-9H- | | 1H), 5.84 (d, 1H), 7.45 (s, 2H), 8.23 (s, | |
| | purine-6-amine | | 1H) | |
| 174 | 2-(2,3,3-trifluoro- | 390 | Acetone-d₆ 1.26 (d, 3H), 2.12 (m, 1H), | 85.37 (d), |
| | cyclobutyl)methoxy-9- | | 2.56 (m, 2H), 3.95 (m, 1H), 4.08 (m, 1H), | 52.95 (d), |
| | β-D-ribofuranosyl-9H- | | 4.34 (m, 2H), 4.68 (m, 1H), 4.99 (m, 1H), | −13.92 |
| | purine-6-amine | | 5.15 (m, 1H), 5.72 (d, 1H), 6.66 (s, 2H), | |
| | | | 7.86 (s, 1H) | |
| 175 | 2-(cyclopentyl) | 350 | 1.36 (d, 3H), 1.36 (m, 2H), 1.63 (m, 4H), | |
| | methoxy-9-(5-deoxy-β- | | 1.80 (m, 2H), 2.35 (m, 1H), 3.23 (d, 1H), | |
| | D-ribofuranosyl)-9H- | | 4.03 (m, 2H), 4.15 (d, 1H), 4.70 (m, 1H), | |
| | purine-6-amine | | 5.18 (d, 1H), 5.45 (d, 1H), 5.79 (d, 1H), | |
| | | | 7.28 (s, 2H), 8.16 (s, 1H) | |
| 176 | 2-(1,2,2-trifluorocyclo- | 404 | CDCl₃ 1.34 (d, 3H), 1.53-2.39 (cp, 6H), | −107.81 (d), |
| | pentyl)methoxy-9-(5- | | 4.08 (s, 2H), 4.23 (s, 2H), 4.50 (m, 1H), | −118.64 (d), |
| | deoxy-β-D- | | 4.68 (m, 1H), 4.74 (m, 1H), 5.78 (m, 1H), | −122.41, |
| | ribofuranosyl)-9H- | | 6.19 (br s, 2H), 7.77 (s, 1H) | −227.36 (d), |
| | purine-6-amine | | | −167.21 (d) |
| 177 | 2-(2-methylcyclo- | 350 | CDCl₃ 0.96 (d, 3H), 1.15 9m, 1H), 1.34 (d, | |
| | pentyloxy)-9-(5-deoxy- | | 3H), 1.64 (m, 3H), 1.88 (m, 1H), 1.99 (m, | |
| | β-D-ribofuranosyl)-9H- | | 1H), 2.10 (m, 1H), 3.42 (s, 2H), 4.06 (m, 1H), | |
| | purine-6-amine | | 4.24 (m, 1H), 4.55 (m, 1H), 4.70 (m, 1H), | |
| | | | 5.76 (m, 1H), 6.00 (s, 2H), 7.69 (s, 1H) | |
| 178 | 2-(3- | 354 | Acetone-d₆ 1.24 (d, 3H), 1.07 (m, 1H), | 9.40, 9.34 |
| | fluorocyclopentyloxy)- | | 1.71-2.37 (cp, 6H), 3.18 (s, 1H), 3.93 (m, | |
| | 9-(5-deoxy-β-D- | | 1H), 4.08 (m, 1H), 4.69 (m, 1H), 4.94 & | |
| | ribofuranosyl)-9H- | | 5.21 (2m, 1H), 5.21 (m, 1H), 5.72 (m, | |
| | purine-6-amine | | 1H), 6.49 (br s, 2H), 7.82 (s, 1H) | |
| 179 | 5′-Acetyloxy-2- | 394 | CDCl₃ 1.51 (m, 2H), 1.72 (m, 6H), | |
| | cyclopentyloxy-9-β-D- | | 1.95 (s, 3H), 4.18 (m, 1H), 4.34 (m, 3H), | |
| | ribofuranosyl-9H- | | 4.62 (m, 1H), 5.15 (m, 1H), 5.82 (d, 1H), | |
| | purine-6-amine | | 6.08 (br s, 2H), 7.70 (s, 1H) | |
| 180 | 2-(Cyclopentyloxy)-9- | 379 | CD₃OD 1.61 (m, 2H), 1.77 (m, 4H), | |
| | β-D-ribofuranosyl-9H- | | 1.93 (m, 2H), 2.63 (m, 2H), 4.31 (m, 2H), | |
| | purine-6-amino-5′- | | 4.70 (m, 1H), 4.85 (s, 5H), 5.34 (m, 1H), | |
| | carboxamide | | 5.82 (d, 1H), 5.98 (d, 1H), 7.99 (s, 1H) | |
| 181 | 5′-Benzoyloxy-2- | 456 | CDCl₃ 1.50 (m, 2H), 1.73 (m, 6H), | |
| | cyclopentyloxy-9-β-D- | | 3.41 (s, 2H), 4.43 (m, 1H), 4.48 (m, 2H), | |
| | ribofuranosyl-9H- | | 4.58 (m, 1H), 4.67 (m, 1H), 5.12 (m, 1H), | |
| | purine-6-amine | | 5.83 (d, 1H), 6.03 (br s, 2H), 7.27 (t, 2H), | |
| | | | 7.44 (t, 1H), 7.71 (s, 1H), 7.78 (d, 2H) | |
| 182 | 5′-Benzyloxy-2- | 442 | CDCl₃ 1.49 (m, 2H), 1.72 (m, 6H), | |
| | cyclopentyloxy-9-β-D- | | 3.40 (s, 2H), 3.63 (m, 2H), 4.28 (m, 1H), | |
| | ribofuranosyl-9H- | | 4.37 (m, 1H), 4.45 (s, 2H), 4.55 (m, 1H), | |
| | purine-6-amine | | 5.12 (m, 1H), 5.87 (d, 1H), 6.02 (br s, 2H), | |
| | | | 7.21 (m, 5H), 7.77 (s, 1H) | |
| 183 | 5′-Methylsulfonyloxy- | 430 | Acetone-d₆ 1.47 (m, 2H), 1.63 (m, 4H), | |
| | 2-cyclopentyloxy-9-β- | | 1.81 (m, 2H), 2.97 (s, 3H), 3.18 (s, 2H), | |
| | D-ribofuranosyl-9H- | | 4.16 (m, 1H), 4.38 (m, 1H), 4.42 (m, 1H), | |
| | purine-6-amine | | 4.46 (m, 1H), 4.78 (m, 1H), 5.22 (m, 1H), | |
| | | | 5.84 (d, 1H), 6.57 (br s, 2H), 7.88 (s, 1H) | |

Using an analogous procedure to that described for Example 172, the following intermediates in Table A were prepared by reaction of 2-chloro-9-[2,3-O-(1-methylethylidene)-β-ribofuranosyl-9H-purine-6-amine and the appropriate alcohol:

TABLE A

| | | | ¹⁹F NMR |
|---|---|---|---|
| IUPAC Name | MH+ | ¹H NMR (300 MHz, CDCl₃) δ | (300 MHz) δ |
| 2-(1-trifluoromethyl- | 444 | 1.30 (s, 3H), 1.30 (d, 3H), 1.53 (s, 3H), | −76.90 |
| cyclobutyl)methoxy-9-[2,3-O- | | 1.97 (m, 2H), 2.17 (m, 2H), 2.27 (m, 2H), | |
| (1-methylethylidene)-β- | | 4.26 (m, 1H), 4.44 (m, 2H), 4.65 (dd, 1H), | |
| ribofuranosyl-9H-purine-6- | | 5.40 (dd, 1H), 5.90 (d, 1H), 6.40 (s, 2H), 7.68 (s, | |
| amine | | 1H) | |
| 2-(2,3,3-trifluoro- | 430 | 1.31 (s, 3H), 1.31 (d, 3H), 1.54 (s, 3H), | −92.08 (d), |
| cyclobutyl)methoxy-9-[2,3-O- | | 2.15 (m, 1H), 2.4-2.7 (cp, 2H), 4.26 (m,, 1H), | −108.73, −124.85 (dd), |
| (1-methylethylidene)-β- | | 4.41 (m, 1H), 4.63 (m, 1H), 4.90 (m, 1H), | −191.18 |
| ribofuranosyl-9H-purine-6- | | 5.07 (m, 1H), 5.32 (m, 1H), 5.90 (s, 2H), | |
| amine | | 7.69 (s, 1H) | |
| 2-(2-methylcyclopentyl-oxy)- | 390 | 1.00 (d, 3H), 1.19 (m, 1H), 1.28 (d, 3H), | |
| 9-[2,3-O-(1-methylethylidene)- | | 1.31 (s, 3H), 1.53 (s, 3H), 1.63 (m, 1H), | |
| β-ribofuranosyl-9H-purine-6- | | 1.74 (m, 2H), 1.97 (m, 2H), 2.18 (m, 1H), | |
| amine | | 4.25 (dd, 1H), 4.67 (m, 1H), 4.81 (m, 1H), | |
| | | 5.49 (m, 1H), 5.89 (d, 1H), 5.94 (s, 2H), | |
| | | 7.65 (s, 1H) | |
| 2-(3-hydroxycyclo-pentyloxy)- | 392 | 1.29 (d, 3H), 1.30 (s, 3H), 1.53 (s, 3H), | |
| 9-[2,3-O-(1-methylethylidene)- | | 1.61 (m, 1H), 1.87 (m, 1H), 2.0-2.25 (m, 4H), | |
| β-ribofuranosyl-9H-purine-6- | | 3.40 (s, 1H), 4.24 (m, 1H), 4.51 (m, 1H), | |
| amine | | 4.68 (dd, 1H), 5.41 (m, 1H), 5.48 (dd, 1H), | |
| | | 5.88 (d, 1H), 6.00 (br s, 2H), 7.65 (s, 1H) | |
| 2-(cyclopentyl)-methoxy-9- | 390 | 1.29 (d, 3H), 1.31 (s, 3H), 1.30 (m, 3H), | |
| [2,3-O-(1-methylethylidene)- | | 1.53 (s, 3H), 1.56 (m, 3H), 1.72 (m, 2H), | |
| β-ribofuranosyl-9H-purine-6- | | 2.33 (quin, 1H), 4.12 (dd, 2H), 4.25 (m, | |
| amine | | 1H), 4.70 (dd, 1H), 5.44 (dd, 1H), 5.89 (d, | |
| | | 1H), 6.19 (s, 2H), 7.65 (s, 1H) | |
| 2-(1,2,2-trifluorocyclo- | 444 | 1.30 (d, 3H), 1.31 (s, 3H), 1.53 (s, 3H), | −109.02 (d), |
| pentyl)methoxy-9-[2,3-O-(1- | | 1.72-2.43 (cp, 6H), 3.28-3.95 (cp, 2H), | −118.11 (d), |
| methylethylidene)-β- | | 4.27 (m, 1H), 4.68 (m, 1H), 4.84 (m, 1H), | −123.00, −125.57, |
| ribofuranosyl-9H-purine-6- | | 5.47 (m, 1H), 5.82 & 5.88 (2s, 2H), 7.65 (s, 1H) | −170.49 |
| amine | | | |

The intermediate for Example 178 was made as follows:—

### 2-(3-fluorocyclopentyl)oxy-9-[2,3-O-(1-methylethylidene)-β-ribofuranosyl-9H-purine-6-amine

Under a nitrogen atmosphere, a solution of 89 mg (0.23 mmol) of 2-(3-hydroxycyclo-pentyloxy)-9-[2,3-O-(1-methylethylidene)-β-ribofuranosyl-9H-purine-6-amine in 1.5 mL of DCM was added to an ice-cold solution of 60 mg (0.27 mmol) of bis-(2-methoxyethyl)amino sulphur trifluoride in 1 mL of DCM. The resulting solution was stirred for 2 h at 5° C., then warmed to rt and stirred for an additional 4 h. The reaction was quenched by addition to 20 mL of a cold solution of aqueous potassium carbonate (evolution of carbon dioxide); the layers were separated and the aqueous layer was extracted with DCM (2×10 mL). The combined organic extract was dried over anhydrous sodium sulfate, then subjected to reverse phase chromatography using 10 mM ammonium acetate with 5% acetonitrile/acetonitrile (20-60%), 14 min, to give 15 mg (17%) of the title compound as a colorless film.

MS (ES+) 394 (MH+) for C₁₈H₂₄FN₅O₄

¹H NMR (300 MHz, CDCl₃) δ 1.29 (d, 3H), 1.31 (s, 3H), 1.53 (s, 3H), 1.70-2.30 (cp, 7H), 3.33 & 3.45 (2m, 1H), 4.25 (m, 1H), 4.64 (m, 1H), 5.22 (m, 1H), 5.44 (m, 1H), 5.89 (s, 2H), 7.66 (s, 1H)

¹⁹F NMR (300 MHz, CDCl₃) δ −169.48, −169.56

The intermediate for Example 180 was made as follows:—

### 2-Cyclopentyloxy-9-[2,3-O-(1-methylethylidene)-β-ribofuranosyl]-9H-purine-6-amine-5′-carboxamide

Hydrogen peroxide (30% aqueous, 0.25 mL) was added to an ice-cold suspension of 209 mg (0.51 mmol) of 5′-cyano-2-cyclopentyloxy-9-[2,3-O-(1-methylethylidene)-β-ribofuranosyl]-9H-purine-6-amine (prepared using an analogous procedure to that described in Example 248) and 30 mg of potassium carbonate in 2.5 mL of dimethylsulfoxide. After 30 min, the reaction was stirred for 18 h at rt, then diluted with 10 mL of water and extracted with EtOAc (3×15 mL). Purification by reverse phase chromatography (10 mM ammonium acetate, pH 8, 5% acetonitrile/acetonitrile, 10-60% over 14 min) provided 50 mg (23%) of the title compound as a white fluffy solid.

MS (ES+) 419 (MH+) for C₁₉H₂₆N₆O₅

¹H NMR (300 MHz, CDCl₃) δ 1.31 (s, 3H), 1.54 (s, 3H), 1.50-1.90 (m, 8H), 2.63 (m, 2H), 4.50 (m, 1H), 4.97 (dd, 1H), 5.24 (m, 1H), 5.44 (dd, 1H), 5.66 (m, 1H), 5.92 (m, 4H), 7.67 (s, 1H)

The intermediate for Example 179 was made as follows:—

### 5′-Acetyloxy-2-cyclopentyloxy-9-[2,3-O-(1-methylethylidene)-β-ribofuranosyl]-9H-purine-6-amine

Under a nitrogen atmosphere, 0.70 mL (0.74 mmol) of acetic anhydride was added to a solution of 117 mg (0.30 mmol) of 2-cyclopentyloxy-9-[2,3-O-(1-methylethylidene)-β-ribofuranosyl]-9H-purine-6-amine and 0.10 mL (0.72 mmol) of triethylamine in 5 mL of anhydrous THF. After 20 h, an additional 0.10 mL of acetic anhydride was added, the reaction was stirred for 48 h, then diluted with 40 mL of EtOAc and extracted with water (2×25 mL) followed by 25 mL of saturated sodium chloride; reverse phase chromatography (10 mM ammonium acetate, pH 8, 5% acetonitrile/acetonitrile, 10-60% over 14 min) provided 58 mg (44%) of the title compound as a colorless film.

MS (ES+) 434 (MH+) for C₂₀H₂₇N₅O₆

¹H NMR (300 MHz, CDCl₃) δ 1.38 (s, 3H), 1.60 (s, 3H), 1.63 (m, 2H), 1.81-1.95 (m, 6H), 2.00 (s, 3H), 4.18 (m, 1H), 4.30 (m, 1H), 4.41 (m, 1H), 5.04 (m, 1H), 5.29 (m, 1H), 5.53 (m, 1H), 6.03 (m, 1H), 6.28 (br s, 2H), 7.71 (s, 1H)

The compounds in Table VII were obtained by a similar procedure to that described for Example 179 by reaction of 2-cyclopentyloxy-9-[2,3-O-(1-methylethylidene)-β-ribofuranosyl]-9H-purine-6-amine with the appropriate commercially available reagent:

TABLE VII

| IUPAC Name | MH+ | ¹H NMR (300 MHz, CDCl₃) δ |
|---|---|---|
| 5′-benzoyloxy-2-cyclopentyloxy-9- | 496 | 1.43 (s, 3H), 1.62 (m, 2H), 1.65 (s, 3H), 1.92 (m, |
| [2,3-O-(1-methylethylidene)-β- | | 6H), 4.50 (m, 1H), 4.57 (m, 1H), 4.61 (m, 1H), |
| ribofuranosyl]-9H-purine-6-amine | | 5.17 (dd, 1H), 5.32 (m, 1H), 5.65 (dd, 1H), 6.10 (d, 1H), |
| | | 6.79 (br s, 2H), 7.47 (m, 3H), 7.82 (s, 1H), 7.90 (m, |
| | | 1H), 8.14 (m, 1H) |
| 5′-methylsulfonyloxy-2- | 470 | 1.32 (s, 3H), 1.55 (s, 3H), 1.62 (m, 2H), 1.86 (m, |
| cyclopentyloxy-9-[2,3-O-(1- | | 6H), 2.85 (s, 3H), 4.36 (m, 3H), 5.05 (m, 1H), |
| methylethylidene)-β-ribofuranosyl]- | | 5.24 (m, 1H), 5.46 (m, 1H), 5.99 (d, 1H), 6.04 (br s, 2H), |
| 9H-purine-6-amine | | 7.69 (s, 1H) |

The intermediate for Example 182 was prepared as follows:—

### 5′-Benzyloxy-2-cyclopentyloxy-9-[2,3-O-(1-methylethylidene)-β-ribofuranosyl]-9H-purine-6-amine

Under a nitrogen atmosphere at 0° C., 197 mg (0.50 mmol) of 2-cyclopentyloxy-9-[2,3-O-(1-methylethylidene)-β-ribofuranosyl]-9H-purine-6-amine was added to a suspension of 34 mg (0.85 mmol) of 60% sodium hydride in 5 mL of THF; after stirring for 20 min, a solution of 0.07 mL (0.58 mmol) of benzyl bromide in 1 mL of THF was added dropwise over 10 min. The ice bath was removed and the reaction was stirred at rt for 18 h. The reaction was quenched by addition of 25 mL of aqueous ammonium chloride, then extracted with 40 mL of EtOAc; the organic extract was washed with 25 mL of water, 25 mL of saturated sodium chloride, dried over anhydrous magnesium sulfate, then subjected to normal phase chromatography (pyridine column, 21×15 mm) using hexane/1:1 MeOH-ethanol (5-18%), to give 59 mg (24%) of the title compound as a colorless film.

MS (ES+) 482 (MH+) for C₂₅H₃₁N₅O₅

¹H NMR (300 MHz, CDCl₃) δ 1.39 (s, 3H), 1.62 (s, 3H), 1.64 (m, 2H), 1.90 (m, 6H), 3.63 (m, 2H), 4.46 (m, 1H), 4.50 (m, 2H), 5.01 (m, 1H), 5.31 (m, 1H), 5.43 (m, 1H), 6.04 (br s, 2H), 6.10 (m, 1H), 7.22-7.34 (m, 5H), 7.79 (s, 1H)

### EXAMPLE 184

### 2-(cyclobutylmethoxy)-9-(5-deoxy-5-fluoro-(3-D-ribofuranosyl)-9H-purin-6-amine

Cyclobutane methanol (549 ul) was added to a sealed-flask containing 2-chloro-9-[5-deoxy-5-fluoro-2,3-O-(1-methylethylidene)-β-D-ribofuranosyl]-9H-purin-6-amine (200 mg, 0.58 mmol) and sodium hydroxide (233 mg, 5.8 mmol). The reaction was heated to 75° C. for 18 h. After cooling to rt, excess sodium hydroxide was filtered off and washed with DCM. The solution was washed with water, dried (sodium sulfate), filtered and concentrated in vacuo. The resulting residue was dissolved in 1:1.5 water/acetic acid (20 mL total). Formic acid (3 ml) was added and the reaction mixture heated to 95° C. for 8 h. The reaction mixture was concentrated in vacuo and the residue was purified using Gilson reverse phase HPLC with 10 mM ammonium acetate and acetonitrile as the mobile phases with a gradient of 5-50% in 10 min. Relevant fractions were combined to give 70 mg of the desired product.

MS (ESP): 354 (MH⁺) for C₁₅H₂₀FN₅O₄

¹H NMR δ: 1.70-1.86 (m, 4H) 1.90-2.03 (m, 2H) 2.62 (m, 1H) 4.05-4.13 (m, 3H) 4.18 (q, 1H) 4.44-4.54 (m, 2H) 4.59-4.68 (m, 1H) 5.35 (d, 1H) 5.53 (d, 1H) 5.76 (d, 1H) 7.21 (s, 2H) 8.00 (s, 1H)

The intermediate was prepared as follows:—

### 2-chloro-9-[5-deoxy-5-fluoro-2,3-O-(1-methylethylidene)-β-D-ribofuranosyl]-9H-purin-6-amine

To a solution of 2-chloro-9-(5-deoxy-5-fluoro-β-D-ribofuranosyl)-9H-purin-6-amine (7 g, 23.1 mmol) and dimethoxypropane (14.3 ml, 115.5 mmol) in dry acetone (150 ml) was added p-toluenesulfonic acid (2.2 g, 11.6 mmol) at rt. The solution was stirred for 3 h at 45° C. The precipitate was filtered off and washed with acetone. The filtrate was concentrated in vacuo and the residue was purified by chromatography eluting with 4% MeOH in DCM to give desired product (3.9 g).

MS (ESP): 344 (MH⁺) for C₁₃H₁₅ClFN₅O₄

¹H NMR (300 MHz, CDCl₃) δ ppm 1.54 (s, 3H) 1.77 (s, 3H) 4.53-4.65 (m, 1H) 4.65-4.77 (m, 1H) 4.80-4.93 (m, 1H) 5.21 (dd, 1H) 5.39 (dt, 1H) 6.26 (s, 2H) 6.31 (d, 1-H) 8.06 (s, 1H)

Using an analogous procedure to that described in Example 184, the appropriate alcohol was reacted with 2-chloro-9-[5-deoxy-5-fluoro-2,3-O-(1-methylethylidene)-β-D-ribofuranosyl]-9H-purin-6-amine then deprotected with acid (either acetic acid/water/formic acid at 90° C. or formic acid/water rt) to give the compounds described in Table VIII.

TABLE VIII

| | | | ¹HNMR δ ppm (300 MHz, CDCl₃ unless |
|---|---|---|---|
| EX | IUPAC Name | MH+ | otherwise specified) |
| 185 | 2-(cyclopentyloxy)-9-(5-deoxy-5- | 354 | 1.50-1.70 (d, 6H) 1.82-1.97 (m, 2H) |
| | fluoro-β-D-ribofuranosyl)-9H- | | 4.02-4.15 (m, 1H) 4.27 (q, 1H) 4.52-4.65 (m, 2H) |
| | purin-6-amine | | 4.68-4.74 (m, 1H) 5.27 (t, 1H) 5.41 (d, 1H) 5.60 (d, 1H) |
| | | | 5.76-5.86 (m, 1H) 7.24 (s, 2H) 8.06 (s, 1H) |
| 186 | 9-(5-deoxy-5-fluoro-β-D- | 382 | 0.83 (d, 3H) 1.00 (m, 2H) 1.30 (m, 3H) 1.63 (m, |
| | ribofuranosyl)-2-[(4- | | 2H) 1.96 (m, 2H) 3.98-4.06 (m, 1H) 4.19 (m, 1H) |
| | methylcyclohexyl)oxy]-9H-purin- | | 4.55-4.70 (m, 5H) 5.59 (m, 1H) 5.73 (d, 1H) |
| | 6-amine | | 7.14 (s, 2H) 7.98 (s, 1H) |
| 187 | 9-(5-deoxy-5-fluoro-β-D- | 382 | 0.83 (d, 3H) 1.00 (m, 2H) 1.30 (m, 3H) 1.64 (m, |
| | ribofuranosyl)-2-[(trans-4- | | 2H) 1.97 (m, 2H) 3.95-4.10 (m, 1H) 4.19 (m, 1H) |
| | methylcyclohexyl)oxy]-9H-purin- | | 4.55-4.67 (m, 4H) 5.35 (m, 1H) 5.58 (m, 1H) |
| | 6-amine | | 5.73 (d, 1H) 7.17 (s, 2H) 7.96-8.11 (m, 1H) |
| 188 | 9-(5-deoxy-5-fluoro-β-D- | 382 | 0.82 (s, 3H) 1.24-1.83 (m, 9H) 4.03 (m, 1H) |
| | ribofuranosyl)-2-[(cis-4- | | 4.18 (m, 1H) 4.51-4.63 (m, 3H) 5.03 (d, 1H) 5.36 (d, 1H) |
| | methylcyclohexyl)oxy]-9H-purin- | | 5.55 (d, 1H) 5.77 (d, 1H) 7.24 (s, 2H) 8.03 (s, |
| | 6-amine | | 1H) |
| 189 | 2-(decahydronaphthalen-2- | 422 | 1.29-1.81 (m, 16H) 4.01 (dd, 1H) 4.17 (m, 1H) |
| | yloxy)-9-(5-deoxy-5-fluoro-β-D- | | 4.51-4.61 (m, 3H) 4.98 (m, 1H) 5.34 (m, 1H) |
| | ribofuranosyl)-9H-purin-6-amine | | 5.58 (m, 1H) 5.73 (d, 1H) 7.13 (s, 2H) 7.97 (s, 1H) |
| 190 | 9-(5-deoxy-5-fluoro-beta-D- | 436 | (MeOD) 1.39-1.60 (m, 4H) 1.94-2.38 (m, 5H) |
| | ribofuranosyl)-2-{[4- | | 4.08-4.28 (m, 1H) 4.41 (t, 1H) 4.51-4.84 (m, 4H) |
| | (trifluoromethyl)cyclohexyl]oxy}- | | 5.92 (d, 1H) 8.01 (s, 1H) |
| | 9H-purin-6-amine | | |

### EXAMPLE 191

### 9-(5-deoxy-5-fluoro-beta-D-ribofuranosyl)-2-[(2-methylcyclopropyl)methoxy]-9H-purin-6-amine

A mixture of 2-chloro-9-(5-deoxy-5-fluoro-β-D-ribofuranosyl)-9H-purin-6-amine (200 mg), and (2-methylcyclopropyl)methanol (1 ml) in the presence of sodium hydroxide (200 mg) was heated at 75° C. for 2 h. The reaction was cooled, diluted with dichloromethane and filtered through diatomaceous earth. The product obtained after concentration of organic solvent was purified using Gilson reverse phase preparative HPLC to give the desired product (25 mg).

MS (ESP): 354 (MH⁺) for C₁₅H₂₀FN₅O₄

¹H NMR (300 MHz, MeOD) δ ppm 0.35-0.60 (m, 2H) 0.74-1.03 (m, 2H) 1.10 (d, 3H) 4.04-4.88 (m, 7H) 5.94 (t, J=3.77 Hz, 1H) 8.00 (s, 1H).

Using analogous procedure to that described in Example 191, the appropriate alcohol was reacted with 2-chloro-9-(5-deoxy-5-fluoro-β-D-ribofuranosyl)-9H-purin-6-amine to give the compounds described in Table IX. Reactions were either done neat or in solvent (THF). The reactions can be heated from 60-100° C. for 30 min to 24 h.

TABLE IX

| | | | ¹H NMR (300 MHz, MeOD unless |
|---|---|---|---|
| EX | IUPAC Name | MH+ | otherwise indicated) δ ppm |
| 192 | 9-(5-deoxy-5-fluoro-β-D- | 354 | (DMSO-d₆) 0.33 (m, 2H) 0.50 (m, 2H) |
| | ribofuranosyl)-2-[(1- | | 1.14 (s, 3H) 3.75-3.88 (m, 3H) 4.23 (m, 1H) |
| | methylcyclopropyl)methoxy]- | | 4.53 (m, 2H) 4.69 (m, 1H) 5.40 (d, 1H) |
| | 9H-purin-6-amine | | 5.57 (d, 1H) 5.80 (d, 1H) 7.27 (s, 2H) |
| | | | 8.05 (s, 1H). |
| 193 | 9-(5-deoxy-5-fluoro-β-D- | 374 | (DMSO-d₆) 3.21-3.34 (m, 8H) |
| | ribofuranosyl)-2-[2-(2- | | 3.46-3.57 (t, 2H) 3.91 (m, 1H) 4.02 (t, 1H) 4.14 (t, 2H) |
| | hydroxyethoxy)ethoxy]-9H- | | 4.36 (m, 1H) 4.51 (m, 1H) 5.63 (d, 1H) |
| | purin-6-amine | | 7.12 (s, 2H) 7.89 (s, 1H) |
| 194 | 2-(cyclopropylmethoxy)-9- | 340 | 0.27-0.45 (m, 2H) 0.52-0.71 (m, 2H) |
| | (5-deoxy-5-fluoro-β-D- | | 1.19-1.37 (m, 1H) 4.10-4.81 (m, 7H) 5.94 (d, |
| | ribofuranosyl)-9H-purin-6- | | 1H) 7.97-8.05 (m, 1H) |
| | amine | | |
| 195 | 9-(5-deoxy-5-fluoro-β-D- | 370 | 0.48-0.71 (m, 4H) 3.47-3.65 (m, 2H) |
| | ribofuranosyl)-2-{[1- | | 4.06-4.27 (m, 1H) 4.23-4.32 (m, 2H) 4.40 (t, 1H) |
| | (hydroxymethyl)cyclopropyl] | | 4.54-4.66 (m, 2H) 4.71-4.82 (m, 1H) |
| | methoxy}-9H-purin-6- | | 5.94 (d, 1H) 8.00 (s, 1H) |
| | amine | | |
| 196 | 9-(5-deoxy-5-fluoro-β-D- | 356 | 2.62-2.83 (m, 2H) 4.13-4.26 (m, 1H) |
| | ribofuranosyl)-2-(oxetan-2- | | 4.36-4.77 (m, 7H) 5.05-5.22 (m, 1H) 5.96 (d, |
| | ylmethoxy)-9H-purin-6- | | 1H) 8.03 (s, 1H) |
| | amine | | |
| 197 | 9-(5-deoxy-5-fluoro-β-D- | 370 | 1.38-1.52 (m, 3H) 4.13-4.32 (m, 1H) |
| | ribofuranosyl)-2-[(2- | | 4.36-4.52 (m, 5H) 4.56-4.71 (m, 4H) |
| | methyloxetan-2- | | 4.73-4.82 (m, 1H) 5.98 (d, 1H) 8.02-8.09 (m, 1H) |
| | yl)methoxy]-9H-purin-6- | | |
| | amine | | |
| 198 | 2-(benzyloxy)-9-(5-deoxy- | 376 | 4.09-4.28 (m, 1H) 4.41 (t, 1H) |
| | 5-fluoro-β-D- | | 4.52-4.78 (m, 3H) 5.36-5.45 (m, 2H) 5.96 (d, 1H) |
| | ribofuranosyl)-9H-purin-6- | | 7.25-7.41 (m, 3H) 7.43-7.54 (m, 2H) |
| | amine | | 8.02 (s, 1H) |
| 199 | 9-(5-deoxy-5-fluoro-β-D- | 394 | 4.11-4.30 (m, 1H) 4.36-4.44 (m, 1H) |
| | ribofuranosyl)-2-[(2- | | 4.54-4.65 (m, 2H) 4.70-4.81 (m, 1H) |
| | fluorobenzyl)oxy]-9H- | | 5.41-5.50 (m, 2H) 5.98 (d, 1H) 7.06-7.24 (m, 2H) |
| | purin-6-amine | | 7.31-7.42 (m, 1H) 7.50-7.62 (m, 1H) |
| | | | 7.99-8.07 (m, 1H) |
| 200 | 9-(5-deoxy-5-fluoro-β-D- | 382 | 4.11-4.30 (m, 1H) 4.42 (t, 1H) |
| | ribofuranosyl)-2-(2- | | 4.54-4.67 (m, 2H) 4.73-4.83 (m, 1H) 5.54-5.59 (m, |
| | thienylmethoxy)-9H-purin- | | 2H) 6.00 (d, 1H) 6.98 (dd, 1H) 7.20 (d, 1H) |
| | 6-amine | | 7.38 (dd, 1H) 7.99-8.10 (m, 1H) |
| 201 | 2-[(2-chlorobenzyl)oxy]-9- | 410 | 4.11-4.28 (m, 1H) 4.39 (t, 1H) |
| | (5-deoxy-5-fluoro-β-D- | | 4.52-4.75 (m, 3H) 5.47-5.54 (m, 2H) 5.97 (d, 1H) |
| | ribofuranosyl)-9H-purin-6- | | 7.26-7.36 (m, 2H) 7.38-7.47 (m, 1H) |
| | amine | | 7.55-7.66 (m, 1H) 8.03 (s, 1H) |
| 202 | 9-(5-deoxy-5-fluoro-β-D- | 444 | 4.11-4.27 (m, 1H) 4.38 (t, 1H) |
| | ribofuranosyl)-2-{[2- | | 4.50-4.73 (m, 3H) 5.58 (s, 2H) 5.96 (d, 1H) |
| | (trifluoromethyl)benzyloxy}- | | 7.36-7.85 (m, 4H) 8.04 (s, 1H) |
| | 9H-purin-6-amine | | |
| 203 | 2-[(4-bromo-2- | 461 | 4.01-4.20 (m, 1H) 4.31 (t, 1H) |
| | thienyl)methoxy]-9-(5- | | 4.45-4.55 (m, 2H) 4.57-4.69 (m, 1H) 5.40-5.46 (m, |
| | deoxy-5-fluoro-β-D- | | 2H) 5.90 (d, 1H) 7.05 (s, 1H) |
| | ribofuranosyl)-9H-purin-6- | | 7.25-7.31 (m, 1H) 7.95 (s, 1H) |
| | amine | | |
| 204 | 9-(5-deoxy-5-fluoro-β-D- | 383 | 4.09-4.30 (m, 1H) 4.40 (t, 1H) |
| | ribofuranosyl)-2-(1,3- | | 4.52-4.78 (m, 3H) 5.70 (s, 2H) 5.97 (d, 1H) 7.61 (d, 1H) |
| | thiazol-2-ylmethoxy)-9H- | | 7.79 (d, 1H) 8.05 (s, 1H) |
| | purin-6-amine | | |
| 205 | 2-(1,3-benzothiazol-2- | 433 | 4.10-4.26 (m, 1H) 4.39 (t, 1H) |
| | ylmethoxy)-9-(5-deoxy-5- | | 4.49-4.77 (m, 3H) 5.81 (s, 2H) 5.95 (d, 1H) |
| | fluoro-β-D-ribofuranosyl)- | | 7.34-7.57 (m, 2H) 7.91-8.01 (m, 2H) 8.05 (s, 1H) |
| | 9H-purin-6-amine | | |
| 206 | 9-(5-deoxy-5-fluoro-β-D- | 459 | 4.11-4.32 (m, 1H) 4.42 (t, 1H) |
| | ribofuranosyl)-2-[(2- | | 4.57-4.66 (m, 2H) 4.70-4.82 (m, 1H) 5.54 (s, 2H) |
| | phenyl-1,3-thiazol-4- | | 5.98 (d, 1H) 7.41-7.54 (m, 3H) |
| | yl)methoxy]-9H-purin-6- | | 7.57-7.67 (m, 1H) 7.88-8.00 (m, 2H) 8.04 (s, 1H) |
| | amine | | |
| 207 | 9-(5-deoxy-5-fluoro-β-D- | 382 | 4.08-4.27 (m, 1H) 4.42 (t, 1H) |
| | ribofuranosyl)-2-(3- | | 4.53-4.65 (m, 2H) 4.71-4.83 (m, 1H) 5.40 (s, 2H) |
| | thienylmethoxy)-9H-purin- | | 5.97 (d, 1H) 7.20 (dd, 1H) 7.31-7.51 (m, 2H) |
| | 6-amine | | 8.02 (s, 1H) |
| 208 | 2-[(4-chlorobenzyl)oxy]-9- | 410 | 4.08-4.28 (m, 1H) 4.40 (t, 1H) |
| | (5-deoxy-5-fluoro-β-D- | | 4.51-4.63 (m, 2H) 4.66-4.79 (m, 1H) 5.34-5.43 (m, |
| | ribofuranosyl)-9H-purin-6- | | 2H) 5.96 (d, 1H) 7.35 (d, 2H) 7.47 (d, 2H) |
| | amine | | 8.02 (s, 1H) |
| 209 | 9-(5-deoxy-5-fluoro-β-D- | 381 | 2.41 (s, 3H) 4.08-4.28 (m, 1H) 4.40 (t, 1H) |
| | ribofuranosyl)-2-[(5- | | 4.53-4.66 (m, 2H) 4.71-4.81 (m, 1H) |
| | methylisoxazol-3- | | 5.40 (s, 2H) 5.95 (d, 1H) 6.25 (s, 1H) |
| | yl)methoxy]-9H-purin-6- | | 8.04 (s, 1H) |
| | amine | | |
| 210 | 2-[(3-chlorobenzyl)oxy]-9- | 410 | 4.10-4.29 (m, 1H) 4.41 (t, 1H) |
| | (5-deoxy-5-fluoro-β-D- | | 4.53-4.64 (m, 2H) 4.69-4.76 (m, 1H) 5.39 (s, 2H) |
| | ribofuranosyl)-9H-purin-6- | | 5.96 (d, 1H) 7.17-7.44 (m, 3H) 7.50 (s, 1H) |
| | amine | | 8.03 (s, 1H) |
| 211 | 9-(5-deoxy-5-fluoro-β-D- | 394 | 4.09-4.30 (m, 1H) 4.41 (t, 1H) |
| | ribofuranosyl)-2-[(4- | | 4.53-4.66 (m, 2H) 4.70-4.78 (m, 1H) 5.37 (s, 2H) |
| | fluorobenzyl)oxy]-9H- | | 5.96 (d, 1H) 7.00-7.17 (m, 2H) |
| | purin-6-amine | | 7.43-7.58 (m, 2H) 8.02 (s, 1H) |
| 212 | 9-(5-deoxy-5-fluoro-β-D- | 368 | (DMSO-d₆) 4.00-4.15 (m, 1H) 4.22 (d, 1H) |
| | ribofuranosyl)-2-(2,2,2- | | 4.48-4.61 (m, 2H) 4.63-4.76 (m, 1H) |
| | trifluoroethoxy)-9H-purin- | | 5.42 (d, 1H) 5.60 (d, 1H) 5.83 (d, 1H) |
| | 6-amine | | 7.54 (s, 2H) 8.13 (s, 1H) |
| 213 | 9-(5-deoxy-5-fluoro-β-D- | 344 | 3.40 (s, 3H) 3.71-3.77 (m, 2H) |
| | ribofuranosyl)-2-(2- | | 4.11-4.28 (m, 1H) 4.39-4.49 (m, 3H) 4.57-4.67 (m, |
| | methoxyethoxy)-9H-purin- | | 2H) 4.72-4.82 (m, 1H) 5.95 (d, 1H) |
| | 6-amine | | 8.02 (s, 1H) |
| 214 | 9-(5-deoxy-5-fluoro-β-D- | 342 | 1.02 (d, 6H) 1.98-2.22 (m, 1H) 4.09 (d, 2H) |
| | ribofuranosyl)-2-isobutoxy- | | 4.12-4.28 (m, 1H) 4.42 (t, 1H) |
| | 9H-purin-6-amine | | 4.53-4.67 (m, 2H) 4.72-4.80 (m, 1H) 5.95 (d, 1H) |
| | | | 8.01 (s, 1H) |
| 215 | 9-(5-deoxy-5-fluoro-β-D- | 400 | (DMSO-d₆) 4.01-4.16 (m, 1H) 4.20 (t, 1H) |
| | ribofuranosyl)-2-(2,2,3,3- | | 4.50-4.60 (m, 2H) 4.64-4.85 (m, 3H) |
| | tetrafluoropropoxy)-9H- | | 5.83 (d, 1H) 6.64 (tt, 1H) 7.52 (s, 2H) 8.13 (s, 1H) |
| | purin-6-amine | | |
| 216 | 9-(5-deoxy-5-fluoro-β-D- | 358 | (DMSOd₆) 1.46-1.58 (m, 2H) |
| | ribofuranosyl)-2-(4- | | 1.64-1.76 (m, 2H) 3.43 (t, 2H) 3.98-4.26 (m, 4H) |
| | hydroxybutoxy)-9H-purin- | | 4.46-4.58 (m, 2H) 4.61-4.74 (m, 1H) |
| | 6-amine | | 5.80 (d, 1H) 7.26 (s, 2H) 8.05 (s, 1H) |
| 217 | 9-(5-deoxy-5-fluoro-β-D- | 350 | (DMSO-d₆) 4.00-4.16 (m, 1H) 4.21 (t, 1H) |
| | ribofuranosyl)-2-(2,2- | | 4.40-4.61 (m, 4H) 4.64-4.75 (m, 1H) |
| | difluoroethoxy)-9H-purin- | | 5.82 (d, 1H) 6.34 (tt, 1H) 7.46 (s, 2H) |
| | 6-amine | | 8.11 (s, 1H) |
| 218 | 9-(5-deoxy-5-fluoro-β-D- | 396 | (DMSO-d6) 1.83-1.99 (m, 2H) |
| | ribofuranosyl)-2-(4,4,4- | | 2.26-2.44 (m, 2H) 3.99-4.15 (m, 1H) 4.17-4.29 (m, |
| | trifluorobutoxy)-9H-purin- | | 3H) 4.45-4.58 (m, 2H) 4.62-4.75 (m, 1H) |
| | 6-amine | | 5.81 (d, 1H) 7.30 (s, 2H) 8.07 (s, 1H) |
| 219 | 9-(5-deoxy-5-fluoro-β-D- | 328 | (DMSO-d₆) 0.94 (t, 3H) 1.60-1.76 (m, 2H) |
| | ribofuranosyl)-2-(4,4,4- | | 4.00-4.18 (m, 3H) 4.24 (t, 1H) |
| | trifluorobutoxy)-9H-purin- | | 4.48-4.61 (m, 2H) 4.63-4.79 (m, 1H) 5.81 (d, 1H) |
| | 6-amine | | 7.26 (s, 2H) 8.05 (s, 1H) |
| 220 | 9-(5-deoxy-5-fluoro-β-D- | 372 | 1.27 (d, 6H) 1.91-2.00 (m, 2H) |
| | ribofuranosyl)-2-(3- | | 4.11-4.26 (m, 1H) 4.39-4.49 (m, 3H) 4.56-4.66 (m, |
| | hydroxy-3-methylbutoxy)- | | 2H) 4.71-4.79 (m, 1H) 5.95 (dd, 1H) |
| | 9H-purin-6-amine | | 8.00 (d, 1H) |
| 221 | 2-(cyclobutyloxy)-9-(5- | 340 | 1.60-1.90 (m, 2H) 2.02-2.23 (m, 2H) |
| | deoxy-5-fluoro-β-D- | | 2.39-2.56 (m, 2H) 4.11-4.28 (m, 1H) 4.41 (t, 1H) |
| | ribofuranosyl)-9H-purin-6- | | 4.53-4.67 (m, 2H) 4.68-4.81 (m, 1H) |
| | amine | | 5.10-5.25 (m, 1H) 5.93 (d, 1H) 8.00 (s, 1H) |
| 222 | 9-(5-deoxy-5-fluoro-β-D- | 360 | 2.18 (s, 3H) 2.86 (t, 2H) 4.11-4.28 (m, 1H) |
| | ribofuranosyl)-2-[2- | | 4.40 (t, 1H) 4.49 (t, 2H) 4.54-4.66 (m, |
| | (methylthio) ethoxy]-9H- | | 2H) 4.70-4.81 (m, 1H) 5.95 (d, 1H) |
| | purin-6-amine | | 8.02 (s, 1H) |
| 223 | 9-(5-deoxy-5-fluoro-β-D- | 372 | 1.15 (d, 6H) 3.62-3.73 (m, 1H) |
| | ribofuranosyl)-2-(2- | | 3.73-3.79 (m, 2H) 4.11-4.26 (m, 1H) 4.38-4.45 (m, |
| | isopropoxy-ethoxy)-9H- | | 3H) 4.55-4.65 (m, 2H) 4.74 (dd, 1H) |
| | purin-6-amine | | 5.94 (d, 1H) 8.00 (s, 1H) |
| 224 | 9-(5-deoxy-5-fluoro-β-D- | 328 | 1.36 (dd, 6H) 4.12-4.28 (m, 1H) 4.42 (t, 1H) |
| | ribo-furanosyl)-2- | | 4.54-4.67 (m, 2H) 4.69-4.81 (m, 1H) |
| | isopropoxy-9H-purin-6- | | 5.20-5.34 (m, 1H) 5.94 (d, 1H) 8.01 (s, 1H) |
| | amine | | |
| 225 | 9-(5-deoxy-5-fluoro-β-D- | 370 | 1.43 (s, 3H) 4.12-4.28 (m, 1H) |
| | ribofuranosyl)-2-[(3- | | 4.39-4.46 (m, 5H) 4.57-4.68 (m, 4H) 4.72-4.82 (m, |
| | methyloxetan-3- | | 1H) 5.97 (d, 1H) 8.03 (s, 1H) |
| | yl)methoxy]-9H-purin-6- | | |
| | amine | | |
| 226 | 9-(5-deoxy-5-fluoro-β-D- | 324 | 2.90 (t, 1H) 4.45 (t, 1H) 4.62 (dd, 1H) |
| | ribofuranosyl)-2-(prop-2- | | 4.68 (t, 1H) 4.77 (dd, 1H) 4.98 (d, 2H) 5.95 (d, 1H) |
| | yn-1-yloxy)-9H-purin-6- | | 8.04 (s, 1H) |
| | amine | | |
| 227 | 2-(2-cyclopropylethoxy)-9- | 354 | 0.01-0.21 (m, 2H) 0.37-0.55 (m, 2H) |
| | (5-deoxy-5-fluoro-β-D- | | 0.74-0.99 (m, 1H) 1.65 (q, 2H) 4.07-4.30 (m, |
| | ribofuranosyl)-9H-purin-6- | | 1H) 4.29-4.48 (m, 3H) 4.52-4.67 (m, 2H) |
| | amine | | 4.75 (dd, 1H) 5.95 (d, 1H) 8.01 (s, 1H) |
| 228 | 9-(5-deoxy-5-fluoro-β-D- | 366 | 4.13-4.28 (m, 1H) 4.43 (t, 1H) |
| | ribofuranosyl)-2-(2- | | 4.57-4.67 (m, 2H) 4.76 (dd, 1H) 5.31 (s, 2H) 5.97 (d, |
| | furylmethoxy)-9H-purin-6- | | 1H) 6.38 (dd, 1H) 6.51 (d, 1H) 7.49 (d, 1H) |
| | amine | | 8.02 (s, 1H) |
| 229 | 9-(5-deoxy-5-fluoro-β-D- | 397 | 1.97-2.09 (m, 2H) 2.35 (t, 2H) 3.60 (t, 2H) |
| | ribofuranosyl)-2-[2-(2- | | 3.66 (t, 2H) 4.11-4.27 (m, 1H) 4.41 (t, |
| | oxopyrrolidin-1-yl)ethoxy]- | | 1H) 4.48 (t, 2H) 4.56-4.67 (m, 2H) |
| | 9H-purin-6-amine | | 4.75 (dd, 1H) 5.95 (d, 1H) 8.02 (s, 1H) |
| 230 | : 9-(5-deoxy-5-fluoro-β-D- | 432 | 1.51 (d, 3H) 4.12-4.28 (m, 1H) 4.38 (t, |
| | ribofuranosyl)-2-(2,2,3,3,3- | | J = 5.18 Hz, 1H) 4.54-4.63 (m, 2H) |
| | pentafluoro-1- | | 4.76 (dd, 1H) 5.83-5.92 (m, 1H) 5.94 (d, 1H) |
| | methylpropoxy)-9H-purin- | | 8.06 (s, 1H) |
| | 6-amine | | |
| 231 | 9-(5-deoxy-5-fluoro-β-D- | 384 | 0.97 (t, 3H) 1.82-1.95 (m, 2H) |
| | ribofuranosyl)-2-[(3- | | 4.12-4.28 (m, 1H) 4.42 (t, 1H) 4.44-4.51 (m, 4H) |
| | ethyloxetan-3-yl)methoxy]- | | 4.57-4.64 (m, 4H) 4.76 (dd, 1H) 5.97 (d, 1H) |
| | 9H-purin-6-amine | | 8.03 (s, 1H) |
| 232 | 9-(5-deoxy-5-fluoro-β-D- | 352 | 0.97 (t, 3H) 1.82-1.95 (m, 2H) |
| | ribofuranosyl)-2-[(1- | | 4.12-4.28 (m, 1H) 4.42 (t, 1H) 4.44-4.51 (m, 4H) |
| | methylbut-2-yn-1-yl)oxy]- | | 4.57-4.64 (m, 4H) 4.76 (dd, 1H) 5.97 (d, 1H) |
| | 9H-purin-6-amine | | 8.03 (s, 1H) |
| 233 | 9-(5-deoxy-5-fluoro-β-D- | 382 | 1.49 (d, 3H) 4.11-4.27 (m, 1H) 4.38 (t, 1H) |
| | ribofuranosyl)-2-(2,2,2- | | 4.53-4.63 (m, 2H) 4.72-4.79 (m, 1H) |
| | trifluoro-1-methylethoxy)- | | 5.71-5.84 (m, 1H) 5.92-5.97 (m, 1H) |
| | 9H-purin-6-amine | | 8.05 (s, 1H) |
| 234 | 9-(5-deoxy-5-fluoro-β-D- | 370 | 1.00 (s, 9H) 1.71 (t, 2H) 4.12-4.28 (m, 1H) |
| | ribofuranosyl)-2-(3,3- | | 4.34-4.45 (m, 3H) 4.57-4.67 (m, 2H) |
| | dimethylbutoxy)-9H-purin- | | 4.73-4.79 (m, 1H) 5.95 (d, 1H) 8.01 (s, 1H) |
| | 6-amine | | |
| 235 | 9-(5-deoxy-5-fluoro-β-D- | 346 | 2.14 (tt, 2H) 4.12-4.27 (m, 1H) |
| | ribofuranosyl)-2-(3- | | 4.38-4.56 (m, 5H) 4.56-4.71 (m, 4H) 4.75 (dd, 1H) |
| | fluoropropoxy)-9H-purin-6- | | 5.48 (s, 1H) 5.95 (d, 1H) 8.02 (s, 1H) |
| | amine | | |
| 236 | 9-(5-deoxy-5-fluoro-β-D- | 382 | 1.51 (dd, 3H) 4.21 (dd, 1H) 4.39 (t, 1H) |
| | ribofuranosyl)-2-(3,3,3- | | 4.55-4.65 (m, 2H) 4.77 (dd, 1H) |
| | trifluoropropoxy)-9H-purin- | | 5.73-5.85 (m, 1H) 5.96 (d, 1H) 8.06 (s, 1H) |
| | 6-amine | | |
| 237 | 9-(5-deoxy-5-fluoro-β-D- | 394 | 4.09-4.30 (m, 1H) 4.41 (t, 1H) |
| | ribofuranosyl)-2-[(3- | | 4.52-4.65 (m, 2H) 4.73 (dd, 1H) 5.40 (s, 2H) 5.96 (d, |
| | fluorobenzyl)oxy]-9H- | | 1H) 6.91-7.10 (m, 1H) 7.14-7.45 (m, 3H) |
| | purin-6-amine | | 8.03 (s, 1H) |
| 238 | 9-(5-deoxy-5-fluoro-β-D- | 396 | 1.01 (d, 6H) 1.19-1.43 (m, 4H) |
| | ribofuranosyl)-2-[(3,3- | | 1.53-1.77 (m, 2H) 1.83 (dd, 1H) 2.09-2.23 (m, 1H) |
| | dimethylcyclohexyl)oxy]- | | 4.11-4.27 (m, 1H) 4.40 (q, 1H) |
| | 9H-purin-6-amine | | 4.52-4.79 (m, 3H) 5.06-5.21 (m, 1H) 5.92 (d, 1H) |
| | | | 8.00 (s, 1H) |
| 239 | 9-(5-deoxy-5-fluoro-β-D- | 390 | 1.63 (dd, 3H) 4.08-4.28 (m, 1H) |
| | ribofuranosyl)-2-(1- | | 4.30-4.46 (m, 1H) 4.49-4.66 (m, 2H) 4.77 (dd, 1H) |
| | phenylethoxy)-9H-purin-6- | | 5.90 (dd, 1H) 6.09-6.24 (m, 1H) |
| | amine | | 7.19-7.28 (m, 1H) 7.27-7.39 (m, 2H) |
| | | | 7.37-7.52 (m, 2H) 7.98 (d, 1H) |
| 240 | 9-(5-deoxy-5-fluoro-β-D- | 410 | 0.91 (d, 6H) 1.09-1.25 (m, 3H) |
| | ribofuranosyl)-2-[(4- | | 1.36-1.51 (m, 3H) 1.83 (s, 2H) 2.15-2.26 (m, 2H) |
| | isopropylcyclohexyl)oxy]- | | 4.12-4.26 (m, 1H) 4.41 (t, 1H) |
| | 9H-purin-6-amine | | 4.57-4.67 (m, 3H) 4.75 (dd, 1H) 5.92 (d, 1H) 8.00 (s, |
| | | | 1H) |
| 241 | 9-(5-deoxy-5-fluoro-β-D- | 3700 | 3.65 (s, 1H) 3.76 (s, 1H) 4.15 (s, 4H) |
| | ribofuranosyl)-2- | | 4.52 (s, 2H) 4.68 (s, 1H) 5.83 (s, 1H) 7.31 (s, 2H) |
| | (tetrahydrofuran-2- | | 8.11 (s, 1H) |
| | ylmethoxy)-9H-purin-6- | | |
| | amine | | |
| 242 | 9-(5-deoxy-5-fluoro-beta- | 444 | 4.09-4.29 (m, 1H) 4.39 (t, 1H) |
| | D-ribofuranosyl)-2-[(2,4- | | 4.55-4.65 (m, 2H) 4.68-4.77 (m, 1H) 5.47 (s, 2H) |
| | dichlorobenzyl)oxy]-9H- | | 5.97 (d, 1H) 7.35 (dd, 1H) 7.47-7.53 (m, 1H) |
| | purin-6-amine | | 7.60 (d, 1H) 8.04 (s, 1H) |

### EXAMPLE 243

### 9-(5-deoxy-beta-D-ribofuranosyl)-2-[(2,2,3,3-tetrafluorocyclobutyl)methoxy]-9H-purin-6-amine

A mixture of 9-[5-deoxy-2,3-O-(1-methylethylidene)-β-D-ribofuranosyl]-2-fluoro-9H-purin-6-amine (200 mg) and (2,2,3,3-tetrafluorocyclobutyl)methanol (0.6 ml) in THF (1 ml) was stirred at rt in the presence of sodium hydroxide (250 mg; solid) for 60 h. The reaction was diluted with DCM and filtered through diatomaceous earth. The organic layer was washed with water, dried, and concentrated. The residue was purified by chromatography using 80-100% EtOAc in hexanes as eluent to give the intermediate, 9-[5-deoxy-2,3-O-(1-methylethylidene)-β-D-ribofuranosyl]-2-[(2,2,3,3-tetrafluorocyclobutyl)methoxy]-9H-purin-6-amine (240 mg). The acetonide protecting group was removed by reacting the intermediate (225 mg) with a 1:1 mixture of formic acid and water (3 mL total) at rt for 24 h. The reaction mixture was concentrated and the residue was purified by chromatography using 10-20% MeOH in EtOAc as eluent to give the desired compound (158 mg).

MS (ESP): 408 (MH⁺) for C₁₅H₁₇F₄N₅O₄

¹H NMR (300 MHz, MeOD) δ ppm 1.40 (d, 3H) 2.24-2.59 (m, 1H) 2.63-2.91 (m, 1H) 3.18-3.29 (m, 1H) 3.95-4.22 (m, 2H) 4.43-4.61 (m, 2H) 4.68 (t, 1H) 5.86 (d, 1H) 8.04 (s, 1H).

The intermediates were made as follows:—

### 9-[5-deoxy-2,3-O-(1-methylethylidene)-(3-D-ribofuranosyl]-2-fluoro-9H-purin-6-amine

Lithium triethylborohydride (1M in THF) (120 ml, 3 eq) was added dropwise via an addition funnel to 2-fluoro-9-{2,3-O-(1-methylethylidene)-5-O-[(4-methylphenyl)sulfonyl]-β-D-ribofuranosyl}-9H-purin-6-amine (21 g) at 0° C. The reaction was warmed to rt and stirred for 4 h, and quenched by careful addition of water. The volatiles were concentrated in vacuo, and the remaining residue was partitioned between DCM and saturated sodium bicarbonate. The organic layer was washed with water and brine and dried over sodium sulfate. After concentration in vacuo, EtOAc/hexane (3:1) was added and the precipitate was collected by filtration and dried overnight. The product was obtained in 2 crops (9.81 g total) and used without further purification.

MS (ESP): 310 (MH⁺) for C₁₃H₁₆FN₅O₃

¹H NMR δ ppm 1.24 (d, 3H); 1.30 (s, 3H); 1.51 (s, 3H); 4.22 (m, 1H); 4.74 (dd, 1H); 5.39 (d, 1H); 5.98 (d, 1H); 7.90 (br s, 2H); 8.30 (s, 1H)

### 2-fluoro-9-{2,3-O-(1-methylethylidene)-5-O-[(4-methylphenyl)sulfonyl]-β-D-ribofuranosyl}-9H-purin-6-amine

2-Fluoro-9-[2,3-O-(1-methylethylidene)-β-D-ribofuranosyl]-9H-purin-6-amine (20 g) was dissolved in pyridine (140 ml) and cooled to −10° C. A solution of tosyl chloride (23 g, 2 equivalents) in pyridine (50 ml) was added dropwise over-0.5 h via addition funnel. The reaction was allowed to slowly warm to rt and stir overnight. The volatiles were concentrated in vacuo and the residue was partitioned between chloroform and saturated sodium bicarbonate. The organic layer was washed with saturated sodium bicarbonate, water, and brine and dried over sodium sulfate. Hexane/EtOAc (3:1) was added to the resulting black syrup, and the precipitate was collected by filtration. The brown solid (21 g) was used without further purification.

MS (ESP): 480 (MH⁺) for C₂₀H₂₂FN₅O₆S

¹H NMR δ: 1.27 (s, 3H); 1.49 (s, 3H); 2.34 (s, 3H); 4.18-4.34 (series of m, 3H); 4.89 (dd, 1H); 5.26 (dd, 1H); 6.09 (d, 1H); 7.23 (d, 2H); 7.56 (d, 2H); 7.93 (br s, 2H); 8.18 (s, 1H)

### 2-fluoro-9-[2,3-O-(1-methylethylidene)-β-D-ribofuranosyl]-9H-purin-6-amine

2-Fluoroadenosine (40 g) (purchased from General Intermediates of Canada) was dissolved in acetone (400 ml)/N-methylpyrrolidinone (400 ml). Dimethoxypropane (35 ml, 2 eq) and p-tolenesulfonic acid (5.3 g, 0.2 eq) were added, and the reaction was stirred at 40° C. overnight. Acetone was removed in vacuo and the remaining residue was diluted with EtOAc and saturated sodium bicarbonate. The aqueous layer was extracted with EtOAc, and the combined organic layers were washed with water and dried over sodium sulfate. The mixture was concentrated in vacuo, then water was added and the solution cooled to 0° C. Solid was collected by filtration and washed with cold water. Product was dried under high vacuum to obtain an off-white solid (45 g).

MS (ESP): 326 (MH⁺) for C₁₃H₁₆FN₅O₄

¹H NMR δ: 1.31 (3H, s); 1.52 (3H, s); 2.49 (2H, m); 4.18 (1H, m); 4.92 (1H, m); 5.08 (1H, m); 6.01 (1H, d); 7.88 (2H, br s); 8.31 (1H, s)

### EXAMPLE 244

### 9-(5-deoxy-beta-D-ribofuranosyl)-2-[(2,2,3,3-tetrafluorocyclobutyl)oxy]-9H-purin-6-amine

This compound was prepared using an analogous procedure to that described for Example 243, by reacting 2,2,3,3-tetrafluorocyclobutanol with 9-[5-deoxy-2,3-O-(1-methylethylidene)-β-D-ribofuranosyl]-2-fluoro-9H-purin-6-amine, followed by removal of the protecting group.

MS (ESP): 394 (MH⁺) for C₁₄H₁₅F₄N₅O₄

¹H NMR (300 MHz, MeOD) d ppm 1.41 (t, 3H) 2.60-2.87 (m, 1H) 3.05-3.24 (m, 1H) 3.92-4.19 (m, 2H) 4.72 (q, 1H) 5.40-5.65 (m, 1H) 5.85 (d, 1H) 8.07 (d, 1H).

### EXAMPLE 245

### 2-[(4-cyanobenzyl)oxy]-9-(5-deoxy-β-D-ribofuranosyl)-9H-purin-6-amine

This compound was prepared using an analogous procedure to that described in Example 243, by reacting 4-(hydroxymethyl)benzonitrile (20 eq) with 9-[5-deoxy-2,3-O-(1-methylethylidene)-β-D-ribofuranosyl]-2-fluoro-9H-purin-6-amine (0.2 g, 0.64 mmol), followed by removal of the protecting group to give desired product after Gilson purification.

MS (ESP): 383 (MH⁺)

¹H NMR (300 MHz, DMSO-d₆) δ ppm 1.22 (d, 3H) 3.85-3.96 (m, 2H) 4.58 (q, 1H) 5.14 (m, 1H) 5.34-5.46 (m, 3H) 5.71 (m, 1H) 7.38 (s, 2H) 7.60 (m, 2H) 7.83 (d, 2H) 8.13 (s, 1H)

### EXAMPLE 246

### 9-(5-deoxy-β-D-ribofuranosyl)-2-{[4-(methoxycarbonyl)benzyl]oxy}-9H-purin-6-amine

To a solution of 9-[5-deoxy-2,3-O-(1-methylethylidene)-β-D-ribofuranosyl]-2-fluoro-9H-purin-6-amine (0.2 g, 0.64 mmol) in DMF (5 ml) was added cesium carbonate (2 g, 6.4 mmol) and methyl 4-(hydroxymethyl)benzoate (1 g, 6.4 mmol). The reaction was stirred at rt, then diluted with chloroform, filtered through diatomaceous earth and concentrated in vacuo. The residue was dissolved in formic acid/water (10 ml, 1:1) and stirred at rt overnight. After concentration in vacuo, the residue was purified by reverse phase HPLC to give the desired product (37 mg).

MS (ESP): 416 (MH⁺)

¹H NMR (300 MHz, DMSO-d₆) δ ppm 1.24 (d, 3H); 3.84 (s, 3H); 3.94 (m, 2H); 4.61 (m, 1H); 5.15 (m, 1H); 5.40 (m, 3H); 5.72 (m, 1H); 7.36 (bs, 2H); 7.56 (m, 2H); 7.95 (m, 2H); 8.13 (s, 1H)

The alcohols used in the above procedures were either commercially available, made using procedures found in the literature, or made using the following procedures:—
(1-Trifluoromethylcyclobutyl)methanol was prepared using the procedure of A Wolniewicz & D Wojciech, J. Fluorine Chem. 2001, 109(2):95-102.

### [1-(fluoromethyl)cyclopropyl]methanol

8 mL of 1M sodium hydroxide was added to 1-(fluoromethyl)cyclopropanyl-methoxy-benzoate (1.2 g, 5.8 mmol) in 20 mL of ethanol. The reaction was stirred overnight at rt. The mixture was concentrated and distributed in 50 mL of EtOAc and 10 mL water. The organic layer was separated and dried over magnesium sulfate. The filtrate was concentrated in vacuo to give a colorless oil (120 mg) as desired product.

¹H NMR δ: 0.27 (m, 4H) 4.02 (s, 1H) 4.17 (s, 1H) 4.45 (m, 3H)

The intermediates for this alcohol were prepared as follows:—

### [1-(fluoromethyl)cyclopropyl]methyl benzoate

The mixture of 1-(tosyloxymethyl)cyclopropanyl-methoxy-benzoate (3.5 g, 9.7 mmol) and 30 mL of 1M tetrabutylammonium fluoride in 5 mL of THF was sealed in a vial and heated at 126° C. for 3 h. The mixture was concentrated in vacuo and purified by column chromatography (hexane/EtOAc=4:1) to yield 1.2 g of colorless oil.

¹H NMR δ: 0.81 (m, 4H) 2.21 (s, 3H) 4.03 (s, 1H) 3.85 (s, 1H) 4.54 (m, 2H) 7.33-7.81 (m, 9H)

### [1-({[(4-methylphenyl)sulfonyl]oxy}methyl)cyclopropyl]methyl benzoate

To a solution 1,1-dihydroxymethylcyclopropane (3.0 g, 29.4 mmol) was added triethylamine (4.0 ml, 1 eq) followed by benzoyl chloride (3.43 ml, 1 equivalent). The resulting mixture was stirred at rt overnight. Tosyl chloride (4.0 g, 21 mmol) was added after additional triethylamine (4.0 ml) and then the mixture was stirred at rt overnight. The mixture was taken up in DCM (100 ml) and washed with water (100 mL×2). The organic layer was isolated and dried over magnesium sulfate. The filtrate was concentrated in vacuo and purified by column chromatography (hexane/EtOAc: 4:1) to yield a white solid (2.7 g).

¹H NMR δ: 0.71 (m, 4H) 4.31 (s, 1. H) 4.38 (s, 1H) 4.54 (m, 2H) 7.61 (m, 2H), 7.74 (m, 1H), 8.05 (m, 2H),

### 3-[(4-methoxybenzyl)oxy]cyclohexanol

1.2 g of sodium hydride (60% dispersion in mineral oil) was slowly added into a solution of 1,3-cyclohexanediol (2.9 g, 25.0 mmol) in DMF (40 ml) at rt, followed by 4-methoxybenzyl bromide (5.0 g, 1 equivalent) The mixture was stirred at rt for 48 h. The mixture was taken up in 100 mL EtOAc, washed with water (3×40 ml), and dried over magnesium sulfate. The mixture was filtered and the filtrate was concentrated and purified by column chromatography (eluting hexane:EtOAc=2:1) to yield 1.7 g colorless oil. It was used without further purification.

MS (ESP): 236 (MH⁻) for C₁₄H₂₀O₃

3,3-difluorocyclohexanol was prepared from the following intermediates:—

### 3-oxocyclohexyl benzoate

To 1.0 g of 1,3-cyclohexanediol in 10 mL of DMF, 0.8 mL of triethylamine and a catalytic amount of dimethylaminopyridine were added followed by 0.6 mL of benzoyl chloride at rt. The reaction was stirred overnight. The mixture was washed with water, dried over magnesium sulfate, and the organics were concentrated to dryness (1.3 g). The resulting residue was subjected to the next step. Dess-Martin reagent (15%, 10 ml) in DCM was added to a solution of 3-(benzoyloxy)cyclohexanol (1.2 g, 5.5 mmol) in DCM (20 ml) at rt. The mixture was stirred at rt for 18 h and concentrated by 80%; the white solid was filtered off. The filtrate was concentrated to dryness to yield a light yellow oil (1.1 g) and was used without purification.

### 3,3-difluorocyclohexyl benzoate

Ishikawa's reagent (N,N-diethyl-1,1,2,3,3,3-hexafluoropropylamine) (0.6 g, 2.7 mmol) was added to a solution of 3-(benzoyloxy)cyclohexanone (0.5 g, 2.3 mmol) in DCM (15 ml) at rt. The mixture was stirred at rt for 18 h. The mixture was washed with water (2×20 ml). The organic layer was separated and dried over magnesium sulfate. After filtration, the filtrate was concentrated and purified by column chromatography (eluting hexane/EtOAc=4:1) to yield a colorless oil (0.9 g) and was used without purification.

¹⁹F NMR (300 MHz, DMSO-d₆): δ ppm −91.14 (d, 1F), 88.43 (d, 1F).

### 3,3-difluorocyclohexanol

To 2.3 g of 3,3-difluorocyclohexyl benzoate in 5 mL of ethanol was added 0.25 g of sodium hydroxide and 5 mL of water. The mixture was stirred at rt for 5 h. The mixture was extracted with DCM (2×30 ml), the organics were dried over magnesium sulfate, concentrated in vacuo and used without further purification.

### (2,3,3-Trifluorocyclobutyl)methanol

Under a nitrogen atmosphere, a solution of 1.83 g (9.03 mmol) of ethyl (2-chloro-2,3,3-trifluoro)cyclobutylcarboxylate in 10 mL of dry THF was added dropwise over 30 min to an ice cold solution of 38 mL of 0.5 M lithium aluminum hydride in THF. After 24 h at rt, the solution was cooled in an ice bath and quenched by dropwise addition of 12 mL of saturated aqueous sodium chloride. The resulting suspension was filtered, the solids were washed with hot EtOAc and the combined filtrate was concentrated in vacuo to a colorless oil, 1.60 g (100%).

¹H NMR (300 MHz, CDCl₃) δ 2.10 (br m, 1H), 2.48 (m, 2H), 3.82 (cp, 2H), 3.50 (s, 1H)

¹⁹F NMR (300 MHz, CDCl₃) δ −81.73, −92.1 (d), −93.1 (d), −98.9 (d), −102.73 (d), −108.85 (d), −113.54 (d), −115.57 (d), −124.37 (d), −136.1, −192.2

### [4-(trifluoromethyl)cyclohexyl]methanol

A solution of 4-(trifluoromethyl)cyclohexane carboxylic acid (2 g) in anhydrous diethyl ether was slowly added to a lithium aluminum hydride (1M in diethyl ether) solution (1.0 eq., 10.20 ml). The reaction was stirred at rt overnight. The reaction was quenched with an ammonium chloride solution and filtered through diatomaceous earth. The ether solution was washed with sodium bicarbonate solution (5%) and most of the solvent was removed in vacuo to yield a colorless oil. This alcohol was used for the displacement reaction without purification.

¹H NMR (300 MHz, CDCl₃) δ ppm 1.42-2.17 (m, 10H) 3.57 (d, 2H)

The following alcohols were synthesized from the corresponding carboxylic acid or ethyl ester by reduction with lithium aluminum hydride using a similar procedure to that described above giving products with analytical characterization identical to that found in the literature references. These alcohols were used for the displacement reactions without further purification.

(2,2-Difluorocyclopropyl)methanol described in Battiste, M. A.; Tian, F.; Baker, J. M.; Bautista, O.; Villalobos, J.; Dolbier Jr., W. R. J. Fluorine Chem. 2003, 119, 39.
[(1S,2S)-2-Fluorocyclopropyl]methanol and [(1S,2R)-2-fluorocyclopropyl]methanol both described in Yukimoto, J.; Ehata, T.; Tojo, T.; Inanaga, M.; Sato, K. (Daiichi Seiyaku Co., Japan) Jpn. Kokai Tokkyo Koho 1995, 11 pp. JP 93-253941 19931012.
[1-(Trifluoromethyl)cyclopropyl]methanol described in Wolniewicz, A.; Wojciech, D. J. Fluorine Chem. 2001, 109, 95.
Spiro[2.2]pent-1-ylmethanol described in Charette, A.; Jolicoeur, E.; Bydlinski, G. A. S. Org. Lett. 2001, 3, 3293.
(4,4-Difluorocyclohexyl)methanol described in MacKvenzie, A. R.; Marchington, A. P.; Middleton, D. S.; Meadows, S. D. PCT Int. Appl. 1997, 79 pp. WO 9727185.
(2,2-dimethylcyclopropyl)methanol described in J. Am. Chem. Soc. 2001, 123, 12160.
(1,2,2-Trifluorocyclopentyl)methanol was prepared as follows:—

The title compound was prepared as above from ethyl (1,2,2-trifluorocyclopentyl)carboxylate by reduction using lithium aluminum hydride.

¹⁹F NMR (300 MHz, CDCl₃) δ −109.03 (d), −118.07 (d), −125.57, −127.60, −170.59

### Ethyl (1,2,2-trifluorocyclopentyl)carboxylate

Under a nitrogen atmosphere, 2.0 mL (3.0 equiv) of diethylamino sulphur trifluoride was added to a solution of 922 mg (5.29 mmol) of ethyl 1-fluoro-2-oxocyclopentanecarboxylate [prepared via the procedure of S T Purrington, et. al., J. Org. Chem. 1987, 52(19): 4307-4310] in 15 mL of DCM. After stirring at rt for 42 h, the reaction was poured into an ice cold solution of 10.7 g of potassium carbonate in 100 mL of water (evolution of carbon dioxide); the layers were separated and the aqueous layer was extracted with 25 mL of DCM. The combined organic extract was dried over anhydrous sodium sulfate and concentrated to give 135 mg (13%) of the title compound as a yellow oil.

¹⁹F NMR (300 MHz, CDCl₃) δ −106.8 (d), −114.6 (d), −136.56, −149.39, −168.25

4,4-Difluorocyclohexanol was synthesized according to the following:—

### 4-hydroxycyclohexyl benzoate

A solution of 1,4-cyclohexanediol (5 g), and triethylamine (1.0 equivalent, 5.982 ml) in anhydrous DCM (20 ml) was stirred at rt for 10 min. Benzoyl chloride (1.0 equivalent, 5 ml) was added slowly by a syringe. The reaction was stirred overnight. The resulting mixture was washed with water (2×20 ml). The organic layer was separated and dried over sodium sulfate. After filtration, the filtrate was condensed and purified on a column (eluting hexane/EtOAc=3:2) to yield colorless oil (5 g).

¹H NMR (300 MHz, CDCl₃) δ ppm 1.42-1.85 (m, 6H) 2.01-2.20 (m, 2H) 3.68-3.90 (m, 1H) 4.93-5.25 (m, 1H) 7.36-7.60 (m, 3H) 7.95-8.11 (m, 2H)

### 4-oxocyclohexyl benzoate

To a solution of 4-hydroxycyclohexyl benzoate (3 g) in anhydrous DCM, 5 g of ground molecular sieves were added followed by pyridinium chlorochromate (1.5 equivalents, 4.40 g). The reaction mixture was stirred at rt overnight. The mixture was filtered through diatomaceous earth and the filtrate was concentrated to dryness, followed by purification on a column (eluting hexane/EtOAc=7:3) to yield colorless oil (2.5 g).

¹H NMR (300 MHz, CDCl₃) δ ppm 2.11-2.32 (m, 4H) 2.38-2.50 (m, 2H) 2.59-2.72 (m, 2H) 5.38-5.48 (m, 1H) 7.42-7.50 (m, 2H) 7.54-7.63 (m, 1H) 8.01-8.10 (m, 2H)

### 4,4-difluorocyclohexyl benzoate

A solution of 4-oxocyclohexyl benzoate (2.3 g) in anhydrous DCM was cooled to 0° C. in an ice bath before addition of diethylamino sulfur trifluoride (3.0 equivalents, 3.88 ml) under nitrogen. After stirring overnight at rt, analysis of an aliquot by GC-MS indicated 100% conversion of ketone had been achieved and that two products had been formed. The mixture was washed with water (2×20 ml). The organic layer was separated and dried over sodium sulfate. After filtration, the filtrate was condensed and dissolved in acetone/water (20 ml, 1:1). Osmium tetroxide (2.5 wt % in water) (1 ml) was added slowly and the reaction mixture was stirred overnight. The mixture was extracted with DCM 3× and the organic layer was dried over sodium sulfate. After filtration, the filtrate was concentrated and purified on column (eluting hexane/EtOAc=4:1) to yield colorless oil (1.8 g).

¹H NMR (300 MHz, CDCl₃) δ ppm 1.89-2.24 (m, 8H) 5.20 (s, 1H) 7.34-7.50 (m, 2H) 7.51-7.64 (m, 1H) 7.96-8.09 (m, 2H)

### 4,4-Difluorocyclohexanol

4,4-Difluorocyclohexyl benzoate (1.8 g) was dissolved in THF (30 ml) and 10% potassium hydroxide (aq) (30 ml). The reaction mixture was stirred at rt overnight. The mixture was extracted with DCM 3× and the organic layer was dried over sodium sulfate. After filtration, the filtrate was concentrated to yield a colorless oil that was used without purification.

Bicyclo[3.1.0]hexan-3-ol was synthesized from the following intermediates:—

### cyclopent-3-en-1-yl benzoate

A solution of 3-cyclopentene-1-ol (2 g), and triethylamine (1.2 equivalents, 3.96 ml) in anhydrous DCM (20 ml) was stirred at rt for 10 min. Benzoyl chloride (1.2 equivalents, 3.28 ml) was added slowly by a syringe. The reaction was allowed to stir overnight. The resulting mixture was washed with water (2×20 ml). The organic layer was separated and dried over sodium sulfate. After filtration, the filtrate was concentrated and purified on column (eluting hexane/EtOAc=9:1) to yield colorless oil (4.3 g).

¹H NMR (300 MHz, CDCl₃) δ ppm 2.45-2.63 (m, 2H) 2.74-2.93 (m, 2H) 5.55-5.67 (m, 1H) 5.76 (s, 2H) 7.41 (2, 2H) 7.48-7.58 (m, 1H) 7.94-8.09 (m, 2H)

### Bicyclo[3.1.0]hex-3-yl benzoate

To a solution of diethylzinc (11.0M in hexanes, 3 equivalents, 48 ml) in DCM (30 ml) at 0° C. was added diiodomethane over 10 min. After another 10 min stirring at 0° C., a solution of cyclopent-3-en-1-yl benzoate (3 g) in DCM (20 ml) was added rapidly by a syringe. The ice bath was removed and the reaction was stirred at rt for 2 d. Analysis of an aliquot by GC-MS indicated 80% conversion of ketone had been achieved and that the desired product had been formed. The mixture was washed with water (2×20 ml). The organic layer was separated and dried over sodium sulfate. After filtration, the filtrate was concentrated in vacuo and dissolved in acetone/water (20 ml, 1:1). Osmium tetroxide (2.5 wt % in water) (1 ml) was added slowly and the reaction mixture was stirred overnight. The mixture was extracted with DCM 3× and the organic layer was dried over sodium sulfate. After filtration, the filtrate was concentrated and purified by column chromatography (eluting hexane/EtOAc=95:5) to yield colorless oil (2.7 g).

¹H NMR (300 MHz, CDCl₃) δ ppm 0.07-0.62 (m, 2H) 1.28-1.47 (m, 2H) 1.85-2.07 (m, 2H) 2.19-2.64 (m, 2H) 4.90-5.70 (m, 1H) 7.37-7.50 (m, 2H) 7.54 (t, J=7.35 Hz, 1H) 7.85-8.09 (m, 2H)

### Bicyclo[3.1.0]hexan-3-ol

Bicyclo[3.1.0]hex-3-yl benzoate (2.5 g) was dissolved in THF (10 ml) and 10% potassium hydroxide (aq) (10 ml). The reaction mixture was stirred at rt overnight. The mixture was extracted with DCM (3×) and the organic layer was dried over sodium sulfate. After filtration, the filtrate was concentrated to yield colorless oil and was used without purification.

(3,3-difluorocyclopentyl)MeOH was synthesized using the following procedures:—

### ethyl-3,3-difluorocyclopentane carboxylate

A solution of ethyl, 3-oxocyclopentanecarboxylate (500 mg) in anhydrous DCM was cooled to 0° C. in a ice bath before addition of diethyl amino sulfur trifluoride (1.5 equivalents, 0.59 ml) under nitrogen. After stirring overnight at rt, analysis of an aliquot by GC-MS indicated 100% conversion of ketone had been achieved. The mixture was washed with water (2×20 ml). The organic layer was separated and dried over sodium sulfate. After filtration, the filtrate was concentrated carefully and the residue was used in the next step without purification.

### (3,3-difluorocyclopentyl)methanol

A solution of ethyl 3,3-difluorocyclopentane carboxylate in anhydrous diethyl ether was slowly added to a lithium aluminum hydride (1M in diethyl ether) solution (1.0 equivalents, 3.20 ml). The reaction was stirred at rt for 4 h. The reaction was quenched by ammonium chloride solution and filtered through diatomaceous earth. The ether solution was washed with sodium bicarbonate solution (5%) and most of solvent was removed to yield colorless oil. This alcohol was used for displacement reaction without purification.

3,3-difluorocyclopentanol was synthesized according to the following procedures:—

### 4-oxocyclopent-2-en-1-yl acetate

To a solution of (1R,4S)-4-hydroxycyclopent-2-en-1-yl acetate (2 g) in anhydrous DCM, 5 g of ground molecular sieves was added followed by pyridinium chlorochromate (1.5 equivalents, 4.55 g). The reaction mixture was stirred at rt overnight. The mixture was filtered through diatomaceous earth and the filtrate was concentrated to dryness, followed by purification by column chromatography (eluting hexane/EtOAc=7:3) to yield colorless oil (1.9 g).

¹H NMR (300 MHz, CDCl₃) δ ppm 2.08 (s, 3H) 2.31 (dd, 1H) 2.81 (dd, 1H) 5.74-5.91 (m, 1H) 6.32 (dd, 1H) 7.55 (dd, 1H)

### 4-hydroxycyclopent-2-en-1-one

Made using the procedure described in Gerdil, Raymond; Liu, Huiyou; Bernardinelli, Gerald. Helvetica Chimica Acta (1999), 82(3), 418-434

### 4-oxocyclopent-2-en-1-yl benzoate

A solution of 4-hydroxycyclopent-2-en-1-one (2.5 g), and triethylamine (2.98 ml) in anhydrous DCM (50 ml) was stirred at rt for 10 min. Benzoyl chloride (2.46 ml) was added slowly by a syringe. The reaction was stirred overnight. The mixture was washed with water (2×20 ml). The organic layer was separated and dried over sodium sulfate. After filtration, the filtrate was concentrated and purified on by column chromatography (eluting hexane/EtOAc=3:2) to yield yellow solid (2.2 g).

### 3-oxocyclopentyl benzoate

To an EtOAc suspension (10 ml) of 10% palladium on carbon, 4-oxocyclopent-2-en-1-yl benzoate (2.2 g) was added and the mixture was stirred at rt for 10 min. A hydrogen-filled balloon was fitted to the flask and the mixture was stirred at rt overnight. The reaction mixture was filtered through diatomaceous earth. The clear solution was dried to yield a white solid (2.0 g).

### 3,3-difluorocyclopentyl benzoate

A solution of 3-oxocyclopentyl benzoate (2.0 g) in anhydrous DCM was cooled to 0° C. in a ice bath before addition of diethyl amino sulfur trifluoride (2.0 ml) under nitrogen. After stirring overnight at rt, analysis of an aliquot by GC-MS indicated 100% conversion of ketone had been achieved and that two products had been formed. The mixture was washed with water (2×20 ml). The organic layer was separated and dried over sodium sulfate. After filtration, the filtrate was concentrated carefully and the residue was used in the next step without purification.

### 3,3-difluorocyclopentanol

3,3-difluorocyclopentyl benzoate (2.0 g) was dissolved in MeOH (5 ml) and 10% potassium hydroxide (aq) (5 ml). The reaction mixture was stirred at rt overnight. The mixture was extracted with DCM 5× and the organic layer was dried over sodium sulfate. After filtration, the filtrate was concentrated to yield a colorless oil and was used without further purification.

6,6-difluorobicyclo[3.1.0]hexan-3-ol was made using the following procedures:—

### cyclopent-3-en-1-yl benzoate

A solution of 3-cyclopentene-1-ol (2 g), and triethylamine (1.2 equivalents, 3.96 ml) in anhydrous DCM (20 ml) was stirred at rt for 10 min. Benzoyl chloride (1.2 equivalents, 3.28 ml) was added slowly by a syringe. The reaction was stirred overnight. The mixture was washed with water (2×20 ml). The organic layer was separated and dried over sodium sulfate. After filtration, the filtrate was concentrated and purified by column chromatography (eluting hexane/EtOAc=9:1) to yield a colorless oil (4.3 g).

¹H NMR (300 MHz, CDCl₃) δ ppm 2.45-2.63 (m, 2H) 2.74-2.93 (m, 2H) 5.55-5.67 (m, 1H) 5.76 (s, 2H) 7.41 (2, 2H) 7.48-7.58 (m, 1H) 7.94-8.09 (m, 2H)

### 6,6-difluorobicyclo[3.1.0]hex-3-yl benzoate

A dry 25 mL two neck round bottom flask, equipped with a magnetic stirring bar, was charged with 4 mg of initiator sodium fluoride and 2.25 g (11.97 mmol) of cyclopent-3-en-1-yl benzoate. Under nitrogen and at 100° C., 7.49 g (29.92 mmol, 2.5 equivalents) of trimethylsilyl 2,2-difluoro-2-(fluorosulfonyl)acetate, was added slowly using a syringe over 4-5 h. Upon the completion of the addition, the reaction mixture was stirred for additional 0.5 h and cooled to rt, then diluted with DCM. The solution was washed with water, 5% sodium bicarbonate, water and brine and dried over sodium sulfate. The solvent was removed in vacuo. Purification of the products on flash column chromatography (eluting hexane/EtOAc=95:5) provided a colorless oil (2.2 g).

### 6,6-difluorobicyclo[3.1.0]hexan-3-ol

6,6-difluorobicyclo[3.1.0]hex-3-yl benzoate (2.2 g) was dissolved in MeOH (15 ml) and 10% potassium hydroxide (aq) (15 ml). The reaction mixture was stirred at rt overnight. The mixture was extracted with DCM 5× and the organic layer was dried over sodium sulfate. After filtration, the filtrate was concentrated to yield a colorless oil and was used without further purification.

### EXAMPLE 247

### 9-(5-deoxy-beta-D-ribofuranosyl)-2-(4-fluorophenoxy)-9H-purin-6-amine

A mixture of 2-chloro-9-[5-deoxy-2,3-O-(1-methylethylidene)-β-D-ribofuranosyl]-9H-purin-6-amine (226 mg), 4-fluorophenol (300 mg), and cesium carbonate (500 mg) in N-methylpyrrolidinone (1.5 ml) was stirred at 100° C. for 16 h. Reaction was diluted with EtOAc and washed sequentially with water, 10% aqueous sodium carbonate, and brine. Organic layer was dried over sodium sulfate, filtered and concentrated. The residue was purified on a silica gel column (elution with 80-100% EtOAc in hexanes) to give the intermediate 9-[5-deoxy-2,3-O-(1-methylethylidene)-β-D-ribofuranosyl]-2-(4-fluorophenoxy)-9H-purin-6-amine (200 mg). Protecting group acetonide was removed by reacting this intermediate (185 mg) with a 1:1 mixture of formic acid and water (6 mL total) at rt for 27 h. At the end of the reaction, the mixture was concentrated and the residue was purified on a silica gel column (elution with 10-20% MeOH in EtOAc) to get the desired compound (120 mg).

MS (ESP): 362 (MH⁺) for C₁₆H₁₆FN₅O₄

¹H NMR (300 MHz, MeOD) δ ppm 1.12 (d, 3H) 3.79 (t, 1H) 3.96 (dd, 5.09 Hz, 1H) 4.64 (t, 1H) 5.71 (d, 1H) 6.98-7.29 (m, 4H) 8.04 (s, 1H)

Using and analogous procedure to Example 247, by reacting the appropriate commercially available phenol with 9-[5-deoxy-2,3-O-(1-methylethylidene)-β-D-ribofuranosyl]-2-chloro-9H-purin-6-amine, followed by deprotection, the compounds described in Table X were obtained. The reactions can be heated from 80-130° C. for 24-72 h.

TABLE X

| EX | IUPAC Name | MH+ | ¹H NMR (300 MHz) MeOD, δ ppm |
|---|---|---|---|
| 248 | 9-(5-deoxy-beta-D-ribofuranosyl)- | 344 | 1.08 (d, 3H) 3.74 (t, 1H) 3.94 (dd, 1H) |
| | 2-phenoxy-9H-purin-6-amine | | 4.64 (t, 1H) 5.70 (d, 1H) 7.03-7.26 (m, 3H) |
| | | | 7.29-7.52 (m, 2H) 8.03 (s, 1H) |
| 249 | 2-(3-chlorophenoxy)-9-(5-deoxy- | 378 | 1.13 (d, 3H) 3.78 (t, 1H) 3.97 (dd, 1H) |
| | beta-D-ribofuranosyl)-9H-purin-6- | | 4.68 (t, 1H) 5.72 (d, 1H) 7.05-7.16 (m, 1H) |
| | amine | | 7.16-7.30 (m, 1H) 7.33-7.49 (m, 1H) 8.05 (s, 1H) |
| 250 | 9-(5-deoxy-beta-D-ribofuranosyl)- | 412 | 1.14 (d, 3H) 3.82 (t, 1H) 4.00 (dd, 1H) |
| | 2-(3,4-dichlorophenoxy)-9H-purin- | | 4.68 (t, 1H) 5.73 (d, 1H) 7.14 (dd, 1H) 7.40 (d, 1H) |
| | 6-amine | | 7.55 (d, 1H) 8.06 (s, 1H) |
| 251 | 2-(2-chloro-4-fluorophenoxy)-9-(5- | 396 | 1.09 (d, 3H) 3.71 (t, 1H) 3.86-4.04 (m, 1H) |
| | deoxy-beta-D-ribofuranosyl)-9H- | | 4.52-4.66 (m, 1H) 5.69 (d, 1H) |
| | purin-6-amine | | 7.05-7.20 (m, 1H) 7.24-7.42 (m, 2H) 8.03 (s, 1H) |
| 252 | 9-(5-deoxy-beta-D-ribofuranosyl)- | 380 | 1.12 (d, 3H) 3.76 (t, 1H) 3.88-4.07 (m, 1H) |
| | 2-(2,4-difluorophenoxy)-9H-purin- | | 4.62 (t, 1H) 5.71 (d, 1H) 6.89-7.18 (m, 2H) |
| | 6-amine | | 7.23-7.39 (m, 1H) 8.04 (s, 1H) |
| 253 | 2-(4-chloro-2-fluorophenoxy)-9-(5- | 396 | 1.12 (d, 3H) 3.78 (t, 1H) 3.90-4.03 (m, 1H) |
| | deoxy-beta-D-ribofuranosyl)-9H- | | 4.62 (t, 1H) 5.71 (d, 1H) 7.16-7.41 (m, 3H) |
| | purin-6-amine | | 8.05 (s, 1H) |

### EXAMPLE 254

### 9-(2,3-anhydro-5-deoxy-5-fluoro-β-D-ribofuranosyl)-2-(cyclopentyloxy)-9H-purin-6-amine

To a solution of 2-(cyclopentyloxy)-9-(5-deoxy-5-fluoro-β-D-ribofuranosyl)-9H-purin-6-amine (prepared as for Example 185) (100 mg, 2.83 mmol) in a mixture of acetonitrile (10 ml) and water (5 μl) was added 1-bromocarbonyl-1-methylethyl acetate (208 μl, 1.4 mmol) at 4° C. The solution was stirred at rt for 1.5 h, then quenched with water and saturated sodium bicarbonate. The reaction mixture was extracted with EtOAc (2×50 ml), dried (sodium sulfate), filtered and concentrated in vacuo to give 9-(3-bromo-3,5-dideoxy-5-fluoro-β-D-xylofuranosyl)-2-(cyclopentyloxy)-9H-purin-6-amine. This intermediate was taken up in dry MeOH then 200 mg of Dowex(OH⁻) was added to the reaction mixture and the stirring was continued for 12 h at rt. The Dowex(OH⁻) was filtered off and the filtrate was concentrated in vacuo. The residue was purified by chromatography eluting with 7% MeOH in DCM to give the desired product (66 mg).

MS (ESP): 336 (MH⁺) for C₁₅H₁₈FN₅O₃

¹H NMR δ: 1.67-2.02 (m, 8H) 4.43 (d, 1H) 4.50-4.53 (m, 1H) 4.63 (d, 1H) 4.64-4.82 (m, 2H) 5.35-5.41 (m, 1H) 6.28 (s, 1H) 7.38 (s, 2H) 8.12 (s, 1H)

### EXAMPLE 255

### 2-(cyclopentyloxy)-9-(3,5-dideoxy-5-fluoro-O-D-erythro-pentofuranosyl)-5-9H-purin-6-amine

To a solution of 9-(2,3-anhydro-5-deoxy-5-fluoro-β-D-ribofuranosyl)-2-(cyclopentyloxy)-9H-purin-6-amine (59 mg, 0.18 mmol) in THF was added a solution of lithium aluminum hydride in THF 1M (1.06 ml, 1.06 mmol) at 4° C. The solution was stirred at 4° C. for 6 h, then at rt overnight. The reaction mixture was quenched with ice water and extracted with DCM. The organic phase was dried (sodium sulfate), filtered and concentrated in vacuo. The residue was purified by chromatography eluting with 7% MeOH in DCM. The product was further purified using Gilson reverse phase HPLC with 10 mM ammonium acetate and acetonitrile as the mobile phases with a gradient of 0-75% in 10 min. Relevant fractions were combined to give 10 mg of the desired product.

MS (ESP): 338 (MH⁺) for C₁₅H₂₀FN₅O₃

¹H NMR (300 MHz, CDCl₃) δ ppm 1.41-1.83 (m, 8H) 2.17 (ddd, 1H) 2.34 (dt, 1H) 4.33 (dd, 1H) 4.49 (dd, 2.92 Hz, 1H) 4.55-4.73 (m, 3H) 5.19 (m, 1H) 5.75 (d, 3H) 7.82 (s, 1H)

### EXAMPLE 256

### 2-(cyclopentyloxy)-9-β-D-ribofuranuronosyl-9H-purin-6-amine

To a solution of N-benzoyl-2-(cyclopentyloxy)-9-[2,3-O-(1-methylethylidene)-β-D-ribofuranosyl]-9H-purin-6-amine prepared as described for Example 129 (400 mg, 0.81 mmol) and sodium periodate (709 mg, 3.31 mmol) in a mixture of acetonitrile/carbon tetrachloride/water 4:4:6 (14 ml) was added ruthenium trichloride hydrate (53 mg, 0.202 mmol) at rt. The reaction mixture was stirred for 5 h at rt then concentrated to dryness. The residue was purified by chromatography eluting with 10% MeOH in DCM to give N-benzoyl-2-(cyclopentyloxy)-9-[2,3-O-(1-methylethylidene)-β-D-ribofuranuronosyl]-9H-purin-6-amine (260 mg). This intermediate was taken up in a 1:1 mixture of methylamine (3 ml, 2 M in MeOH) and ammonia (3 ml, 30% in water), then stirred overnight. The reaction mixture was concentrated to dryness and the residue was dissolved in acetic acid (5 ml, 80% in water) then heated at 80° C. for 7 h. The reaction mixture was concentrated to dryness and the residue was purified using Gilson reverse phase HPLC with 10 mM ammonium acetate and acetonitrile as the mobile phase with a gradient of 10-50% in 15 min. Relevant fractions were combined to give 20 mg of the desired product.

MS (ESP): 366 (MH⁺) for C₁₅H₁₉N₅O₆

¹H NMR δ: 1.63-1.84 (m, 8H) 4.06 (m, 2H) 4.31 (m, 1H) 5.24 (m, 2H) 5.81 (d, 1H) 7.10 (s, 2H) 8.74 (s, 1H)

### EXAMPLE 257

### (2S,3S,4R,5R)-5-[6-amino-2-(cyclopentyloxy)-9H-purin-9-yl]-3,4-dihydroxytetrahydrofuran-2-carboxamide

To a solution of N-benzoyl-2-(cyclopentyloxy)-9-[2,3-O-(1-methylethylidene)-β-D-ribofuranuronosyl]-9H-purin-6-amine (150 mg, 0.29 mmol) in THF was added triethylamine (123 μl, 0.88 mmol) and ethyl chloroformate (42 μl, 0.44 mmol) at 4° C. successively. The solution was stirred for 10 min, then ammonia gas was bubbled thought the solution for 30 min at 4° C. The reaction mixture was concentrated to dryness and the residue was purified by chromatography eluting with 8% MeOH in DCM to give (3aS,4S,6R,6aR)-6-[6-(benzoylamino)-2-(cyclopentyloxy)-9H-purin-9-yl]-2,2-dimethyltetrahydrofuro[3,4-d][1,3]dioxole-4-carboxamide) (75 mg). This intermediate was dissolved in acetic acid (5 ml, 80% in water), then heated at 80° C. for 7 h. The reaction mixture was concentrated to dryness and the residue was taken up in up in a 1:1 mixture of methylamine (3 ml, 2 M in MeOH) and ammonia (3 ml, 30% in water), then stirred overnight. The reaction mixture was concentrated to dryness and the residue was purified using Gilson reverse phase HPLC with 10 mM ammonium acetate and acetonitrile as the mobile phase with a gradient of 5-25% in 15 min. Relevant fractions were combined to give 3 mg of the desired product.

MS (ESP): 365 (MH⁺) for C₁₅H₂₀N₆O₅

¹H NMR δ: 1.49-1.64 (m, 6H) 1.75-1.88 (m, 2H) 4.17 (d, 2H) 4.52-4.60 (m, 1H) 5.16-5.22 (m, 1H) 5.48 (d, 1H) 5.61 (d, 1H) 5.78 (d, 1H) 7.23 (s, 2H) 7.45 (s, 1H) 7.86 (s, 1H) 8.14 (s, 1H)

### EXAMPLE 258

### 9-(5-amino-5-deoxy-β-D-ribofuranosyl)-2-(cyclopentyloxy)-9H-purin-6-amine

To a solution of 9-(5-azido-5-deoxy-β-D-ribofuranosyl)-2-(cyclopentyloxy)-9H-purin-6-amine (32 mg, 0.09 mmol) in ethanol (92 ml) was added 10% palladium on charcoal (20 mg). The reaction mixture was stirred under hydrogen (1 atm) for 4 h. At the end of this period, the reaction mixture was diluted with ethanol, filtered through diatomaceous earth and evaporated. The residue was purified using Gilson reverse phase HPLC with 10 mM ammonium acetate and acetonitrile as the mobile phases with a gradient of 5-95% in 15 min. Relevant fractions were combined to give 13 mg of the desired product.

MS (ESP): 351 (MH⁺) for C₁₂H₂₂N₆O₄

¹H NMR δ: 1.52-1.83 (m, 8H) 2.70 (d, 2H) 3.76 (m, 1H) 4.08 (m, 1H) 4.61 (m, 1H) 5.21 (m, 1H) 5.67 (d, 1H) 7.13 (s, 2H) 8.07 (s, 1H)

The intermediates were prepared as follows:—

### 9-(5-azido-5-deoxy-β-D-ribofuranosyl)-2-(cyclopentyloxy)-9H-purin-6-amine

A solution of 9-[5-azido-5-deoxy-2,3-O-(1-methylethylidene)-β-D-ribofuranosyl]-2-(cyclopentyloxy)-9H-purin-6-amine (47 mg, 0.11 mmol) in acetic acid (5 ml, 80% in water) was heated at 80° C. for 36 h. The reaction mixture was concentrated to dryness and the residue was purified by chromatography eluting with 5% MeOH in DCM to give the desired product (32 mg).

MS (ESP): 376 (MH⁺) for C₁₅H₂₀N₈O₄

### 9-[5-azido-5-deoxy-2,3-O-(1-methylethylidene)-β-D-ribofuranosyl]-2-(cyclopentyloxy)-9H-purin-6-amine

A solution of 9-[5-azido-5-deoxy-2,3-O-(1-methylethylidene)-β-D-ribofuranosyl]-N-benzoyl-2-(cyclopentyloxy)-9H-purin-6-amine (99 mg, 0.19 mmol) in a 1:1 mixture of methylamine (3 ml, 2 M in MeOH) and ammonia (3 ml, 30% in water) was stirred for 4 h. The reaction mixture was concentrated to dryness and the residue was purified by chromatography eluting with 65% EtOAc in hexane to give the desired product (47 mg).

MS (ESP): 417 (MH⁺) for C₁₈H₂₄N₈O₄

### 9-[5-azido-5-deoxy-2,3-O-(1-methylethylidene)-β-D-ribofuranosyl]-N-benzoyl-2-(cyclopentyloxy)-9H-purin-6-amine

A suspension of N-benzoyl-2-(cyclopentyloxy)-9-[2,3-O-(1-methylethylidene)-5-O-(methylsulfonyl)-β-D-ribofuranosyl]-9H-purin-6-amine (169 mg, 0.29 mmol) and sodium azide (38 mg, 0.59 mmol) in DMF (1 ml) was heated in a microwave reactor for 10 min at 80° C. The reaction mixture was diluted with DCM (40 ml) and washed with water. The organic phase was dried (sodium sulfate) and evaporated to dryness. The residue was purified by chromatography eluting with 4% MeOH in DCM to give the desired product (99 mg).

MS (ESP): 521 (MH⁺) for C₂₅H₂₈N₈O₅

### N-benzoyl-2-(cyclopentyloxy)-9-[2,3-O-(1-methylethylidene)-5-O-(methylsulfonyl)-β-D-ribofuranosyl]-9H-purin-6-amine

To a solution of N-benzoyl-2-(cyclopentyloxy)-9-[2,3-O-(1-methylethylidene)-β-D-ribofuranosyl]-9H-purin-6-amine (200 mg, 0.40 mmol) in dry pyridine (2 ml) was added methanesulfonyl chloride (47 μl, 0.61 mmol) at 4° C. The solution was stirred for 2 h at rt then diluted with DCM (50 ml). The reaction mixture was washed successively with cold water, saturated sodium bicarbonate and brine. The organic phase was dried (sodium sulfate) and concentrated to dryness. The residue was purified by chromatography eluting with 4% MeOH in DCM to give the desired product (200 mg).

MS (ESP): 574 (MH⁺) for C₂₆H₃₁N₅O₈S

¹H NMR (300 MHz, CDCl₃) δ ppm 1.36 (s, 3H) 1.51-1.66 (m, 2H) 1.58 (s, 3H) 1.78-1.93 (m, 6H) 2.92 (s, 3H) 4.29-4.42 (m, 2H) 4.42-4.50 (m, 1H) 5.06 (dd, 1H) 5.35 (m, 1H) 5.43 (dd, 1H) 6.09 (d, 1H) 7.44-7.59 (m, 3H) 7.87-8.01 (m, 3H) 8.76 (s, 1H)

### EXAMPLE 259

### 2-(cyclopentyloxy)-9-(5,6-dideoxy-β-D-ribo-hex-5-enofuranosyl)-9H-purin-6-amine

N-benzoyl-2-(cyclopentyloxy)-9-[5,6-dideoxy-2,3-O-(1-methylethylidene)-β-D-ribo-hex-5-enofuranosyl]-9H-purin-6-amine (13.0 mg) was dissolved in a methanolic ammonia solution (7M, 2 ml) in a microwave vial. The resulting mixture was heated in a microwave reactor at 120° C. for 30 min. LC-MS showed disappearance of the starting material and total conversion to the N-benzoyl deprotected product (MH⁺ 405). The solvent was evaporated and the residue dissolved in a 4:6:1 mixture of acetic acid, water and formic acid (3 ml). This mixture was heated at 90° C. for 6 h. The reaction mixture was neutralized to pH 7 by slow addition of aqueous ammonium hydroxide (29%) and evaporated to dryness. The resulting residue was re-dissolved in DMSO and purified by reverse-phase HPLC using a gradient of aqueous ammonium acetate (pH 8.0) and acetonitrile (20 to 95%) over 10 min. The relevant fractions were combined and solvents evaporated to dryness furnishing the desired product as a thin film. The product was then dissolved in water and freeze-dried to give a white solid (2.9 mg).

MS (ESP): 348 (MH⁺) for C₁₆H₂₁N₅O₄

¹H NMR δ: 1.48-1.63 (m, 2H) 1.68 (m, 4H) 1.81-1.95 (m, 2H) 4.19 (t, 1H) 4.24-4.34 (m, 1H) 4.66 (ddd, 1H) 5.01-5.10 (m, 1H) 5.12-5.26 (m, 2H) 5.71-5.81 (m, 1H) 5.84-5.99 (m, 1H) 6.56 (s, 2H) 7.14 (s, 2H) 8.04 (s, 1H)

The intermediate for this product was prepared as follows:—

### N-benzoyl-2-(cyclopentyloxy)-9-[5,6-dideoxy-2,3-O-(1-methylethylidene)-β-D-ribo-hex-5-enofuranosyl]-9H-purin-6-amine

N-benzoyl-2-(cyclopentyloxy)-9-[2,3-O-(1-methylethylidene)-β-D-ribofuranosyl]-9H-purin-6-amine (100 mg) was dissolved in 5 mL of anhydrous DMSO. N,N-dicyclohexylcarbodiimide (144 mg) was added in one portion. At 0° C., under a nitrogen atmosphere, dichloroacetic acid was added dropwise (9 μl) and the resulting solution stirred at rt for 2.5 h. Simultaneously in a separate flask, sodium hydride (30 mg) was added to 2 mL of DMSO and the solution stirred at 80° C. for 1 h. To this was added a solution of methyltriphenylphosphonium bromide (143 mg) in DMSO (2 ml) at 0° C. After stirring at rt for 15 min, the resulting ylide was added to the solution containing the aldehyde previously formed as indicated above. The mixture was stirred at rt for 15 h. The reaction mixture was filtered and purified by reverse-phase HPLC using a gradient of aqueous ammonium acetate (pH 8.0) and acetonitrile over 14 min. The relevant fractions were combined and solvents evaporated to dryness to give the desired product as a thin film (13.0 mg).

MS (ESP): 492 (MH⁺) and 490 (M-1)⁻ for C₂₆H₂₉N₅O₅

### EXAMPLE 260

### (2R,3S,4R,5R)-5-[6-amino-2-(cyclopentyloxy)-9H-purin-9-yl]-3,4-dihydroxytetrahydrofuran-2-carbaldehyde oxime

N-(2-(cyclopentyloxy)-9-{(3aR,4R,6R,6aR)-6-[(E)-(hydroxyimino)methyl]-2,2-dimethyltetrahydrofuro[3,4-d][1,3]dioxol-4-yl}-9H-purin-6-yl)benzamide (23.0 mg) was dissolved in methanolic ammonia (7M, 2 ml) in a microwave vial. The resulting mixture was heated in a microwave reactor at 120° C. for 30 min. LC-MS showed disappearance of the starting material and total conversion to the N-benzoyl deprotected product (MH⁺ 404). The solvent was evaporated and the residue dissolved in a 4:6:1 mixture of acetic acid, water and formic acid (3 ml). This mixture was heated at 90° C. for 6 h. The reaction mixture was neutralized to pH 7 by addition of aqueous ammonium hydroxide solution (29%) and evaporated to dryness. The resulting residue was dissolved in DMSO and purified by reverse-phase HPLC using a gradient of aqueous ammonium acetate (pH 8.0) and acetonitrile (17 to 25%) over 10 min. The relevant fractions were combined and solvents evaporated to dryness to give the desired product as a thin film. The product was then dissolved in water and freeze-dried to give a white solid (3.9 mg).

MS (ESP): 365 (MH⁺) and 363 (M-1)⁻ for C₁₅H₂₀N₆O₅

¹H NMR δ: 1.48-1.63 (m, 2H) 1.63-1.77 (m, 4H) 1.82 (m, 2H) 4.31-4.47 (m, 1H) 4.51-4.65 (m, 1H) 4.71 (dd, 1H) 5.27 (d, 1H) 5.76-5.90 (m, 1H) 6.32 (s, 1H) 6.54-6.68 (m, 3H) 7.18 (s, 2H) 8.04-8.14 (m, 1H)

The intermediate for this compound was prepared as follows:—

### N-(2-(cyclopentyloxy)-9-{(3aR,4R,6R,6aR)-6-[(E)-(hydroxyimino methyl]-2,2-dimethyltetrahydrofuro[3,4-d][1,3]dioxol-4-yl}-9H-purin-6-yl)benzamide

N-Benzoyl-2-(cyclopentyloxy)-9-[2,3-O-(1-methylethylidene)-β-D-ribofuranosyl]-9H-purin-6-amine (100 mg) was dissolved in 5 mL of anhydrous DMSO. N,N-dicyclohexylcarbodiimide (144 mg) was added in one portion. At 0° C., under a nitrogen atmosphere, dichloroacetic acid was added dropwise (9 μl) and the resulting solution stirred at rt for 2.5 h. Pyridine (1 ml) was then added followed by hydroxylamine hydrochloride (140 mg). The resulting mixture was stirred overnight at rt. The reaction mixture was filtered and purified by reverse-phase HPLC using a gradient of aqueous ammonium acetate (pH 8.0) and acetonitrile on an YMC-Pack ODS-Aq column (100×20 mm ID, S-5 μm, 12 nm) over 14 min. The relevant fractions were combined and solvents evaporated to dryness to give the desired product as a white solid (23.0 mg).

MS (ESP): 509 (MH⁺) and 507 (M-1)⁻ for C₂₅H₂₈N₆O₆. The product was used directly in the next step without additional characterization.

Examples 261-264 in Table XI were made according to the following procedures:—

A solution of 100 mg (0.25 mmol, 1.0 equivalent) of 2-(cyclopentyloxy)-9-[2,3-O-(1-methylethylidene)-β-D-ribofuranosyl]-9H-purin-6-amine or 2-(cyclobutylmethoxy)-9-[2,3-O-(1-methylethylidene)-β-D-ribofuranosyl]-9H-purin-6-amine in DCM/DMSO (5:1, 5 mL) was added at 0° C. and under a positive pressure of nitrogen over a solution of Dess-Martin reagent in DCM (15% w/w solution, 0.37 mmol, 1.5 equivalents, 780 μL). The resulting solution was stirred at 0° C. for 1 h and then at rt for 3 h. Anhydrous pyridine was then added (1 mL) followed by the corresponding commercially available O-substituted hydroxylamine hydrochloride in one portion (1.25 mmol, 5.0 equivalents). The reaction mixture was stirred at rt overnight and monitored by LC/MS. Solvents were evaporated to dryness. The resulting material was used directly in the next step without additional purification.

### Acetonide Deprotection

The material obtained as described above was dissolved in a 1:6:4 mixture of formic acid:acetic acid and water and heated at 90° C. The reaction was closely monitored by LC/MS and quenched with ammonia/MeOH at rt. After quenching, the solvents were evaporated to dryness and the resulting mixture purified by HPLC on reverse phase using ammonium acetate/acetonitrile or ammonium acetate/MeOH mixtures (at pH=8). After purification, the relevant fractions were combined and the solvents evaporated to dryness. The resulting product was dissolved in MeOH/water and lyophilized giving a white or off-white solid (usual yields ranging between 2.0 and 20 mg).

TABLE XI

| | | | 1HNMR (300 MHz, CDCl₃ unless |
|---|---|---|---|
| EX | IUPAC name | MH+ | otherwise indicated)) δ ppm |
| 261 | {[((1Z,E)-{(2R,3S,4R,5R)-5-[6- | 423 | (DMSO-d₆) 1.57 (m, 2H) 1.67 (m, 4H) |
| | amino-2-(cyclopentyloxy)-9H- | | 1.88 (m, 2H) 4.34 (m, 2H) 4.45 and |
| | purin-9-yl]-3,4- | | 4.49 (2 s, 2H total, ratio 3:2) 4.77 (bs, 1H) |
| | dihydroxytetrahydrofuran-2- | | 5.25 (bs, 1H) 5.63 (bs, 1H) 5.83 and 5.85 (2s, 1H |
| | yl}methylene)amino]oxy}acetic | | total, 2:3 ratio) 7.22 (bs, 2H) 7.75 and |
| | acid | | 7.76 (2s, 1H total, ratio 3:2) 8.10 and |
| | | | 8.11 (2s, 1H total, ratio 3:2) |
| 262 | (2R,3S,4R,5R)-5-[6-amino-2- | 369 | 1.55 (m, 2H) 1.78 (m, 6H) 3.80 (m, 4H) |
| | (cyclopentyloxy)-9H-purin-9- | | 4.52 (m, 1H) 4.68 (m, 1H) 4.79 (m, 1H) |
| | yl]-3,4- | | 5.21 (m, 2H) 5.91 (s, 1H) 7.43 (s, 1H) |
| | dihydroxytetrahydrofuran-2- | | 7.97 (s, 1H) |
| | carbaldehyde O-methyloxime | | |
| 263 | (2R,3S,4R,5R)-5-[6-amino-2- | 393 | (DMSO-d₆) 1.16 (m, 3H) 1.57-1.81 (m, 8H) |
| | (cyclopentyloxy)-9H-purin-9- | | 4.03 (m, 3H) 4.53 (m, 1H) 4.63 (m, 1H) |
| | yl]-3,4- | | 5.02 (m, 1H) 5.35 (m, 1H) 5.99 (m, 1H) |
| | dihydroxytetrahydrofuran-2- | | 7.50 (bs, 1H) 8.20 (bs, 1H) |
| | carbaldehyde O-ethyloxime | | |
| 264 | (2R,3S,4R,5R)-5-[6-amino-2- | 405 | 1.55 (m, 2H) 1.77 (m, 6H) 4.50 (m, 2H) |
| | (cyclopentyloxy)-9H-purin-9- | | 4.66 (bs, 1H) 4.87 (m, 1H) 5.17 (m, 4H) |
| | yl]-3,4- | | 5.86 (m, 2H) 7.49 (bs, 1H) 8.06 (bs, 1H) |
| | dihydroxytetrahydrofuran-2- | | |
| | carbaldehyde O-allyloxime | | |

### EXAMPLE 265

### (2R,3R,4S,5R)-2-[6-amino-2-(cyclopentyloxy)-9H-purin-9-yl]-5-[(E)-2-isocyanovinyl]tetrahydrofuran-3,4-diol and (2R,3R,4S,5R)-2-[6-amino-2-(cyclopentyloxy)-9H-purin-9-yl]-5-[(Z)-2-isocyanovinyl]tetrahydrofuran-3,4-diol

2-(Cyclopentyloxy)-9-[2,3-O-(1-methylethylidene)-β-D-ribofuranosyl]-9H-purin-6-amine (200 mg, 0.51 mmol, 1 equivalent), (triphenylphosphoranylidene)acetonitrile (1.0 mmol, 2.0 equivalents) and benzoic acid (1.0 mmol; 2.0 equivalents) were mixed and dissolved in a DCM/DMSO mixture (10:1, 5.5 mL) under nitrogen. At rt, a solution of Dess-Martin periodinane (15% w/w in DCM, 1.0 mmol, 2.0 equivalents) was added slowly. Stirring was continued at rt for 4-6 h and monitored by LC/MS. Solvents were evaporated to dryness and the resulting residue used directly in next step.

The residue was dissolved in a 1:6:4 mixture of formic acid:acetic acid and water and heated at 90° C. The reaction was closely monitored by LC/MS and quenched with ammonia/MeOH at rt. After quenching, the solvents were evaporated to dryness and the resulting residue purified by HPLC on reverse phase using ammonium acetate/acetonitrile or ammonium acetate/MeOH mixtures (at pH 8). After purification, the relevant fractions were combined and the solvents evaporated to dryness. The resulting products were dissolved in MeOH/water and lyophilized giving white to off-white solids. Two fractions were separated.

Fraction I: mixture of the E and Z isomers (1:2 by LC/MS).

MS (ESP): 373 (MH⁺) for C₁₇H₂₀N₆O₄

¹H NMR δ: 1.68 (m, 2H) 1.88 (m, 4H) 2.16 (m, 2H) 4.48 (m, 1H) 4.65 (m, 1H) 4.77 (m, 1H) 5.25 (bs, 1H) 5.80 (m, 1H) 6.68 (m, 1H) 7.17 (bs, 2H) 8.09 (bs, 1H).

Fraction II: mostly E isomer by LC/MS

MS (ESP): 373 (MH⁺) for C₁₇H₂₀N₆O₄

¹H NMR δ: 1.70 (m, 2H) 1.90 (m, 4H) 2.15 (m, 2H) 4.48 (m, 1H) 4.67 (m, 1H) 5.25 (bs, 1H) 5.65 (m, 1H) 5.94 (m, 1H) 7.00 (m, 1H) 7.17 (bs, 2H) 8.09 (s, 1H).

Examples 266-271 shown in Table XII were made using the following procedures:—

2-Chloro-9-{2,3-O-(1-methylethylidene)-5-O-[(4-methylphenyl)sulfonyl]-β-D-ribofuranosyl}-9H-purin-6-amine (J. Med. Chem. 1974, 17(11), 1197-1207) (300 mg, 0.6 mmol, 1 equivalent) and the corresponding azole (3.0 mmol, 5.0 equivalents) were dissolved in DMSO at rt. Potassium hydroxide (170 mg, 3.0 mmol, 5.0 equivalents) was added in one portion and stirring continued at rt until no starting material was detected by LC/MS (1-4 d). In some cases heating at 70° C. was used. Upon reaction completion, the mixture was partitioned between EtOAc or chloroform and water. The organic layer was separated and washed twice with brine, dried and the solvents evaporated to give product. This was used directly in the next step without additional purification.

The product obtained as described above was dissolved in a 1:1 mixture of THF and the corresponding alcohol (cyclopentanol or cyclobutylmethanol) and two pellets of sodium hydroxide were added in one portion. The resulting mixture was heated at 70° C. until reaction completion. Solvents were evaporated to dryness and the resulting material was used without purification.

The material obtained as described above was dissolved in a 1:6:4 mixture of formic acid:acetic acid and water and heated at 90° C. The reaction was closely monitored by LC/MS and quenched with ammonia/MeOH at rt. After quenching, the solvents were evaporated to dryness and the resulting material was purified by HPLC on reverse phase using ammonium acetate/acetonitrile or ammonium acetate/MeOH mixtures (at pH=8). After purification, the relevant fractions were combined and the solvents evaporated to dryness. The resulting products were dissolved in MeOH/water and lyophilized giving white or off-white solids.

TABLE XII

| EX | IUPAC name | MH+ | ¹H NMR δ ppm |
|---|---|---|---|
| 266 | 1-({(2R,3S,4R,5R)-5-[6-amino-2- | 446 | 2.16 (s, 2H of mixture) 2.26 (s, 6H of |
| | (cyclopentyloxy)-9H-purin-9-yl]-3,4- | | mixture) 4.65 (t, 1H major) 4.75 (m, 1H |
| | dihydroxytetrahydrofuran-2-yl}methyl)- | | of mixture) 4.93 (m, 1H minor) 5.15 (t, 1H |
| | 1H-imidazole-5-carboxylic acid & 1- | | major) 5.29 (m, 2H mixture) 5.85 (m, |
| | ({(2R,3S,4R,5R)-5-[6-amino-2- | | 1H mixture) 6.37 (d, 1H major) 6.40 (d, |
| | (cyclopentyloxy)-9H-purin-9-yl]-3,4- | | 1H minor) 7.73 (s, 1H major), 7.78 (bs, |
| | dihydroxytetrahydrofuran-2-yl}methyl)- | | 2H mixture) 7.89 (s, 1H major) 8.20 (s, |
| | 1H-imidazole-4-carboxylic acid (1:5) | | 1H minor), 8.30 (s, 1H minor), 8.38 (s, 1H |
| | | | major), 8.62 (s, 1H minor). |
| 267 | (2R,3R,4S,5R)-2-[6-amino-2- | 470 | 1.56 (m, 2H) 1.67 (m, 4H) 1.88 (m, 2H) |
| | (cyclopentyloxy)-9H-purin-9-yl]-5-{[3- | | 4.25 (m, 2H) 4.55 (m, 2H) 4.67 (m, 1H) |
| | (trifluoromethyl)-1H-pyrazol-1- | | 5.27 (m, 1H) 5.41 (m, 1H) 5.54 (m, 1H) |
| | yl]methyl}tetrahydrofuran-3,4-diol | | 5.78 (d, 1H) 6.63 (s, 1H) 7.19 (s, 2H) |
| | | | 7.80 (s, 1H) 8.00 (s, 1H) |
| 268 | (2R,3R,4S,5R)-2-[6-amino-2- | 403 | 1.51 (m, 2H) 1.63 (m, 4H) 1.82 (m, 2H) |
| | (cyclopentyloxy)-9H-purin-9-yl]-5-(4H- | | 4.21 (m, 2H) 4.32 (m, 1H) 4.54 (m, 1H) |
| | 1,2,4-triazol-4- | | 4.65 (m, 1H) 5.27 (m, 1H) 5.41 and |
| | ylmethyl)tetrahydrofuran-3,4-diol- | | 5.52 (2d, 1H, 1:1) 5.79 (m, 1H) 7.14 and |
| | (2R,3R,4S,5R)-2-[6-amino-2- | | 7.18 (2x bs, 2H, 1:1) 7.90 (d, 1H, one isomer) |
| | (cyclopentyloxy)-9H-purin-9-yl]-5-(1H- | | 7.97 (d, 1H, one isomer) 8.00 (s, 1H, one |
| | 1,2,4-triazol-1- | | isomer), 8.29 (d, 1H, one isomer) 8.36 (s, |
| | ylmethyl)tetrahydrofuran-3,4-diol (1:1) | | 1H, one isomer) |
| 269 | (2R,3R,4S,5R)-2-[6-amino-2- | 470 | 1.83 (m, 4H mixture) 2.02 (m, 2H |
| | (cyclobutylmethoxy)-9H-purin-9-yl]-5- | | mixture) 2.69 (ms, 1H mixture) 4.27 (m, |
| | {[5-(trifluoromethyl)-1H-pyrazol-1- | | 3H mixture) 4.62 (m, 3H mixture) |
| | yl]methyl}tetrahydrofuran-3,4-diol- | | 5.84 (2d, 1H mixture) 6.66 (s, 1H major), |
| | (2R,3R,4S,5R)-2-[6-amino-2- | | 6.86 (s, 1H minor) 7.07 (s, 1H major), 7.09 (s, |
| | (cyclobutylmethoxy)-9H-purin-9-yl]-5- | | 1H minor) 7.24 (s, 1H major) 7.26 (s, 1H |
| | {[3-(trifluoromethyl)-1H-pyrazol-1- | | minor) 7.40 (s, 1H major) 7.43 (s, 1H |
| | yl]methyl}tetrahydrofuran-3,4-diol (1:5) | | minor) 7.68 (s, 1H minor), 7.89 (s, 1H |
| | | | major) 7.91 (s, 2H) 8.05 (s, 1H) 8.06 (m, |
| | | | 1H minor) 8.16 (s, 1H major) 8.34 (s, 1H |
| | | | major) 8.56 (m, 1H minor) 8.93 (m, 1H |
| | | | minor) |
| 270 | 1-({(2R,3S,4R,5R)-5-[6-amino-2- | 447 | 1.51 (m, 2H) 1.692 (m, 4H) 1.83 (m, 2H) |
| | (cyclopentyloxy)-9H-purin-9-yl]-3,4- | | 4.13-4.33 (m, 4H) 4.67 (m, 1H) |
| | dihydroxytetrahydrofuran-2-yl}methyl)- | | 5.26 (m, 1H) 5.73 and 5.79 (2d, 1H total, 1:1) |
| | 1H-1,2,4-triazole-3-carboxylic acid-1- | | 7.14 (bs, 2H) 7.89-7.98 (m, 2H) 8.27 and |
| | ({(2R,3S,4R,5R)-5-[6-amino-2- | | 8.23 (2s, 1H total, 1:1) |
| | (cyclopentyloxy)-9H-purin-9-yl]-3,4- | | |
| | dihydroxytetrahydrofuran-2-yl}methyl)- | | |
| | 1H-1,2,4-triazole-5-carboxylic acid | | |
| | (1:1) | | |
| 271 | (2R,3R,4S,5R)-2-[6-amino-2- | 404 | 1.70-1.85 (m, 4H of mixture) 1.97 (s, 2H |
| | (cyclobutylmethoxy)-9H-purin-9-yl]-5- | | of mixture) 2.55-2.67 (m, 1H of |
| | (2H-tetrazol-2- | | mixture) 4.09-4.19 (m, 4H of mixture) |
| | ylmethyl)tetrahydrofuran-3,4-diol- | | 4.54-4.64 (m, 3H of mixture) |
| | (2R,3R,4S,5R)-2-[6-amino-2- | | 5.67-5.78 (m, 1H of mixture) 7.18 and |
| | (cyclobutylmethoxy)-9H-purin-9-yl]-5- | | 7.20 (2s, 2H total, 2:1) 7.93 and 8.03 (2s, 1H |
| | (1H-tetrazol-1- | | total, 2:1) 9.16 and 9.23 (2s, 1H total, |
| | ylmethyl)tetrahydrofuran-3,4-diol (2:1) | | 1:2) |

### EXAMPLE 272

### (2R,3R,4S,5R)-2-[6-amino-2-(cyclopentyloxy)-9H-purin-9-yl]-5-(azidomethyl)tetrahydrofuran-3,4-diol

2-(Cyclopentyloxy)-9-[5-deoxy-2,3-O-(1-methylethylidene)-5-azido-β-D-ribofuranosyl]-9H-purin-6-amine (155 mg) was treated with acid as described for Examples 266-271. After acetonide cleavage and lyophilization, the desired product was obtained as a white solid (86.0 mg).

MS (ESP): 377 (MH⁺) for C₁₇H₂₀N₈O₄

¹H NMR δ: 1.57 (m, 2H), 1.70 (m, 4H), 1.90 (m, 2H), 3.57 (m, 2H), 3.99 (m, 1H), 4.15 (m, 1H), 4.74 (m, 1H), 5.30 (m, 1H), 5.35 (d, 1H), 5.54 (d, 1H), 5.80 (d, 1H), 7.20 (bs, 2H), 8.12 (s, 1H).

The precursor for this compound was prepared as follows:—

### 2-(Cyclopentyloxy)-9-[5-deoxy-2,3-O-(1-methylethylidene)-5-azido-β-D-ribofuranosyl]-9H-purin-6-amine

2-(Cyclopentyloxy)-9-[2,3-O-(1-methylethylidene)-β-D-ribofuranosyl]-9H-purin-6-amine (1.6 g, 4.0 mmol, 1.0 equivalent) and triphenylphosphine (1.6 g, 6.0 mmol, 1.5 equivalent) were mixed under nitrogen. Anhydrous THF was added (50 mL) and the resulting mixture cooled to 0° C. A mixture of diphenylphosphoryl azide (1.3 mL, 6.0 mmol, 1.5 eq) and diisopropylazodicarboxyate (1.2 mL, 6.0 mmol, 1.5 equivalents) in THF (10 mL) was then added dropwise. The reaction mixture was allowed to reach rt and stirred at this temperature overnight. Solvents were evaporated to dryness and the resulting oil purified by preparative HPLC on reverse phase using ammonium acetate/acetonitrile or MeOH mixtures (pH 8). Relevant fractions were combined to give a thick oil. This was dissolved in MeOH/water and lyophilized to a white fluffy solid (620 mg).

MS (ESP): 417 (MH⁺) for C₁₉H₂₄N₈O₄

¹H NMR (300 MHz, CDCl₃) δ: 1.38 (s, 3H), 1.61 (s, 3H), 1.64 (m, 2H), 1.86 (m, 2H), 1.97 (m, 4H), 3.54 (dd, 1H), 3.70 (dd, 1H), 4.37 (m, 1H), 4.94 (m, 1H), 5.25 (m, 1H), 5.44 (t, 1H), 6.05 (d, 1H), 8.04 (s, 1H).

### EXAMPLE 273

### 5′-N-Phthalimidyl-2-cyclopentyloxy-9-β-D-ribofuranosyl-9H-purine-6-amine

The title compound was made using a procedure analogous to that described for Example 272 by reaction of 2-(cyclopentyloxy)-9-[2,3-O-(1-methylethylidene)-β-D-ribofuranosyl]-9H-purin-6-amine with phthalimide under Mitsunobu conditions. The intermediate was deprotected with formic acid/water giving the desired product.

MS (ESP): 481 (MH⁺) for C₂₃H₂₄N₆O₆

¹H NMR (300 MHz, CDCl₃) δ: 1.49 (m, 2H), 1.63 (m, 2H), 1.86 (m, 4H), 2.74 (s, 2H), 3.84 (m, 1H), 3.96 (m, 1H), 4.17 (m, 1H), 4.40 (m, 1H), 4.92 (m, 1H), 5.27 (m, 1H), 5.76 (d, 1H), 6.34 (br s, 2H), 7.72 (s, 4H), 7.89 (s, 1H)

### EXAMPLE 274

### {(2R,3S,4R,5R)-5-[6-amino-2-(cyclobutylmethoxy)-9H-purin-9-yl]-3,4-dihydroxytetrahydrofuran-2-yl}acetonitrile

{(3aR,4R,6R,6aR)-6-[6-amino-2-(cyclobutylmethoxy)-9H-purin-9-yl]-2,2-dimethyltetrahydrofuro[3,4-d][1,3]dioxol-4-yl}acetonitrile) was treated with acid as described for Examples 266-271. After purification and lyophilization, the desired product was obtained as a white solid.

MS (ESP): 361 (MH⁺) for C₁₆H₂₀N₆O₄

¹H NMR δ: 1.85 (m, 4H), 2.03 (m, 2H), 2.26 (m, 1H), 3.03 (dd, 2H), 4.11 (m, 1H), 4.17 (m, 4H), 4.71 (m, 1H), 5.49 (d, 1H), 5.59 (d, 1H), 5.80 (d, 1H), 7.28 (bs, 2H), 8.12 (s, 1H).

The precursor to this compound was prepared as follows:—

### {(3aR,4R,6R,6aR)-6-[6-amino-2-(cyclobutylmethoxy)-9H-purin-9-yl]-2,2-dimethyltetrahydrofuro[3,4-d][1,3]-dioxol-4-yl}acetonitrile

2-(cyclobutylmethoxy)-9-[2,3-O-(1-methylethylidene)-β-D-ribofuranosyl]-9H-purin-6-amine (1.0 g, 1.0 equivalent, 2.56 mmol) and triphenylphosphine (1.7 g, 2.5 equivalents) were dissolved in anhydrous THF (100 mL) under nitrogen. At 0° C., acetone cyanohydrin (585 μL, 2.5 equivalents) was added dropwise. After 5 min at 0° C., diisopropylazodicarboxylate (1.3 mL, 2.5 equivalents) was added. The resulting solution was allowed to reach rt and stirred overnight. When the reaction was complete as determined by LC/MS, the solvent was evaporated to dryness to give a thick orange oil. The desired product was isolated by preparative HPLC on using ammonium acetate (pH 8)/MeOH or acetonitrile mixtures. Relevant fractions were combined and solvent evaporated to dryness. The resulting thick oil was dissolved in water/MeOH and lyophilized to give an off-white fluffy solid (750 mg)

MS (ESP): 373 (MH⁺) for C₁₇H₂₀N₆O₄

¹H NMR δ: 1.68 (m, 2H) 1.88 (m, 4H) 2.16 (m, 2H) 4.48 (m, 1H) 4.65 (m, 1H) 4.77 (m, 1H) 5.25 (bs, 1H) 5.80 (m, 1H) 6.68 (m, 1H) 7.17 (bs, 2H) 8.09 (bs, 1H).

### EXAMPLE 275

### 1-([(2R,3S,4R,5R)-5-[6-amino-2-(cyclopentyloxy)-9H-purin-9-yl]-3,4-dihydroxytetrahydrofuran-2-yl]methyl)-1H-1,2,3-triazole-4-carboxylic acid

9-(5-azido-5-deoxy-β-D-ribofuranosyl)-2-(cyclopentyloxy)-9H-purin-6-amine (210 mg, 0.5 mmol, 1 equivalent), ethyl propionic ester (2.0 ml, 1.0 mmol, 2 equivalents), diisopropylethylamine (4.3 mmol, 50 equivalents, 25 mmol) and copper (I) iodide (190 mg, 2 equivalents, 1.0 mmol) were dissolved in anhydrous THF (5 mL) and the resulting mixture stirred overnight at rt. The reaction mixture was filtered and the precipitate washed several times with chloroform. The solvents were evaporated and the resulting material used directly in the next step without additional purification.

MS (ESP): 515 (MH⁺) for C₂₃H₃₀N₈O₆.

The material prepared above was dissolved in a 2:1 mixture of water and MeOH and two pellets of sodium hydroxide were added in one portion. The mixture was stirred at rt overnight. Solvents were evaporated to dryness and the resulting material was used in next step without additional purification.

MS (ESP): 487 (MH⁺) for C₂₁H₂₆N₈O₆.

The material obtained as described above was dissolved in a 1:6:4 mixture of formic acid:acetic acid:water and heated at 90° C. The reaction was closely monitored by LC/MS and quenched with ammonia/MeOH at rt. After quenching, the solvents were evaporated to dryness and the resulting material purified by HPLC on reverse phase using ammonium acetate/acetonitrile or ammonium acetate/MeOH mixtures (at pH 8). After purification, the relevant fractions were combined and the solvents evaporated to dryness. The resulting product was dissolved in MeOH/water and lyophilized giving an off-white solid (96.0 mg).

MS (ESP): 447 (MH⁺) for C₁₈H₂₂N₈O₆

¹H NMR δ: ppm 2.19 (m, 2H) 2.32 (m, 4H) 2.45 (m, 2H) 4.86 (m, 2H) 5.31 (m, 2H) 5.92 (m, 1H) 6.42 (d, 1H) 7.83 (s, 2H) 8.54 (s, 1H) 8.64 (s, 1H).

### EXAMPLE 276

### (2R,3R,4S,5R)-2-[6-amino-2-(cyclopentyloxy)-9H-purin-9-yl]-5-(1H-1,2,3-triazol-1-ylmethyl)tetrahydrofuran-3,4-diol

9-(5-azido-5-deoxy-β-D-ribofuranosyl)-2-(cyclopentyloxy)-9H-purin-6-amine (150 mg, 0.35 mmol, 1 equivalent) and vinyl acetate (20 equivalents), were dissolved in anhydrous toluene (10 mL) and the resulting mixture stirred at 70° C. Reaction was monitored by LC/MS. The solvent was evaporated and the resulting material used in the next step without additional purification.

MS (ESP): 443 (MH⁺) for C₂₀H₂₆N₈O₄.

The material prepared above, was dissolved in a 1:1 mixture of formic acid and water and stirred at rt. The reaction was closely monitored by LC/MS. The solvents were then evaporated to dryness and the desired product isolated by reverse-phase HPLC using ammonium acetate aqueous solution (pH 8)/acetonitrile or MeOH mixtures as the eluent. Relevant fractions were combined and solvents evaporated. The resulting product was dissolved in MeOH/water and lyophilized to give a white solid (15.5 mg).

MS (ESP): 403 (MH⁺) for C₁₇H₂₂N₈O₄

¹H NMR δ: 1.53 (m, 2H), 1.62 (m, 4H), 1.84 (m, 2H), 4.17 (m, 2H), 4.65 (m, 2H), 5.19 (m, 1H), 5.73 (s, 1H), 7.18 (bs, 2H), 7.61 (s, 1H), 7.93 (s, 1H), 7.93 (s, 1H).

### EXAMPLE 277

### 1-([(2R,3S,4R,5R)-5-[6-amino-2-(cyclopentyloxy)-9H-purin-9-yl]-3,4-dihydroxytetrahydrofuran-2-yl]methyl)-1H-tetrazole-5-carboxylic acid

9-(5-azido-5-deoxy-β-D-ribofuranosyl)-2-(cyclopentyloxy)-9H-purin-6-amine (160 mg, 0.38 mmol, 1 equivalent) and O-ethyl cyanoformate (190 μL, 1.9 mmol, 5.0 equivalents) were mixed in a small pressure flask and the mixture heated at 120° C. (neat). After LC/MS confirmed consumption of starting material, MeOH and sodium bicarbonate (saturated) were added and stirred at rt for 2 h. Then chloroform was added and the organic layer was separated, washed twice with sodium bicarbonate (saturated), dried over magnesium sulfate, filtered and evaporated to give a brown residue. This was used in the next step without additional purification.

MS (ESP): 516 (MH⁺) for C₂₂H₂₉N₉O₆

The ester obtained as described above, was dissolved in a 2:1 mixture of water and MeOH and two pellets of sodium hydroxide were added in one portion. The mixture was stirred at rt overnight. Solvents were evaporated to dryness and used in the next step without additional purification.

MS (ESP): 488 (MH⁺) for C₂₀H₂₅N₉O₆

The material obtained as described above was dissolved in a 6:1 mixture of formic acid and water and stirred at rt. The reaction was closely monitored by LC/MS. The solvents were evaporated to dryness and the resulting material purified by HPLC on reverse phase using ammonium acetate/acetonitrile or ammonium acetate/MeOH mixtures (at pH=8). After purification, the relevant fractions were combined and the solvents evaporated to dryness. The resulting product was dissolved in MeOH/water and lyophilized giving an off-white solid (7.8 mg).

MS (ESP): 448 (MH⁺) for C₁₇H₂₁N₉O₆

¹H NMR δ: 1.53 (m, 2H) 1.64 (m, 4H) 1.90 (m, 2H) 4.28 (m, 1H) 4.67 (m, 1H) 4.82 (m, 1H), 4.23 (m, 1H) 5.55 (m, 1H) 5.79 (m, 1H) 7.21 (m, 2H) 7.93 (s, 1H) 8.03 (m, 1H) 9.25 (m, 1H).

### EXAMPLE 278

### (2R,3R,4S,5R)-2-[6-amino-2-(cyclopentyloxy)-9H-purin-9-yl]-5-(2H-tetrazol-5-ylmethyl)tetrahydrofuran-3,4-diol

{(3aR,4R,6R,6aR)-6-[6-amino-2-(cyclobutylmethyloxy)-9H-purin-9-yl]-2,2-dimethyltetrahydrofuro[3,4-d][1,3]dioxol-4-yl}acetonitrile (150 mg, 0.37 mmol), sodium azide (244 mg, 3.75 mmol, 10 equivalents) and ammonium chloride (400 mg, 7.50 mmol, 20 equivalents) were mixed in dry DMF in a small pressure vial. This was sealed and heated at 120° C. for two days. The resulting material was partitioned between water and chloroform, the organic layer was washed with water (2×), dried over magnesium sulfate, filtered and solvent evaporated to give a mixture which was used in next step without additional purification.

MS (ESP): 444 (MH⁺) for C₁₉H₂₅N₉O₄.

The mixture was dissolved in a 1:1 mixture of formic acid and water and stirred at rt. The reaction was closely monitored by LC/MS. The solvents were then evaporated to dryness and the desired product isolated by reverse-phase HPLC using ammonium acetate aqueous solution (pH 8)/acetonitrile or MeOH mixtures as the eluent. Relevant fractions were combined and solvents evaporated. The resulting product was dissolved in MeOH/water and lyophilized to give an off-white solid (5.4 mg).

MS (ESP): 404 (MH⁺) for C₁₆H₂₁N₉O₄.

¹H NMR δ: 1.84 (m, 4H) 2.05 (m, 2H) 2.67 (m, 1H) 3.13 (m, 3H) 4.19 (m, 4H) 4.54 (m, 1H), 5.27 (m, 1H) 5.56 (d, 1H) 7.20 (m, 2H) 8.07 (s, 1H).

### EXAMPLE 279

### (2R,3R,4S,5R)-2-[6-amino-2-(cyclobutylmethoxy)-9H-purin-9-yl]-5-(2H-1,2,3-triazol-2-ylmethyl)tetrahydrofuran-3,4-diol

2-Chloroadenosine (200 mg, 0.66 mmol, 1 equivalent), triphenylphosphine (175 mg, 0.66 mmol, 1 equivalent), and 1,2,3-triazole (138 μL, 2.4 mmol, 3.6 equivalents) were dissolved in anhydrous dioxane under a positive pressure of nitrogen. Diisopropylazodicarboxylate (392 μL, 2.0 mmol, 3.0 equivalents) was then added dropwise at 0° C. The resulting mixture was stirred at 120° C. for 15 h. The solvents were evaporated to give a yellow material that was used directly in next step without additional purification.

MS (ESP): 353 (MH⁺) for C₁₂H₁₃ClN₈O₃

The yellow material was dissolved in a 1:1 mixture of THF and cyclobutyl MeOH and two pellets of sodium hydroxide were added in one portion. The resulting mixture was heated at 70° C. until reaction completion. Solvents were evaporated to dryness the pure product was isolated by HPLC on reverse phase using ammonium acetate/acetonitrile or ammonium acetate/MeOH mixtures (at pH=8). After purification, the relevant fractions were combined and the solvents evaporated to dryness. The resulting product was dissolved in MeOH/water and lyophilized giving a white solid (23.0 mg).

MS (ESP): 353 (MH⁺) for C₁₇H₂₂N₈O₃

¹H NMR δ: 1.84 (m, 4H) 2.234 (m, 2H) 2.68 (m, 1H) 4.20 (m, 31H) 4.37 (m, 1H) 4.70 (m, 3H), 5.43 (d, 1H) 5.55 (d, 1H) 5.79 (d, 1H) 7.27 (m, 2H) 7.78 (s, 2H) 8.03 (s, 1H).

Examples 280-282, shown in Table XIII were made using the following procedures:—

2-(Cyclopentyloxy)-9-[2,3-O-(1-methylethylidene)-β-D-ribofuranosyl]-9H-purin-6-amine (200 mg, 0.5 mmol, 1 equivalent) and the appropriate commercially available disulfide (1 mmol, 2 equivalents) were dissolved in dry pyridine under a positive pressure of nitrogen. Tributylphosphine (250 μL, 1.0 mmol, 2 equivalents) was then added dropwise at 0° C. After addition, the mixture was allowed to reach rt over 1 h and stirring continued for 15 h. The mixture was partitioned between chloroform and water and the organic layer was washed several times with saturated sodium bicarbonate, dried over magnesium sulfate, filtered and solvents evaporated to dryness. The resulting material was used directly in the next step without additional purification.

The material prepared above, was dissolved in a 1:1 mixture of formic acid and water and stirred at rt. The reaction was closely monitored by LC/MS. The solvents were then evaporated to dryness and the desired product isolated by reverse-phase HPLC using ammonium acetate aqueous solution (pH 8)/acetonitrile or MeOH mixtures as the eluent. Relevant fractions were combined and solvents evaporated. The resulting product was dissolved in MeOH/water and lyophilized to give an off-white solid (20 to 150 mg).

TABLE XIII

| EX | IUPAC name | MH+ | ¹H NMR δ ppm (DMSO-d₆) |
|---|---|---|---|
| 280 | (2R,3R,4S,5S)-2-[6-amino-2- | 444 | 1.81 (m, 4H) 2.00 (m, 2H) 2.66 (m, 1H) |
| | (cyclobutylmethoxy)-9H-purin-9-yl]-5- | | 3.28 (dd, 1H) 3.42 (dd, 1H) 3.97 (d, |
| | [(phenylthio)methyl]tetrahydrofuran-3,4- | | 1H) 4.14 (m, 3H) 4.75 (qua, 1H) |
| | diol | | 5.31 (d, 1H), 5.42 (d, 1H) 5.76 (d, 1H) |
| | | | 7.29 (s, 7H) 8.11 (s, 1H) |
| 281 | (2R,3R,4S,5S)-2-[6-amino-2- | 445 | 1.78 (m, 6H) 2.00 (m, 2H) 2.66 (m, 1H) |
| | (cyclobutylmethoxy)-9H-purin-9-yl]-5- | | 3.47 (dd, 1H) 3.68 (dd, 1H) |
| | [(pyridin-2-ylthio)methyl]tetrahydrofuran- | | 4.06 (m, 1H) .16 (m, 3H) 4.91 (qua, 1H) |
| | 3,4-diol | | 5.37 (d, 1H) 5.47 (d, 1H) 5.78 (d, 1H) |
| | | | 7.10 (dd, 1H) 7.27 (bs, 2H) 7.29 (d, |
| | | | 1H), 7.61 (dt, 1H) 8.14 (s, 1H) 8.43 (d, |
| | | | 1H) |
| 282 | (2R,3R,4S,5S)-2-[6-amino-2- | 512 | 1.56 (m, 4H) 1.98 (m, 2H) 2.64 (m, 1H) |
| | (cyclobutylmethoxy)-9H-purin-9-yl]-5- | | 3.67 (dds, 1H) 3.85 (dd, 1H) |
| | {[(1-phenyl-1H-tetrazol-5- | | 4.25 (m, 4H) 4.82 (t, 1H) 5.77 (d, 1H) |
| | yl)thio]methyl}tetrahydrofuran-3,4-diol | | 7.27 (bs, 2H) 7.61 (bs, 5H) 8.09 (s, 1H) |

### EXAMPLE 283

### 2-(cyclopentyloxy)-9-[(5E,Z)-5,6-dideoxy-β-D-ribo-hept-5-enofuranosyl]-9H-purin-6-amine

2-(cyclopentyloxy)-9-[(5E,Z)-5,6-dideoxy-2,3-O-(1-methylethylidene)-β-D-ribo-hept-5-enofuranosyl]-9H-purin-6-amine was dissolved in a 6:4:1 mixture of acetic acid, water and formic acid and heated with stirring at 90° C. The reaction was closely monitored by LC/MS and stopped by azeotroping the excess formic acid/water with ethanol. The resulting mixture was purified by HPLC on reverse phase using ammonium acetate/acetonitrile or ammonium acetate/methanol mixtures (at pH=8). After purification, the relevant fractions were combined and the solvents evaporated to dryness. The resulting product was dissolved in MeOH/water and lyophilized giving a white solid (3.0 mg, >95% purity, mixture of Z/E isomers 1:1).

MS (ESP): 378 (MH⁺) for C₁₇H₂₃N₅O₅

¹H NMR (300 MHz, DMSO-d₆) δ ppm 1.59 (m, 2H of mixture), 1.69 (m, 4H of mixture), 1.90 (m, 2H of mixture), 4.27 and 4.10 (2×m, 2H of mixture), 3.93 (m, 2H of mixture), 4.76 and 4.67 (2×m, 2H of mixture), 5.28 (m, 2H of mixture), 5.66-5.81 (m, 2H of mixture 1+1H one isomer), 5.49 (m, 1H one isomer), 7.20 and 7.16 (2×bs, 2H of mixture), 8.08 and 8.04 (2×s, 1H of mixture).

The intermediates used to prepare this analog where obtained as follows:—

### 2-(cyclopentyloxy)-9-[(5E,Z)-5,6-dideoxy-2,3-O-(1-methylethylidene)-β-D-ribo-hept-5-enofuranosyl]-9H-purin-6-amine

A portion of 2-(cyclopentyloxy)-9-[(5E)-5,6-dideoxy-7-ethyl-2,3-O-(1-methylethylidene)-β-D-ribo-hept-5-enofuranosyluronosyl]-9H-purin-6-amine (415 mg) was dissolved in dry DCM. At −78° C. and under a positive pressure of nitrogen, a diisobutylaluminum hydride solution (20% in toluene, 7.2 mmol, 8.0 equivalents) was added dropwise. The mixture was stirred at −78° C. until no starting material was detected by LC/MS. The reaction was quenched by slow addition of MeOH followed by water. The resulting material was partitioned between EtOAc and water, dried and the solvent evaporated to give a yellow oil. This was used directly in next step without additional purification.

MS (ESP): 418 (MH⁺) for C₂₀H₂₇N₅O₅

### 2-(cyclopentyloxy)-9-[(5E)-5,6-dideoxy-7-ethyl-2,3-O-(1-methylethylidene)-(3-D-ribo-hept-5-enofuranosyluronosyl]-9H-purin-6-amine

2-(cyclopentyloxy)-9-[2,3-O-(1-methylethylidene)-β-D-ribofuranosyl]-9H-purin-6-amine (300 mg, 0.75 mmol), benzoic acid (180 mg, 1.5 mmol, 2.0 equiv), ethyl (triphenylphosphoranylidene)acetate (523 mg, 1.5 mmol, 2.0 equivalents) were mixed in a 10:1 anhydrous DCM and DMSO under a nitrogen atmosphere. Dess-Martin periodinane (15% w/w solution in DCM, 2.3 mL, 1.5 equivalents) was then added at rt. It was stirred at rt overnight. The reaction was quenched by addition of saturated sodium bicarbonate and extracted with chloroform, dried over magnesium sulfate, filtered and the solvent evaporated to give an oil (776 mg). This was purified by quick filtration through a plug of silica and used directly in the next step.

MS (ESP): 460 (MH⁺) for C₂₂H₂₉N₅O₆

### EXAMPLE 284

### (2E)-3-[(2R,3S,4R,5R)-5-[6-amino-2-(cyclopentyloxy)-9H-purin-9-yl]-3,4-dihydroxytetrahydrofuran-2-yl]-N-methoxy-N-methylacrylamide

The material obtained as described below was dissolved in a 6:4:1 mixture of acetic acid, water and formic acid and heated with stirring at 90° C. The reaction was closely monitored by LC/MS and stopped by azeotroping the excess formic acid/water with ethanol. The resulting material was purified by HPLC on reverse phase using ammonium acetate/acetonitrile or ammonium acetate/methanol mixtures (at pH=⁸). After purification, the relevant fractions were combined and the solvents evaporated to dryness. The resulting product was dissolved in MeOH/water and lyophilized giving a white solid (2.8 mg, one isomer, unknown stereochemistry).

MS (ESP): 435 (MH⁺) for C₁₉H₂₆N₆O₆

¹H NMR (300 MHz, DMSO-d₆) δ ppm 1.59 (m, 2H), 1.69 (m, 4H), 1.90 (m, 2H), 3.61 (s, 3H), 4.0 (s, 3H), 4.90 (m, 1H), 4.94 (m, 1H), 5.39 (m, 1H), 5.59 (m, 1H), 6.25 (bs, 2H), 6.30 (d, 1H), 7.18 (d, 1H), 7.39 (dd, 1H), 8.31 (s, 1H).

The intermediate to prepare this compound was obtained as follows:—

### 2-(cyclopentyloxy)-9-[(5E)-5,6-dideoxy-7-[methoxy(methyl)amino]-2,3-O-(1-methylethylidene)-β-D-ribo-hept-5-enodialdo-1,4-furanosyl]-9H-purin-6-amine

2-(cyclopentyloxy)-9-[2,3-O-(1-methylethylidene)-β-D-ribofuranosyl]-9H-purin-6-amine (150 mg, 0.38 mmol), and N-methoxy-N-methyl-2-(triphenylphosphoranylidene) acetamide (280 mg, 0.77 mmol, 2 equivalents) were mixed in anhydrous DCM:DMSO 10:1 (4 mL) under a nitrogen atmosphere. At 0° C., Dess-Martin periodinane (15% w/w solution in DCM, 2.3 mL, 1.5 equivalents) was then added. The mixture was allowed to reach rt and was stirred overnight at this temperature. The reaction was quenched by addition of saturated sodium bicarbonate and extracted with chloroform, dried over anhydrous magnesium sulfate, filtered and the solvent evaporated to give an oil. This was used directly in the next step without additional purification.

MS (ESP): 475 (MH⁺) for C₂₂H₃₀N₆O₆

### EXAMPLE 285

### 9-[5-(5-amino-1H-1,2,4-triazol-1-yl)-5-deoxy-β-D-ribofuranosyl]-2-(cyclopentyloxy)-9H-purin-6-amine and 9-[5-(3-amino-1H-1,2,4-triazol-1-yl)-5-deoxy-β-D-ribofuranosyl]-2-(cyclopentyloxy)-9H-purin-6-amine (1:1 mixture of regioisomers)

9-[5-(5-amino-1H-1,2,4-triazol-1-yl)-5-deoxy-2,3-O-(1-methylethylidene)-β-D-ribofuranosyl]-2-(cyclopentyloxy)-9H-purin-6-amine and 9-[5-(3-amino-1H-1,2,4-triazol-1-yl)-5-deoxy-2,3-O-(1-methylethylidene)-β-D-ribofuranosyl]-2-(cyclopentyloxy)-9H-purin-6-amine was dissolved in a 6:4:1 mixture of acetic acid, water and formic acid and heated with stirring at 90° C. The reaction was closely monitored by LC/MS and stopped by azeotroping the excess formic acid/water with ethanol. The resulting material was purified by HPLC using ammonium acetate/acetonitrile or ammonium acetate/MeOH mixtures (at pH=8). After purification, the relevant fractions were combined and the solvents evaporated to dryness. The resulting product was dissolved in MeOH/water and lyophilized giving a white solid (1.9 mg, mixture of two regioisomers 1:1).

MS (ESP): 318 (MH⁺) for C₁₇H₂₃N₉O₄

¹H NMR (300 MHz, DMSO-d₆) δ ppm 1.59 (m, 2H of mixture), 1.69 (m, 4H of mixture), 1.85 (m, 2H of mixture), 4.07-4.20 (m, 3H of mixture), 4.33 (m, 2H of mixture), 4.66 (m, 1H of mixture), 5.28 (m, 2H of mixture), 5.71-5.85 (m, 2H of mixture), 7.14 (bs, 2H of mixture), 7.30 and 7.27 (2×s, 1H of mixture), 8.05 and 8.99 (2×s, 1H of mixture).

The intermediates were obtained as follows:—

### 9-[5-(5-amino-1H-1,24-triazol-1-yl)-5-deoxy-2,3-O-(1-methylethylidene)-(3-D-ribofuranosyl]-2-(cyclopentyloxy)-9H-purin-6-amine and 9-[5-(3-amino-1H-1,2,4-triazol-1-yl)-5-deoxy-2,3-O-(1-methylethylidene)-β-D-ribofuranosyl]-2-(cyclopentyloxy)-9H-purin-6-amine

9-[5-(5-amino-1H-1,2,4-triazol-1-yl)-5-deoxy-2,3-O-(1-methylethylidene)-(3-D-ribofuranosyl]-2-chloro-9H-purin-6-amine and 9-[5-(3-amino-1H-1,2,4-triazol-1-yl)-5-deoxy-β-D-ribofuranosyl]-2-chloro-9H-purin-6-amine was dissolved in cyclopentanol and 1 pellet of sodium hydroxide was added. The mixture was stirred at 70° C. for 15 h. Then chloroform was added and the solution filtered through diatomaceous earth. Solvents were evaporated to dryness and dissolved in chloroform, then filtered through a pad of silica using chloroform, chloroform/MeOH 5% or 10% as the eluants. The relevant fractions were combined and the solvent evaporated to dryness. The resulting oil was used in the final step without additional purification.

MS (ESP): 458 (MH⁺) for C₂₀H₂₇ClN₉O₄

### 9-[5-(5-amino-1H-1,2,4-triazol-1-yl)-5-deoxy-2,3-O-(1-methylethylidene)-β-D-ribofuranosyl]-2-chloro-9H-purin-6-amine and 9-[5-(3-amino-1H-1,24-triazol-1-yl)-5-deoxy-β-D-ribofuranosyl]-2-chloro-9H-purin-6-amine

2-chloro-9-{2,3-O-(1-methylethylidene)-5-O-[(4-methylphenyl)sulfonyl]-β-D-ribofuranosyl}-9H-purin-6-amine (see Examples 266-271) (150 mg, 0.3 mmol) and 3-amino-1,2,4-triazole (50 mg, 0.6 mmol, 2 equivalents) were dissolved in DMSO. Excess potassium t-butoxide (2-5 equivalents) was added in one portion. After stirring at rt for 1 week, solvent was evaporated to dryness. The resulting oil was used directly in the next step without additional purification.

MS (ESP): 410, 408 (MH⁺) for C₁₅H₁₈ClN₉O₃

### EXAMPLE 286

### 9-[5-[4-(carboxymethyl)-1H-imidazol-1-yl]-5-deoxy-β-D-ribofuranosyl]-2-(cyclopentyloxy)-9H-purin-6-amine

2-chloro-9-[5-[4-(cyanomethyl)-1H-imidazol-1-yl]-5-deoxy-2,3-O-(1-methylethylidene)-β-D-ribofuranosyl]-9H-purin-6-amine was dissolved in cyclopentanol and 1 pellet of sodium hydroxide was added. The mixture was stirred at 70° C. for 15 h, Solvents were evaporated to dryness and the resulting oil (displacement and hydrolysis had occurred) was used without additional purification. The final acetonide deprotection was carried out as described above using a 1:2 mixture of water and formic acid at rt. The product was obtained as a hygroscopic beige solid (18.0 mg).

MS (ESP): 460 (MH⁺) for C₂₀H₂₅N₇O₆

¹H NMR (300 MHz, DMSO-d₆) δ ppm 1.58 (m, 2H), 1.64 (m, 4H), 1.88 (m, 2H), 4.12 (m, 2H), 4.22 (m, 2H), 4.55 (m, 1H), 5.26 (1H, m), 5.78 (1H, m) 6.92 (s, 1H) 7.20 (bs, 2H) 7.40 (s, 1H), 8.0 (s, 1H).

The intermediate for compound was obtained as follows:—

### 2-chloro-9-[5-[4-(cyanomethyl)-1H-imidazol-1-yl]-5-deoxy-2,3-O-(1-methylethylidene)-β-D-ribofuranosyl]-9H-purin-6-amine

2-chloro-9-{2,3-O-(1-methylethylidene)-5-O-[(4-methylphenyl)sulfonyl]-β-D-ribofuranosyl}-9H-purin-6-amine (300 mg, 0.6 mmol) and 1H-imidazol-4-ylacetonitrile (3.0 mmol, 5 equivalents) were dissolved in DMSO. Excess potassium hydroxide (5 equivalents) was added in one portion. After stirring at rt for 15 h the mixture was partitioned between EtOAc and water. The organic layer was separated, dried and filtered giving an off-white foam. This was used directly in the next step without additional purification.

MS (ESP): 431 (MH⁺) for Cl₈H₁₉ClN₈O₃

### EXAMPLE 287

### 9-(5-S-butyl-5-thio-β-D-ribofuranosyl)-2-(cyclobutylmethoxy)-9H-purin-6-amine and 9-(5-S-isobutyl-5-thio-β-D-ribofuranosyl)-2-(cyclobutylmethoxy)-9H-purin-6-amine (2:1 mixture)

2-(Cyclobutylmethoxy)-9-[2,3-O-(1-methylethylidene)-β-D-ribofuranosyl]-9H-purin-6-amine (250 mg, 0.65 mmol) was dissolved in dry N-methylpyrrolidinone (3 ml) in a microwave vial. Dibutyldisulfide (1:1 mixture of n and iso isomers) (450 mg, 4 equivalents) was added dropwise. The mixture was placed in a bath at 0° C. and tri-n-butylphosphine (740 μL, 4 equivalents) was added under a positive pressure of nitrogen. The solution was then heated in a microwave reactor at 180° C. for 1 h. The dark mixture was partitioned between EtOAc and saturated sodium bicarbonate and worked up as usual to give an oil. This was used without additional purification.

MS (ESP): 464 (MH⁺) for C₂₂H₃₃N₅O₄S

Acetonide deprotection was carried out as described above using a 2:1 mixture of formic acid and water at rt. The product was obtained as a white solid (43.8 mg, 2:1 mixture of isomers)

¹H NMR (300 MHz, DMSO-d₆) δ ppm 0.8 (m, 6H), 1.0-2.0 (several m, 10H), 2.68-2.86 (m, 3H), 3.97 (m, 1H), 4.14 (m, 3H), 4.73 (m, 1H), 5.32 (m, 1H), 5.47 (m, 1H), 5.74 (1H, d), 7.23 (2×bs, 2H), 8.10 (s, 1H).

MS (ESP): 424 (MH⁺) for C₁₉H₂₉N₅O₄S

### EXAMPLE 288

### 2-(cyclobutylmethoxy)-9-(5-S-methyl-5-thio-β-D-ribofuranosyl)-9H-purin-6-amine

The title compound was made using an analogous procedure to Example 287 by reaction of 2-(cyclobutylmethoxy)-9-[2,3-O-(1-methylethylidene)-β-D-ribofuranosyl]-9H-purin-6-amine with dimethyl disulfide as the thiol source.

MS (ESP): 382 (MH⁺) for C₁₆H₂₃N₅O₄S

¹H NMR (300 MHz, DMSO-d₆) δ ppm 1.86 (m, 5H), 2.02 (m, 4H), 2.68-2.86 (m, 3H), 3.97 (m, 1H), 4.14 (m, 3H), 4.73 (m, 1H), 5.32 (m, 1H), 5.47 (m, 1H), 5.74 (1H, d), 7.23 (bs, 2H), 8.10 (s, 1H).

### EXAMPLE 289

### 9-(5-chloro-5-deoxy-β-D-ribofuranosyl)-2-(cyclopentyloxy)-9H-purin-6-amine

Hexamethylphosphoramide (0.5 mL) and thionyl chloride (110 μL, 3 equivalents) were mixed slowly at 0° C. under nitrogen. After stirring at 0° C. for 30 min, 2-(cyclopentoxy)-9-[2,3-O-(1-methylethylidene)-β-D-ribofuranosyl]-9H-purin-6-amine (200 mg, 0.5 mmol) dissolved in hexamethylphosphoramide (1 ml) was added. Stirring was continued at 0° C. for 4 h. The reaction was quenched by slow addition of saturated sodium bicarbonate and extracted with EtOAc giving a brown oil. This was used without additional purification.

MS (ESP): 412, 410 (MH⁺) for C₁₈H₂₄ClN₅O₄

Acetonide deprotection was carried out as described above using a 2:1 mixture of formic acid and water at rt. After HPLC purification, the product was obtained as a white solid (17.6 mg).

MS (ESP): 372, 370 (MH⁺) for C₁₅H₂₀ClN₅O₄

¹H NMR (300 MHz, DMSO-d₆) δ ppm 1.56 (m, 2H), 1.70 (m, 4H), 1.90 (m, 2H), 3.80 (m, 1H), 3.91 (m, 1H), 4.03 (m, 1H), 4.20 (m, 1H), 4.73 (m, 1H), 5.20 (m, 1H), 5.43 (d, 1H), 5.45 (d, 1H), 5.80 (d, 1H), 7.21 (bs, 2H), 8.10 (s, 1H)

### EXAMPLE 290

### 9-(5-bromo-5-deoxy-β-D-ribofuranosyl)-2-(cyclopentyloxy)-9H-purin-6-amine

The title compound was prepared using an analogous procedure to Example 289 by reaction of 2-(cyclopentoxy)-9-[2,3-O-(1-methylethylidene)-β-D-ribofuranosyl]-9H-purin-6-amine with thionyl bromide.

MS (ESP): 414, 416 (MH⁺) for C₁₅H₂₀BrN₅O₄

¹H NMR (300 MHz, DMSO-d₆) δ ppm 1.56 (m, 2H), 1.70 (m, 4H), 1.90 (m, 2H), 3.80 (m, 1H), 3.83 (m, 1H), 4.06 (m, 1H), 4.21 (m, 1H), 4.77 (m, 1H), 5.26 (m, 1H), 5.41 (d, 1H), 5.56 (d, 1H), 5.79 (d, 1H), 7.22 (bs, 2H), 8.12 (s, 1H)

### EXAMPLE 291

### 2-(cyclopentyloxy)-9-(5-methyl-β-D-ribofuranosyluronosyl)-9H-purin-6-amine

2-(Cyclopentyloxy)-9-[5-methyl-2,3-O-(1-methylethylidene)-β-D-ribofuranbsyluronosyl]-9H-purin-6-amine was suspended in 2:1 mixture of acetic acid:water, then stirred at rt for 24 h. Formic acid was added and stirred for an additional 24 h. The reaction mixture was concentrated to dryness and the residue purified using Gilson reverse phase HPLC with 10 mM ammonium acetate and acetonitrile as the mobile phase with a gradient of 5-50% for 14 min. Relevant fractions were combined and concentrated to give a thin film. The product was dissolved in water and freeze-dried to give a white solid (28.2 mg).

MS (ESP): 380 (MH⁺) for C₁₆H₂₁N₅O₆

¹H NMR (300 MHz, DMSO-d₆) δ ppm 1.51 (s, 1H) 1.61 (s, 4H) 1.82 (s, 2H) 3.63 (s, 3H) 4.37 (s, 2H) 4.53 (s, 1H) 5.23 (s, 1H) 5.62 (s, 1H) 5.82 (s, 2H) 7.18 (s, 2H) 8.14 (s, 1H)

The intermediates for this compound were prepared as follows:—

### 2-(cyclopentyloxy)-9-[5-methyl-2,3-O-(1-methylethylidene)-β-D-ribofuranosyluronosyl]-9H-purin-6-amine

To a solution of 2-(cyclopentyloxy)-9-[2,3-O-(1-methylethylidene)-β-D-ribofuranuronosyl]-9H-purin-6-amine (100 mg, 0.247 mmol) in DCM was added catalytic dimethylaminopyridine (0.61 mg, 0.005 mmol), dicyclohexylcarbodiimide (1M in DCM, 0.271 mL, 0.271 mmol), and MeOH (18 μL, 0.741 mmol) at 0° C. The solution was warmed to rt and stirred overnight, then quenched with saturated sodium bicarbonate. The reaction mixture was extracted with EtOAc. Organic layers were combined and extracted with sodium bicarbonate, brine, and concentrated in vacuo.

MS (ESP): 420 (MH⁺) for C₁₉H₂₅N₅O₆

### 2-(cyclopentyloxy)-9-[2,3-O-(1-methylethylidene)-β-D-ribofuranuronosyl]-9H-purin-6-amine

2-(Cyclopentyloxy)-9-[2,3-O-(1-methylethylidene)-β-D-ribofuranosyl]-9H-purin-6-amine (400 mg, 1.02 mmol), 2,2,6,6-tetramethyl-1-piperidinyloxy, free radical (TEMPO) (32 mg, 0.204 mmol), and iodobenzene diacetate (722 mg, 2.24 mmol) was dissolved in 1:1 mixture of water and acetonitrile. The solution was stirred at rt overnight. The precipitate was filtered off and washed with water, acetone, and diethyl ether.

MS (ESP): 406 (MH⁺) for C₁₈H₂₃N₅O₆

### EXAMPLE 292

### (2S,3S,4R,5R)-5-[6-amino-2-(cyclopentyloxy)-9H-purin-9-yl]-N-cyclopropyl-3,4-dihydroxytetrahydrofuran-2-carboxamide

(3aS,4S,6R,6aR)-6-[6-amino-2-(cyclopentyloxy)-9H-purin-9-yl]-N-cyclopropyl-2,2-dimethyltetrahydrofuro[3,4-d][1,3]dioxole-4-carboxamide was suspended in 2:1 mixture of acetic acid:water, then stirred at rt for 24 h. Formic acid was added and stirred for an additional 24 h. The reaction mixture was concentrated to dryness and the residue purified using Gilson reverse phase HPLC with 10 mM ammonium acetate and acetonitrile as the mobile phase was a gradient of 5-50% for 14 min. Relevant fractions were combined and concentrated to give a thin film. The product was dissolved in water and freeze-dried to give a white solid (18.6 mg).

MS (ESP): 405 (MH⁺) for C₁₈H₂₄N₆O₅

¹H NMR (300 MHz, DMSO-d₆) δ ppm 0.44 (s, 1H) 0.56 (s, 1H) 1.51 (s, 1H) 1.62 (s, 2H) 1.86 (s, 1H) 2.63 (s, 1H) 4.14 (s, 1H) 4.55 (s, 1H) 5.25 (s, 1H) 5.47 (s, 1H) 5.59 (s, 1H) 5.77 (s 1H) 7.24 (s, 1H) 8.19 (s, 1H)

The intermediate for this Example was prepared as follows:—

### (3aS,4S,6R,6aR)-6-[6-amino-2-(cyclopentyloxy)-9H-purin-9-yl]-N-cyclopropyl-2,2-dimethyltetrahydrofuro[3,4-d][1,3]dioxole-4-carboxamide

To a solution of 2-(cyclopentyloxy)-9-[2,3-O-(1-methylethylidene)-β-D-ribofuranuronosyl]-9H-purin-6-amine (prepared as for Example 291) (100 mg, 0.247 mmol) in DMF was added O-(7-hydroxyazabenzotriazol-1-yl)-N,N,N′,N′-tetramethyluronium hexafluorophosphate (HATU) (112.5 mg, 0.296 mmol) and triethylamine (54 μL, 0.741 mmol) at 0° C. Cyclopropylamine (12 μL, 0.296 mmol) was added dropwise to the cooled solution. The solution was warmed to rt and stirred overnight, then quenched with saturated sodium bicarbonate. The reaction mixture was extracted with EtOAc. Organic layers were combined and extracted with sodium bicarbonate, brine, and concentrated in vacuo.

MS (ESP): 445 (MH⁺) for C₂₁H₂₄N₆O₅

### EXAMPLE 293

### (2R,3R,4S,5S)-2-[6-amino-2-(cyclopentyloxy)-9H-purin-9-yl]-5-(azetidin-1-ylcarbonyl)tetrahydrofuran-3,4-diol

The title compound was prepared using a procedure analogous to that described for Example 292 by reacting 2-(cyclopentyloxy)-9-[2,3-O-(1-methylethylidene)-β-D-ribofuranuronosyl]-9H-purin-6-amine with azetidine followed by deprotection of the acetonide.

MS (ESP): 405 (MH⁺) for C₁₈H₂₄N₆O₅

¹H NMR (300 MHz, DMSO-d₆) δ ppm 1.13 (s, 1H) 1.52 (s, 1H) 1.63 (s, 5H) 1.85 (s, 3H) 2.14 (s, 3H) 3.84 (s, 3H) 4.16 (s, 4H) 4.40 (s, 3H) 5.28 (s, 1H) 5.83 (s, 1H) 7.33 (s, 2H) 8.37 (s, 1H)

### EXAMPLE 294

### (2S,3S,4R,5R)-5-[6-amino-2-(cyclopentyloxy)-9H-purin-9-yl]-3,4-dihydroxy-N-methyltetrahydrofuran-2-carboxamide

The title compound was prepared using a procedure analogous to that described for Example 292 by reacting 2-(cyclopentyloxy)-9-[2,3-O-(1-methylethylidene)-β-D-ribofuranuronosyl]-9H-purin-6-amine with methylamine followed by deprotection of the acetonide.

MS (ESP): 379 (MH⁺) for C₁₆H₂₂N₆O₅

¹H NMR (300 MHz, DMSO-d₆) δ ppm 1.60 (m, 2H), 1.70 (m, 4H), 1.90 (m, 2H), 2.73 (d, 3H), 4.09 (1H, m), 4.27 (1H, m), 4.55 (m, 1H), 5.33 (m, 1H), 5.48 (m, 1H), 5.72 (1H, d), 5.82 (1H, d), 6.92 (s, 1H), 7.42 (bs, 2H), 8.12 (s, 1H), 9.18 (s, 1H)

### EXAMPLE 295

### 2-(cyclopentyloxy)-9-(5-O-methyl-3-D-ribofuranosyl)-9H-purin-6-amine

2-(Cyclopentyloxy)-N-[(1Z)-(dimethylamino)methylene]-9-(5-O-methyl-β-D-ribofuranosyl)-9H-purin-6-amine was dissolved in 7N ammonia in MeOH. The solution was stirred at rt for 1 h. The reaction mixture was concentrated to dryness and the residue purified using Gilson reverse phase HPLC with 10 mM ammonium acetate and acetonitrile as the mobile phase with a gradient of 10-40% for 14 min. Relevant fractions were combined and concentrated to give a thin film. Normal phase HPLC using hexanes and MeOH further purified the mixture. Relevant fractions were combined and concentrated to give a thin film. The product was dissolved in water and freeze-dried to give a white solid.

MS (ESP): 366 (MH⁺) for C₁₆H₂₃N₅O₅

¹H NMR (300 MHz, DMSO-d⁶) δ ppm 1.67 (s, 6H) 1.88 (s, 2H) 3.32 (s, 3H) 3.99 (s, 2H) 4.15 (s, 2H) 4.61 (s, 2H) 5.29 (s, 2H) 5.46 (s, 1H) 5.77 (s, 1H) 7.23 (s, 2H) 8.08 (s, 1H)

The intermediates for this compound were made as follows:—

### 2-(cyclopentyloxy)-N-[(1Z)-(dimethylamino)methylene]-9-(5-O-methyl-β-D-ribofuranosyl)-9H-purin-6-amine

2-(Cyclopentyloxy)-N-[(1Z)-(dimethylamino)methylene]-9-[5-O-methyl-2,3-O-(1-methylethylidene)-β-D-ribofuranosyl]-9H-purin-6-amine was suspended in 2:1 mixture of acetic acid:water, then stirred at rt for 24 h. The reaction mixture was concentrated to dryness.

MS (ESP): 420 (MH⁺) for C₁₉H₂₈N₆O₅

### 2-(cyclopentyloxy)-N-[(1Z)-(dimethylamino)methylene]-9-[5-O-methyl-2,3-O-(1-methylethylidene)-β-D-ribofuranosyl]-9H-purin-6-amine

2-(Cyclopentyloxy)-N-[(1Z)-(dimethylamino)methylene]-9-[2,3-O-(1-methylethylidene)-β-D-ribofuranosyl]-9H-purin-6-amine (200 mg, 0.448 mmol) was dissolved in DMF and cooled to −20° C. Sodium hydride (11 mg, 0.448 mmol) was added and stirred for 30 min. Methyl iodide (42 μL, 0.448 mmol) was added. The solution was stirred at −20° C. for several hours, allowed to warm to rt, and stirred overnight. The reaction mixture was dissolved in EtOAc and extracted with sodium bicarbonate and brine and concentrated.

MS (ESP): 461 (MH⁺) for C₂₂H₃₂N₆O₅

### 2-(cyclopentyloxy)-N-[(1Z)-(dimethylamino)methylene]-9-[2,3-O-(1-methylethylidene)-β-D-ribofuranosyl]-9H-purin-6-amine

2-(cyclopentyloxy)-9-[2,3-O-(1-methylethylidene)-β-D-ribofuranosyl]-9H-purin-6-amine (1 g, 2.56 mmol) was dissolved in DMF and (dimethoxymethyl)dimethylamine (514 μL, 3.84 mmol) was added. The reaction mixture was stirred at rt for 6 h. EtOAc was added to the solution and extracted with sodium bicarbonate and brine, dried over magnesium sulfate, and concentrated.

MS (ESP): 447 (MH⁺) for C₂₁H₃₀N₆O₅

The compounds in Table XIV were made using an analogous procedure to that described in Example 295 by reaction 2-(cyclopentyloxy)-N-[(1Z)-(dimethylamino)methylene]-9-[2,3-O-(1-methylethylidene)-β-D-ribofuranosyl]-9H-purin-6-amine with the appropriate commercially available electrophile (iodide, bromide, or tosylate) followed by the deprotection reactions.

TABLE XIV

| EX | IUPAC name | MH+ | ¹H NMR δ ppm (300 MHz, DMSO-d₆) |
|---|---|---|---|
| 296 | 2-(cyclopentyloxy)-9-[5-O-(2,2,2- | 434 | 1.52 (s, 2H) 1.62 (s, 6H) 1.83 (s, 2H) |
| | trifluoroethyl)-β-D-ribofuranosyl]-9H- | | 3.76 (s, 1H) 3.95 (s, 1H) 4.04 (s, 2H) |
| | purin-6-amine | | 4.50 (s, 1H) 5.23 (s, 2H) 5.50 (s, 1H) |
| | | | 5.75 (s, 1H) 7.16 (s, 2H) 8.06 (s, 1H) |
| 297 | 2-(cyclopentyloxy)-9-[5-O-(3- | 470 | 1.56 (s, 2H) 1.67 (s, 4H) 1.82 (s, 2H) |
| | phenylpropyl)-β-D-ribofuranosyl]-9H- | | 2.60 (s, 2H) 3.50 (s, 1H) 3.55 (s, 1H) |
| | purin-6-amine | | 3.97 (s, 1H) 4.15 (s, 1H) 4.25 (s, 1H) |
| | | | 4.68 (s, 1H) 5.27 (s, 1H) 5.50 (s, 1H) |
| | | | 5.75 (s, 1H) 7.21 (s, 6H) 8.06 (s, 1H) |
| 298 | 2-(cyclopentyloxy)-9-[5-O- | 406 | 1.00 (s, 2H) 1.18 (s, 2H) 1.57 (s, 2H) |
| | (cyclopropylmethyl)-β-D-ribofuranosyl]- | | 1.67 (s, 4H) 1.86 (s, 3H) 3.53 (s, 1H) |
| | 9H-purin-6-amine | | 3.63 (s, 1H) 3.97 (s, 1H) 4.15 (s, 1H) |
| | | | 4.68 (s, 2H) 5.29 (s, 1H) 5.75 (s, 1H) |
| | | | 7.21 (s, 2H) 8.05 (s, 1H) |
| 299 | 2-(cyclopentyloxy)-9-[5-O-(2-fluoroethyl)- | 397 | 0.75 (s, 2H) 1.20 (s, 2H) 1.68 (s, 4H) |
| | β-D-ribofuranosyl]-9H-purin-6-amine | | 3.61 (s, 1H) 3.71 (s, 1H) 3.97 (s, 1H) |
| | | | 4.10 (s, 1H) 4.45 (s, 1H) 4.59 (s, 1H) |
| | | | 5.27 (s, 1H) 5.50 (s, 1H) 5.75 (s, 1H) |
| | | | 7.21 (s, 2H) 8.06 (s, 1H) |
| 300 | 9-(5-O-allyl-β-D-ribofuranosyl)-2- | 392 | 1.65 (m, 2H), 1.69 (m, 4H), 1.90 (m, |
| | (cyclopentyloxy)-9H-purin-6-amine | | 2H), 3.56 (dd, 1H), 3.64 (dd, 1H), |
| | | | 3.97 (m, 3H), 4.16 (m, 1H), 4.57 (m, 1H), |
| | | | 5.13 (d, 1H), 5.26 (m, 3H), 5.48 (d, 1H), |
| | | | 5.76 (d, 1H), 5.84 (m, 1H), 7.19 (bs, |
| | | | 2H), 8.06 (s, 1H). |

### EXAMPLE 301

### 2-(cyclopentyloxy)-9-(5-O-phenyl-β-D-ribofuranosyl)-9H-purin-6-amine

2-(Cyclopentyloxy)-9-[2,3-O-(1-methylethylidene)-β-D-ribofuranosyl]-9H-purin-6-amine (200 mg, 0.51 mmol) and phenol (192 mg, 2.04 mmol) were dissolved in N-methylpyrrolidinone. The solution was cooled to 0° C. Tributylphosphine (509 μL, 2.04 mmol) was added followed by addition of diisopropylazodicarboxylate (402 μL, 2.04 mmol). The reaction mixture was heated in a microwave reactor at 120° C. for 1 h. The solution was further dissolved in chloroform and washed with sodium bicarbonate and brine. The mixture was purified by flash chromatography on silica gel using a hexane/EtOAc gradient. The relevant fractions were combined and concentrated.

MS (ESP): 468 (MH⁺) for C₂₄H₂₉N₅O₅

2-(Cyclopentyloxy)-9-[2,3-O-(1-methylethylidene)-5-O-phenyl-β-D-ribofuranosyl]-9H-purin-6-amine was suspended in 2:1 mixture of acetic acid:water, then stirred at rt for 24 h. The reaction mixture was concentrated to dryness and the residue purified using Gilson reverse phase HPLC with 10 mM ammonium acetate and acetonitrile as the mobile phase with a gradient of 20-50% for 14 min. Relevant fractions were combined and concentrated to give a thin film. The product was dissolved in water and freeze-dried to give a white solid.

MS (ESP): 429 (MH⁺) for C₂₁H₂₅N₅O₅

¹H NMR (300 MHz, DMSO-d₆) δ ppm 1.20 (s, 2H) 1.56 (s, 2H) 1.67 (s, 4H) 1.87 (s, 2H) 4.20 (s, 3H) 4.75 (s, 2H) 5.25 (s, 2H) 5.56 (s, 1H) 5.75 (s, 1H) 6.93 (s, 2H) 7.24 (s, 5H) 8.06 (s, 1H)

### EXAMPLE 302

### 9-(5-S-acetyl-5-thio-β-D-ribofuranosyl)-2-(cyclopentyloxy)-9H-purin-6-amine

9-[5-S-acetyl-2,3-O-(1-methylethylidene)-5-thio-β-D-ribofuranosyl]-2-(cyclopentyloxy)-9H-purin-6-amine was suspended in 2:1 mixture of acetic acid:water, then stirred at rt for 24 h. The reaction mixture was concentrated to dryness and the residue purified using Gilson reverse phase HPLC with 10 mM ammonium acetate and acetonitrile as the mobile phase with a gradient of 20-40% for 14 min. Relevant fractions were combined and concentrated to give a thin film. The product was dissolved in water and freeze-dried to give a white solid.

MS (ESP): 409 (MH⁺) for C₁₇H₂₃N₅O₅S

¹H NMR (300 MHz, DMSO-d₆) δ ppm 1.20 (s, 2H) 1.68 (s, 4H) 2.35 (s, 3H) 3.10 (s, 2H) 3.90 (s, 2H) 4.10 (s, 2H) 4.75 (s, 2H) 5.27 (s, 2H) 5.75 (s, 2H) 7.21 (s, 2H) 8.06 (s, 1H)

The intermediate for this compound was prepared as follows:—

### 9-[5-S-acetyl-2,3-O-(1-methylethylidene)-5-thio-β-D-ribofuranosyl]-2-(cyclopentyloxy)-9H-purin-6-amine

9-[5-chloro-5-deoxy-2,3-O-(1-methylethylidene)-β-D-ribofuranosyl]-2-(cyclopentyloxy)-9H-purin-6-amine (prepared as for Example 289) (300 mg, 0.73 mmol) was dissolved in DMF. Ethanethioic S-acid (152 μL, 2.20 mmol) and cesium carbonate (717 mg, 2.20 mmol) are added to the solution and stirred at rt overnight. EtOAc was added to the solution and extracted with sodium bicarbonate and brine.

MS (ESP): 450 (MH⁺) for C₂₀H₂₇N₅O₅S

### EXAMPLE 303

### 2-(cyclopentyloxy)-9-{5-[(cyclopropylmethyl)sulfinyl]-5-deoxy-β-D-ribofuranosyl}-9H-purin-6-amine

9-(5-S-acetyl-5-thio-9-D-ribofuranosyl)-2-(cyclopentyloxy)-9H-purin-6-amine (200 mg, 0.49 mmol) was dissolved in DMF. Cesium carbonate (477 mg, 1.46 mmol) was added followed by addition of (bromomethyl)cyclopropane (136 μL, 1.46 mmol). The reaction mixture was stirred overnight at rt. The solution was dissolved in EtOAc and extracted with sodium bicarbonate and brine. The reaction mixture was concentrated to dryness and the residue purified using Gilson reverse phase HPLC with 10 mM ammonium acetate and acetonitrile as the mobile phase with a gradient of 30-60% for 14 min. Relevant fractions were combined and concentrated to give a thin film. The product was dissolved in water and freeze-dried to give a white solid.

MS (ESP): 422 (MH⁺) for C₁₉H₂₇N₅O₄S

¹H NMR (300 MHz, DMSO-d₆) δ ppm 1.18 (s, 2H) 1.58 (s, 1H) 1.68 (s, 3H) 1.90 (s, 2H) 2.48 (s, 8H) 3.12 (s, 1H) 4.25 (s, 2H) 5.27 (s, 1H)

### EXAMPLE 304

### 2-(cyclopentyloxy)-9-[5-thio-5-S-(2,2,2-trifluoroethyl)-β-D-ribofuranosyl]-9H-purin-6-amine

9-(5-S-acetyl-5-thio-β-D-ribofuranosyl)-2-(cyclopentyloxy)-9H-purin-6-amine (200 mg, 0.49 mmol) was dissolved in DMF. Cesium carbonate (479 mg, 1.47 mmol) was added followed by addition of 2,2,2-trifluoroethyl 4-methylbenzenesulfonate (373 mg, 1.47 mmol). The reaction mixture was stirred overnight at rt. The solution was dissolved in EtOAc and extracted with sodium bicarbonate and brine. The reaction mixture was concentrated to dryness and the residue purified using Gilson reverse phase HPLC with 10 mM ammonium acetate and acetonitrile as the mobile phase with a gradient of 30-70% for 14 min. Relevant fractions were combined and concentrated to give a thin film. The product was dissolved in water and freeze-dried to give a white solid.

MS (ESP): 450 (MH⁺) for C₁₇H₂₂N₅O₄SF₃

¹H NMR (300 MHz, DMSO-d₆) δ ppm 1.20 (s, 2H) 1.58 (s, 2H) 1.68 (s, 4H) 1.88 (s, 2H) 3.01 (s, 2H) 3.48 (s, 2H) 4.0 (s, 1H) 4.15 (s, 1H) 4.75 (s, 2H) 5.25 (s, 2H) 5.55 (s, 1H) 5.75 (s, 1H) 7.21 (s, 2H) 8.06 (s, 1H)

### EXAMPLE 305

### 2-(cyclopentyloxy)-9-[5-deoxy-5-(methylsulfinyl)-β-D-ribofuranosyl]-9H-purin-6-amine and

### EXAMPLE 306

### 2-(cyclopentyloxy)-9-[5-deoxy-5-(methylsulfonyl)-β-D-ribofuranosyl]-9H-purin-6-amine

To a solution of 2-(cyclopentyloxy)-9-[5-S-methyl-2,3-O-(1-methylethylidene)-5-thio-β-D-ribofuranosyl]-9H-purin-6-amine prepared using an analogous procedure to that described for Example 288 (300 mg, 0.71 mmol) in DCM was added m-chloroperbenzoic acid (162 mg, 0.71 mmol). The solution was stirred overnight at rt. The reaction mixture was diluted with chloroform and washed with sodium bicarbonate and brine and concentrated.

The resulting mixture of sulfoxide and sulfone was suspended in 2:1 mixture of acetic acid:water, then stirred at rt for 24 h. The reaction mixture was concentrated to dryness and the residue purified using Gilson reverse phase HPLC with 10 mM ammonium acetate and acetonitrile as the mobile phase with a gradient of 10-60% for 14 min. Relevant fractions were combined and concentrated to give a thin film. The products were dissolved in water and freeze-dried to give white solids.

Example 305: MS (ESP): 398 (MH⁺) for C₁₆H₂₃N₅O₅S

¹H NMR (300 MHz, DMSO-d₆) δ ppm 1.68 (s, 6H) 1.88 (s, 4H) 2.51 (s, 3H) 4.26 (s, 3H) 4.73 (s, 1H) 5.26 (s, 1H) 5.49 (s, 2H) 5.79 (s, 1H) 7.22 (s, 2H) 8.06 (s, 1H)

Example 306: MS (ESP): 414 (MH⁺) for C₁₆H₂₃N₅O₆S

¹H NMR (300 MHz, DMSO-d₆) δ ppm 1.62 (s, 5H) 1.82 (s, 3H) 2.76 (s, 3H) 4.19 (s, 1H) 3.90 (s, 1H) 4.25 (s, 2H) 4.75 (s, 1H) 5.30 (s, 1H) 5.50 (s, 1H) 5.85 (s, 1H) 7.18 (s, 2H) 8.06 (s, 1H)

### EXAMPLE 307

### 1:1 mixture of (1S,2S)-2-[6-amino-2-(cyclopentyloxy)-9H-purin-9-yl]cyclopentanol and (1R,2R)-2-[6-amino-2-(cyclopentyloxy)-9H-purin-9-yl]cyclopentanol

2-(Cyclopentyloxy)-9H-purin-6-amine (TFA salt, 70 mg) was mixed with DMF (3 ml), cyclopentene oxide (390 μl) and potassium carbonate (excess) in a microwave vial containing a magnetic stirring bar. The reaction vessel was sealed and the mixture heated with stirring in a microwave reactor at 120° C. for 2 h. After cooling to rt, the mixture was filtered and the filtrate was purified by reverse-phase HPLC using a gradient of aqueous ammonium acetate (pH 8.0) and acetonitrile. The relevant fractions were combined and solvents evaporated to dryness to give the desired product as a white solid (17.5 mg).

MS (ESP): 304 (MH⁺) for C₁₅H₂₁N₅O₂.

¹H NMR δ: 1.53-1.60 (m, 2H) 1.63-1.83 (m, 8H) 1.85-1.92 (m, 2H) 1.98-2.12 (m, 2H) 4.30-4.39 (m, 1H) 4.44 (dt, 1H) 5.14 (d, 1H) 5.24 (m, 1H) 7.09 (s, 2H) 7.92 (s, 1H)

The intermediate for this compound was prepared as follows:—

### 2-(cyclopentyloxy)-9H-purin-6-amine (TFA salt)

2-(Cyclopentyloxy)-9-(tetrahydrofuran-2-yl)-9H-purin-6-amine prepared as for Example 29 (3.6 g) was dissolved in a 1:2:0.5 mixture of trifluoroacetic acid:DCM:triethylsilane. The mixture was stirred at rt for 3 h. Solvents were evaporated to dryness and the resulting oil dried under high vacuum until it solidified. The resulting solid was triturated with diethyl ether furnishing the desired product as a light beige solid (3.8 g). This material was used in the next step without additional purification. For characterization purposes, a small amount of product was dissolved in DCM and treated with Amberlite IR-743 (strong base) to give the free base. The solution was filtered and solvent evaporated to give an off-white solid.

MS (ESP): 220 (MH⁺) for C₁₀H₁₃N₅O₄

¹H NMR δ: 1.56 (m, 2H) 1.769 (m, 4H) 1.86 (s, 2H) 5.24 (m, 1H) 7.02 (s, 2H) 7.86 (s, 1H) 12.58 (s, 1H)

### EXAMPLE 308

### 9-(3-bromo-3,5-dideoxy-5-fluoro-(3-D-xylofuranosyl)-2-(cyclopentyloxy)-9H-purin-6-amine

To a solution of 2-(cyclopentyloxy)-9-(5-deoxy-5-fluoro-β-D-ribofuranosyl)-9H-purin-6-amine (1.1 g, 3.1 mmol) in a mixture of acetonitrile (10 ml) and water (56 μl) was added 1-bromocarbonyl-1-methylethyl acetate (2.3 ml, 15.6 mmol) at 4° C. The solution was stirred at rt for 1.5 h, then quenched with water and saturated sodium bicarbonate. The reaction mixture was extracted with EtOAc (2×100 ml), dried (sodium sulfate), filtered and concentrated in vacuo. The residue was purified by chromatography eluting with 4% MeOH in DCM to give 9-(3-bromo-3,5-dideoxy-5-fluoro-β-D-xylofuranosyl)-2-(cyclopentyloxy)-9H-purin-6-amine (990 mg). This intermediate (300 mg, 0.66 mmol) was taken up in 0.5N ammonia in MeOH (5 ml) at 4° C. and the stirring was continued for 2 h at 4° C. The reaction mixture was concentrated in vacuo to give the desired product as a white solid (265 mg).

MS (ESP): 417 (MH⁺) for C₁₅H₁₉BrFN₅O₃

¹H NMR δ ppm 1.37-1.78 (m, 8H) 4.44-4.59 (m, 3H) 4.72 (d, 1H) 4.82-4.92 (m, 1H) 5.11-5.20 (m, 1H) 5.64 (d, 1H) 6.31 (d, 1H) 7.15 (s, 2H) 7.95 (s, 1H)

### EXAMPLE 309

### 9-[3-(benzylamino)-3,5-dideoxy-5-fluoro-β-D-ribofuranosyl]-2-(cyclopentyloxy)-9H-purin-6-amine

To a solution of 9-(3-bromo-3,5-dideoxy-5-fluoro-β-D-xylofuranosyl)-2-(cyclopentyloxy)-9H-purin-6-amine (150 mg, 0.36 mmol) in DMF (3 ml) was added successively benzyl isocyanate (89 μl, 0.72 mmol) and triethylamine (100 μl, 0.72 mmol) at rt. The solution was stirred for 2 h, and then quenched with MeOH. The reaction mixture was concentrated in vacuo and the residue was purified by chromatography eluting with 5% MeOH in DCM to give the carbamate derivative. This intermediate was taken up in THF (3 ml) then sodium hydride (109 mg, 2.73 mmol) was added at −40° C. The solution was stirred for 15 min then quenched with water. The reaction mixture was extracted with DCM (2×50 ml). The organic phases were combined and washed with saturated sodium bicarbonate, dried (sodium sulfate) and evaporated to dryness. This residue was taken up in 1N sodium hydroxide (5 ml) then the solution was stirred for 6 h at 100° C. The reaction mixture was neutralized with Amberlite IR-120⁺, filtered and concentrated in vacuo. The residue was purified by chromatography eluting with 5% MeOH in DCM to give desired product as a white solid (23 mg).

MS (ESP): 443 (MH⁺) for C₂₂H₂₇FN₆O₃

¹H NMR (300 MHz, CDCl₃) δ ppm 1.18-1.77 (m, 8H) 3.82 (s, 2H) 4.08 (s, 1H) 4.15 (d, 1H) 4.37 (s, 1H) 4.49 (dd, 2H) 4.67 (s, 1H) 5.12-5.24 (m, 1H) 5.50 (s, 2H) 5.94 (s, 1H) 7.18-7.32 (m, 6H) 7.72 (s, 1H)

### EXAMPLE 310

### 2-(cyclopentyloxy)-9-O-D-xylofuranosyl-9H-purin-6-amine

A suspension of N-[2-(cyclopentyloxy)-9H-purin-6-yl]benzamide (0.5 g, 1.5 mmol) (preparation described for Example 127), 1,2,3,5-tetra-O-acetyl-D-xylofuranose (commercially available) (1 g, 3.1 mmol) and N,O-bis(trimethylsilyl)acetamide (1 ml, 3.9 mmol) in 10 mL dry acetonitrile was warmed to 60° C. After stirring for 30 min, 0.6 mL (5.1 mmol) tin (IV) chloride was added dropwise and stirring was continued for another 90 min. The reaction mixture was cooled to rt and poured into a mixture of cold saturated sodium bicarbonate and EtOAc (1:1, v/v, 250 ml). The aqueous phase was extracted with EtOAc (150 ml). The organic phases were combined and washed with saturated sodium bicarbonate, dried (sodium sulfate) and evaporated to dryness. The residue was purified by chromatography eluting with 65% EtOAc in hexane to give N-benzoyl-2-(cyclopentyloxy)-9-(2,3,5-tri-O-acetyl-β-D-xylofuranosyl)-9H-purin-6-amine (356 mg). This intermediate was taken up in 7N ammonia in MeOH (10 ml) and the stirring was continued overnight. The reaction mixture was concentrated in vacuo and the residue was purified by chromatography eluting with 8% MeOH in DCM to give the desired product (156 mg).

MS (ESP): 352 (MH⁺) for C₁₅H₂₁N₅O₅

¹H NMR (400 MHz, DMSO-d₆) δ ppm 1.77-1.94 (m, 6H) 2.05-2.19 (m, 2H) 3.85 (dt, 1H) 3.90-3.99 (m, 1H) 4.17-4.26 (m, 1H) 4.28-4.37 (m, 1H) 4.48-4.56 (m, 1H) 4.95 (t, 1H) 5.48-5.57 (m, 1H) 5.94 (d, 1H) 5.97 (d, 1H) 6.07 (d, 1H) 7.47 (s, 2H) 8.28 (s, 1H)

### EXAMPLE 311

### 9-(3-bromo-3,5-dideoxy-β-D-xylofuranosyl)-2-(cyclopentyloxy)-9H-purin-6-amine and

### EXAMPLE 312

### 9-(2-bromo-2,5-dideoxy-β-D-arabinofuranosyl)-2-(cyclopentyloxy)-9H-purin-6-amine

To a solution of 2-(cyclopentyloxy)-9-(5-deoxy-β-D-ribofuranosyl)-9H-purin-6-amine (2.88 g, 8.59 mmol) in a mixture of acetonitrile (150 ml) and water (155 μl) was added 1-bromocarbonyl-1-methylethyl acetate (6.33 ml, 42.9 mmol) at 4° C. The solution was stirred at rt for 1 h, then quenched with water (10 ml) and saturated sodium bicarbonate (200 ml). The reaction mixture was extracted with EtOAc (300 ml), dried (sodium sulfate), filtered and concentrated in vacuo to give 3.46 g of a mixture of 9-(2-O-acetyl-3-bromo-3,5-dideoxy-β-D-xylofuranosyl)-2-(cyclopentyloxy)-9H-purin-6-amine and 9-(3-O-acetyl-2-bromo-2,5-dideoxy-β-D-arabinofuranosyl)-2-(cyclopentyloxy)-9H-purin-6-amine. This mixture (2.0 g, 4.55 mmol) was taken up in 7N ammonia in MeOH (20 ml) and MeOH (50 ml) at 4° C. and the stirring was continued for 3 h at 4° C. The reaction mixture was concentrated in vacuo and the residue was purified by Gilson reverse phase HPLC with 10 mM ammonium acetate and acetonitrile as the mobile phases with a gradient of 25-50% in 15 min. Relevant fractions were combined to give 1.2 g of 9-(3-bromo-3,5-dideoxy-β-D-xylofuranosyl)-2-(cyclopentyloxy)-9H-purin-6-amine and 0.25 g of 9-(2-bromo-2,5-dideoxy-β-D-arabinofuranosyl)-2-(cyclopentyloxy)-9H-purin-6-amine.

Example 311: MS (ESP): 400 (MH⁺) for C₁₅H₂₀BrN₅O₃

¹H NMR (400 MHz, DMSO-d₆) δ ppm 1.40 (d, 3H) 1.54 1.98 (m, 8H) 4.38-4.41 (qt, 1H) 4.54 (dd, 1H) 5.02 (t, 1H) 5.32 (dq, 1H) 5.70 (d, 1H) 6.40 (s, 1H) 7.23 (s, 2H) 8.04 (s, 1H).

Example 312: MS (ESP): 400 (MH⁺) for C₁₅H₂₀BrN₅O₃

1H NMR (400 MHz, DMSO-D6) δ ppm 1.41 (d, 3H) 1.54-1.64 (m, 2H) 1.65-1.76 (m, 4H) 1.91 (s, 2H) 3.75-3.85 (dt, 1H) 4.46-4.55 (t, 1H) 4.71 (dd, 1H) 5.25-5.32 (m, 1H) 6.01 (s, 1H) 6.24 (d, 1H) 7.19 (s, 1H) 8.01 (s, 1H).

### EXAMPLE 313

### 9-(3-cyano-3,5-dideoxy-O-D-xylofuranosyl)-2-(cyclopentyloxy)-9H-purin-6-amine

To a solution of 9-(2,3-anhydro-5-deoxy-β-D-ribofuranosyl)-2-(cyclopentyloxy)-9H-purin-6-amine (100 mg, 0.15 mmol) in THF (1.5 ml) was added a 2 M solution of diethylaluminum cyanide in toluene (1.58 ml, 1.58 mmol) at rt. The resulting clear solution was heated at 80° C. for 30 min, allowed to warm to rt, diluted with ethanol (1 ml), and stirred for 30 min. The solution was diluted with DCM (100 ml), washed successively with water and brine, dried over sodium sulfate, and concentrated in vacuo. The residue was purified by Gilson reverse phase HPLC with 10 mM ammonium acetate and acetonitrile as the mobile phases. Relevant fractions were combined to give 16 mg of the desired product.

MS (ESP): 345 (MH⁺) for C₁₆H₂₀N₆O₃

¹H NMR (400 MHz, DMSO-d₆) δ ppm 1.37 (d, 3H) 1.51-1.86 (m, 8H) 3.56 (t, 1H) 4.43 (t, 1H) 5.25 (m, 2H) 5.59 (d, 1H) 6.38 (s, 1H) 7.17 (s, 2H) 8.03 (s, 1H).

The intermediates for this compound were prepared as follows:

### 9-(2,3-anhydro-5-deoxy-β-D-ribofuranosyl)-2-(cyclopentyloxy)-9H-purin-6-amine

To a mixture of 9-(2-O-acetyl-3-bromo-3,5-dideoxy-β-D-xylofuranosyl)-2-(cyclopentyloxy)-9H-purin-6-amine and 9-(3-O-acetyl-2-bromo-2,5-dideoxy-β-D-arabinofuranosyl)-2-(cyclopentyloxy)-9H-purin-6-amine (preparation described in previous Example) (1.45 g, 3.29 mmol) in MeOH (12 ml) was added potassium carbonate (0.91 g, 6.59 mmol) at rt. The suspension was stirred for 1 h, then diluted with MeOH (20 ml) and DCM (30 ml), and filtered through diatomaceous earth. The filtrate was concentrated in vacuo and the residue was purified by chromatography eluting with 5% MeOH in DCM to give the desired product (0.91 g).

MS (ESP): 318 (MH⁺) for C₁₅H₁₉N₅O₃

¹H NMR (400 MHz, DMSO-d₆) δ ppm 1.19 (d, 3H) 1.54-1.96 (m, 8H) 4.11 (d, 1H) 4.30 (q, 1H) 4.48 (d, 1H) 5.26-5.33 (m, 1H) 6.09 (s, 1H) 7.21 (s, 2H) 8.07 (s, 1H).

### EXAMPLE 314

### 2-(cyclopentyloxy)-9-(3,5-dideoxy-3-methyl-β-D-xylofuranosyl)-9H-purin-6-amine

To methyl magnesium iodide (2.63 ml, 3M in diethylether) was added copper (I) iodide (75.0 mg, 0.39 mmol) at −20° C. The suspension was brought to 0° C. and 9-(2,3-anhydro-5-deoxy-β-D-ribofuranosyl)-2-(cyclopentyloxy)-9H-purin-6-amine (250 mg, 0.79 mmol) dissolved in THF (5 ml), was added dropwise. The solution was stirred for 5 h at 0° C., then at rt overnight. The reaction mixture was diluted with DCM (100 ml), washed successively with water and saturated sodium bicarbonate, dried over sodium sulfate, and concentrated in vacuo. The residue was purified by Gilson reverse phase HPLC with 10 mM ammonium acetate and acetonitrile as the mobile phases with a gradient of 45-85% in 15 min. Relevant fractions were combined to give 10 mg of the desired product.

MS (ESP): 334 (MH⁺) for C₁₆H₂₃N₅O₃

¹H NMR (400 MHz, DMSO-d₆) δ ppm 1.04 (d, 3H) 1.21 (d, 3H) 1.59-1.90 (m, 8H) 2.31 (m, 1H) 4.33 (t, 1H) 4.62 (t, 1H) 5.28 (m, 1H) 5.55 (d, 1H) 7.17 (s, 2H) 8.09 (s, 1H)

### EXAMPLE 315

### 9-(3-azido-3,5-dideoxy-β-D-xylofuranosyl)-2-(cyclopentyloxy)-9H-purin-6-amine

A suspension of 9-(2,3-anhydro-5-deoxy-β-D-ribofuranosyl)-2-(cyclopentyloxy)-9H-purin-6-amine (175 mg, 0.55 mmol), sodium azide (179 mg, 2.76 mmol) and ammonium chloride (147 mg, 2.76 mmol) in DMF (2 ml) was heated in a microwave reactor for 3 h at 100° C. The reaction mixture was diluted with DCM (40 ml) and MeOH (4 ml), and then washed with water. The organic phase was dried (sodium sulfate) and evaporated to dryness. The residue was purified by Gilson reverse phase HPLC with 10 mM ammonium acetate and acetonitrile as the mobile phases with a gradient of 20-60% in 15 min. Relevant fractions were combined to give 108 mg of the desired product.

MS (ESP): 361 (MH⁺) for C₁₅H₂₀N₈O₅

¹H NMR (400 MHz, DMSO-d₆) δ ppm 1.31 (s, 3H) 1.59-1.92 (m, 8H) 4.26 (dd, 1H) 4.43 (qt, 1H) 4.85 (t, 1H) 5.30 (m, 1H) 5.69 (d, 1H) 6.21 (s, 1H) 7.22 (s, 2H) 8.03 (s, 1H).

### EXAMPLE 316

### 9-(3-amino-3,5-dideoxy-O-D-xylofuranosyl)-2-(cyclopentyloxy)-9H-purin-6-amine

To a solution of 9-(5-azido-5-deoxy-β-D-ribofuranosyl)-2-(cyclopentyloxy)-9H-purin-6-amine (87 mg, 0.242 mmol) in ethanol (3 ml) was added 10% palladium on charcoal (50 mg). The reaction mixture was stirred under hydrogen (1 atm) for 5 h. Then the reaction mixture was diluted with ethanol, filtered through diatomaceous earth and evaporated. The residue was purified using Gilson reverse phase HPLC with 10 mM ammonium acetate and acetonitrile as the mobile phases with a gradient of 5-95% in 15 min. Relevant fractions were combined to give 52 mg of the desired product.

MS (ESP): 335 (MH⁺) for C₁₅H₂₂N₆O₃

¹H NMR (400 MHz, DMSO-d₆) δ ppm 1.16 (d, 3H) 1.51-1.84 (m, 8H) 4.18 (qt, 1H) 4.24 (t, 1H) 5.23 (m, 1H) 5.57 (d, 1H) 7.09 (s, 2H) 8.18 (s, 1H).

### EXAMPLE 317

### 9-[3-(acetylamino)-3,5-dideoxy-β-D-xylofuranosyl]-2-(cyclopentyloxy)-9H-purin-6-amine

To a solution of 9-(3-amino-3,5-dideoxy-β-D-xylofuranosyl)-2-(cyclopentyloxy)-9H-purin-6-amine (39.4 mg, 0.118 mmol) in dry pyridine (2 ml) was added acetic anhydride (0.1 ml) at 4° C. The solution was stirred at rt overnight, then quenched with MeOH and concentrated in vacuo. The residue was taken up in 7N ammonia in MeOH (5 ml) at rt and the stirring was continued overnight. The reaction mixture was concentrated in vacuo and the residue was purified by chromatography eluting with 6% MeOH in DCM to give the desired product (37 mg).

MS (ESP): 377 (MH⁺) for C₁₇H₂₄N₆O₄

¹H NMR (400 MHz, DMSO-d₆) δ ppm 1.02 (d, 3H) 1.51-1.91 (m, 8H) 1.9 (s, 3H) 4.13 (dd, 1H) 4.15 (t, 1H) 4.41 (t, 1H) 5.29 (m, 1H) 5.53 (d, 1H) 5.86 (d, 1H) 7.35 (s, 2H) 8.03 (s, 1H) 9.21 (d, 1H).

### EXAMPLE 318

### 9-[(3ξ)-3-azido-3,5-dideoxy-5-fluoro-O-D-erythro-pentofuranosyl]-2-(cyclopentyloxy)-9H-purin-6-amine

A suspension of 9-[3-bromo-3,5-dideoxy-5-fluoro-2-O-(triisopropylsilyl)-β-D-xylofuranosyl]-2-(cyclopentyloxy)-9H-purin-6-amine (100 mg, 0.18 mmol), sodium azide (57 mg, 0.87 mmol) and ammonium chloride (47 mg, 0.87 mmol) in DMF (2 ml) was heated in a microwave reactor for 1 h at 110° C. The reaction mixture was diluted with DCM (40 ml) and MeOH (4 ml), and then washed with water. The organic phase was dried (sodium sulfate) and evaporated to dryness. The residue was purified by Gilson reverse phase HPLC with 10 mM ammonium acetate and acetonitrile as the mobile phases with a gradient of 20-50% in 15 min. Relevant fractions were combined to give 6.6 mg of the desired product.

MS (ESP): 379 (MH⁺) for C₁₅H₁₉FN₈O₃

¹H NMR (400 MHz, DMSO-d₆) δ ppm 1.47-1.86 (m, 8H) 4.05-4.16 (m, 1H) 4.39 (t, 1H) 4.46 (dt, 1H) 4.52-4.57 (m, 1H) 4.61-4.70 (m, 1H) 4.80 (s, 1H) 5.00 (s, 1H) 5.16-5.25 (m, 1H) 5.70 (d, 1H) 5.77 (d, 1H) 6.29 (m, 1H) 7.18 (s, 2H) 7.98-8.06 (m, 1H)

The intermediate was prepared as follows:—

### 9-[3-bromo-3,5-dideoxy-5-fluoro-2-O-(triisopropylsilyl)-D-D-xylofuranosyl]-2-(cyclopentyloxy)-9H-purin-6-amine

To a solution of 9-(3-bromo-3,5-dideoxy-5-fluoro-β-D-xylofuranosyl)-2-(cyclopentyloxy)-9H-purin-6-amine (prepared as for Example 254) (0.9 g, 2.2 mmol) in DMF (10 ml) was added successively imidazole (519 mg, 7.6 mmol) and triisopropylsilyl chloride (1.5 ml, 6.5 mmol) at rt. The solution was stirred for 48 h, quenched with saturated aqueous sodium bicarbonate (2 ml), and concentrated in vacuo. The residue was dissolved in DCM (200 ml), washed with water, dried over sodium sulfate, and concentrated in vacuo. The residue was purified by flash chromatography eluting with 60% hexane in EtOAc to give the desired product (700 mg).

MS (ESP): 573 (MH⁺) for C₂₄H₃₉FBrN₅O₃Si

¹H NMR (300 MHz, DMSO-d₆) δ ppm 0.71-0.86 (m, 21H) 1.42-1.73 (m, 8H) 4.43-4.74 (m, 4H) 5.10-5.23 (m, 2H) 5.63-5.72 (d, 1H) 7.13 (s, 2H) 7.94 (s, 1H).

### EXAMPLE 319

### 2-(cyclopentyloxy)-9-(5-deoxy-3-S-ethyl-3-thio-β-D-xylofuranosyl)-9H-purin-6-amine and

### EXAMPLE 320

### 2-(cyclopentyloxy)-9-(5-deoxy-2-S-ethyl-2-thio-β-D-arabinofuranosyl)-9H-purin-6-amine

A solution of 25% wt sodium methoxide (171 μl, 0.75 mmol) and ethanethiol (110 μl 1.49 mmol) in MeOH (2 ml) was stirred at rt for 20 min. 9-(2,3-anhydro-5-deoxy-β-D-ribofuranosyl)-2-(cyclopentyloxy)-N-(2,2-dimethylpropanoyl)-9H-purin-6-amine (150 mg, 0.37 mmol) was added to this solution, and the mixture was stirred at reflux for 1 h. The reaction mixture was concentrated in vacuo and the residue was purified by chromatography eluting with 10% MeOH in DCM to give 2-(cyclopentyloxy)-9-(5-deoxy-3-S-ethyl-3-thio-β-D-xylofuranosyl)-9H-purin-6-amine (67 mg) and 2-(cyclopentyloxy)-9-(5-deoxy-2-S-ethyl-2-thio-β-D-arabinofuranosyl)-9H-purin-6-amine.

Example 319: MS (ESP): 380 (MH⁺) for C₁₇H₂₅N₅O₃S

¹H NMR (400 MHz, DMSO-d₆) δ ppm 1.22 (t, 3H) 1.34 (d, 3H) 1.59-1.92 (m, 8H) 2.66 (qt, 2H) 3.42 (t, 1H) 4.52 (dt, 1H) 4.85 (q, 1H) 5.29 (m, 1H) 5.64 (d, 1H) 5.95 (d, 1H) 7.19 (s, 2H) 8.09 (s, 1H)

Example 320: MS (ESP): 380 (MH⁺) for C₁₇H₂₅N₅O₃S

¹H NMR (400 MHz, DMSO-d₆) δ ppm 0.95 (t, 3H) 1.26 (d, 3H) 1.51 (m, 2H) 1.63 (m, 4H) 1.82 (m, 2H) 2.30-2.39 (m, 2H) 3.51-3.60 (dd, 1H) 3.63-3.71 (q, 1H) 4.07 (t, 1H) 5.17-5.25 (m, 1H) 5.60 (d, 1H) 6.21 (d, 1H) 7.06 (s, 2H) 7.84 (s, 1H).

### EXAMPLE 321

### 2-(cyclopentyloxy)-9-[3,5-dideoxy-3-(ethylsulfonyl)-D-D-xylofuranosyl]-9H-purin-6-amine

To a solution of 2-(cyclopentyloxy)-9-(5-deoxy-3-S-ethyl-3-thio-β-D-xylofuranosyl)-9H-purin-6-amine (58 mg, 0.153 mmol) in dry pyridine (1 ml) was added chlorotrimethylsilane (38 μl) at rt. After stirring for 1 h, the reaction mixture was cooled to 4° C., and benzoyl chloride (27 μl, 0.229 mmol) was added dropwise. The ice bath was then removed and the reaction mixture stirred at rt for 4 h. The reaction was quenched by the addition of water (5 ml), stirred for 10 min, and then diluted with DCM (50 ml). The organic phase was separated, dried (sodium sulfate) and concentrated in vacuo. The residue was taken up in DCM (2 ml), and 3-chloroperoxybenzoic acid (146 mg, 0.85 mmol) was added at 4° C. The solution was stirred for 1 h, quenched with triethylamine (2 drops), and then concentrated in vacuo. The residue was dissolved in DCM (50 ml), washed with saturated sodium bicarbonate, dried (sodium sulfate) and concentrated to dryness. This intermediate was taken up in 7N ammonia in MeOH (10 ml) and the stirring was continued overnight at rt. The reaction mixture was concentrated in vacuo and the residue was purified by chromatography eluting with 4% MeOH in DCM to give the desired product (25 mg).

MS (ESP): 412 (MH⁺) for C₁₇H₂₅N₅O₅S

¹H NMR (400 MHz, DMSO-d₆) δ ppm 1.20 (t, 3H) 1.51 (d, 3H) 1.62-1.85 (m, 8H) 3.09 (qt, 2H) 4.15 (t, 1H) 4.57 (dd, 1H) 5.30 (m, 1H) 5.49 (s, 1H) 5.62 (d, 1H) 6.32 (d, 1H) 7.16 (s, 2H) 8.04 (s, 1H).

### EXAMPLE 322

### 2-(cyclopentyloxy)-9-(5-deoxy-3-S-phenyl-3-thio-3-D-xylofuranosyl)-9H-purin-6-amine and

### EXAMPLE 323

### 2-(cyclopentyloxy)-9-(5-deoxy-2-S-phenyl-2-thio-D-D-arabinofuranosyl)-9H-purin-6-amine

A solution of 25% wt sodium methoxide (228 μl, 0.997 mmol) and thiophenol (205 μl, 1.99 mmol) in MeOH (2 ml) was stirred at rt for 20 min. 9-(2,3-anhydro-5-deoxy-β-D-ribofuranosyl)-2-(cyclopentyloxy)-N-(2,2-dimethylpropanoyl)-9H-purin-6-amine (150 mg, 0.37 mmol) was added to this solution, and the mixture was stirred at reflux for 1 h. The reaction mixture was concentrated in vacuo and the residue was purified using Gilson reverse phase HPLC with 10 mM ammonium acetate and acetonitrile as the mobile phases with a gradient of 5-95% in 15 min. Relevant fractions were combined to give 2-(cyclopentyloxy)-9-(5-deoxy-3-S-phenyl-3-thio-β-D-xylofuranosyl)-9H-purin-6-amine (100 mg) and 2-(cyclopentyloxy)-9-(5-deoxy-2-S-phenyl-2-thio-β-D-arabinofuranosyl)-9H-purin-6-amine.

Example 322: MS (ESP): 428 (MH⁺) for C₂₁H₂₅N₅O₃S

¹H NMR (400 MHz, DMSO-d₆) δ ppm 1.31 (d, 3H) 1.51-1.84 (m, 8H) 3.90 (t, 1H) 4.57 (qt, 1H) 4.78 (t, 1H) 5.21 (m, 1H) 5.65 (d, 1H) 6.08 (d, 1H) 7.08-7.19 (m, 3H) 7.26 (t, 2H) 7.31-7.35 (m, 2H) 8.04 (s, 1H).

Example 323: MS (ESP): 428 (MH⁺) for C₂₁H₂₅N₅O₃S

¹H NMR (400 MHz, DMSO-D6) δ ppm 1.29 (d, 3H) 1.52 (m, 2H) 1.62 (m, 4H) 1.79 (m, 2H) 3.74 (q, 1H) 4.07 (t, 1H) 4.26 (t, 1H) 5.14 (m, 1H) 5.81 (d, 1H) 6.36 (d, 1H) 7.05 (m, 3H) 7.14 (m, 4H) 7.85 (s, 1H).

### EXAMPLE 324

### 2-(cyclopentyloxy)-9-[3,5-dideoxy-3-(phenylsulfonyl)-β-D-xylofuranosyl]-9H-purin-6-amine

To a solution of 2-(cyclopentyloxy)-9-(5-deoxy-3-S-phenyl-3-thio-β-D-xylofuranosyl)-9H-purin-6-amine (82.5 mg, 0.193 mmol) in dry pyridine (2 ml) was added successively 4-(dimethylamino) pyridine (10 mg) and benzoyl chloride (189 μl, 1.15 mmol) at 4° C. The solution was stirred at rt overnight, then quenched with water (10 ml) and diluted with DCM (100 ml). The organic phase was separated, washed with an aqueous solution of hydrochloric acid (0.5N), dried (sodium sulfate) and concentrated to dryness. The residue was purified by chromatography eluting with 50% hexane in EtOAc to give the desired product (121 mg), which was taken up in DCM. 3-Chloroperoxybenzoic acid (146 mg, 0.85 mmol) was added at 4° C. and the solution was stirred for 2 h, quenched with triethylamine (2 drops), and then concentrated in vacuo. The residue was dissolved in DCM (50 ml), washed with saturated sodium bicarbonate, dried (sodium sulfate) and concentrated to dryness. This intermediate was taken up in 7N ammonia in MeOH (10 ml) and the stirring was continued overnight at rt. The reaction mixture was concentrated in vacuo and the residue was purified using Gilson reverse phase HPLC with 10 mM ammonium acetate and acetonitrile as the mobile phases with a gradient of 20-60% in 15 min. Relevant fractions were combined to give 13 mg of the desired product.

MS (ESP): 460 (MH⁺) for C₂₁H₂₅N₅O₅S

¹H NMR (400 MHz, DMSO-d₆) δ ppm 1.49 (d, 3H) 1.63-1.84 (m, 8H) 4.08 (t, 1H) 4.43 (t, 1H) 4.54 (qt, 1H) 5.28 (m, 1H) 5.49 (d, 1H) 7.14 (s, 2H) 7.62 (d, 2H) 7.70-7.92 (d, 3H) 7.97 (s, 1H).

### EXAMPLE 325

### 2-(cyclopentyloxy)-9-(5-deoxy-3-O-isopropyl-β-D-ribofuranosyl)-9H-purin-6-amine and

### EXAMPLE 326

### 2-(cyclopentyloxy)-9-(5-deoxy-2-O-isopropyl-3-D-ribofuranosyl)-9H-purin-6-amine

To a solution of 2-(cyclopentyloxy)-9-[5-deoxy-2,3-O-(1-methylethylidene)-β-D-ribofuranosyl]-9H-purin-6-amine (300 mg, 0.8 mmol) in dry DCM (3 ml) and diethyl ether (3 ml) were added successively lithium aluminum hydride (61 mg, 1.6 mmol) and a solution of aluminum trichloride (213 mg, 1.6 mmol) in diethyl ether (2 ml) at rt. After stirring for 2 h, the reaction mixture was cooled to 4° C., and EtOAc (100 ml) and water (100 ml) were added successively. The organic phase was separated, dried (sodium sulfate) and concentrated to dryness. The residue was purified using Gilson reverse phase HPLC with 10 mM ammonium acetate and acetonitrile as the mobile phases with a gradient of 25-50% in 15 min. Relevant fractions were combined to give 2-(cyclopentyloxy)-9-(5-deoxy-3-O-isopropyl-β-D-ribofuranosyl)-9H-purin-6-amine (130 mg) and 2-(cyclopentyloxy)-9-(5-deoxy-2-O-isopropyl-β-D-ribofuranosyl)-9H-purin-6-amine.

Example 325: MS (ESP): 378 (MH⁺) for C₁₈H₂₇N₅O₄

¹H NMR (400 MHz, DMSO-d₆) δ ppm 1.17 (dd, 6H) 1.30 (d, 3H) 1.58-1.95 (m, 8H) 3.74 (dd, 1H) 3.95 (d, 1H) 3.96 (t, 1H) 4.75 (d, 1H) 5.18 (d, 1H) 5.26 (m, 1H) 5.34 5.71 (d, 1H) 7.19 (s, 2H) 8.10 (s, 1H).

Example 326: MS (ESP): 378-(MH⁺) for C₁₈H₂₇N₅O₄

¹H NMR (400 MHz, DMSO-d₆) δ ppm 0.99 (d, 3H) 1.10 (d, 3H) 1.32 (d, 3H) 1.58 (m, 2H) 1.70 (m, 2H) 1.71 (m, 2H) 1.84-1.96 (m, 2H) 3.71 (dt, 1H) 3.96 (dd, 1H) 4.04 (q, 1H) 4.67 (t, 1H) 4.95 (d, 1H) 5.22-5.31 (m, 1H) 5.77 (d, 1H) 7.19 (s, 2H) 8.12 (s, 1H).

### EXAMPLE 327

### 9-(3-O-benzyl-5-deoxy-β-D-ribofuranosyl)-2-(cyclopentyloxy)-9H-purin-6-amine and

### EXAMPLE 328

### 9-(2-O-benzyl-5-deoxy-β-D-ribofuranosyl)-2-(cyclopentyloxy)-9H-purin-6-amine

To a solution of 2-(cyclopentyloxy)-9-(5-deoxy-β-D-ribofuranosyl)-9H-purin-6-amine (200 mg, 0.597 mmol) and benzaldehyde dimethyl acetal (449 μl, 2.98 mmol) in dry acetonitrile (2 ml) was added phosphorusoxychloride at 4° C. After stirring for 1 h at 4° C. then at rt for 2 h, the solution was diluted with EtOAc (100 ml), and washed successively with saturated sodium bicarbonate and brine, dried (sodium sulfate) and concentrated in vacuo. The residue was purified using Gilson reverse phase HPLC with 10 mM ammonium acetate and acetonitrile as the mobile phases with a gradient of 50-95% in 15 min. Relevant fractions were combined to give 116 mg of the desired product which was taken up in dry DCM (3 ml) and diethyl ether (3 ml). Lithium aluminum hydride (38 mg, 0.99 mmol) and a solution of aluminum trichloride (132 mg, 0.99 mmol) in diethyl ether (2 ml) were added successively at rt. After stirring for 1 h, the reaction mixture was cooled to 4° C., and EtOAc (100 ml) and water (100 ml) were added successively. The organic phase was separated, dried (sodium sulfate) and concentrated to dryness. The residue was purified using Gilson reverse phase HPLC with 10 mM ammonium acetate and acetonitrile as the mobile phases with a gradient of 35-50% in 15 min. Relevant fractions were combined to give 9-(3-O-benzyl-5-deoxy-β-D-ribofuranosyl)-2-(cyclopentyloxy)-9H-purin-6-amine (30 mg) and 9-(2-O-benzyl-5-deoxy-β-D-ribofuranosyl)-2-(cyclopentyloxy)-9H-purin-6-amine.

Example 327: MS (ESP): 426 (MH⁺) for C₂₂H₂₇N₅O₄

¹H NMR (400 MHz, DMSO-d₆) δ ppm 1.24 (d, 3H) 1.49-1.79 (m, 8H) 3.88 (t, 1H) 4.07 (dt, 1H) 4.53 (d, 1H) 4.71 (d, 1H) 4.85 (t, 1H) 5.18 (m, 1H) 5.72 (d, 1H) 7.24-7.37 (m, 7H) 8.07 (s, 1H).

Example 328: MS (ESP): 426 (MH⁺) for C₂₂H₂₇N₅O₄

¹H NMR (400 MHz, DMSO-d₆) δ ppm 1.24 (d, 3H) 1.48 (m, 2H) 1.60 (m, 4H) 1.77 (m, 2H) 3.88-3.98 (q, 1H) 4.10 (t, 1H) 4.49 (d, 1H) 4.55 (t, 1H) 4.63 (d, 1H) 5.09-5.18 (m, 1H) 5.85 (d, 1H) 7.13-7.24 (m, 7H) 8.01 (s, 1H).

### EXAMPLE 329

### 9-(2,3-anhydro-β-D-ribofuranosyl)-2-(cyclopentyloxy)-9H-purin-6-amine

To a solution of 2-(cyclopentyloxy)-9-β-D-ribofuranosyl-9H-purin-6-amine (1.4 g, 3.98 mmol) in a mixture of acetonitrile (150 ml) and water (72 μl) was added 1-bromocarbonyl-1-methylethyl acetate (2.93 ml, 19.9 mmol) at 4° C. The solution was stirred at rt for 2 h, then quenched with water (10 ml) and saturated sodium bicarbonate (200 ml). The reaction mixture was extracted with EtOAc (300 ml), dried (sodium sulfate), filtered and concentrated in vacuo. The residue was taken up in MeOH (20 ml) and potassium carbonate (0.91 g, 6.59 mmol) was added at rt. The suspension was stirred for 1 h, then diluted with MeOH (20 ml) and DCM (50 ml), and filtered through diatomaceous earth. The filtrate was concentrated in vacuo and the residue was purified by chromatography eluting with 8% MeOH in DCM to give the desired product (0.8 g).

MS (ESP): 334 (MH⁺) for C₁₅H₁₉N₅O₄

¹H NMR (300 MHz, DMSO-d₆) δ ppm 1.72-2.20 (m, 8H) 3.66-3.82 (dq, 1H) 4.36 (t, 1H) 4.41 (d, 1H) 4.65 (d, 1H) 5.21 (t, 1H) 5.50 (m, 1H) 6.32 (s, 1H) 7.45 (s, 2H) 8.32 (s, 1H).

### EXAMPLE 330

### 2-(cyclopentyloxy)-9-(3-deoxy-3-fluoro-O-D-xylofuranosyl)-9H-purin-6-amine and

### EXAMPLE 331

### 2-(cyclopentyloxy)-9-(2-deoxy-2-fluoro-β-D-arabinofuranosyl)-9H-purin-6-amine

A solution of 9-(2,3-anhydro-β-D-ribofuranosyl)-2-(cyclopentyloxy)-9H-purin-6-amine (250 mg, 0.75 mmol) and tetrabutylammonium fluoride (1.5 ml, 1M THF) was heated in a microwave for 1 h at 130° C. The reaction mixture was concentrated in vacuo and the residue was purified by chromatography eluting with 6% MeOH in DCM to give 70 mg of 2-(cyclopentyloxy)-9-(3-deoxy-3-fluoro-β-D-xylofuranosyl)-9H-purin-6-amine and 27 mg of 2-(cyclopentyloxy)-9-(2-deoxy-2-fluoro-β-D-arabinofuranosyl)-9H-purin-6-amine.

Example 330: MS (ESP): 354 (MH⁺) for C₁₅H₂₀FN₅O₄

¹H NMR δ: 1.51-1.85 (m, 8H) 3.64 (m, 2H) 4.15-4.25 (dq, 1H) 4.71 (dt, 1H) 4.95 (d, 1H) 4.97 (t, 1H) 5.24 (m, 1H) 5.73 (d, 1H) 6.18 (d, 1H) 7.19 (s, 2H) 7.82 (s, 1H).

Example 331: MS (ESP): 354 (MH⁺) for C₁₅H₂₀FN₅O₄

¹H NMR δ: 1.51-1.83 (m, 8H) 3.57 (m, 1H) 3.74 (qt, 1H) 4.36 (m, 1H) 4.97 (m, 2H) 5.21 (m, 2H) 5.88 (d, 1H) 6.21 (dd, 1H) 7.19 (s, 2H) 7.92 (s, 1H).

### EXAMPLE 332

### 2-(cyclopentyloxy)-9-(5-deoxy-5-fluoro-5-D-xylofuranosyl)-9H-purin-6-amine and

### EXAMPLE 333

### 9-(3,5-anhydro-O-D-xylofuranosyl)-2-(cyclopentyloxy)-9H-purin-6-amine

To a solution of 2-(cyclopentyloxy)-9-β-D-xylofuranosyl-9H-purin-6-amine (prepared as described for Example 310) (150 mg, 0.427 mmol) in dry pyridine (4 ml) was added tosylchloride (156 mg, 0.8 mmol) at 4° C. The solution was stirred at 4° C. overnight, and then diluted with DCM (60 ml). The reaction mixture was washed with saturated sodium bicarbonate and the aqueous phase was extracted with DCM (60 ml). The organic phases were combined and concentrated in vacuo. The residue was purified by chromatography eluting with 6% MeOH in DCM to give the desired product (156 mg) which was taken up in tetrabutylammonium fluoride (891 μl, 1M THF), and the solution was heated in a microwave reactor for 20 min at 100° C. The reaction mixture was diluted with DCM then washed with saturated sodium bicarbonate, dried (sodium sulfate) and concentrated in vacuo. The residue was purified by chromatography eluting with 5% MeOH in DCM to give 20 mg of 2-(cyclopentyloxy)-9-(5-deoxy-5-fluoro-β-D-xylofuranosyl)-9H-purin-6-amine and 9 mg of 9-(3,5-anhydro-β-D-xylofuranosyl)-2-(cyclopentyloxy)-9H-purin-6-amine.

Example 332: MS (ESP): 354 (MH⁺) for C₁₅H₂₀FN₅O₅

¹H NMR δ: 1.51-1.84 (m, 8H) 4.09 (t, 1H) 4.30 (m, 2H) 4.52-4.80 (m, 2H) 5.24 (m, 1H) 5.74 (d, 1H) 5.84-5.95 (m, 2H) 7.20 (s, 2H) 8.00 (s, 1H).

Example 333: MS (ESP): 334 (MH⁺) for C₁₅H₁₉N₅O₄

¹H NMR δ: 1.51-1.85 (m, 8H) 3.91 (dd, 1H) 4.64 (dd, 1H) 4.92 (d, 1H) 5.03-5.10 (m, 1H) 5.14 (d, 1H) 5.26 (m, 1H) 5.87 (d, 1H) 6.14 (s, 1H) 7.19 (s, 2H) 8.13 (s, 1H).

### EXAMPLE 334

### 2-(cyclopentyloxy)-9-(3,5-dideoxy-3-fluoro-5-D-xylofuranosyl)-9H-purin-6-amine

A solution of 9-(2,3-anhydro-5-deoxy-β-D-ribofuranosyl)-2-(cyclopentyloxy)-9H-purin-6-amine (prepared as described for Example 262) (300 mg, 0.95 mmol) and tetrabutylammonium fluoride (2 ml, 1M THF) was heated in a microwave reactor for 1 h at 120° C. The reaction mixture was concentrated in vacuo and the residue was purified by chromatography over silica gel eluting with 3% MeOH in DCM to give desired product (151 mg).

MS (ESP): 338 (MH⁺) for C₁₅H₂₀FN₅O₃

¹H NMR (400 MHz, DMSO-d₆) δ ppm 1.27 (d, 3H) 1.47-1.90 (m, 8H) 4.26-4.36 (qd, 1H) 4.71 (s, 1H) 4.82-4.93 (dd, 1H) 5.25 (dq, 1H) 5.68 (d, 1H) 6.15 (d, 1H) 7.18 (s, 2H) 7.79 (s, 1H).

### EXAMPLE 335

### 9-(3-chloro-3,5-dideoxy-β-D-xylofuranosyl)-2-(cyclopentyloxy)-9H-purin-6-amine and

### EXAMPLE 336

### 9-(2-chloro-2,5-dideoxy-β-D-arabinofuranosyl)-2-(cyclopentyloxy)-9H-purin-6-amine

To a solution of 2-(cyclopentyloxy)-9-(5-deoxy-β-D-ribofuranosyl)-9H-purin-6-amine (196 mg, 0.59 mmol) in a mixture of acetonitrile (15 ml) and water (10 μl) was added 1-chlorocarbonyl-1-methylethyl acetate (424 μL, 2.9 mmol) at 4° C. The solution was stirred at rt overnight, then quenched with water and saturated sodium bicarbonate. The reaction mixture was extracted with EtOAc (2×100 ml), dried (sodium sulfate), filtered and concentrated in vacuo. The residue was taken up in 3.5N ammonia in MeOH (10 ml) at 4° C. and the stirring was continued for 4 h at rt. The reaction mixture was concentrated in vacuo and the residue was purified using Gilson reverse phase HPLC with 10 mM ammonium acetate and acetonitrile as the mobile phases with a gradient of 25-50% in 15 min. Relevant fractions were combined to give 66 mg of 9-(3-chloro-3,5-dideoxy-β-D-xylofuranosyl)-2-(cyclopentyloxy)-9H-purin-6-amine and 14 mg of 9-(2-chloro-2,5-dideoxy-β-D-arabinofuranosyl)-2-(cyclopentyloxy)-9H-purin-6-amine.

Example 335: MS (ESP): 354 (MH⁺) for C₁₅H₂₀ClN₅O₃

¹H NMR (400 MHz, DMSO-d₆) δ ppm 1.31 (d, 3H) 1.46-1.56 (m, 2H) 1.57-1.67 (m, 4H) 1.80-1.90 (m, 2H) 4.39 (dd, 1H) 4.48 (dt, 1H) 4.79 (q, 1H) 5.24 (dq, 1H) 5.64 (d, 1H) 6.30 (d 1H) 7.16 (s, 2H) 7.93 (s, 1H).

Example 336: MS (ESP): 354 (MH⁺) for C₁₅H₂₀ClN₅O₃

¹H NMR (400 MHz, DMSO-d₆) δ ppm 1.33 (d, 3H) 1.47-1.58 (m, 2H) 1.63 (m, 4H) 1.84 (m, 2H) 3.72-3.80 (dt, 1H) 4.33 (t, 1H) 4.60 (t, 1H) 5.19-5.26 (m, 1H) 5.95 (s, 1H) 6.22 (d, 1H) 7.15 (s, 2H) 7.94 (s, 1H).

### EXAMPLE 337

### 9-(3-chloro-3-deoxy-O-D-xylofuranosyl)-2-(cyclopentyloxy)-9H-purin-6-amine and

### EXAMPLE 338

### 9-(2-chloro-2-deoxy-β-D-arabinofuranosyl)-2-(cyclopentyloxy)-9H-purin-6-amine

To a solution of 2-(cyclopentyloxy)-9-β-D-ribofuranosyl-9H-purin-6-amine (200 mg, 0.57 mmol) in a mixture of acetonitrile (15 ml) and water (10 μl) was added 1-chlorocarbonyl-1-methylethyl acetate (424 μL, 2.9 mmol) at 4° C. The solution was stirred at rt overnight (15 h), then quenched with water and saturated sodium bicarbonate. The reaction mixture was extracted with ethyl acetate (2×100 ml), dried (sodium sulfate), filtered and concentrated in vacuo. The residue was taken up in 3.5N ammonia in methanol (10 ml) at 4° C. and the stirring was continued for 1 h at rt. The reaction mixture was concentrated in vacuo and the residue was purified using Gilson reverse phase HPLC with 10 mM ammonium acetate and acetonitrile as the mobile phases with a gradient of 15-30% in 15 min. Relevant fractions were combined to give 65 mg of 9-(3-chloro-3,5-dideoxy-β-D-xylofuranosyl)-2-(cyclopentyloxy)-9H-purin-6-amine and 13.5 mg of 9-(2-chloro-2,5-dideoxy-β-D-arabinofuranosyl)-2-(cyclopentyloxy)-9H-purin-6-amine.

Example 337: MS (ESP): 370 (MH⁺) for C₁₅H₂₀ClN₅O₄

¹H NMR (400 MHz, DMSO-d₆) δ ppm 1.55-1.91 (m, 8H) 3.72 (s, 2H) 4.42 (t, 1H) 4.56 (dt, 1H) 4.82 (t, 1H) 5.12 (s, 1H) 5.30 (m, 1H) 5.74 (d, 1H) 6.37 (s, 1H) 7.24 (s, 2H) 8.03 (s, 1H).

Example 338: MS (ESP): 370 (MH⁺) for C₁₅H₂₀ClN₅O₄

¹H NMR (400 MHz, DMSO-d₆) δ ppm 1.50-1.83 (m, 8H) 3.62-3.74 (m, 2H) 4.41 (t, 1H) 4.63 (t, 1H) 5.13 (s, 1H) 5.22 (dt, 2H) 6.04 (s, 1H) 6.26 (d, 1H) 7.14 (s, 2H) 8.05

### EXAMPLE 339

### 2-(cyclopentyloxy)-9-[(3aR,4R,6R,6aR)-6-methyl-2,2-dioxidotetrahydrofuro[3,4-d][1,3,2]dioxathiol-4-yl]-9H-purin-6-amine

To a solution of 2-(cyclopentyloxy)-9-(5-deoxy-β-D-ribofuranosyl)-9H-purin-6-amine (1.92 g, 5.73 mmol) in pyridine (20 ml) was added slowly thionyl chloride at 4° C. The reaction mixture was stirred for 10 min, diluted with DCM (250 ml), and washed successively with cold water and brine. The organic phase was dried over sodium sulfate, filtered and concentrated in vacuo. The residue was purified by chromatography over silica gel eluting with 70% EtOAc in hexane to give desired product (0.81 g) which was taken up in a mixture of carbon tetrachloride/acetonitrile/water 15 ml: 15 ml: 20 ml. Ruthenium(III) chloride (30 mg) and sodium metaperiodate (898 mg, 4.2 mmol) were added successively at 4° C., and stirring was continued for 6 h at 4° C. then at rt overnight. The reaction mixture was diluted with diethylether (100 ml) and water (50 ml). The organic phase was separated, dried, filtered and concentrated in vacuo. The residue was purified by chromatography over silica gel eluting with 65% EtOAc in hexane to give desired product (0.41 g).

MS (ESP): 398 (MH⁺) for C₁₅H₁₉N₅O₆S

¹H NMR (400 MHz, DMSO-d₆) δ ppm 1.39 (d, 3H) 1.57-1.64 (m, 2H) 1.66-1.78 (m, 4H) 1.86-1.97 (m, 2H) 4.55 (dt, 1H) 5.29 (dt, 1H) 5.70-5.77 (dd, 1H) 6.39-6.44 (d, 1H) 6.51 (ddI H) 7.33 (s, 2H) 8.14 (s, 1H).

### EXAMPLE 340

### 2-(cyclopentyloxy)-9-(3,5-dideoxy-3-fluoro-2-O-sulfo-β-D-xylofuranosyl)-9H-purin-6-amine

A solution of 2-(cyclopentyloxy)-9-[(3aR,4R,6R,6aR)-6-methyl-2,2-dioxidotetrahydrofuro[3,4-d][1,3,2]dioxathiol-4-yl]-9H-purin-6-amine (138 mg, 0.35 mmol) and tetrabutylammonium fluoride (1 ml, 1M THF) was heated in a microwave reactor for 30 min at 100° C. The reaction mixture was concentrated in vacuo and the residue was purified using Gilson reverse phase HPLC with 10 mM ammonium acetate and acetonitrile as the mobile phases with a gradient of 20-75% in 15 min. Relevant fractions were combined to give desired product (5.2 mg).

MS (ESP): 418 (MH⁺) for C₁₅H₂₀FN₅O₄S

¹H NMR (400 MHz, DMSO-d₆) δ ppm 1.27 (d, 3H) 1.50-1.87 (m, 8H) 4.19 (dq, 1H) 4.23-4.32 (m, 1H) 5.09-5.16 (dd, 1H) 5.21 (t, 1H) 5.28 (m, 1H) 5.83 (d, 1H) 7.39 (s, 2H) 7.86 (s, 1H).

### EXAMPLE 341

### 9-(3-chloro-3,5-dideoxy-β-D-xylofuranosyl)-2-(spiro[2.2]pent-1-ylmethoxy)-9H-purin-6-amine

To a solution of 9-(5-deoxy-β-D-ribofuranosyl)-2-(spiro[2.2]pent-1-ylmethoxy)-9H-purin-6-amine (Example 153) (70 mg, 0.20 mmol) in a mixture of acetonitrile (3 ml) and water (5 μl) was added 1-chlorocarbonyl-1-methylethyl acetate (145 μL, 2.9 mmol) at 4° C. The solution was stirred at rt overnight, then quenched with water and saturated sodium bicarbonate. The reaction mixture was extracted with EtOAc (2×50 ml), dried (sodium sulfate), filtered and concentrated in vacuo. The residue was taken up in 3.5N ammonia in MeOH (6 ml) at 4° C. and the stirring was continued for 3 h at rt. The reaction mixture was concentrated in vacuo and the residue was purified using Gilson reverse phase HPLC with 10 mM ammonium acetate and acetonitrile as the mobile phases with a gradient of 25-50% in 15 min. Relevant fractions were combined to give desired product (66 mg).

MS (ESP): 354 (MH⁺) for C₁₆H₂₀ClN₅O₃

¹H NMR (400 MHz, DMSO-d₆) δ ppm 0.64-0.75 (m, 5H) 0.98 (m, 1H) 1.30 (d, 3H) 1.52 (m, 1H) 4.08-4.14 (m, 2H) 4.40 (s, 1H) 4.49 (t, 1H) 4.77 (s, 1H) 5.65 (d, 1H) 6.32 (s, 1H) 7.21 (s, 2H) 7.94 (s, 1H).

### EXAMPLE 342

### 9-(3-chloro-3,5-dideoxy-5-fluoro-D-D-xylofuranosyl)-2-(cyclobutylmethoxy)-9H-purin-6-amine

To a solution of 2-(cyclobutylmethoxy)-9-(5-deoxy-5-fluoro-β-D-ribofuranosyl)-9H-purin-6-amine (200 mg, 0.57 mmol) in a mixture of acetonitrile (5 ml), DMF (0.5 ml) and water (5 μl) was added 1-chlorocarbonyl-1-methylethyl acetate (145 μL, 2.9 mmol) at 4° C. The solution was stirred at rt overnight, then quenched with water and saturated sodium bicarbonate. The reaction mixture was extracted with EtOAc (2×50 ml), dried (sodium sulfate), filtered and concentrated in vacuo. The residue was taken up in 3.5N of a solution of ammonia in MeOH (5 ml) at 4° C. and the stirring was continued for 4 h at rt. The reaction mixture was concentrated in vacuo and the residue was purified using Gilson reverse phase HPLC with 10 mM ammonium acetate and acetonitrile as the mobile phases with a gradient of 25-50% in 15 min. Relevant fractions were combined to give desired product (50 mg).

MS (ESP): 372 (MH⁺) for C₁₅H₁₉ClFN₅O₃

¹H NMR (400 MHz, DMSO-d₆) δ ppm 1.74-1.98 (m, 6H) 2.57-2.68 (dt, 1H) 4.13 (d, 2H) 4.56-4.68 (m, 3H) 4.77 (s, 2H) 5.74 (d, 1H) 6.47 (s, 1H) 7.25 (s, 2H) 8.00 (s, 1H).

### EXAMPLE 343

### 2-(cyclopentyloxy)-9-[5-deoxy-3-O-(pyridin-3-ylmethyl)-β-D-ribofuranosyl]-9H-purin-6-amine

To a solution of 2-(cyclopentyloxy)-9-[5-deoxy-2-O-(triisopropylsilyl)-β-D-ribofuranosyl]-N-[(1Z)-(dimethylamino)methylene]-9H-purin-6-amine (0.3 g, 0.55 mmol) in DMF (4 ml) was added successively sodium hydride (131 mg, 3.3 mmol) and 3-(bromomethyl)pyridine hydrobromide (559 mg, 2.2 mmol) at 4° C. The reaction mixture was stirred for 6 h at 4° C. then at rt overnight, quenched with MeOH, and concentrated to dryness. The residue was partitioned between DCM (100 ml) and water (50 ml). The organic phase was separated, dried, filtered and concentrated in vacuo. The residue was purified by chromatography over silica gel eluting with 6% MeOH in DCM to give alkylated intermediate (151 mg) which was taken up in 7N ammonia in MeOH (10 ml) at 4° C. and the stirring was continued overnight at rt. The reaction mixture was concentrated in vacuo and the residue was dissolved in THF (4 ml). Acetic acid (10 μl) and tetrabutylammonium fluoride (50 μl, 1M THF) were added successively at rt, and stirring was continued overnight. The reaction mixture was concentrated in vacuo and the residue was purified using Gilson reverse phase HPLC with 10 mM ammonium acetate and acetonitrile as the mobile phases with a gradient of 25-50% in 15 min. Relevant fractions were combined to give desired product (6.5 mg).

MS (ESP): 372 (MH⁺) for C₂₁H₂₆N₆O₄

¹H NMR (400 MHz, DMSO-d₆) δ ppm 1.28 (d, 3H) 1.51-1.84 (m, 8H) 3.95 (m, 1H) 4.11 (t, 1H) 4.66-4.88 (dd, 2H) 4.91 (t, 1H) 5.21 (dq, 1H) 5.73 (d, 1H) 7.37 (s, 2H) 7.65-7.73 (dd, 1H) 8.11 (s, 1H) 8.15 (dd, 1H) 8.63 (s, 1H) 8.73 (s, 1H).

The intermediates were prepared as follows:—

### 2-(cyclopentyloxy)-9-[5-deoxy-2-O-(triisopropylsilyl)-β-D-ribofuranosyl]-N-[(1Z)-(dimethylamino)methylene]-9H-purin-6-amine

A solution of 2-(cyclopentyloxy)-9-[5-deoxy-2-O-(triisopropylsilyl)-β-D-ribofuranosyl]-9H-purin-6-amine (1.55 g, 3.2 mmol) and N,N-dimethylformamide dimethylacetal (2.1 ml, 15.8 mmol) in DMF (15 ml) was heated at 40° C. for 1 h. The reaction mixture was concentrated in vacuo to give desired product (1.65 g).

MS (ESP): 547 (MH⁺) for C₂₇H₄₆N₅O₄Si

### 2-(cyclopentyloxy)-9-[5-deoxy-2-O-(triisopropylsilyl)-β-D-ribofuranosyl]-9H-purin-6-amine

To a solution of 2-(cyclopentyloxy)-9-(5-deoxy-β-D-ribofuranosyl)-9H-purin-6-amine (8.13 g, 24.3 mmol) in DMF was added successively imidazole (6.6 g, 97.1 mmol) and triisopropylsilylchloride (20.7 ml, 97.1 mmol) at rt. The reaction mixture was stirred overnight, quenched with saturated sodium bicarbonate (20 ml), and concentrated in vacuo. The resulting residue was partitioned between DCM (400 ml) and saturated sodium bicarbonate (200 ml). The organic phase was separated, washed with brine, dried over sodium sulfate, filtered and concentrated in vacuo. The residue was purified using Gilson reverse phase HPLC with 10 mM ammonium acetate and acetonitrile as the mobile phases with a gradient of 75-95% in 40 min. Relevant fractions were combined to give 6.98 g of 2-(cyclopentyloxy)-9-[5-deoxy-2-O-(triisopropylsilyl)-β-D-ribofuranosyl]-9H-purin-6-amine and 2.15 g of 2-(cyclopentyloxy)-9-[5-deoxy-3-O-(triisopropylsilyl)-β-D-ribofuranosyl]-9H-purin-6-amine.

MS (ESP): 492 (MH⁺) for C₂₄H₄₁N₅O₄Si

¹H NMR (400 MHz, CDCl₃) δ ppm 0.84-0.96 (m, 21H) 1.33-1.41 (d, 3H) 1.49-1.59 (m, 2H) 1.73-1.85 (m, 6H) 2.64-2.70 (d, 1H) 3.94 (dt, 1H) 4.04 (dt, 1H) 5.12 (dd, 1H) 5.21-5.28 (dq, 1H) 5.55 (s, 2H) 5.63-5.68 (d, 1H) 7.57 (s, 1H).

### EXAMPLE 344

### 2-(cyclopentyloxy)-9-(3,5-dideoxy-3,5-difluoro-O-D-xylofuranosyl)-9H-purin-6-amine

A solution of 9-(2,3-anhydro-5-deoxy-5-fluoro-β-D-ribofuranosyl)-2-(cyclopentyloxy)-9H-purin-6-amine (prepared as the intermediate for Example 313) (180 mg, 0.537 mmol) and tetrabutylammonium fluoride (1.1 ml, 1M THF) was heated in a microwave reactor for 1 h at 120° C. The reaction mixture was concentrated in vacuo and the residue was purified by chromatography eluting with 4% MeOH in DCM to give desired product (16 mg).

MS (ESP): 356 (MH⁺) for C₁₅H₁₉F₂N₅O₃

¹H NMR δ: 1.40-1.72 (m, 8H) 4.43-4.96 (m, 5H) 5.13 (m, 2H) 5.67 (d, 1H) 6.15 (d, 1H) 7.08 (s, 2H) 7.76 (s, 1H).

### EXAMPLE 345

### (3aS,4S,6R,6aR)-6-[6-amino-2-(cyclopentyloxy)-9H-purin-9-yl]-4-(fluoromethyl)-3-isopropyltetrahydrofuro[3,4-d][1,3]oxazol-2(3H)-one

To a solution of 9-(3-bromo-3,5-dideoxy-5-fluoro-β-D-xylofuranosyl)-2-(cyclopentyloxy)-9H-purin-6-amine (Example 308) (200 mg, 0.481 mmol) in DMF (4 ml) was added successively isopropyl isocyanate (188 μl, 2.41 mmol) and triethylamine (333 μl, 2.41 mmol) at rt. The solution was stirred for 5 h, and then quenched with MeOH. The reaction mixture was concentrated in vacuo and the residue was purified by chromatography eluting with 5% MeOH in DCM to give the carbamate derivative. This intermediate (207 mg) was taken up in THF (5 ml) then sodium hydride (66 mg, 1.65 mmol) was added at −40° C. The solution was stirred for 3 h then quenched with water. The reaction mixture was extracted with DCM (2×50 ml). The organic phases were combined and washed with saturated sodium bicarbonate, dried (sodium sulfate) and evaporated to dryness. The residue was purified using Gilson reverse phase HPLC with 10 mM ammonium acetate and acetonitrile as the mobile phases with a gradient of 0-95% in 15 min. Relevant fractions were combined to give 39 mg of the desired product.

MS (ESP): 421 (MH⁺) for C₁₉H₂₅FN₆O₄

¹H NMR δ: 1.25 (t, 6H) 1.59-1.89 (m, 8H) 3.90 (dt, 1H) 4.50-4.81 (m, 5H) 5.26 (m, 1H) 5.81 (dd, 1H) 6.27 (d, 1H) 7.32 (s, 2H) 8.10 (s, 1H).

### EXAMPLE 346

### 9-[2-(benzylamino)-2,5-dideoxy-β-D-ribofuranosyl]-2-(cyclopentyloxy)-9H-purin-6-amine

To a solution of 9-(2-bromo-2,5-dideoxy-β-D-arabinofuranosyl)-2-(cyclopentyloxy)-9H-purin-6-amine prepared as for Example 312 (250 mg, 0.63 mmol) in a mixture of THF/acetonitrile/DMF 3:3:1 (7 ml) was added successively benzyl isocyanate (930 μl, 7.54 mmol) and triethylamine (1.05 ml, 7.54 mmol) at rt. The solution was stirred for 24 h, and then quenched with MeOH. The reaction mixture was concentrated in vacuo and the residue was taken up in THF (3 ml); sodium hydride (109 mg, 2.73 mmol) was added at −20° C. After stirring at 4° C. for 4 h, the reaction mixture was quenched with MeOH (1 ml), and concentrated in vacuo. This residue was taken up in 6N sodium hydroxide (5 ml) and ethanol (5 ml), stirred for 1 h at 95° C. The reaction mixture was neutralized with amberlite IR-120⁺, filtered and concentrated in vacuo. The residue was purified using Gilson reverse phase HPLC with 10 mM ammonium acetate and acetonitrile as the mobile phases with a gradient of 5-95% in 15 min. Relevant fractions were combined to give 25 mg of the desired product.

MS (ESP): 425 (MH⁺) for C₂₂H₂₈N₆O₃

¹H NMR (400 MHz, DMSO-d₆) δ ppm 1.29 (d, 3H) 1.56-1.81 (m, 8H) 3.71 (dt, 2H) 3.93 (t, 1H) 4.04-4.06 (m, 2H) 5.14 (m, 1H) 5.55 (d, 1H) 5.73 (d, 1H) 7.10-7.18 (m, 7H) 8.01 (s, 1H).

### EXAMPLE 347

### 9-(2-amino-2,5-dideoxy-β-D-ribofuranosyl)-2-(cyclopentyloxy)-9H-purin-6-amine

A suspension of 9-[2-(benzylamino)-2,5-dideoxy-β-D-ribofuranosyl]-2-(cyclopentyloxy)-9H-purin-6-amine (18.7 mg, 0.044 mmol), 20% palladium hydroxide on charcoal (20 mg, 50% wet) and ammonium formate (60 mg) in a mixture of MeOH/water 9:1 (2 ml) was heated at 88° C. for 1.5 h. After cooling to rt, the reaction mixture was filtered through diatomaceous earth and the filtrate was concentrated in vacuo. The residue was purified using Gilson reverse phase HPLC with 10 mM ammonium acetate and acetonitrile as the mobile phases with a gradient of 5-95% in 15 min. Relevant fractions were combined to give 13 mg of the desired product.

MS (ESP): 435 (MH⁺) for C₁₅H₂₂N₆O₃

¹H NMR (400 MHz, DMSO-d₆) δ ppm 1.22 (d, 3H) 1.50-1.83 (m, 8H) 3.75 (m, 1H) 3.92 (dt, 1H) 4.02 (m, 1H) 5.19 (m, 1H) 5.47 (d, 1H) 7.08 (s, 2H) 8.00 (s, 1H).

### EXAMPLE 348

### 2-(butylthio)-9-(3-methylcyclopentyl)-9H-purin-6-amine

2-(Butylthio)-9H-purin-6-amine (made using the procedure found in J. Org. Chem. 2001, 66, 5463-81) (0.11 g, 0.5 mmol), triphenylphosphine resin (0.37 g, loading ˜3 mmol/g) and 3-methylcyclopentanol (0.07 ml, 0.65 mmol) were suspended in toluene/DCM (15:3 ml) and stirred at rt. Diisopropyl azodicarboxylate (0.48 ml, 2.5 mmol) was added and the reaction was stirred at rt overnight. The resin was filtered off and the resulting yellow solution was concentrated in vacuo. The residue was purified by flash chromatography using 2% MeOH in DCM as eluent. Relevant fractions were combined to give a yellow foam (12 mg).

MS (ESP): 306.24 (MH⁺) for C₁₅H₂₃N₅S

¹H NMR δ: 0.89 (t, 3H) 1.02 (m, 3H) 1.23 (m, 1H) 1.41 (m, 2H) 1.66 (m, 3H) 2.10 (m, 4H) 2.37 (m, 1H) 3.04 (m, 2H) 4.82 (m, 1H) 7.22 (bs, 2H) 8.02 (s, 1H)

### EXAMPLE 349

### 2-(butylthio)-9-cyclopentyl-9H-purin-6-amine

2-(Butylthio)-9H-purin-6-amine (0.11 g, 0.5 mmol) and sodium hydride (0.048 g, 60% dispersion in mineral oil, 1.2 mmol) were suspended in DMF (1 ml) and stirred overnight at rt. Cyclopentylchloride (0.06 ml, 0.57 mmol) was added and the reaction was heated to 100° C. for 5 h. When LC/MS indicated that reaction was complete, it was cooled, diluted with water (2 ml) and extracted with DCM (3×5 ml). The organic layer was dried over sodium sulfate and concentrated in vacuo, and the resulting residue was purified by flash chromatography using 2% MeOH in DCM as eluent. Relevant fractions were combined to give a yellow solid (13 mg).

MS (ESP): 306.24 (MH⁺) for C₁₄H₂₁N₅S

¹H NMR δ: 0.90 (t, 3H) 1.42 (m, 2H) 1.65 (m, 4H) 1.85 (m, 2H) 2.04 (m, 4H) 3.05 (t, 2H) 4.75 (m, 1H) 7.21 (bs, 2H) 8.03 (s, 1H).

### EXAMPLE 350

### 2-(benzylthio)-9-cyclopentyl-9H-purin-6-amine

2-(Benzylthio)-9H-purin-6-amine (made using an analogous procedure to that found in J. Org. Chem. 2001, 66, 5463-81) (0.129 g, 0.5 mmol) and sodium hydride (0.048 g 60% dispersion in mineral oil, 1.2 mmol) were suspended in DMF (1 ml) and stirred overnight at rt. Cyclopentylchloride (0.06 ml, 0.57 mmol) was added and the reaction was heated to 100° C. for 8 h, and stirred at rt overnight. When LC/MS indicated that reaction was complete, it was cooled, diluted with water (2 ml) and extracted with DCM (3×5 ml). The organic layer was dried over sodium sulfate and concentrated in vacuo, and the resulting residue was purified by flash chromatography using 2% MeOH in DCM as eluent. Relevant fractions were combined to give yellow foamy solid (52 mg, 32%).

MS (ESP): 326.19 (MH⁺) for C₁₇H₁₉N₅S

¹H NMR: δ 1.91 (m, 8H) 4.35 (s, 2H) 4.82 (m, 1H) 7.24 (m, 5H) 7.44 (d, 2H) 8.07 (s, 1H)

### EXAMPLE 351

### (1S,2R,3S,4R)-4-[6-amino-2-(butylthio)-9H-purin-9-yl]cyclopentane-1,2,3-triol

(1S,4R)-4-[6-amino-2-(butylthio)-9H-purin-9-yl]cyclopent-2-en-1-ol (0.1 g, 0.33 mmol) was dissolved in 10:1 tetrahydrofuran/water (5, 0.5 ml). N-methylmorpholine-N-oxide (50% in water) (50 μL, 0.2 mmol) and osmium tetroxide were added. The reaction was stirred at rt for 24 h, then volatiles were removed in vacuo. The residue was purified by flash chromatography using 10-15% MeOH in chloroform as eluent. Relevant fractions were combined to give 65 mg of the desired product.

MS (ESP): 340 (MH⁺) for C₁₄H₂₁N₅O₃S

¹H NMR δ: 0.89 (t, 3H) 1.33-1.47 (m, 2H) 1.54-1.68 (m, 2H) 1.75-1.90 (m, 1H) 2.51-2.62 (m, 1H) 2.98-3.13 (m, 2H) 3.74 (m, 1H) 3.87 (m, 1H) 4.47-4.63 (m, 2H) 4.82 (d, 1H) 4.96 (d, 1H) 5.12 (d, 1H) 7.23 (s, 2H) 8.00 (s, 1H).

The intermediate for this compound was prepared as follows:—

### (1S,4R)-4-[6-amino-2-(butylthio)-9H-purin-9-yl]cyclopent-2-en-1-ol

2-(Butylthio)-9H-purin-6-amine (0.22 g, 1 mmol) was added to a suspension of sodium hydride (60% in mineral oil) (40 mg, 1 mmol) in DMF (1.5 ml). The reaction mixture was stirred at rt for 20 min then at 50° C. for 10 min. The resulting brown solution was added via cannula to a suspension of palladium tetrakis(triphenylphosphine) (115 mg, 0.1 mmol) and (1S,4R)-cis-4-acetoxy-2-cyclopenten-1-ol (Aldrich) (156 mg, 1.1 mmol) in DMF (1.5 ml). The reaction mixture was stirred at 50° C. for 3 h then cooled to rt. The reaction was quenched by addition of 10 mL water. The aqueous solution was extracted with DCM (3×) and the combined organic layers were dried over magnesium sulfate. The residue was purified by flash chromatography using 100% EtOAc followed by 10% MeOH in EtOAc as eluent. The relevant fractions were combined giving 180 mg of the desired product.

MS (ESP): 306 (MH⁺) for C₁₄H₁₉N₅OS

¹H NMR δ: 0.90 (t, 3H) 1.34-1.47 (m, 2H) 1.58-1.73 (m, 3H) 2.87 (m, 1H) 3.00-3.15 (m, 2H) 4.70 (m, 1H) 5.25-5.38 (m, 2H) 5.98 (d, 1H) 6.15 (m, 1H) 7.27 (s, 2H) 7.92 (s, 1H)

### EXAMPLE 352

### 9-[(4ξ)-3-O-(3-chlorobenzyl)-5-deoxy-D-erythro-pentofuranosyl]-2-(cyclopentyloxy)-9H-purin-6-amine

To a mixture of 2-(cyclopentyloxy)-9-(5-deoxy-β-D-ribofuranosyl)-9H-purin-6-amine (150 mg, 0.45 mmol) and zinc chloride (0.3 g, 2.19 mmol) was added 3-chlorobenzaldehyde (0.63 g, 4.5 mmol). After stirring at 80° C. for 20 min in a microwave reactor, the solution was diluted with 5% MeOH in DCM (1 ml), and purified by column chromatography (DCM/MeOH, 19:1). Relevant fractions were combined to give 93 mg of the desired product which was taken up in dry DCM (1 ml) and diethyl ether (1 ml). Lithium aluminum hydride (82 mg, 2.16 mmol) and a solution of aluminum trichloride (260 mg, 1.94 mmol) in diethyl ether (1 ml) were added successively at rt. After stirring for 4 h, the reaction mixture was cooled to 4° C., and EtOAc (100 ml) and water (100 ml) were added successively. The organic phase was separated, dried (sodium sulfate) and concentrated to dryness. The residue was purified using Gilson reverse phase HPLC with 10 mM ammonium acetate and acetonitrile as the mobile phases with a gradient of 35-60% in 15 min. Relevant fractions were combined to give 9.5 mg of the desired product.

MS (ESP): 460 (MH⁺) for C₂₂H₂₆ClN₅O₄

¹H NMR (400 MHz, DMSO-d₆) δ ppm 1.32 (d, 3H) 1.55-1.84 (m, 8H) 3.97 (t, 1H) 4.15 (dt, 1H) 4.65 (d, 1H) 4.76 (d, 1H) 4.95 (m, 1H) 5.21 (m, 1H) 5.60 (d, 1H) 5.78 (d, 1H) 7.19 (s, 2H) 7.38-7.50 (m, 4H) 8.07 (s, 1H).

Using an analogous procedure to that described in Example 352, by reacting the appropriate commercially available aldehyde with 2-(cyclopentyloxy)-9-(5-deoxy-β-D-ribofuranosyl)-9H-purin-6-amine, followed reduction, the following compounds described in Table XV were obtained.

TABLE XV

| EX | IUPAC name | MH+ | ¹H NMR (400 MHz, DMSO-d₆) δ ppm |
|---|---|---|---|
| 353 | 2-(cyclopentyloxy)-9-[5- | 444 | 1.30 (d, 3H) 1.54-1.84 (m, 8H) 3.99 (t, 1H) |
| | deoxy-3-O-(2- | | 4.11 (dt, 1H) 4.69 (d, 1H) 4.80 (d, 1H) 4.95 (m, |
| | fluorobenzyl)-β-D- | | 1H) 5.23 (m, 1H) 5.56 (d, 1H) 5.77 (d, 1H) |
| | ribofuranosyl]-9H-purin-6- | | 7.19 (s, 2H) 7.36-7.56 (m, 4H) 8.09 (s, 1H). |
| | amine | | |
| 354 | 2-(cyclopentyloxy)-9-[5- | 494 | 1.25 (d, 3H) 1.49-1.76 (m, 8H) 3.90 (t, 1H) |
| | deoxy-3-O-(3,4- | | 4.07 (dt, 1H) 4.58 (d, 1H) 4.69 (d, 1H) 4.87 (m, 1H) |
| | dichlorobenzyl)-β-D- | | 5.14 (m, 1H) 5.54 (d, 1H) 5.71 (d, 1H) 7.11 (s, 2H) |
| | ribofuranosyl]-9H-purin-6- | | 7.34-7.60 (m, 3H) 8.04 (s, 1H) |
| | amine | | |
| 355 | 2-(cyclopentyloxy)-9-[5- | 444 | 1.31 (d, 3H) 1.44-1.91 (m, 8H) 3.90-4.01 (m, |
| | deoxy-3-O-(3- | | 1H) 4.14 (m, 1H) 4.63 (d, 1H) 4.77 (d, 1H) |
| | fluorobenzyl)-β-D- | | 4.90-4.98 (m, 1H) 5.18-5.25 (m, 1H) 5.57 (d, 1H) |
| | ribofuranosyl]-9H-purin-6- | | 5.77 (d, 1H) 7.08-7.28 (m, 4H) 7.37-7.44 (m, |
| | amine | | 1H) 8.09 (s, 1H) |
| 356 | 2-(cyclopentyloxy)-9-[5- | 456 | 1.30 (d, 3H) 1.45-1.88 (m, 8H) 3.75 (s, 3H) |
| | deoxy-3-O-(3- | | 3.93 (t, 1H) 4.12 (m, 1H) 4.57 (d, 1H) 4.73 (d, 1H) |
| | methoxybenzyl)-β-D- | | 4.85-4.98 (m, 1H) 5.20 (s, 1H) 5.54 (d, 1H) |
| | ribofuranosyl]-9H-purin-6- | | 5.77 (d, 1H) 6.76-7.04 (m, 3H) 7.09-7.22 (s, 2H) |
| | amine | | 7.27 (t, 1H) 8.09 (s, 1H) |
| 357 | 2-(cyclopentyloxy)-9-[5- | 444 | 1.30 (d, 3H) 1.48-1.92 (m, 8H) 3.94 (t, 1H) |
| | deoxy-3-O-(4- | | 4.06-4.15 (m, 1H) 4.57 (d, 2H) 4.73 (d, 2H) |
| | fluorobenzyl)-β-D- | | 4.87-4.95 (m, 1H) 5.21 (dd, 1H) 5.55 (d, 1H) |
| | ribofuranosyl]-9H-purin-6- | | 5.76 (d, 1H) 7.13-7.23 (m, 3H) 7.45 (dd, 2H) |
| | amine | | 8.09 (s, 1H) |
| 358 | 9-(3-O-cyclohexyl-5- | 418 | 1.11-1.2 (m, 6H) 1.30 (d, 3H) 1.44-1.89 (m, 12H) |
| | deoxy-β-D-ribofuranosyl)- | | 3.44 (s, 1H) 3.93 (t, 1H) 4.00 (m, 1H) 4.71 (d, |
| | 2-(cyclopentyloxy)-9H- | | 1H) 5.13 (d, 1H) 5.25-5.31 (m, 1H) 5.70 (d, 1H) |
| | purin-6-amine | | 7.17 (s, 2H) 8.08 (s, 1H) |
| 359 | 9-[3-O-(cyclohexylmethyl)- | 432 | 0.92-1.18 (m, 5H) 1.28 (d, 3H) 1.56-1.77 (m, |
| | 5-deoxy-β-D- | | 13H) 1.89 (m, 2H) 3.48 (t, 1H) 3.81 (t, 1H) |
| | ribofuranosyl]-2- | | 4.01 (m, 1H) 4.81 (m, 2H) 5.71 (d, 1H) 7.17 (s, 1H) |
| | (cyclopentyloxy)-9H-purin- | | 8.07 (s, 1H) |
| | 6-amine | | |
| 360 | 2-(cyclopentyloxy)-9-{5- | 494 | 1.22 (s, 1H) 1.32 (d, 3H) 1.47-1.88 (m, 8H) |
| | deoxy-3-O-[3- | | 3.98 (t, 1H) 4.10-4.18 (m, 1H) 4.70 (d, 1H) |
| | (trifluoromethyl)benzyl]-β- | | 4.85 (d, 1H) 4.91-4.97 (m, 1H) 5.17-5.25 (m, |
| | D-ribofuranosyl}-9H-purin- | | 1H) 5.61 (d, 1H) 5.78 (d, 1H) 7.18 (s, 1H) |
| | 6-amine | | 7.58-7.74 (m, 4H) 7.77 (s, 1H) 8.10 (s, 1H) |
| 361 | 2-(cyclopentyloxy)-9-[5- | 416 | 1.30 (d, 3H) 1.45-1.88 (m, 8H) 3.93 (t, 1H) |
| | deoxy-3-O-(2-furylmethyl)- | | 4.57 (d, 1H) 4.73 (d, 1H) 4.85-4.98 (m, 1H) |
| | β-D-ribofuranosyl]-9H- | | 5.20 (s, 1H) 5.54 (d, 1H) 5.77 (d, 1H) |
| | purin-6-amine | | 6.76-7.04 (m, 3H) 7.09-7.22 (m, 2H) 7.27 (t, 1H) |
| | | | 8.09 (s, 1H) |

### EXAMPLE 362

### 6-amino-9-(tetrahydrofuran-2-yl)-9H-purin-2-yl dimethylcarbamate

2-hydroxy-9-(tetrahydrofuran-2-yl)-9H-purin-6-amine (260 mg, 1.2 mmol) was dissolved in 6 mL of pyridine followed by 450 uL of triethylamine and then N,N-dimethylcarbamyl chloride (4 equivalents, 4.8 mmol). The reaction was stirred overnight at rt. LC/MS indicated consumption of the starting material. The reaction was then concentrated in vacuo, dissolved in MeOH to quench any residual carbamyl chloride and concentrated in vacuo to provide a brown oil. A portion of the oil was purified by Gilson Preparative HPLC eluting with aqueous ammonium acetate/acetonitrile at pH 8 in a 5-75% gradient over 15 minutes. 1.1 mg of material was isolated.

MS (APCI-pos): 293.4 (MH⁺) C₁₂H₁₆N₆O₃ Exact Mass: 292.13

NMR (400 MHz, MeOD) δ: 2.1-2.3 m, 2H), 2.5 (m, 2H), 4.0 m, 1H), 4.3 (m, 1H), 3.0 (s, 3H), 3.15 (s, 3H), 6.25 d (2H), 8.2 (s, 1H).

The intermediate for this compound was made as follows:—

### 2-hydroxy-9-(tetrahydrofuran-2-yl)-9H-purin-6-amine

2-Benzyloxy-9-(tetrahydrofuran-2-yl)-9H-purin-6-amine (Example 25) (1.2 g, 3.9 mmol) was dissolved in 6 mL of ethanol followed by ˜10 mg of 5% palladium on carbon. The reaction was purged under vacuum and filled with hydrogen at 1 atm. The process was repeated 3× and then the reaction was stirred overnight at room temperature. The reaction was not complete and was resubjected as above with fresh catalyst. After 6 h, the reaction was complete and was purged of hydrogen. The product was isolated by filtering off the catalyst through a glass fiber filter and concentration in vacuo, to provide the product as a pale yellow oil, 420 mg (50%).

MS (APCI-pos): MH 222 for C₉H₁₁N₅O₂

### EXAMPLE 363

### 9-[3-O-(anilinocarbonyl)-5-deoxy-β-D-ribofuranosyl]-2(cyclopentyloxy)-9H-purin-6-amine and

### EXAMPLE 364

### 9-[2-O-(anilinocarbonyl)-5-deoxy-β-D-ribofuranosyl]-2-(cyclopentyloxy)-9H-purin-6-amine

200 mg (0.59 mmol) of 2-(cyclopentyloxy)-9-(5-deoxy-β-D-ribofuranosyl)-9H-purin-6-amine, phenyl isocyanate (60 mg, 1.0 equivalent), and 0.5 mL (3.5 mmol) triethylamine in 6 mL of THF are stirred at rt overnight. The mixture is concentrated and purified on HPLC (15-95% acetonitrile in water) to give 9-[3-O-(anilinocarbonyl)-5-deoxy-β-D-ribofuranosyl]-2(cyclopentyloxy)-9H-purin-6-amine (27 mg) and 9-[2-O-(anilinocarbonyl)-5-deoxy-β-D-ribofuranosyl]-2-(cyclopentyloxy)-9H-purin-6-amine.

Example 363: MS (ESP): 455 (MH⁺) for C₂₂H₂₆N₆O₅

¹H NMR (400 MHz, DMSO-d₆) δ ppm 1.39 (d, 3H) 1.59-1.89 (m, 9H) 4.20 (m, 1H) 5.06 (m, 1H) 5.13 (m, 1H) 5.37 (m, 1H) 5.80 (m, 1H) 5.89 (m, 1H) 7.0 (m, 1H) 7.29 (m, 2H) 7.50 (m, 2H) 8.17 (s, 2H) 9.86 (s, 1H)

Example 364: MS (ESP): 455 (MH⁺) for C₂₂H₂₆N₆O₅

¹H NMR (400 MHz, DMSO-d₆) δ ppm 1.34 (d, 3H) 1.57-1.89 (m, 9H) 3.99 (m, 1H) 4.38 (m, 1H) 5.27 (m, 1H) 5.70 (m, 1H) 5.76 (m, 1H) 5.99 (m, 1H) 6.97 (m, 1H) 7.23 (m, 2H) 7.44 (m, 2H) 8.16 (s, 2H) 9.85 (s, 1H)

The compounds in Table XVI were made using an analogous procedure to that described for Example 363 by reaction of 2-(cyclopentyloxy)-9-(5-deoxy-β-D-ribofuranosyl)-9H-purin-6-amine with the appropriate commercially available isocyanate:

TABLE XVI

| EX | IUPAC name | MH+ | ¹H NMR (400 MHz, DMSO-d₆) δ ppm |
|---|---|---|---|
| 365 | 9-(3-O-{[(4- | 480 | 1.39 (d, 3H) 1.58-1.88 (m 9H) 4.24 (m, 1H) |
| | cyanophenyl)amino]carbonyl}-5- | | 5.15 (m, 1H) 5.36 (m, 1H) 5.78 (m, 1H) |
| | deoxy-β-D-ribofuranosyl)-2- | | 5.94 (m, 1H) 6.01 (m, 1H) 7.23 (s, 2H) 7.67 (m, |
| | (cyclopentyloxy)-9H-purin-6-amine | | 2H), 7.76 (m, 2H), 9.68 (s, 1H) |
| 366 | 2-(cyclopentyloxy)-9-(5-deoxy-3- | 491 | 1.31 (d, 3H) 1.51-1.83 (m, 9H) 4.13 (m, 1H) |
| | O-{[(3,4- | | 4.99 (m, 2H) 5.27 (m, 1H) 5.72 (m, 1H) 5.80 (m, |
| | difluorophenyl)amino]carbonyl}-β- | | 1H) 6.98 (m, 1H) 7.21 (m, 1H) 7.54 (m, 1H) |
| | D-ribofuranosyl)-9H-purin-6-amine | | 8.09 (s, 2H) 9.44 (s, 1H) |

### EXAMPLE 367

### 9-[3-(benzylamino)-3,5-dideoxy-O-D-xylofuranosyl]-2-(cyclopentyloxy)-9H-purin-6-amine

A solution of 9-(3-amino-3,5-dideoxy-β-D-xylofuranosyl)-2-(cyclopentyloxy)-9H-purin-6-amine prepared as for Example 316 (70 mg, 0.21 mmol) and benzaldehyde (23 μl, 0.23 mmol) in MeOH (1 ml) was stirred at rt 3 h. Acetic acid (12 μl, 0.21 mmol) and sodium triacetoxyborohydride (67 mg, 0.31 mmol) were added successively. The reaction mixture was stirred overnight and concentrated in vacuo. The residue was purified using Gilson reverse phase HPLC with 10 mM ammonium acetate and acetonitrile as the mobile phases with a gradient of 5-95% in 15 min. Relevant fractions were combined to give 28 mg of the desired product.

MS (ESP): 425 (MH⁺) for C₂₂H₂₈N₆O₃

¹H NMR (400 MHz, DMSO-d₆) δ ppm 1.22 (d, 3H) 1.43-1.54 (m, 2H) 1.54-1.65 (m, 4H) 1.75-1.86 (m, 2H) 2.97-3.06 (t, 1H) 3.73 (d, 1H) 3.82 (d, 1H) 4.24-4.33 (dt, 1H) 4.55 (q, 1H) 5.16-5.25 (m, 1H) 5.59 (d, 1H) 5.63 (d, 1H) 7.17-7.31 (m, 7H) 8.12 (s, 1H).

### EXAMPLE 368

### 9-{3-[(cyclohexylmethyl)amino]-3,5-dideoxy-β-D-xylofuranosyl}-2-(cyclopentyloxy)-9H-purin-6-amine

A solution of 9-(3-amino-3,5-dideoxy-β-D-xylofuranosyl)-2-(cyclopentyloxy)-9H-purin-6-amine prepared as for Example 316 (71 mg, 0.21 mmol) and cyclohexane carboxaldehyde (28 μl, 0.23 mmol) in MeOH (1 ml) was stirred at rt 3 h. Acetic acid (12 μl, 0.21 mmol) and sodium triacetoxyborohydride (67 mg, 0.31 mmol) were added successively. The reaction mixture was stirred overnight and concentrated in vacuo. The residue was purified using Gilson reverse phase HPLC with 10 mM ammonium acetate and acetonitrile as the mobile phases with a gradient of 40-95% in 15 min. Relevant fractions were combined to give 15 mg of the desired product.

MS (ESP): 431 (MH⁺) for C₂₂H₃₄N₆O₃

¹H NMR (400 MHz, DMSO-d₆) δ ppm 0.81-1.85 (m, 22H) 2.29 (m, 2H) 2.92 (dt, 1H) 4.27 (q, 1H) 4.42 (t, 1H) 5.23 (m, 1H) 5.59 (m, 2H) 7.10 (s, 2H) 8.10 (s, 1H).

### EXAMPLE 369

### 2-(cyclopentyloxy)-9-[3,5-dideoxy-3-(1H-1,2,3-triazol-1-yl)-O-D-xylofuranosyl]-9H-purin-6-amine

A solution of 9-(3-azido-3,5-dideoxy-β-D-xylofuranosyl)-2-(cyclopentyloxy)-9H-purin-6-amine prepared as for Example 315 (60 mg, 0.17 mmol) and norbornadiene (200 μL) in DMF (200 μL) was heated in a microwave reactor for 15 min at 130° C. The reaction mixture was concentrated in vacuo and the residue was purified using Gilson reverse phase HPLC with 10 mM ammonium acetate and acetonitrile as the mobile phases with a gradient of 5-95% in 15 min. Relevant fractions were combined to give 34 mg of the desired product.

MS (ESP): 387 (MH⁺) for C₁₇H₂₂N₈O₃

¹H NMR (400 MHz, DMSO-d₆) δ ppm 0.71 (d, 3H) 1.49-1.61 (m, 3H) 1.65-1.85 (m, 5H) 4.57 (dt, 1H) 5.22-5.27 (m, 1H) 5.27-5.34 (m, 1H) 5.48 (q, 1H) 5.77 (d, 1H) 6.22 (d, 1H) 7.21 (s, 2H) 7.79 (s, 1H) 8.25 (s, 1H) 8.46 (s, 1H).

### EXAMPLE 370

### 2-(cyclopentyloxy)-9-{3,5-dideoxy-3-[4-(methoxycarbonyl)-1H-1,2,3-triazol-1-yl]-β-D-xylofuranosyl}-9H-purin-6-amine

A mixture of 9-(3-azido-3,5-dideoxy-β-D-xylofuranosyl)-2-(cyclopentyloxy)-9H-purin-6-amine prepared as for Example 315 (125 mg, 0.35 mmol) and methyl propiolate (300 μL) was heated in a microwave reactor for 10 min at 80° C. The reaction mixture was concentrated in vacuo and the residue was purified using Gilson reverse phase HPLC with 10 mM ammonium acetate and acetonitrile as the mobile phases with a gradient of 20-30% in 15 min. Relevant fractions were combined to give 50 mg of the desired product.

MS (ESP): 445 (MH⁺) for C₁₉H₂₄N₈O₅

¹H NMR (400 MHz, DMSO-d₆) δ ppm 0.84 (d, 3H) 1.54-1.64 (m, 2H) 1.65-1.76 (m, 4H) 1.86 (m, 2H) 3.88 (s, 3H) 4.65-4.71 (dt, 1H) 5.35 (m, 1H) 5.41-5.45 (dd, 1H) 5.66 (t, 1H) 5.88 (d, 1H) 6.35 (s, 1H) 7.29 (s, 2H) 8.34 (s, 1H) 9.12 (s, 1H).

### EXAMPLE 371

### 9-[3-(4-carboxy-1H-1,23-triazol-1-yl)-3,5-dideoxy-O-D-xylofuranosyl]-2-(cyclopentyloxy)-9H-purin-6-amine

A mixture of 2-(cyclopentyloxy)-9-{3,5-dideoxy-3-[4-(methoxycarbonyl)-1H-1,2,3-triazol-1-yl]-β-D-xylofuranosyl}-9H-purin-6-amine (22 mg, 0.05 mmol) and 2 mL of aqueous sodium hydroxide (1N) was stirred overnight. The reaction mixture was neutralized with amberlite IR-120+, filtered and concentrated to dryness to give desired product.

MS (ESP): 431 (MH⁺) for C₁₈H₂₂N₈O₅

¹H NMR (400 MHz, DMSO-d₆) δ ppm 0.76 (d, 3H) 1.51-1.84 (m, 8H) 4.58 (m, 1H) 5.30 (m, 2H) 5.53 (t, 1H) 5.79 (d, 1H) 6.26 (s, 1H) 7.20 (s, 2H) 8.27 (s, 1H) 8.83 (s, 1H).

### EXAMPLE 372

### 9-{3-bromo-3,5-dideoxy-5-fluoro-2-O-[(isopropylamino)carbonyl]-β-D-xylofuranosyl}-2-(cyclopentyloxy)-9H-purin-6-amine

To a solution of 9-(3-bromo-3,5-dideoxy-5-fluoro-β-D-xylofuranosyl)-2-(cyclopentyloxy)-9H-purin-6-amine prepared as for Example 308 (200 mg, 0.48 mmol) in dimethylformamide (4 ml) was added successively isopropyl isocyanate (236 μl, 2.4 mmol) and triethylamine (133 μl, 0.92 mmol) at rt. The solution was stirred for 5 h, and then quenched with MeOH. The reaction mixture was concentrated in vacuo and the residue was purified by flash chromatography eluting with 5% MeOH in DCM to give desired product as a white solid (220 mg).

MS (ESP): 502 (MH⁺) for C₁₉H₂₆BrFN₆O₄

¹H NMR (300 MHz, DMSO-d₆) δ ppm 0.94-1.09 (m, 6H) 1.56 (s, 2H) 1.69 (s, 4H) 1.83-1.97 (m, 2H) 3.63 (dq, 1H) 4.58-4.74 (m, 1H) 4.83-4.94 (m, 2H) 5.22-5.35 (m, 1H) 5.49 (d, 1H) 5.71-5.84 (m, 1H) 5.98 (d, 1H) 7.28 (s, 2H) 7.54 (d, 1H) 8.07 (s, 1H)

It should be clear that some of the intermediates used to prepare the aforementioned Examples are themselves compounds within the scope of the instant invention.

The compounds in Table XVII below are compounds within the scope of formula I. These compounds can be made using procedures described hereinbefore or by procedures analogous to those described in Chem. Pharm. Bull. (1975), 23(4), 759-74, WO 03/035662 A1, J. Med. Chem. (1991), 34(4), 1334-9 and 1340-4, J. Med. Chem. (1973), 16(12), 1381-8, Eur. J. Pharm. (1972), 19(2), 246-50.

TABLE XVII

| EX | IUPAC name | MH+ | ¹HNMR (DMSO-d₆, 300 MHz) ppm δ |
|---|---|---|---|
| 373 | 2-(butylthio)-9-beta-D-ribofuranosyl- | 356.2 | 0.90 (t, 3H) 1.35-1.48 (m, 2H) |
| | 9H-purin-6-amine | | 1.57-1.69 (m, 2H) 3.07 (m, 2H) 3.57 (m, 2H) |
| | | | 3.90 (d, 1H) 4.12 (m, 1H) 4.60 (m, 1H) 5.02 (m, |
| | | | 1H) 5.17 (m, 1H) 5.41 (d, 1H) 5.80 (d, 1H) |
| | | | 7.35 (s, 2H) 8.21 (s, 1H) |
| 374 | 2-(benzylthio)-9-beta-D-ribofuranosyl- | 390.1 | 3.58 (m, 2H) 3.93 (m, 1H) 4.13 (m, 1H) |
| | 9H-purin-6-amine | | 4.36 (s, 2H) 4.55 (m, 1H) 5.06 (m, 1H) |
| | | | 5.20 (d, 1H) 5.44 (d, 1H) 5.87 (d, 1H) |
| | | | 7.19-7.34 (m, 3H) 7.46 (d, 4H) 8.26 (s, 1H) |
| 375 | 2-(cyclopentylthio)-9-beta-D- | 368.2 | 1.60 (m, 6H); 2.17 (m, 2H); 3.57 (m, 2H); |
| | ribofuranosyl-9H-purin-6-amine | | 3.90 (m, 2H); 4.11 (m, 1H); 4.61 (q, 1H); |
| | | | 5.01 (t, 1H); 5.15 (d, 1H); 5.42 (d, 1H); |
| | | | 5.79 (d, 1H); 7.32 (br s, 2H); 8.20 (s, 1H) |
| 376 | 2-(cyclopentyloxy)-9-beta-D- | 352.0 | 1.57-1.68 (m, 6H); 1.88 (m, 2H); 3.55 (m, |
| | ribofuranosyl-9H-purin-6-amine | | 2H); 3.88 (m, 1H); 4.11 (m, 1H); 4.56 (t, |
| | | | 1H); 5.13 (m, 1H); 5.26 (m, 1H); 5.72 (d, |
| | | | 1H); 7.22 (br s, 2H); 8.11 (s, 1H) |
| 377 | 9-beta-D-ribofuranosyl-2- | 368.0 | 1.64-1.93 (series of m, 5H) 3.51 (m, 1H); |
| | (tetrahydrofuran-2-ylmethoxy)-9H- | | 3.61-3.68 (2 m, 2H); 3.76 (m, 1H); 3.89 (m, |
| | purin-6-amine | | 1H); 4.13 (m, 4H); 4.57 (t, 1H); 4.88-5.64 (2 |
| | | | br s, 2H); 5.76 (d, 1H); 7.32 (br s, 2H); |
| | | | 8.14 (s, 1H) |
| 378 | 2-[(4-methylcyclohexyl)oxy]-9-beta-D- | 380.2 | 0.88 (d, 3H) 1.06 (m, 2H) 1.35 (m, 3H) |
| | ribofuranosyl-9H-purin-6-amine | | 1.69 (m, 2H) 2.02 (m, 2H) 3.52 (m, 1H) |
| | | | 3.61 (m, 1H) 3.89 (s, 1H) 4.12 (s, 1H) |
| | | | 4.59 (m, 1H) 4.77 (m, 1H) 5.12 (m, 2H) |
| | | | 5.43 (m, 1H) 5.74 (d, 1H) 7.23 (br s, 2H) |
| | | | 8.11 (s, 1H) |
| 379 | 2-(cyclobutylmethoxy)-9-beta-D- | 352.2 | 1.76-2.06 (series of m, 7H); 2.67 (m, 1H); |
| | ribofuranosyl-9H-purin-6-amine | | 3.57 (m, 2H); 3.89 (q, 1H); 4.11 (m, 2H); |
| | | | 4.17 (d, 1H); 4.56 (t, 1H); 5.12-5.46 (br, 2H); |
| | | | 5.76 (d, 1H); 7.28 (br s, 2H); 8.13 (s, 1H) |
| 380 | 2-(decahydronaphthalen-2-yloxy)-9- | 420.3 | 0.78-2.09 (seies of m, 16H); 3.52 (m, 1H); |
| | beta-D-ribofuranosyl-9H-purin-6-amine | | 3.61 (m, 1H); 3.88 (m, 1H); 4.12 (m, 1H); |
| | | | 4.57-4.85 (br, 2H); 5.13 (m, 2H); 5.42 (d, |
| | | | 1H); 5.73 (d, 1H); 7.22 (br s, 2H); 8.10 (s, |
| | | | 1H) |
| 381 | 9-beta-D-ribofuranosyl-2-(2,2,2- | 366 | 3.46-3.58 (m, 1H) 3.58-3.70 (m, 1H) |
| | trifluoroethoxy)-9H-purin-6-amine | | 3.90 (q, 1H) 4.07-4.19 (m, 1H) 4.54 (t, 1H) |
| | | | 4.92 (q, 2H) 5.07 (s, 1H) 5.20 (s, 1H) |
| | | | 5.45 (s, 1H) 5.78 (d, 1H) 7.54 (s, 2H) |
| | | | 8.22 (s, 1H) |
| 382 | 2-(benzyloxy)-9-beta-D-ribofuranosyl- | 374 | (MeOD) 3.63-3.79 (m, 1H); 3.80-3.92 (m, |
| | 9H-purin-6-amine | | 1H); 4.03-4.15 (m, 1H); 4.32 (dd, 3.58 Hz, 1H) |
| | | | 4.71 (t, 1H) 5.39 (s, 2H) 5.90 (d, 1H) |
| | | | 7.20-7.54 (m, 5H) 8.14 (s, 1H) |

1. A compound according to formula II 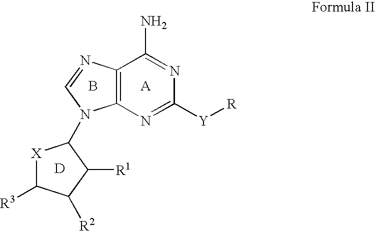
and pharmaceutically acceptable salts thereof wherein:
A, B and D are used to designate the particular ring;
X is selected from O and —CH₂—;
Y is selected from O, S, —CO—, —CH₂—, —CH═CH—, —SO—, and —SO₂— or Y and R taken together form a heterocyclic radical provided that the atom of the heterocyclic radical which is directly attached to ring A is not a nitrogen atom and wherein said heterocyclic radical may be optionally substituted on one or more carbon atoms by one or more R³³ and wherein if heterocyclyl contains an —NH— moiety, the nitrogen of said moiety may be optionally substituted by a group selected from R³⁴;
R is selected from C₁₋₁₀alkyl, C₂₋₁₀alkenyl, C₂₋₁₀alkynyl, C₃₋₁₂carbocyclyl, —S(O)ₚR⁴, —C(O)R⁵, and heterocyclyl wherein R may be optionally substituted on one or more carbon atoms by one or more R′ and wherein if heterocyclyl contains an —NH— moiety, the nitrogen of said moiety may be optionally substituted by a group selected from R″;
p is independently at each occurrence 0, 1 or 2;
R¹, R² and R³ are each independently selected from hydrogen, hydroxy, cyano, azido, C₁₋₁₀alkyl, C₃₋₁₂carbocyclyl, halo, —C(O)R^{5′}, —OC(O)R¹², S(O)ₚR^{4′}, ═N—O—R⁹, C₂₋₁₀alkenyl, C₂₋₁₀alkynyl, heterocyclyl, —OR²⁴, and NR¹⁰R¹¹ alternatively, R¹ and R² or R² and R³ taken together form a cyclic ring containing 3-6 atoms and further wherein R¹, R² and R³ may be optionally substituted on one or more carbon atoms by one or more R^{1′} and wherein if heterocyclyl and/or said cyclic ring contains an —NH— moiety, the nitrogen of said moiety may be optionally substituted by a group selected from R^{3′};
R⁴, R^{4′}, and R^{4″} are each independently selected from hydrogen, hydroxy, —NR⁷R⁸, C₁₋₆alkyl, C₂₋₆alkenyl, C₁₋₆alkoxy, C₃₋₁₀cycloalkyl, heterocyclyl, and aryl wherein R⁴, R^{4′}, and R^{4″} may be optionally substituted on one or more carbon atoms by one or more R¹³ and wherein if heterocyclyl contains an —NH— moiety, the nitrogen of said moiety may be optionally substituted by a group selected from R¹⁴;
R⁵, R^{5′}, R^{5″}, R¹² and R^{12′} are each independently selected from hydrogen, —NR^{7′}R^{8′}, —OR^{24′}, C₁₋₆alkyl, C₂₋₆alkenyl, C₃₋₁₀cycloalkyl, C₃₋₁₀cycloalkenyl, heterocyclyl, and aryl wherein R⁵, R^{5′}, R^{5″}, R¹² and R^{12′} may be optionally substituted on one or more carbon atoms by one or more R¹⁵ and wherein if heterocyclyl contains an —NH— moiety, the nitrogen of said moiety may be optionally substituted by a group selected from R¹⁶;
R⁷, R^{7′}, R^{7″}, R⁸, R^{8′} and R^{8″} are each independently selected from hydrogen, C₁₋₆alkyl, C₂₋₆alkenyl, C₂₋₆alkynyl, —OR^{24′}, C₃₋₁₀cycloalkyl, C₃₋₁₀cycloalkenyl, heterocyclyl, and aryl wherein R⁷, R^{7′}, R^{7″}, R⁸, R^{8′} and R^{8″} may be optionally substituted on one or more carbon atoms by one or more R¹⁷ and wherein if heterocyclyl contains an —NH— moiety, the nitrogen of said moiety may be optionally substituted by a group selected from R¹⁸;
R⁹ and R^{9′} are each independently selected from hydrogen, C₁₋₆alkyl, C₂₋₆alkenyl, C₂₋₆alkynyl, C₃₋₁₀cycloalkyl, C₃₋₁₀cycloalkenyl, heterocyclyl and aryl wherein R⁹ and R^{9′} may be optionally substituted on one or more carbon atoms by one or more R¹⁹;
R¹⁰ and R¹¹ are each independently selected from hydrogen, C₁₋₆alkyl, C₂₋₆alkenyl, C₂₋₆alkynyl, —OR^{24′}, C₃₋₁₀cycloalkyl, C₃₋₁₀cycloalkenyl, heterocyclyl and aryl, wherein R¹⁰ and R¹¹ independently of each other may be optionally substituted on one or more carbon by one or more R²⁰, and wherein if said heterocyclyl contains a —NH— moiety, the nitrogen of said moiety may be optionally substituted by a group selected from R²¹;
R′, R^{1′}, R¹³, R¹⁵, R¹⁷, R¹⁹, R²⁰, R²⁵ and R³³ are each independently selected from halo, nitro, —NR^{7″}R^{8″}, azido, cyano, isocyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, keto(═O), —OR^{24′}, —C(O)R^{5″}, —OC(O)R^{12′}, S(O)ₓR^{4″}; ═N—O—R^{9′}, —NHC(O)NR^{7′}R^{8′}, —N(C₁₋₆alkyl)C(O)NR^{7′}R^{8′}, NHC(O)R^{24″}, —NHCO₂R^{24″}, —NHSO₂(R^{24″}), -amidino i.e. —NHC(NH)NH₂, wherein x is independently 0, 1 or 2 and further wherein R′, R^{1′}, R¹³, R¹⁵, R¹⁷, R¹⁹, R²⁰, R²⁵ and R³³ independent of each other may be optionally substituted on one or more carbon by one or more R²² and wherein if heterocyclyl contains a —NH— moiety, the nitrogen of said moiety may be optionally substituted by a group selected from R²³,
R″, R^{3′}, R¹⁴, R¹⁶, R¹⁸, R²¹, R²³, R²⁶, R²⁸ and R³⁴ are each independently selected from cyano, C₁₋₆alkyl, C₂₋₆alkenyl, C₂₋₆alkynyl, aryl, cycloalkyl, cycloalkenyl, heterocyclyl, hydroxy, —OR^{24′}, —C(O)R^{5″}, —OC(O)R^{12′}, S(O)ₓR^{4″}, —NHC(NH)NH₂, wherein x is independently 0, 1 or 2 wherein R″, R^{3′}, R¹⁴, R¹⁶, R¹⁸, R²¹, R²³, R²⁶, R²⁸ and R³⁴ independently of each other may be optionally substituted on one or more carbon by one or more R²⁷ and wherein if said heterocyclyl contains a —NH— moiety, the nitrogen of said moiety may be optionally substituted by a group selected from R⁶;
R²⁴, R^{24′} and R^{24″} are each independently selected from hydrogen, C₁₋₆alkyl, C₂₋₆alkenyl, C₃₋₁₂cycloalkyl, C₃₋₁₂cycloalkenyl, aryl, S(O)ₓR^{4″}, and heterocyclyl wherein x is independently 0, 1 or 2 and further wherein R²⁴, R^{24′} and R^{24″} may be optionally substituted on one or more carbon by one or more R²⁵ and wherein if said heterocyclyl contains a —NH— moiety, the nitrogen of said moiety may be optionally substituted by a group selected from R²⁶;
R²² and R²⁷ are each independently selected from halo, nitro, —NR^{7′}R^{8′}, azido, cyano, isocyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, keto(═O), —OR^{24′}, —C(O)R^{5″}, —OC(O)R^{12′}, S(O)ₓR^{4″}; ═N—O—R^{9′}, NHC(O)NR^{7′}R^{8′}, —N(C₁₋₆alkyl)C(O)NR^{7′}R^{8′}, —NHC(O)R^{24″}, —NHCO₂R^{24″}, —NHSO₂(R^{24″}), -amidino i.e. —NHC(NH)NH₂, wherein x is independently 0, 1 or 2 and further wherein R²² and R²⁷ independently of each other may be optionally substituted on one or more carbon by one or more R²⁹ and wherein if heterocyclyl contains a —NH— moiety, the nitrogen of said moiety may be optionally substituted by a group selected from R³⁰;
R⁶ and R²³ are each independently selected from cyano, C₁₋₆alkyl, C₂₋₆alkenyl, C₂₋₆alkynyl, aryl, cycloalkyl, cycloalkenyl, heterocyclyl, hydroxy, —OR^{24′}, —C(O)R^{5″}, —OC(O)R^{12′}, S(O)ₓR^{4″} and —NHC(NH)NH₂, wherein x is independently 0, 1 or 2 and further wherein R⁶ and R²³ independently of each other may be optionally substituted on one or more carbon by one or more R³¹ and wherein if said heterocyclyl contains a —NH— moiety, the nitrogen of said moiety may be optionally substituted by a group selected from R³²;
R²⁹ and R³¹ are each independently selected from halo, nitro, —NR^{7′}R^{8′}, azido, cyano, isocyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, keto(═O), —OR^{24′}, —C(O)R^{5″}, —OC(O)R^{12′}, S(O)ₓR^{4″}, ═N—O—R^{9′}, NHC(O)NR^{7′}R^{8′}, —N(C₁₋₆alkyl)C(O)NR^{7′}R^{8′}, —NHC(O)R^{24″}, —NHCO₂R^{24″}, —NHSO₂(R^{24″}), -amidino i.e. —NHC(NH)NH₂, wherein x is independently 0, 1 or 2;
R³⁰ and R³² are each independently selected from cyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, —OR^{24′}, —C(O)R^{5″}, —OC(O)R^{12′}, S(O)ₓR^{4″}, and —NHC(NH)NH₂ wherein x is independently 0, 1 or 2;
provided that when ring D is an unsubstituted tetrahydrofuranyl ring, i.e. when X is O and R¹, R² and R³ are hydrogen, and Y is O, then R cannot be a 3-pyrrolidinyl radical or a 7-methylindan-4-yl radical; provided further when ring D is an unsubstituted tetrahydrofuranyl ring and Y is S, then R cannot be an unsubstituted 2-naphthyl radical; and further provided when ring D is an unsubstituted tetrahydrofuranyl ring, Y and R taken together cannot be an unsubstituted 3-pyridyl radical; and provided further the compound is not
9-{5-[4-(carboxymethyl)-1H-imidazol-1-yl]-5-deoxy-β-D-ribofuranosyl}-2-(cyclopentyloxy)-9H-purin-6-amine,
9-[5-(4-acetyl-1H-1,2,3-triazol-1-yl)-5-deoxy-β-D-ribofuranosyl]-2-(cyclopentyloxy)-9H-purin-6-amine,
3-(6-amino-2-propylthio-9H-purin-9-yl)-5-(hydroxymethyl)-1,2-cyclopentanediol, or
9-cyclopentyl-2-(cyclopentylthio)-9H-purin-6-amine;
and further provided that when X is O and R¹ and R² are both hydroxy, and ring D has the stereochemistry indicated in formulas IIIa or IIIb, 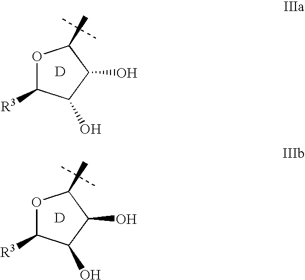
then R³ is not HO—CH₂— or CH₃CH₂NHC(O)—.

2. A compound of claim 1 according to formula IIa 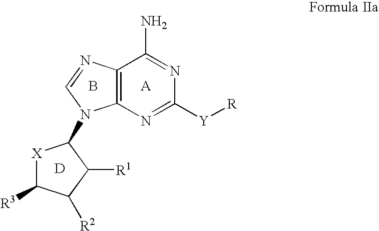
wherein R, R₁, R₂, R₃, X and Y are as defined in claim 1, and pharmaceutically acceptable salts thereof.

3. A compound of claim 1 according formula IIb 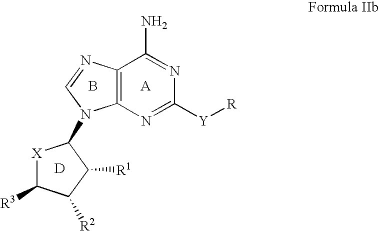
wherein R, R₁, R₂, R₃, X and Y are as defined in claim 1, provided R₁ and R₂ are not both H, and pharmaceutically acceptable salts thereof.

4. A compound of claim 1 according to formula IIc 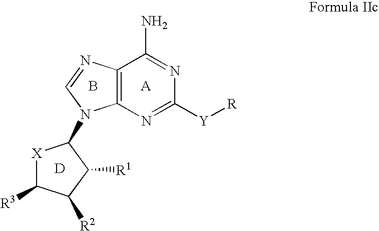
wherein R, R₁, R₂, R₃, X and Y are as defined in claim 1 provided R₂ is not H, and pharmaceutically acceptable salts thereof.

5. A compound according to claim 1 wherein R₁, R₂ and R₃ are all H, and pharmaceutically acceptable salts thereof.

6. A compound according to claim 1 provided further that when X is O and R¹ and R² are both hydroxy, then R³ is not HOCH₂—.

7. A compound according to claim 1 and pharmaceutically acceptable salts thereof wherein when R¹ and R² are both hydroxy, R³ is methyl or fluoromethyl.

8. A compound according to claim 1 and pharmaceutically acceptable salts thereof wherein Y is O and R is selected from C₃₋₁₂cycloalkyl and C₃₋₁₂cycloalkylC₁₋₆alkyl wherein said C₃₋₁₂cycloalkyl and C₃₋₁₂cycloalkylC₁₋₆alkyl are optionally substituted on one or more carbons by R′.

9. A compound according to claim 1 and pharmaceutically acceptable salts thereof wherein Y is O and R is selected from C₃₋₁₀cycloalkyl wherein said C₃₋₁₀cycloalkyl is optionally substituted on one or more carbon by R′.

10. A compound according to claim 1 wherein
X and Y are O;
R is selected from C₃₋₁₀cycloalkyl and C₃₋₁₀cycloalkenyl wherein said R is optionally substituted on one or more carbon atoms by one or more C₁₋₄alkyl, halo, haloC₁₋₄alkyl, C₁₋₄alkoxy and haloC₁₋₄alkoxy, cyano, S(O)ₓR^{4″}, and ═N—OR^{9′};
R¹ is hydroxy;
R² is selected from hydroxy, halo, NR¹⁰R¹¹, cyano and C₁₋₃alkoxy wherein said C₁₋₃alkoxy is optionally substituted on one or more carbon by one or more halo, hydroxy, heteroaryl, and aryl and wherein said aryl and heteroaryl are optionally substituted on one or more carbon by one or more halo, C₁₋₄alkyl, halo substituted C₁₋₄alkyl and C₁₋₃alkoxy;
R³ is C₁₋₃alkyl wherein said C₁₋₃alkyl is optionally substituted on one or more carbon by one or more halo or hydroxy; and pharmaceutically acceptable salts thereof.

11. A process for the preparation of a compound of formula II or a pharmaceutically acceptable salt thereof, as defined in claim 1 which comprises:
a) reacting a purine base of formula (1): 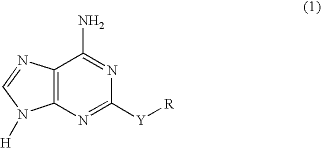
or a suitably protected derivative thereof with an electrophile of formula (2) 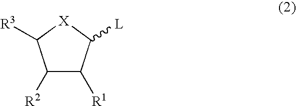
wherein X, Y, R, R¹, R² and R³ are as defined in claim 1 and L is a suitable leaving group such as acetate, methoxy, benzoyl, or chloro to yield a compound of formula II, and
b) optionally, converting said compound of formula II into another compound of formula II and removing any protecting groups; and
c) optionally forming a pharmaceutically acceptable salt thereof.

12. A method for producing an antibacterial effect in a warm blooded animal, such as man, in need of such treatment, which comprises administering to said animal an effective amount of a compound of formula I, or a pharmaceutically-acceptable salt thereof 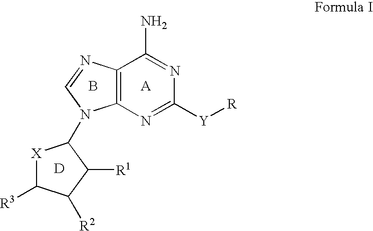
wherein:
A, B and D are used to designate the particular ring;
X is selected from O and —CH₂—;
Y is selected from O, S, —CO—, —CH₂—, —CH═CH—, —C≡C—, —SO—, and —SO₂— or Y and R taken together form a heterocyclic radical provided that the atom of the heterocyclic radical which is directly attached to ring A is not a nitrogen atom and wherein said heterocyclic radical may be optionally substituted on one or more carbon atoms by one or more R³³ and wherein if heterocyclyl contains an —NH— moiety, the nitrogen of said moiety may be optionally substituted by a group selected from R³⁴;
R is selected from C₁₋₁₀alkyl, C₂₋₁₀alkenyl, C₂₋₁₀alkynyl, C₃₋₁₂carbocyclyl, —S(O)ₚR⁴, —C(O)R⁵, and heterocyclyl wherein R may be optionally substituted on one or more carbon atoms by one or more R′ and wherein if heterocyclyl contains an —NH— moiety, the nitrogen of said moiety may be optionally substituted by a group selected from R″;
p is independently at each occurrence 0, 1 or 2;
R¹, R² and R³ are independently selected from hydrogen, hydroxy, cyano, azido, C₁₋₁₀alkyl, C₃₋₁₂carbocyclyl, halo, —C(O)R^{5′}, —OC(O)R¹², S(O)ₚR^{4′}, ═N—O—R⁹, C₂₋₁₀alkenyl, C₂₋₁₀alkynyl, heterocyclyl, —OR²⁴, NR¹⁰R¹¹, alternatively, R¹ and R² or R² and R³ taken together form a cyclic ring containing 3-6 atoms wherein R¹, R² and R³ may be optionally substituted on one or more carbon atoms by one or more R^{1′} and wherein if heterocyclyl contains an —NH— moiety, the nitrogen of said moiety may be optionally substituted by a group selected from R^{3′};
R⁴, R^{4′}, and R^{4″} are each independently selected from hydrogen, hydroxy, —NR⁷R⁸, C₁₋₆alkyl, C₂₋₆alkenyl, C₁₋₆alkoxy, C₃₋₁₀cycloalkyl, heterocyclyl, and aryl wherein R⁴, R^{4′}, and R^{4″} may be optionally substituted on one or more carbon atoms by one or more R¹³ and wherein if heterocyclyl contains an —NH— moiety, the nitrogen of said moiety may be optionally substituted by a group selected from R¹⁴;
R⁵, R^{5′}, R^{5″}, R¹² and R^{12′} are each independently selected from hydrogen, —NR^{7′}R^{8′}, —OR^{24′}, C₁₋₆alkyl, C₂₋₆alkenyl, C₃₋₁₀cycloalkyl, C₃₋₁₀cycloalkenyl, heterocyclyl, and aryl wherein R⁵, R^{5′}, R^{5″}, R¹², and R^{12′} may be optionally substituted on one or more carbon atoms by one or more R¹⁵ and wherein if heterocyclyl contains an —NH— moiety, the nitrogen of said moiety may be optionally substituted by a group selected from R¹⁶;
R⁷, R^{7′}, R^{7″}, R⁸, R^{8′} and R^{8″} are each independently selected from hydrogen, C₁₋₆alkyl, C₂₋₆alkenyl, C₂₋₆alkynyl, —OR^{24′}, C₃₋₁₀cycloalkyl, C₃₋₁₀cycloalkenyl, heterocyclyl, and aryl wherein R⁷, R^{7′}, R^{7″}, R⁸, R^{8′} and R^{8″} may be optionally substituted on one or more carbon atoms by one or more R¹⁷ and wherein if heterocyclyl contains an —NH— moiety, the nitrogen of said moiety may be optionally substituted by a group selected from R¹⁸;
R⁹ and R^{9′} are each independently selected from hydrogen, C₁₋₆alkyl, C₂₋₆alkenyl, C₂₋₆alkynyl, C₃₋₁₀cycloalkyl, C₃₋₁₀cycloalkenyl, heterocyclyl and aryl wherein R⁹ and R^{9′} may be optionally substituted on one or more carbon atoms by one or more R¹⁹;
R¹⁰ and R¹¹ are each independently selected from hydrogen, C₁₋₆alkyl, C₂₋₆alkenyl, C₂₋₆alkynyl, —OR^{24′}, C₃₋₁₀cycloalkyl, C₃₋₁₀cycloalkenyl, heterocyclyl and aryl, wherein R¹⁰ and R¹¹ independently of each other may be optionally substituted on one or more carbon by one or more R²⁰, and wherein if said heterocyclyl contains a —NH— moiety, the nitrogen of said moiety may be optionally substituted by a group selected from R²¹;
R′, R^{1′}, R¹³, R¹⁵, R¹⁷, R¹⁹, R²⁰, R²⁵ and R³³ are each independently selected from halo, nitro, —NR^{7″}R^{8″}, azido, cyano, isocyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, keto(═O), —OR^{24′}, —C(O)R^{5″}, —OC(O)R^{12′}, S(O)ₓR^{4″}; ═N—O—R^{9′}, —NHC(O)NR^{7′}R^{8′}, —N(C₁₋₆alkyl)C(O)NR^{7′}R^{8′}, NHC(O)R^{24″}, —NHCO₂R^{24″}, —NHSO₂(R^{24″}), -amidino i.e. —NHC(NH)NH₂, wherein x is independently 0, 1 or 2 and further wherein R′, R^{1′}, R¹³, R¹⁵, R¹⁷, R¹⁹, R²⁰, R²⁵ and R³³ independent of each other may be optionally substituted on one or more carbon by one or more R²² and wherein if heterocyclyl contains a —NH— moiety, the nitrogen of said moiety may be optionally substituted by a group selected from R²³,
R″, R^{3′}, R¹⁴, R¹⁶, R¹⁸, R²¹, R²³, R²⁶, R²⁸ and R³⁴ are each independently selected from cyano, C₁₋₆alkyl, C₂₋₆alkenyl, C₂₋₆alkynyl, aryl, cycloalkyl, cycloalkenyl, heterocyclyl, hydroxy, —OR^{24′}, —C(O)R^{5″}, —OC(O)R^{12′}, S(O)ₓR^{4″}, —NHC(NH)NH₂, wherein x is independently 0, 1 or 2 wherein R″, R^{3′}, R¹⁴, R¹⁶, R¹⁸, R²¹, R²³, R²⁶, R²⁸ and R³⁴ independently of each other may be optionally substituted on one or more carbon by one or more R²⁷ and wherein if said heterocyclyl contains a —NH— moiety, the nitrogen of said moiety may be optionally substituted by a group selected from R⁶;
R²⁴, R^{24′} and R^{24″} are each independently selected from hydrogen, C₁₋₆alkyl, C₂₋₆alkenyl, C₃₋₁₂cycloalkyl, C₃₋₁₂cycloalkenyl, aryl, S(O)ₓR^{4″}, and heterocyclyl wherein x is independently 0, 1 or 2 and further wherein R²⁴, R^{24′} and R^{24″} may be optionally substituted on one or more carbon by one or more R²⁵ and wherein if said heterocyclyl contains a —NH— moiety, the nitrogen of said moiety may be optionally substituted by a group selected from R²⁶.
R²² and R²⁷ are each independently selected from halo, nitro, —NR^{7′}R^{8′}, azido, cyano, isocyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, keto(═O), —OR^{24′}, —C(O)R^{5″}, —OC(O)R^{12′}, S(O)ₓR^{4″}; ═N—O—R^{9′}, NHC(O)NR^{7′}R^{8′}, —N(C₁₋₆alkyl)C(O)NR^{7′}R^{8′}, —NHC(O)R^{24″}, —NHCO₂R^{24″}, —NHSO₂(R^{24″}), -amidino i.e. —NHC(NH)NH₂, wherein x is independently 0, 1 or 2 and further wherein R²² and R²⁷ independently of each other may be optionally substituted on one or more carbon by one or more R²⁹ and wherein if heterocyclyl contains a —NH— moiety, the nitrogen of said moiety may be optionally substituted by a group selected from R³⁰;
R⁶ and R²³ are each independently selected from cyano, C₁₋₆alkyl, C₂₋₆alkenyl, C₂₋₆alkynyl, aryl, cycloalkyl, cycloalkenyl, heterocyclyl, hydroxy, —OR^{24′}, —C(O)R^{5″}, —OC(O)R^{12′}, S(O)ₓR^{4″} and —NHC(NH)NH₂, wherein x is independently 0, 1 or 2 and further wherein R⁶ and R²³ independently of each other may be optionally substituted on one or more carbon by one or more R³¹ and wherein if said heterocyclyl contains a —NH— moiety, the nitrogen of said moiety may be optionally substituted by a group selected from R³²;
R²⁹ and R³¹ are each independently selected from halo, nitro, —NR^{7′}R^{8′}, azido, cyano, isocyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, keto(═O), —OR^{24′}, —C(O)R^{5″}, —OC(O)R^{12′}, S(O)ₓR^{4″}, ═N—O—R^{9″}, —NHC(O)NR^{7′}R^{8′}, —N(C₁₋₆alkyl)C(O)NR^{7′}R^{8′}, —NHC(O)R^{24″}, —NHCO₂R^{24∝}, —NHSO₂(R^{24″}), -amidino i.e. —NHC(NH)NH₂, wherein x is independently 0, 1 or 2;
R³⁰ and R³² are each independently selected from cyano, C₁₋₆alkyl, C₂₋₆alkenyl, C₂₋₆alkynyl, aryl, C₃₋₁₂cycloalkyl, C₃₋₁₂cycloalkenyl, heterocyclyl, hydroxy, —OR^{24′}, —C(O)R^{5″}, —OC(O)R^{12′}, S(O)ₓR^{4″}, and —NHC(NH)NH₂ wherein x is independently 0, 1 or 2;
provided that when ring D is an unsubstituted tetrahydrofuranyl ring, i.e. when X is O and R¹, R² and R³ are hydrogen, and Y is O, then R cannot be a 3-pyrrolidinyl radical or a 7-methylindan-4-yl radical; provided further when ring D is an unsubstituted tetrahydrofuranyl ring and Y is S, then R cannot be an unsubstituted 2-naphthyl radical; and further provided when ring D is an unsubstituted tetrahydrofuranyl ring and Y is a bond, then R cannot be an unsubstituted 3-pyridyl radical; and provided further the compound of formula I is not 9-{5-[4-(carboxymethyl)-1H-imidazol-1-yl]-5-deoxy-β-D-ribofuranosyl}-2-(cyclopentyloxy)-9H-purin-6-amine or 9-[5-(4-acetyl-1H-1,2,3-triazol-1-yl)-5-deoxy-β-D-ribofuranosyl]-2-(cyclopentyloxy)-9H-purin-6-amine.

13. A method for producing an antibacterial effect in a warm blooded animal, such as man, in need of such treatment, which comprises administering to said animal an effective amount of a compound of formula II, or a pharmaceutically-acceptable salt thereof as claimed in claim 1.

14. A method for inhibition of bacterial DNA ligase in a warm-blooded animal in need of such treatment which comprises administering to said animal an effective amount of a compound of formula I according to claim 12 or a pharmaceutically acceptable salt thereof.

15. A method for inhibition of bacterial DNA ligase in a warm-blooded animal in need of such treatment which comprises administering to said animal an effective amount of a compound of formula II, or a pharmaceutically acceptable salt thereof as claimed in claim 1.

16. A method of treating a bacterial infection in a warm-blooded animal in need of such treatment which comprises administering to said animal an effective amount of a compound of formula I according to claim 12 or a pharmaceutically acceptable salt thereof.

17. A method of treating a bacterial infection in a warm-blooded animal in need of such treatment which comprises administering to said animal an effective amount of a compound of any one of formula II, or a pharmaceutically acceptable salt thereof as claimed in claim 1.

18-25. (canceled)

26. A pharmaceutical composition which comprises a compound of the formula II, or a pharmaceutically acceptable salt thereof as claimed in claim 1, and a pharmaceutically acceptable diluent or carrier.

27. A pharmaceutical composition which comprises a compound of formula I or a pharmaceutically acceptable salt thereof as claimed in claim 12, in association with a pharmaceutically acceptable excipient or carrier for use in the production of an anti-bacterial effect in an warm-blooded animal, such as a human being.
